(19) United States
(12) Patent Application Publication
(10) Pub. No.: US 2023/0234937 A1
(43) Pub. Date: Jul. 27, 2023

(54) INDENOAZANAPHTHALENES

(71) Applicant: Merck Patent GmbH, Darmstadt (DE)

(72) Inventors: Jens ENGELHART, Darmstadt (DE); Margarita WUCHERER-PLIETKER, Darmstadt (DE); Sebastian MEYER, Darmstadt (DE); Christian EICKHOFF, Darmstadt (DE)

(21) Appl. No.: 18/010,474

(22) PCT Filed: Jun. 15, 2021
(86) PCT No.: PCT/EP2021/066022

§ 371 Date: Dec. 15, 2022

(30) Foreign Application Priority Data
Jun. 18, 2020 (DE) 10 2020 003 647.3

(51) Int. Cl. C07D 401/10; C07D 471/04; C07D 405/14; C07D 401/04; C07D 401/14; C07D 221/20; C07D 491/107; C07F 9/46; H10K 50/15; H10K 50/165; H10K 85/60

(52) U.S. Cl. CPC C07D 401/10; C07D 471/04; C07D 405/14; C07D 401/04; C07D 401/14; C07D 221/20; C07D 491/107; C07F 9/46; H10K 50/15; H10K 50/165; H10K 85/6572

(57) ABSTRACT

The present invention relates indenoazanaphthalenes, particularly for use in electronic devices. The invention further relates to a method for producing the indenoazanaphthalenes according to the invention, and to electronic devices comprising the same.

The present invention describes indenoazanaphthalenes, especially for use in electronic devices. The invention further relates to a process for preparing the compounds of the invention and to electronic devices comprising these compounds.

The structure of organic electroluminescent devices (OLEDs) in which organic semiconductors are used as functional materials is described, for example, in U.S. Pat. Nos. 4,539,507, 5,151,629, EP 0676461, WO 98/27136, JP 2007-161934 A and JP 2008-010649 A. Emitting materials used are frequently organometallic complexes which exhibit phosphorescence. For quantum-mechanical reasons, up to four times the energy efficiency and power efficiency is possible using organometallic compounds as phosphorescent emitters. In general terms, there is still a need for improvement in OLEDs, especially also in OLEDs which exhibit phosphorescence, for example with regard to efficiency, operating voltage and lifetime.

The properties of organic electroluminescent devices are not only determined by the emitters used. Also of particular significance here are especially the other materials used, such as host and matrix materials, hole blocker materials, electron transport materials, hole transport materials and electron or exciton blocker materials. Improvements to these materials can lead to distinct improvements to electroluminescent devices.

Frequently used according to the prior art as matrix materials for phosphorescent compounds and as hole transport materials are aromatic or heteroaromatic compounds, for example triarylamine derivatives or carbazole derivatives. In addition, triazine derivatives or pyrimidine derivatives are also used as matrix materials and as electron transport materials.

In general terms, in the case of these materials, for example for use as matrix materials, there is still a need for improvement, particularly in relation to the lifetime, but also in relation to the efficiency, operating voltage and color purity of the device.

It is therefore an object of the present invention to provide compounds which are suitable for use in an organic electronic device, especially in an organic electroluminescent device, and which lead to good device properties when used in this device, and to provide the corresponding electronic device.

More particularly, the problem addressed by the present invention is that of providing compounds which lead to a high lifetime, good efficiency and low operating voltage. Particularly the properties of the matrix materials too have a major influence on the lifetime and efficiency of the organic electroluminescent device.

A further problem addressed by the present invention can be considered that of providing compounds suitable for use in a phosphorescent or fluorescent OLED, especially as a matrix material. A particular problem addressed by the present invention is that of providing matrix materials suitable for red- and green-phosphorescing OLEDs and possibly also for blue-phosphorescing OLEDs.

In addition, the compounds, especially when they are used as matrix materials, as hole conductor materials or as electron transport materials in organic electroluminescent devices, should lead to devices having excellent color purity.

Moreover, the compounds should be processible in a very simple manner, and especially exhibit good solubility and film formation. For example, the compounds should exhibit elevated oxidation stability and an improved glass transition temperature.

A further problem can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, these problems are solved by particular compounds described in detail hereinafter. The use of the compounds leads to very good properties of organic electronic devices, especially of organic electroluminescent devices, especially with regard to lifetime, color purity, efficiency and operating voltage. The present invention therefore provides electronic devices, especially organic electroluminescent devices, comprising compounds of this kind, and the corresponding preferred embodiments.

The present invention therefore provides a compound comprising at least one structure of the formula (Ia) and/or (Ib), preferably a compound of the formula (Ia) and/or (Ib):

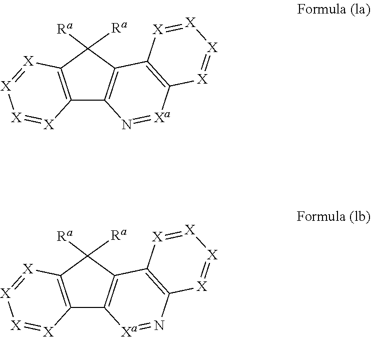

where the symbols used are as follows:
X is the same or different at each instance and is N or CR, preferably CR;
X^{a} is the same or different at each instance and is N or CR^{a}, preferably CR^{a};
R^{a} is the same or different at each instance and is, D, OH, F, Cl, Br, I, CN, NO₂, N(Ar^{a})₂, N(R)₂, C(═O) Ar^{a}, C(═O)R², P(═O)(Ar^{a})₂, P(Ar^{a})₂, B(Ar^{a})₂, B(OR)₂, Si(Ar^{a})₃, Si(R)₃, Ge(R)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R radicals, where one or more nonadjacent CH₂ groups may be replaced by —RC═CR—, —C≡C—, Si(R)₂, Ge(R)₂, Sn(R)₂, C═O, C═S, —O—, —Se—, —S—, C═Se, —C(═O)O—, —C(═O)NR—, C═NR, NR, P(═O)(R), SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R radicals, or an aryloxy or heteroaryloxy group which has 5 to 60 aromatic ring atoms and may be substituted by one or more R radicals, or an aralkyl or heteroaralkyl group which has 5 to 60 aromatic ring atoms and may be substituted by one or more R radicals, or a combination of these systems; at the same time, two or more, preferably adjacent R^{a} radicals may form a ring system with one another or with an R radical;
Ar^{a} is the same or different at each instance and is an aromatic or heteroaromatic ring system which has 5 to 30 aromatic ring atoms and may be substituted by one or more, preferably nonaromatic R radicals; at the same time, it is possible for two Ar^{a} radicals bonded to the same silicon atom, nitrogen atom, phosphorus atom or boron atom also to be joined together via a bridge by a single bond or a bridge selected from B(R), C(R)₂, Si(R)₂, Ge(R)₂, C═O, C═NR, C═C(R)₂, O, S, Se, S═O, SO₂, N(R), P(R) and P(═O)R;
R is the same or different at each instance and is H, D, OH, F, Cl, Br, I, CN, NO₂, N(Ar)₂, N(R¹)₂, C(═O)Ar, C(═O)R¹, P(═O)(Ar)₂, P(Ar)₂, B(Ar)₂, B(OR¹)₂, Si(Ar)₃, Si(R¹)₃, Ge(R¹)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R¹C═CR¹—, —C≡C—, Si(R¹)₂, Ge(R¹)₂, Sn(R¹)₂, C═O, C═S, C═Se, —C(═O)O—, —C(═O)NR¹—, C═NR¹, NR¹, P(═O)(R¹), —O—, —S—, —Se—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R¹ radicals, or an aryloxy or heteroaryloxy group which has 5 to 60 aromatic ring atoms and may be substituted by one or more R¹ radicals, or an aralkyl or heteroaralkyl group which has 5 to 60 aromatic ring atoms and may be substituted by one or more R¹ radicals, or a combination of these systems; at the same time, two or more, preferably adjacent R radicals together may form a ring system;
Ar is the same or different at each instance and is an aromatic or heteroaromatic ring system which has 5 to 30 aromatic ring atoms and may be substituted by one or more, preferably nonaromatic R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, Se, S═O, SO₂, N(R¹), P(R¹) and P(═O)R¹;
R¹ is the same or different at each instance and is H, D, OH, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R²C═CR²—, —C≡C—, Si(R²)₂, Ge(R²)₂, Sn(R²)₂, C═O, C═S, C═Se, C═NR², —C(═O)O—, —C(═O)NR²—, NR², P(═O)(R²), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more, preferably adjacent R¹ radicals together may form a ring system;
Ar¹ is the same or different at each instance and is an aromatic or heteroaromatic ring system which has 5 to 30 aromatic ring atoms and may be substituted by one or more, preferably nonaromatic R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, Ge(R²)₂, C═O, C═NR², C═C(R²)₂, O, S, Se, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSOR³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, C≡C—, Si(R³)₂, Ge(R³)₂, Sn(R³)₂, C═O, C═S, C═NR³, —C(═O)O—, —C(═O)NR³—, NR³, P(═O)(R³), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has to 40 aromatic ring atoms and may be substituted by one or more R³ radicals, or a combination of these systems; at the same time, two or more, preferably adjacent substituents R² together may form a with one another;
R³ is the same or different at each instance and is selected from the group consisting of H, D, F, CN, an aliphatic hydrocarbyl radical having 1 to 20 carbon atoms, and an aromatic or heteroaromatic ring system having 5 to 30 aromatic ring atoms in which one or more hydrogen atoms may be replaced by D, F, Cl, Br, I or CN and which may be substituted by one or more alkyl groups each having 1 to 4 carbon atoms; at the same time, it is possible for two or more, preferably adjacent R³ substituents to form a ring system with one another.

Adjacent carbon atoms in the context of the present invention are carbon atoms bonded directly to one another. In addition, “adjacent radicals” in the definition of the radicals means that these radicals are bonded to the same carbon atom or to adjacent carbon atoms. These definitions apply correspondingly, inter alia, to the terms “adjacent groups” and “adjacent substituents”.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

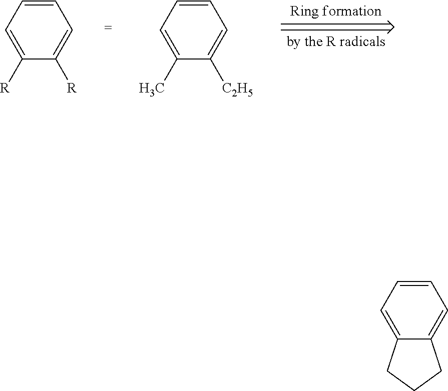

In addition, however, the abovementioned wording shall also be understood to mean that, if one of the two radicals is hydrogen, the second radical binds to the position to which the hydrogen atom was bonded, forming a ring. This will be illustrated by the following scheme:

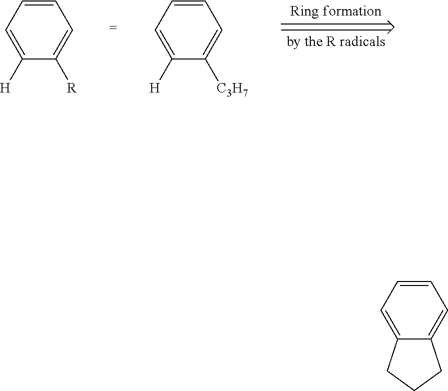

A fused aryl group, a fused aromatic ring system or a fused heteroaromatic ring system in the context of the present invention is a group in which two or more aromatic groups are fused, i.e. annelated, to one another along a common edge, such that, for example, two carbon atoms belong to the at least two aromatic or heteroaromatic rings, as, for example, in naphthalene. By contrast, for example, fluorene is not a fused aryl group in the context of the present invention, since the two aromatic groups in fluorene do not have a common edge. Corresponding definitions apply to heteroaryl groups and to fused ring systems which may but need not also contain heteroatoms.

An aryl group in the context of this invention contains 6 to 60 carbon atoms, preferably 6 to 40 carbon atoms; a heteroaryl group in the context of this invention contains 2 to 60 carbon atoms, preferably 2 to 40 carbon atoms, and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused aryl or heteroaryl group, for example naphthalene, anthracene, phenanthrene, quinoline, isoquinoline, etc.

An aromatic ring system in the context of this invention contains 6 to 60 carbon atoms, preferably 6 to 40 carbon atoms, in the ring system. A heteroaromatic ring system in the context of this invention contains 1 to 60 carbon atoms, preferably 1 to 40 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon, nitrogen or oxygen atom or a carbonyl group. For example, systems such as 9,9′-spirobifluorene, 9,9-diarylfluorene, triarylamine, diary) ethers, stilbene, etc. shall thus also be regarded as aromatic ring systems in the context of this invention, and likewise systems in which two or more aryl groups are interrupted, for example, by a linear or cyclic alkyl group or by a silyl group. In addition, systems in which two or more aryl or heteroaryl groups are bonded directly to one another, for example biphenyl, terphenyl, quaterphenyl or bipyridine, shall likewise be regarded as an aromatic or heteroaromatic ring system.

A cyclic alkyl, alkoxy or thioalkoxy group in the context of this invention is understood to mean a monocyclic, bicyclic or polycyclic group.

In the context of the present invention, a C₁- to C₂₀-alkyl group in which individual hydrogen atoms or CH₂ groups may also be substituted by the abovementioned groups is understood to mean, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals. An alkenyl group is understood to mean, for example, ethenyl, propenyl, butenyl, pentenyl, cyclopentenyl, hexenyl, cyclohexenyl, heptenyl, cycloheptenyl, octenyl, cyclooctenyl or cyclooctadienyl. An alkynyl group is understood to mean, for example, ethynyl, propynyl, butynyl, pentynyl, hexynyl, heptynyl or octynyl. A C₁- to C₄₀-alkoxy group is understood to mean, for example, methoxy, trifluoromethoxy, ethoxy, n-propoxy, i-propoxy, n-butoxy, i-butoxy, s-butoxy, t-butoxy or 2-methylbutoxy.

An aromatic or heteroaromatic ring system which has 5-60 aromatic ring atoms, preferably 5-40 aromatic ring atoms, and may also be substituted in each case by the abovementioned radicals and which may be joined to the aromatic or heteroaromatic system via any desired positions is understood to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

It may preferably be the case that at least one, preferably at least two, of the R^{a} radicals is/are an aromatic or heteroaromatic ring system which has 5 to 60 aromatic ring atoms and may be substituted in each case by one or more R radicals.

In a further configuration, it may be the case that at least one, preferably at least two, of the R^{a} radicals is/are a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R radicals. There may preferably be two methyl groups present on the fluorene bridge, such that the adjacent R^{a} groups in formula (Ia) or (Ib) each represent a methyl group.

In a preferred configuration, the compounds of the invention may contain at least one structure of the formula (II-1), (II-2), (II-3), (II-4), (II-5), (II-6), (II-7), (II-8), (II-9), (II-10), (II-11), (II-12), (II-13), (II-14), (II-15) and/or (II-16), where the compounds can preferably be represented by structures of the formula (II-1), (II-2), (II-3), (II-4), (II-5), (II-6), (II-7), (II-8), (II-9), (II-10), (II-11), (II-12), (II-13), (II-14), (II-15) and/or (II-16):

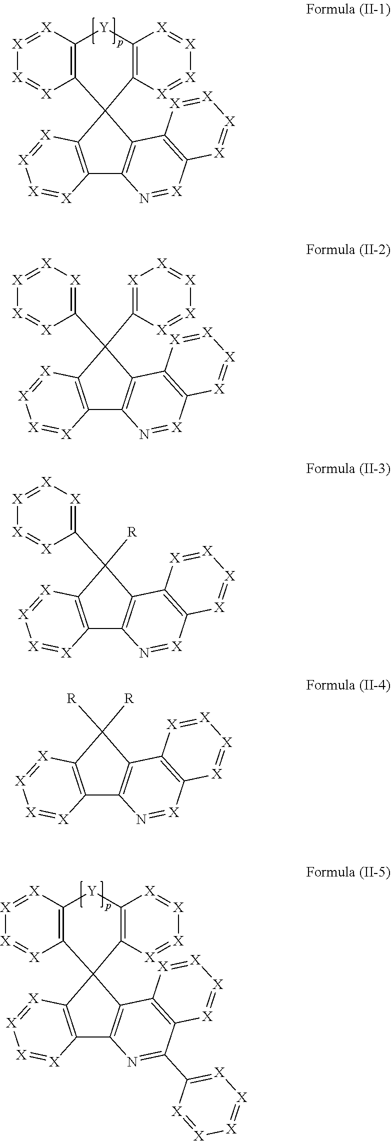

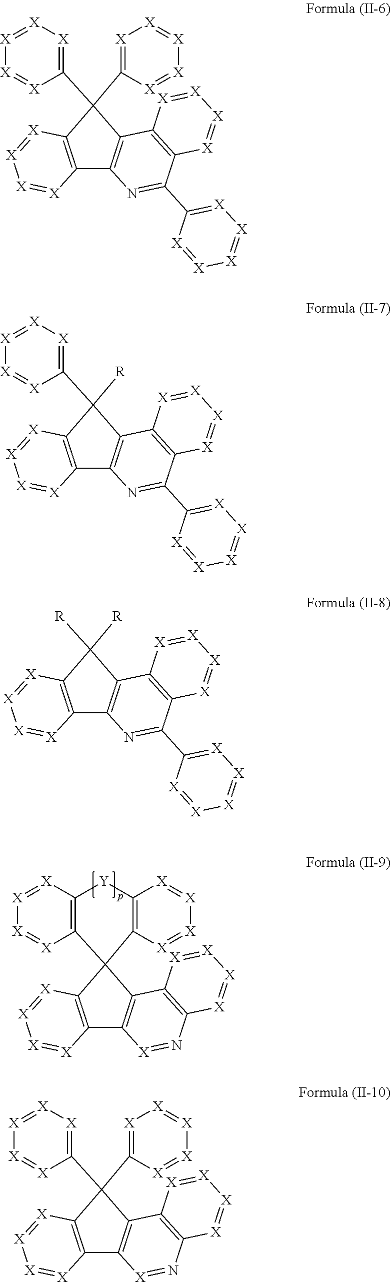

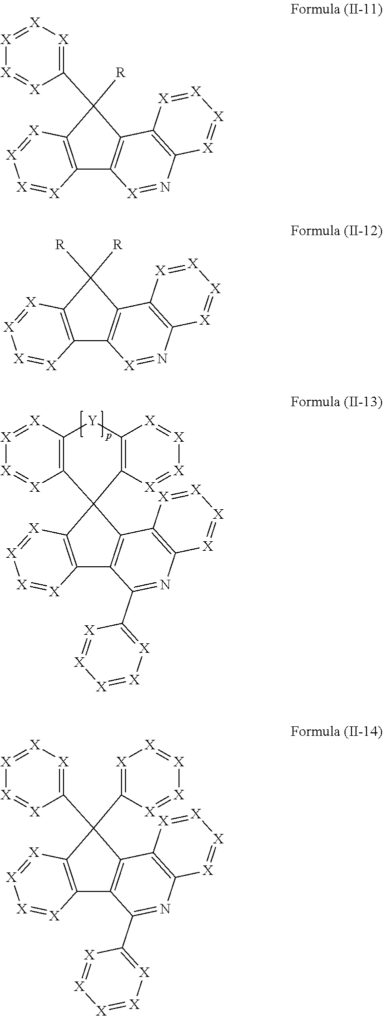

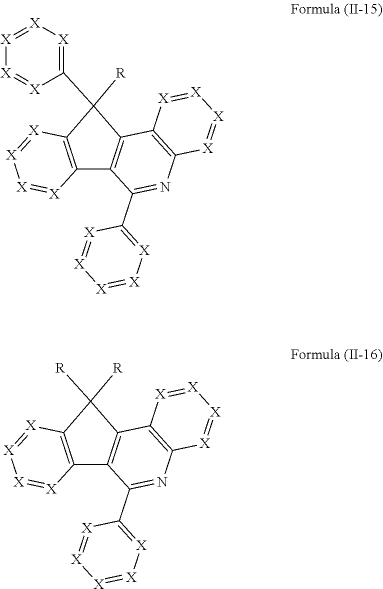

where the symbols R and X used have the definition given above, especially for formula (Ia) and/or (Ib), p is 0 or 1 and Y is B(R), C(R)₂, Si(R)₂, Ge(R)₂, C═O, C═NR, C═C(R)₂, O, S, Se, S═O, SO₂, N(R), P(R) and P(═O)R, preferably B(R), C(R)₂, Si(R)₂, O, S, Se, S═O, SO₂, N(R), P(R) and P(═O)R, more preferably O or N(R), where, when p=0, there is a bond between the aromatic or heteroaromatic rings shown. Preference is given here to the structures of the formulae (II-5), (II-6), (II-7), (II-8), (II-13), (II-14), (II-15) and (II-16), and particular preference to structures of the formulae (II-13), (II-14), (II-15) and (II-16).

In addition, particular preference is given to structures (preferably compounds) of the formulae (II-9) and (II-13):

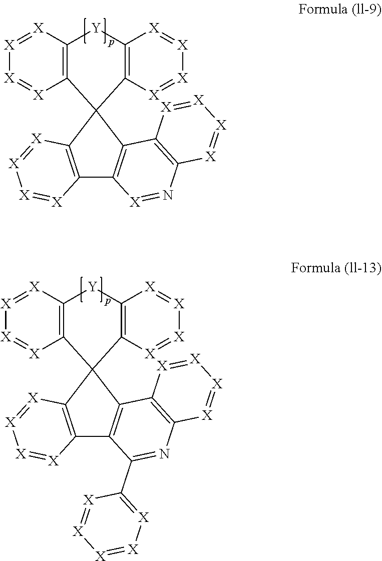

where the symbols R and X used may have the definition given above, especially for formula (Ia) and/or (Ib), p is 0 or 1 and Y is B(R), C(R)₂, Si(R)₂, Ge(R)₂, C═O, C═NR, C═C(R)₂, O, S, Se, S═O, SO₂, N(R), P(R) and P(═O)R, preferably B(R), C(R)₂, Si(R)₂, O, S, Se, S═O, SO₂, N(R), P(R) and P(═O)R, more preferably O or N(R), where, when p=0, there is a bond between the aromatic or heteroaromatic rings shown. In relation to the formulae (II-9) and (II-13), preference is given to structures (preferably compounds) in which p=0, such that a bond is formed between the two rings.

It may preferably be the case that, in formulae (Ia), (Ib) and/or (II-1) to (II-16), not more than two X groups per ring are N; preferably at least one, more preferably at least two of the X groups per ring are selected from C—H and C-D.

Preferably, in formulae (Ia), (Ib) and/or (II-1) to (II-16), not more than four and preferably not more than two X groups are N; more preferably, all X groups are CR, where preferably at most 4, more preferably at most 3 and especially preferably at most 2 of the CR groups that X represents are not the CH group.

It may further be the case that the compounds of the invention comprise at least one structure of the formula (III-1), (III-2), (III-3), (III-4), (III-5), (III-6), (III-7), (III-8), (III-9), (III-10), (III-11), (III-12), (III-13), (III-14), (III-15) and/or (III-16), where the compounds can preferably be represented by structures of the formula (III-1), (III-2), (III-3), (III-4), (III-5), (III-6), (III-7), (III-8), (III-9), (III-10), (III-11), (III-12), (III-13), (III-14), (III-15) and/or (III-16):

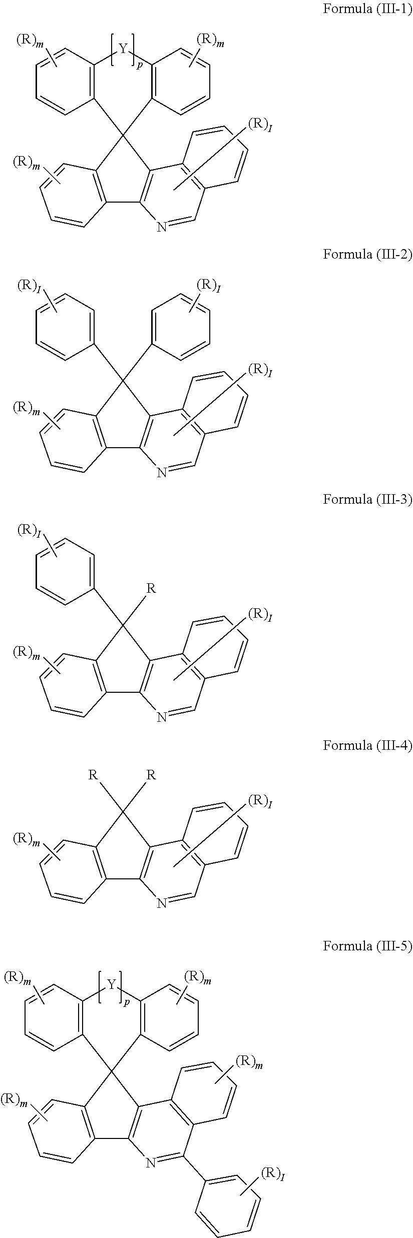

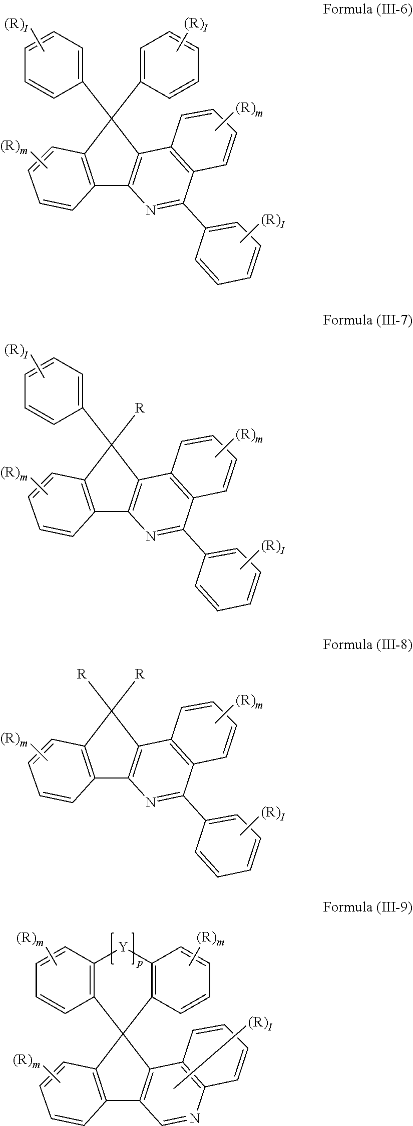

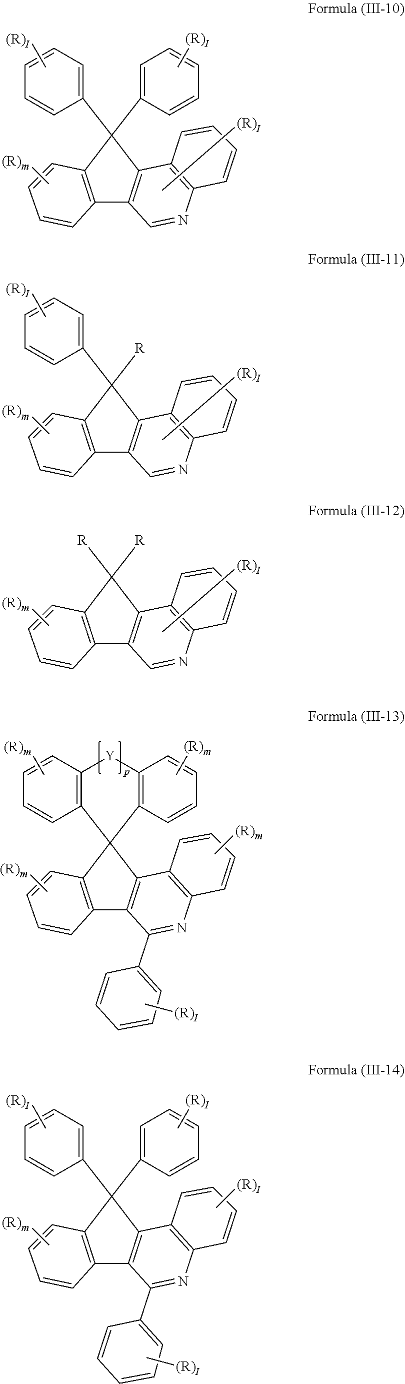

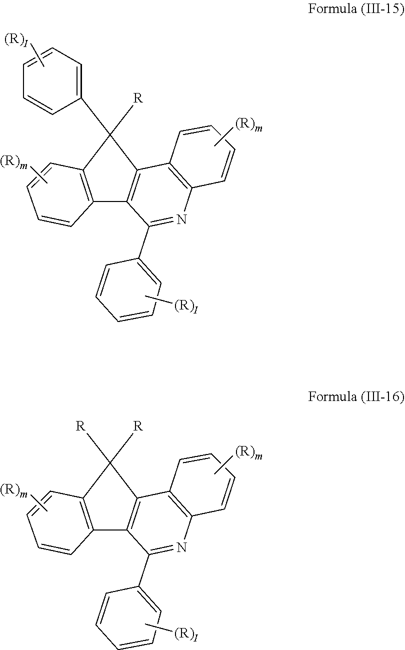

where R has the definition given above, especially for formula (Ia) and/or (Ib), the symbols Y and p have the definition given above, especially for formula (II-1) to (II-16), I is 1, 2, 3, 4 or 5, preferably 0, 1 or 2, and m is the same or different at each instance and is 0, 1, 2, 3 or 4, preferably 0, 1, 2 or 3, more preferably 0, 1 or 2. Preference is given here to structures of the formulae (III-5), (III-6), (III-8), (III-13), (III-14) and (III-16).

In addition, particular preference is given to structures (preferably compounds) of the formulae (III-9) and (III-13):

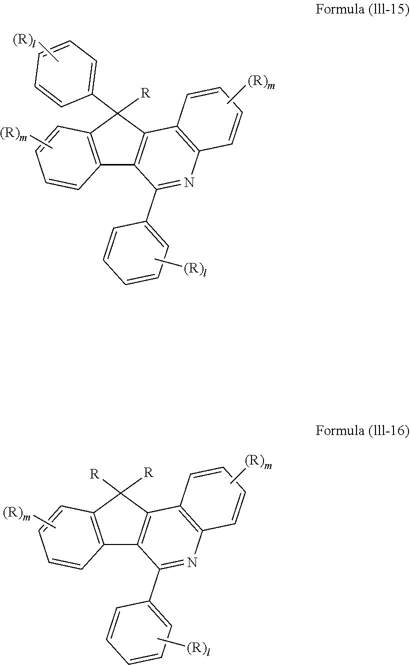

where R has the definition given above, especially for formula (Ia) and/or (Ib), the symbols Y and p have the definition given above, especially for formula (I1-1) to (II-16), I is 1, 2, 3, 4 or 5, preferably 0, 1 or 2, and m is the same or different at each instance and is 0, 1, 2, 3 or 4, preferably 0, 1, 2 or 3, more preferably 0, 1 or 2. In relation to the formulae (III-9) and (III-13), preference is given to structures (preferably compounds) in which p=0, such that a bond is formed between the two rings.

Preferably, the sum total of the indices I and m in structures, preferably in compounds of the formulae (III-1) to (III-16), is not more than 6, preferably not more than 4 and more preferably not more than 2.

It may further be the case that the compound comprises a hole transport group, where preferably at least one of the R^{a} groups and/or an R group in a structure/compound of the formulae (Ia), (Ib), (II-1) to (II-16) and/or (III-1) to (III-16) comprises and preferably is a hole transport group.

Hole transport groups are known in the technical field, and they preferably include triarylamine or carbazole groups.

In a further embodiment, it may be the case that the compound usable for production of functional layers of electronic devices comprises an electron transport group-comprising radical.

It may further be the case that the compound comprises an electron transport group-comprising radical, where preferably at least one of the R^{a} groups or an R group in a structure/compound of the formulae (Ia), (Ib), (II-1) to (II-16) and/or (III-1) to (III-16) comprises and preferably is an electron transport group-comprising radical.

Electron transport groups are widely known in the technical field and promote the ability of compounds to transport and/or to conduct electrons.

In addition, surprising advantages are shown by compounds usable for production of functional layers of electronic devices that comprise at least one structure selected from the group of the pyridines, pyrimidines, pyrazines, pyridazines, triazines, quinazolines, quinoxalines, quinolines, isoquinolines, imidazoles and/or benzimidazoles, particular preference being given to pyrimidines, triazines and quinazolines. These structures generally promote the ability of compounds to transport and/or to conduct electrons.

In a preferred configuration of the present invention, it may be the case that the electron transport group-comprising radical is a group that can be represented by the formula (QL)

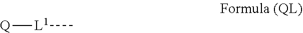

in which L¹ represents a bond or an aromatic or heteroaromatic ring system which has 5 to 40, preferably 5 to 30, aromatic ring atoms and may be substituted by one or more R¹ radicals, Q is an electron transport group, where R¹ has the definition given above, especially for formulae (Ia) and/or (Ib), and the dotted bond marks the position of attachment.

Preferably, the L¹ group may form through-conjugation with the Q group and the atom, preferably the carbon or nitrogen atom, to which the L¹ group of formula (QL) is bonded. Through-conjugation of the aromatic or heteroaromatic systems is formed as soon as direct bonds are formed between adjacent aromatic or heteroaromatic rings. A further bond between the aforementioned conjugated groups, for example via a sulfur, nitrogen or oxygen atom or a carbonyl group, is not detrimental to conjugation. In the case of a fluorene system, the two aromatic rings are bonded directly, where the spa-hybridized carbon atom in position 9 does prevent fusion of these rings, but conjugation is possible since this sp³-hybridized carbon atom in position 9 does not necessarily lie between the electron-transporting Q group and the atom via which the group of formula (QL) is bonded to further structural elements of a compound of the invention. In contrast, in the case of a second spirobifluorene structure, through-conjugation can be formed if the bond between the Q group and the aromatic or heteroaromatic radical to which the L¹ group of formula (QL) is bonded is via the same phenyl group in the spirobifluorene structure or via phenyl groups in the spirobifluorene structure that are bonded directly to one another and are in one plane. If the bond between the Q group and the aromatic or heteroaromatic radical to which the L¹ group of formula (QL) is bonded is via different phenyl groups in the second spirobifluorene structure bonded via the spa-hybridized carbon atom in position 9, the conjugation is interrupted.

In a further preferred embodiment of the invention, L¹ is a bond or an aromatic or heteroaromatic ring system which has 5 to 14 aromatic or heteroaromatic ring atoms, preferably an aromatic ring system which has 6 to 12 carbon atoms, and which may be substituted by one or more R¹ radicals, but is preferably unsubstituted, where R¹ may have the definition given above, especially for formulae (Ia) and/or (Ib). More preferably, L¹ is an aromatic ring system having 6 to 10 aromatic ring atoms or a heteroaromatic ring system having 6 to 13 heteroaromatic ring atoms, each of which may be substituted by one or more R² radicals, but is preferably unsubstituted, where R² may have the definition given above, especially for formula (Ia) and/or (Ib).

Further preferably, the symbol L¹ shown in formula (QL) inter alia is the same or different at each instance and is a bond or an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded to the respective atom of the further group directly, i.e. via an atom of the aromatic or heteroaromatic group.

It may additionally be the case that the L¹ group shown in formula (QL) comprises an aromatic ring system having not more than two fused aromatic and/or heteroaromatic 6-membered rings, preferably does not comprise any fused aromatic or heteroaromatic ring system. Accordingly, naphthyl structures are preferred over anthracene structures. In addition, fluorenyl, spirobifluorenyl, dibenzofuranyl and/or dibenzothienyl structures are preferred over naphthyl structures.

Particular preference is given to structures having no fusion, for example phenyl, biphenyl, terphenyl and/or quaterphenyl structures.

Examples of suitable aromatic or heteroaromatic ring systems L¹ are selected from the group consisting of ortho-, meta- or para-phenylene, ortho-, meta- or para-biphenylene, terphenylene, especially branched terphenylene, quaterphenylene, especially branched quaterphenylene, fluorenylene, spirobifluorenylene, dibenzofuranylene, dibenzothienylene and carbazolylene, each of which may be substituted by one or more R¹ radicals, but are preferably unsubstituted.

It may further be the case that the L¹ group shown in formula (QL) inter alia has not more than 1 nitrogen atom, preferably not more than 2 heteroatoms, especially preferably not more than one heteroatom and more preferably no heteroatom.

Preferably, the Q group shown in the formula (QL) inter alia, or the electron transport group, may be selected from structures of the formulae (Q-1), (Q-2), (Q-4), (Q-4), (Q-5), (Q-6), (Q-7), (Q-8), (Q-9) and/or (Q-10):

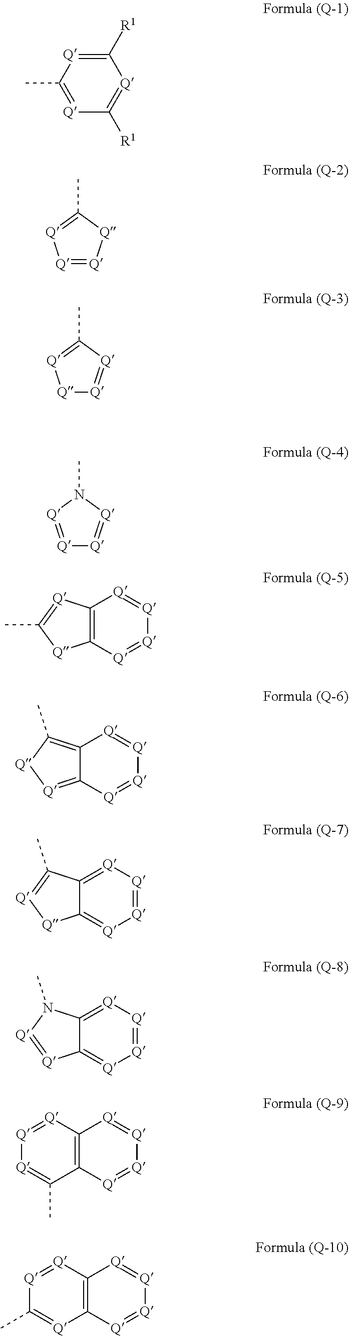

where the dotted bond marks the position of attachment,

Q′ is the same or different at each instance and is CR¹ or N, and

Q″ is NR¹, O or S;

where at least one Q′ is N and

R¹ is as defined above, especially in formula (Ia) or (Ib).

In addition, the Q group shown in the formula (QL) inter alia, or the electron transport group, may preferably be selected from a structure of the formulae (Q-11), (Q-12), (Q-13), (Q-14) and/or (Q-15):

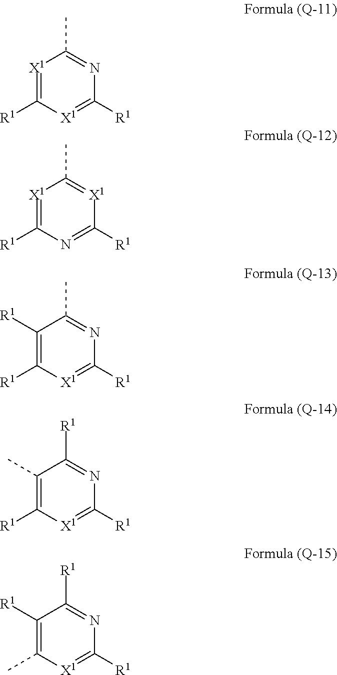

where the symbol R¹ has the definition given for formula (Ia) and/or (Ib) inter alia, X¹ is N or CR¹ and the dotted bond marks the attachment position, where X¹ is preferably a nitrogen atom.

In a further embodiment, the Q group shown in the formula (QL) inter alia, or the electron transport group, may be selected from structures of the formulae (Q-16), (Q-17), (Q-18), (Q-19), (Q-20), (Q-21) and/or (Q-22):

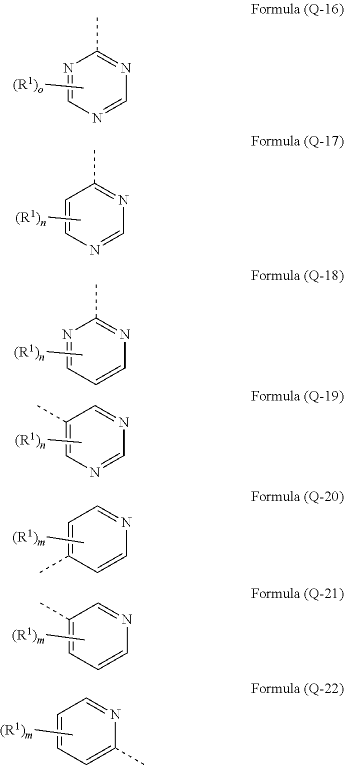

in which the symbol R¹ has the definition detailed above for formulae (Ia) and/or (Ib) inter alia, the dotted bond marks the position of attachment and m is 0, 1, 2, 3 or 4, preferably 0, 1 or 2, n is 0, 1, 2 or 3, preferably 0, 1 or 2, and o is 0, 1 or 2, preferably 1 or 2. Preference is given here to the structures of the formulae (Q-16), (Q-17), (Q-18) and (Q-19).

In a further embodiment, the Q group shown in the formula (QL) inter alia, or the electron transport group, may be selected from structures of the formulae (Q-23), (Q-24) and/or (Q-25):

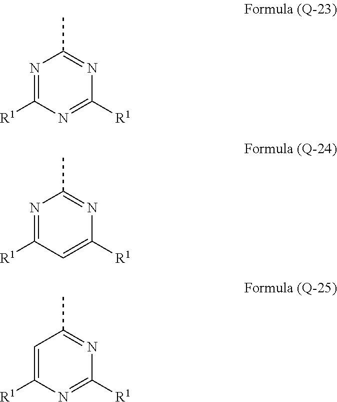

in which the symbol R¹ has the definition set out above for formula (Ia) and/or (Ib) inter alia, and the dotted bond marks the position of attachment.

In a further embodiment, the Q group shown in the formula (QL) inter alia, or the electron transport group, may be selected from structures of the formulae (Q-26), (Q-27), (Q-28), (Q-29) and/or (Q-30):

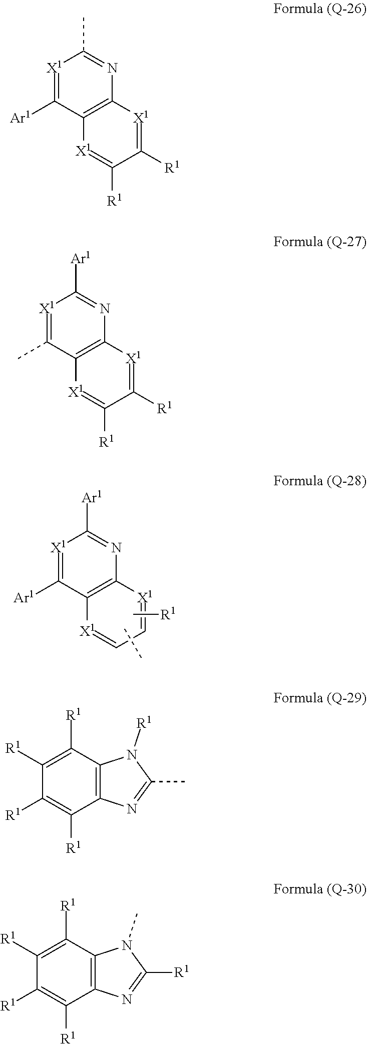

where symbols Ar¹ and R¹ have the definition given above for formula (Ia) and/or (Ib) inter alia, X¹ is N or CR¹ and the dotted bond marks the position of attachment. Preferably, in the structures of the formulae (Q-26), (Q-27) and (Q-28), exactly one X¹ is a nitrogen atom.

Preferably, the Q group shown in the formula (QL) inter alia, or the electron transport group, may be selected from structures of the formulae (Q-31), (Q-32), (Q-33), (Q-34), (Q-35), (Q-36), (Q-37), (Q-38), (Q-39), (Q-40), (Q-41), (Q-42), (Q-43) and/or (Q-44):

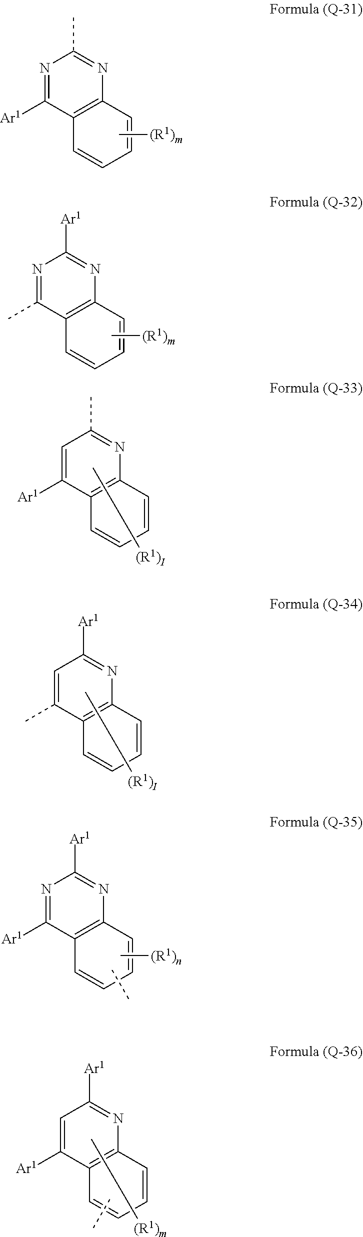

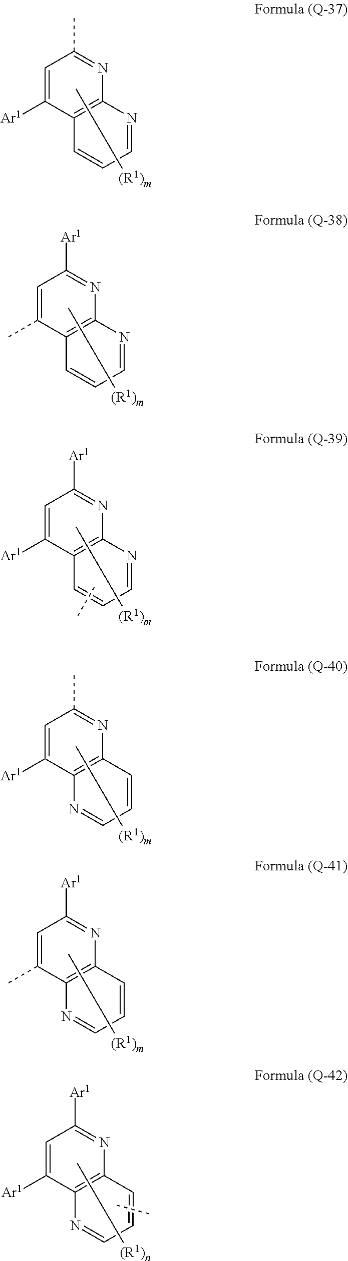

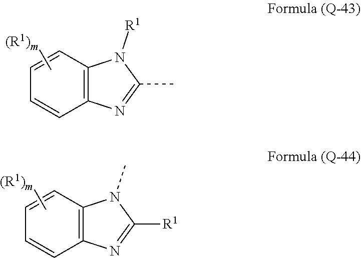

in which the symbols Ar¹ and R¹ have the definition set out above for formula (Ia) and/or (Ib) inter alia, the dotted bond marks the position of attachment and m is 0, 1, 2, 3 or 4, preferably 0, 1 or 2, n is 0, 1, 2 or 3, preferably 0 or 1, n is 0, 1, 2 or 3, preferably 0, 1 or 2, and I is 1, 2, 3, 4 or 5, preferably 0, 1 or 2.

In a further preferred embodiment of the invention, Ar¹ is the same or different at each instance and is an aromatic or heteroaromatic ring system, preferably an aryl or heteroaryl radical having 5 to 24 aromatic ring atoms, preferably having 6 to 18 aromatic ring atoms, and is more preferably an aromatic ring system, preferably an aryl radical having 6 to 12 aromatic ring atoms, or a heteroaromatic ring system, preferably a heteroaryl group having 5 to 13 aromatic ring atoms, each of which may be substituted by one or more R² radicals, but is preferably unsubstituted, where R² may have the definition detailed above, especially in formulae (Ia) and/or (Ib).

Preferably, the symbol Ar¹ is an aryl or heteroaryl radical, such that an aromatic or heteroaromatic group of an aromatic or heteroaromatic ring system is bonded directly, i.e. via an atom of the aromatic or heteroaromatic group, to the respective atom of the further group, for example a carbon or nitrogen atom of the (H-1) to (H-26) or (Q-26) to (Q-44) groups shown above.

Advantageously, Ar¹ in the formulae (H-1) to (H-26) or (Q-26) to (Q-44) is an aromatic ring system which has 6 to 12 aromatic ring atoms and may be substituted by one or more R² radicals, but is preferably unsubstituted, where R² may have the definition detailed above, especially for formulae (Ia) and/or (Ib).

Preferably, the R¹ or R² radicals in the formulae (H-1) to (H-26) or (Q-1) to (Q-44) do not form a fused ring system with the ring atoms of the aryl group or heteroaryl group Ar¹, Ar², Ar³ and/or Ar⁴ to which the R¹ or R² radicals are bonded. This includes the formation of a fused ring system with possible substituents R², R³ which may be bonded to the R¹ or R² radicals.

These electron transport groups can achieve unexpected effects.

It may also be the case that the Ar, Ar¹, Ar², Ar³ and/or Ar⁴ group is selected from the group consisting of phenyl, ortho-, meta- or pare-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, pyrenyl, triazinyl, imidazolyl, benzimidazolyl, benzoxazolyl, benzothiazolyl, 1-, 2-, 3- or 4-carbazolyl, indenocarbazolyl, 1- or 2-naphthyl, anthracenyl, preferably 9-anthracenyl, phenanthrenyl and/or triphenylenyl, each of which may be substituted by one or more R¹ and/or R² radicals, but are preferably unsubstituted, particular preference being given to phenyl, spirobifluorene, fluorene, dibenzofuran, dibenzothiophene, anthracene, phenanthrene, triphenylene groups.

In addition, it may be the case that the substituents R of the heteroaromatic ring system of the formulae (Ia), (Ib), (II-1) to (II-16) and/or (III-1) to (III-16) do not form a fused aromatic or heteroaromatic ring system with the ring atoms of the heteroaromatic ring system, preferably any fused ring system. This includes the formation of a fused ring system with possible R¹, R², R³ substituents which may be bonded to the R radicals. It may preferably be the case that the substituents R in the formulae (Ia), (Ib), (II-1) to (II-16) and/or (III-1) to (III-16) do not form any ring system with the ring atoms of the aromatic or heteroaromatic ring system. This includes the formation of a ring system with possible R¹, R², R³ substituents which may be bonded to the R radicals.

When two radicals that may especially be selected from R, R¹, R², R and/or R³ form a ring system with one another, this ring system may be mono- or polycyclic, aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another.

In a preferred configuration, compounds of the invention are defined by structures of the formula (Ia), (Ib), (II-1), (II-2), (II-3), (II-4), (II-5), (II-6), (II-7), (II-8), (II-9), (II-10), (II-11), (II-12), (II-13), (II-14), (II-15), (II-16), (III-1), (III-2), (III-3), (III-4), (III-5), (III-6), (III-7), (III-8), (III-9), (III-10), (III-11), (III-12), (III-13), (III-14), (III-15) or (III-16), or by preferred embodiments of these structures that will be set out later. Accordingly, preference is given to compounds of a structure of the formula (Ia), (Ib), (II-1), (II-2), (II-3), (II-4), (II-5), (II-6), (II-7), (II-8), (II-9), (II-10), (II-11), (II-12), (II-13), (II-14), (II-15), (II-16), (III-1), (III-2), (III-3), (III-4), (III-5), (III-6), (III-7), (III-8), (III-9), (III-10), (III-11), (III-12), (III-13), (III-14), (III-15) or (III-16), or of one of the preferred embodiments of these structures that will be set out later. Preferably, compounds comprising structures of formula (Ia), (Ib), (II-1), (II-2), (II-3), (II-4), (II-5), (II-6), (II-7), (II-8), (II-9), (II-10), (II-11), (II-12), (II-13), (II-14), (II-15), (II-16), (III-1), (III-2), (III-3), (III-4), (III-5), (III-6), (III-7), (III-8), (III-9), (III-10), (III-11), (III-12), (III-13), (III-14), (III-15) or (III-16), or compounds comprising preferred embodiments of these structures, have a molecular weight of not more than 5000 g/mol, preferably not more than 4000 g/mol, particularly preferably not more than 3000 g/mol, especially preferably not more than 2000 g/mol and most preferably not more than 1200 g/mol.

In addition, it is a feature of preferred compounds of the invention that they are sublimable. These compounds generally have a molar mass of less than about 1200 g/mol.

In a further preferred embodiment, it may be the case that the R group in the structures shown above is selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, pyrenyl, triazinyl, imidazolyl, benzimidazolyl, benzoxazolyl, benzothiazolyl, 1-, 2-, 3- or 4-carbazolyl, 1- or 2-naphthyl, anthracenyl, preferably 9-anthracenyl, phenanthrenyl and/or triphenylenyl, each of which may be substituted by one or more R or R¹ radicals, except fluorenyl and carbazolyl, but are preferably unsubstituted, particular preference being given to spirobifluorene, fluorene, dibenzofuran, dibenzothiophene, anthracene, phenanthrene, triphenylene groups.

When X is CR or when the aromatic and/or heteroaromatic groups are substituted by substituents R, these substituents R are preferably selected from the group consisting of H, D, F, CN, N(Ar)₂, C(═O)Ar, P(═O)(Ar)₂, a straight-chain alkyl or alkoxy group having 1 to 10 carbon atoms or a branched or cyclic alkyl or alkoxy group having 3 to 10 carbon atoms or an alkenyl group having 2 to 10 carbon atoms, each of which may be substituted by one or more R¹ radicals, where one or more nonadjacent CH₂ groups may be replaced by O and where one or more hydrogen atoms may be replaced by D or F, an aromatic or heteroaromatic ring system which has 5 to 24 aromatic ring atoms and may be substituted in each case by one or more R¹ radicals, but is preferably unsubstituted, or an aralkyl or heteroaralkyl group which has 5 to 25 aromatic ring atoms and may be substituted by one or more R¹ radicals; at the same time, it is optionally possible for two substituents R bonded to the same carbon atom or to adjacent carbon atoms to form a monocyclic or polycyclic, aliphatic, aromatic or heteroaromatic ring system that may be substituted by one or more R¹ radicals; where Ar is the same or different at each instance and represents an aromatic or heteroaromatic ring system which has 5 to 40 aromatic ring atoms and may be substituted in each case by one or more R¹ radicals, an aryloxy group which has 5 to 40 aromatic ring atoms and may be substituted by one or more R¹ radicals, or an aralkyl group which has 5 to 40 aromatic ring atoms and may be substituted in each case by one or more R¹ radicals, where it is optionally possible for two or more, preferably adjacent substituents R¹ to form a mono- or polycyclic, aliphatic, heteroaliphatic, aromatic or heteroaromatic ring system, preferably a mono- or polycyclic, aliphatic ring system, which may be substituted by one or more R² radicals, where the symbol R² may have the definition given above, especially for formula (Ia) and/or (Ib). Preferably, Ar is the same or different at each instance and is an aryl or heteroaryl group which has 5 to 24 and preferably 5 to 12 aromatic ring atoms, and which may be substituted in each case by one or more R¹ radicals, but is preferably unsubstituted.

Examples of suitable Ar groups are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, each of which may be substituted by one or more R¹ radicals, but are preferably unsubstituted.

More preferably, these substituents R are selected from the group consisting of H, D, F, CN, N(Ar)₂, a straight-chain alkyl group having 1 to 8 carbon atoms, preferably having 1, 2, 3 or 4 carbon atoms, or a branched or cyclic alkyl group having 3 to 8 carbon atoms, preferably having 3 or 4 carbon atoms, or an alkenyl group having 2 to 8 carbon atoms, preferably having 2, 3 or 4 carbon atoms, each of which may be substituted by one or more R¹ radicals, but is preferably unsubstituted, or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more nonaromatic R¹ radicals, but is preferably unsubstituted; at the same time, it is optionally possible for two substituents R bonded to the same carbon atom or to adjacent carbon atoms to form a monocyclic or polycyclic aliphatic ring system which may be substituted by one or more R¹ radicals, but is preferably unsubstituted, where Ar may have the definition set out above.

Most preferably, the substituents R are selected from the group consisting of H and an aromatic or heteroaromatic ring system having 6 to 18 aromatic ring atoms, preferably having 6 to 13 aromatic ring atoms, each of which may be substituted by one or more nonaromatic R¹ radicals, but is preferably unsubstituted.

In a preferred configuration, it may be the case that at least one of the R and/or R^{a} groups in the formulae (Ia), (Ib), (II-1), (II-2), (II-3), (II-4), (II-5), (II-6), (II-7), (II-8), (II-9), (II-10), (II-11), (II-12), (II-13), (II-14), (II-15), (II-16), (III-1), (III-2), (III-3), (III-4), (III-5), (III-6), (III-7), (III-8), (III-9), (III-10), (III-11), (III-12), (III-13), (III-14), (III-15) or (III-16) is a group that can be represented by the formula L¹-Z in which L¹ represents a bond or an aromatic or heteroaromatic ring system which has 5 to 40, preferably 5 to 30, aromatic ring atoms and may be substituted by one or more R¹ radicals, Z represents R¹, Ar or a group of the formula Z^{a} or Z^{b} in which the symbols Ar and R¹ have the definition given above, especially for formula (Ia) and/or (Ib), and Z^{a} or Z^{b} are

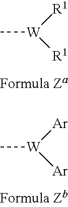

in which W is the same or different at each instance and is an aromatic or heteroaromatic ring system which has 5 to 30 aromatic ring atoms and may be substituted by one or more R¹ radicals, a nitrogen atom, a boron atom, a phosphorus atom or a phosphane oxide group, the dotted bond marks the position of attachment and the symbols Ar and R¹ have the definition given above, especially for formula (Ia) and/or (Ib).

It may preferably be the case that the compounds of the invention comprise at least one structure of the formula (IV-1), (IV-2), (IV-3), (IV-4), (IV-5), (IV-6), (IV-7), (IV-8), (IV-9), (IV-10), (IV-11), (IV-12), (IV-13), (IV-14), (IV-15), (IV-16), (IV-17), (IV-18), (IV-19), (IV-20), (IV-21), (IV-22), (IV-23) and/or (IV-24), where the compounds can preferably be represented by structures of the formula (IV-1), (IV-2), (IV-3), (IV-4), (IV-5), (IV-6), (IV-7), (IV-8), (IV-9), (IV-10), (IV-11), (IV-12), (IV-13), (IV-14), (IV-15), (IV-16), (IV-17), (IV-18), (IV-19), (IV-20), (IV-21), (IV-22), (IV-23) and/or (IV-24):

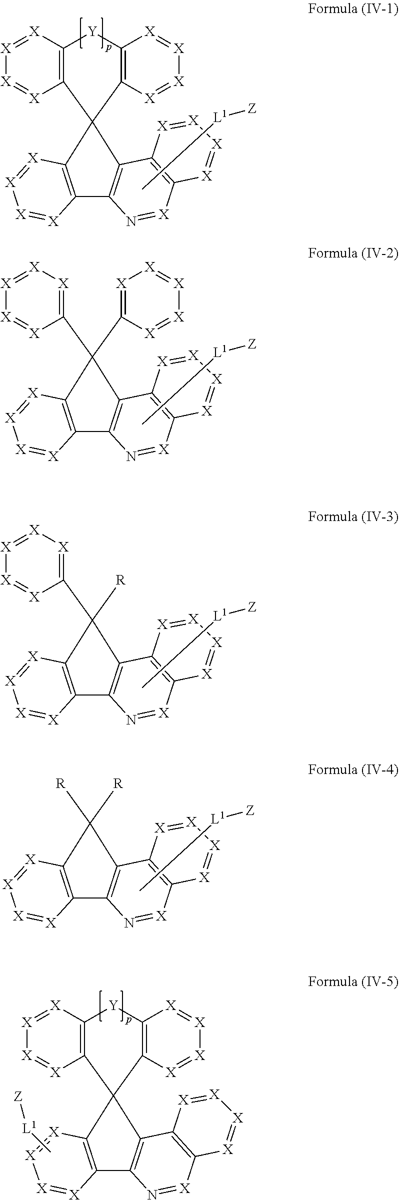

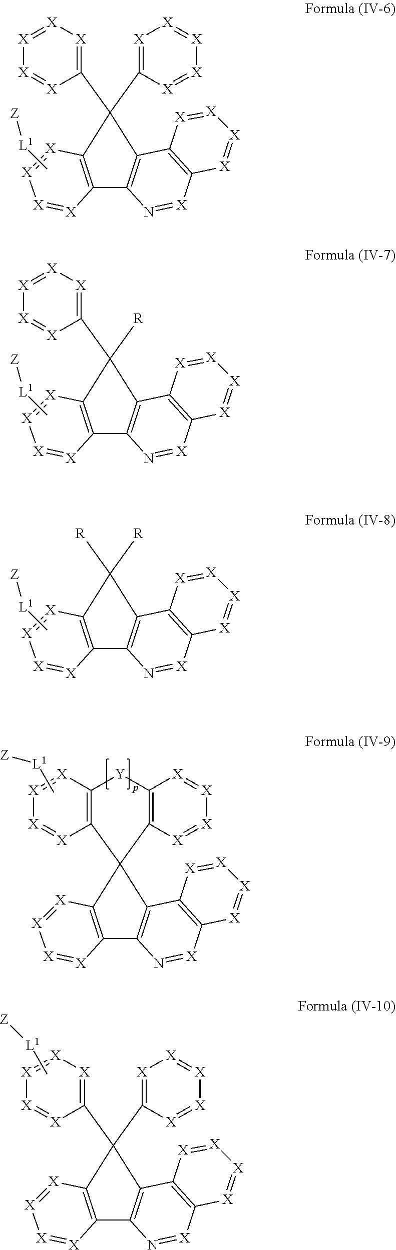

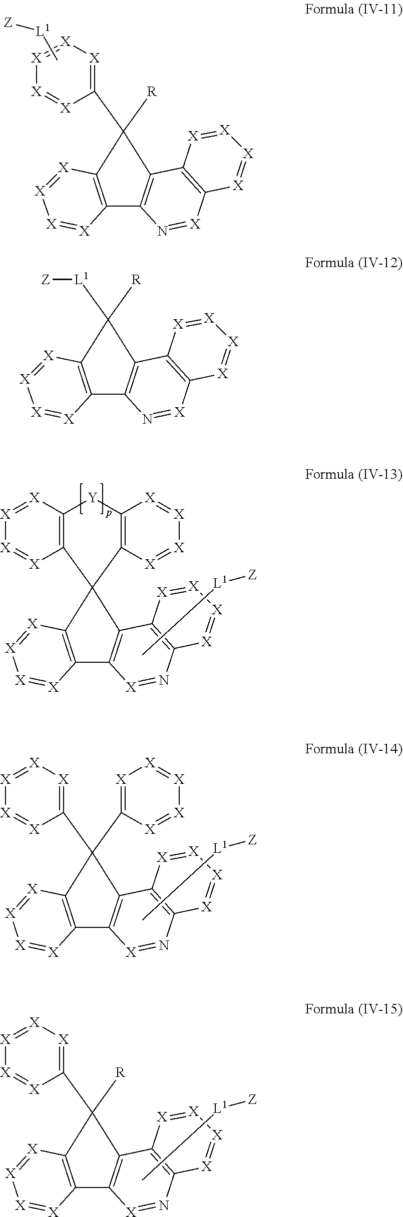

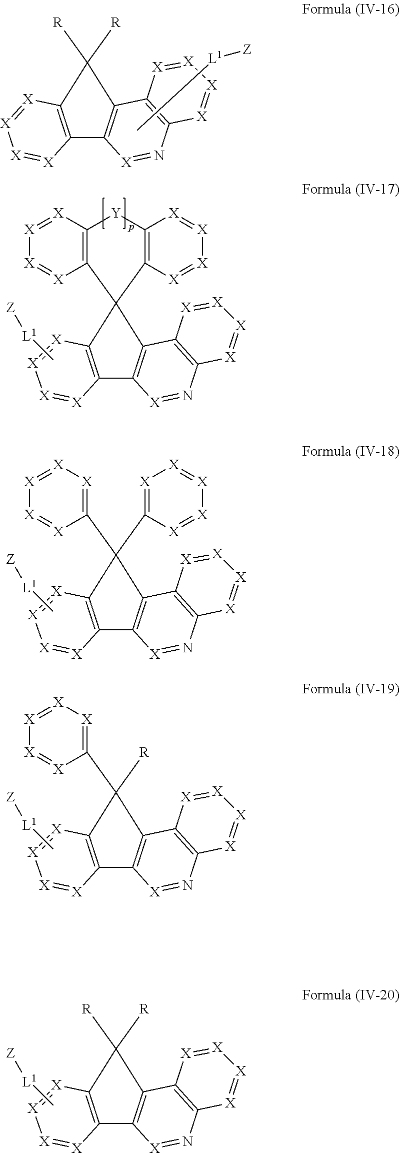

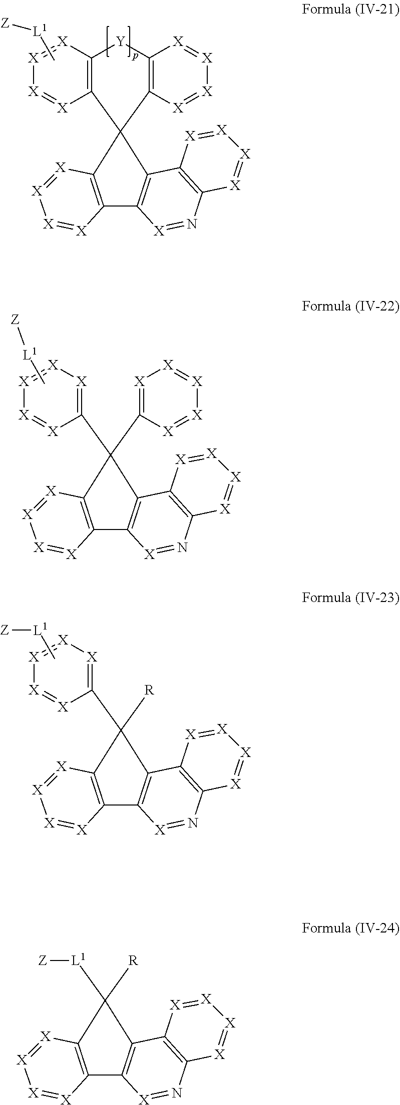

where the symbol X represents N, CR or C if the -L¹-Z group is bonded to X, and the symbols R, Y, p, L¹ and Z used have the definition given above, especially for formulae (Ia), (Ib), (II-1) to (II-16) and/or (L¹-Z).

In addition, particular preference is given to structures (preferably compounds) of the formulae (IV-9), (IV-13), (IV-17) and (IV-21):

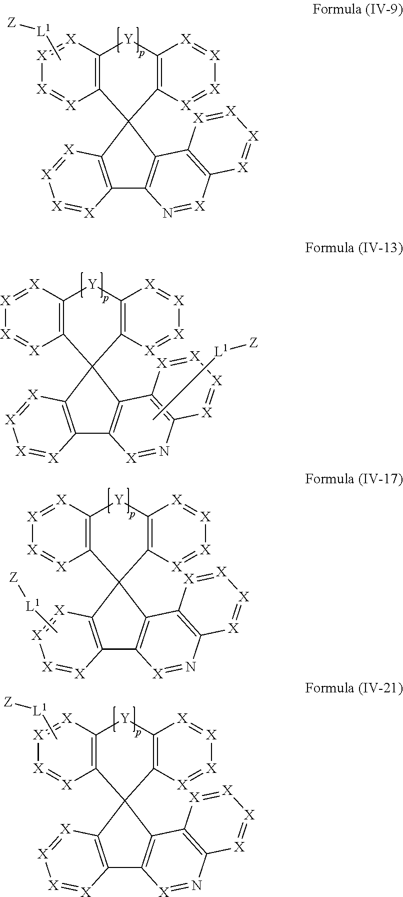

where the symbol X represents N, CR or C if the -L¹-Z group is bonded to X, and the symbols R, Y, p, L¹ and Z used have the definition given above, especially for formulae (Ia), (Ib), (II-1) to (II-16) and/or (L¹-Z). In relation to the formulae (IV-9), (IV-13), (IV-17) and (IV-21), preference is given to structures (preferably compounds) in which p=0, such that a bond is formed between the two rings.

In a further embodiment, it may be the case that the compounds of the invention comprise at least one structure of the formula (V-1), (V-2), (V-3), (V-4), (V-5), (V-6), (V-7), (V-8), (V-9), (V-10), (V-11), (V-12), (V-13), (V-14), (V-15), (V-16), (V-17), (V-18), (V-19), (V-20), (V-21), (V-22), (V-23), (V-24), (V-25), (V-26), (V-27), (V-28), (V-29), (V-30), (V-31) and/or (V-32), where the compounds can preferably be represented by structures of the formula (V-1), (V-2), (V-3), (V-4), (V-5), (V-6), (V-7), (V-8), (V-9), (V-10), (V-11), (V-12), (V-13), (V-14), (V-15), (V-16), (V-17), (V-18), (V-19), (V-20), (V-21), (V-22), (V-23), (V-24), (V-25), (V-26), (V-27), (V-28), (V-29), (V-30), (V-31) and/or (V-32):

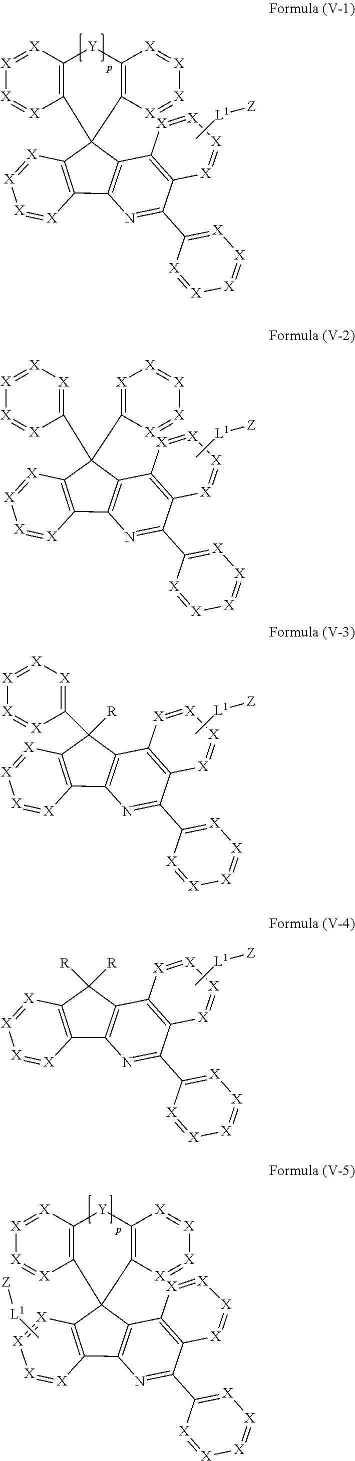

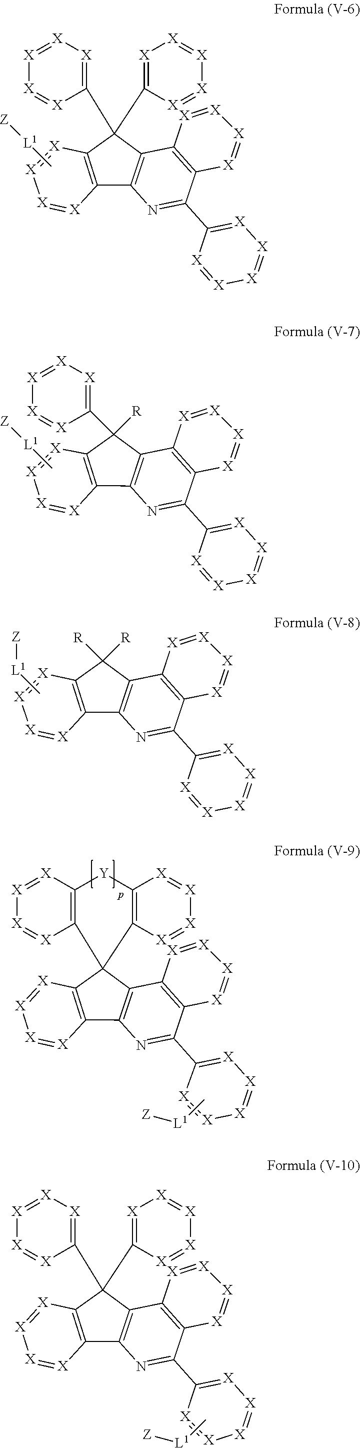

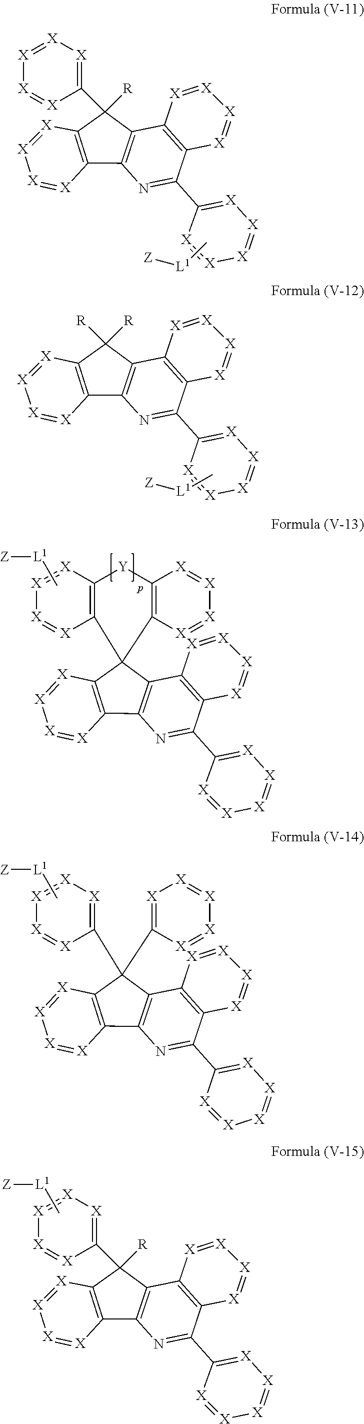

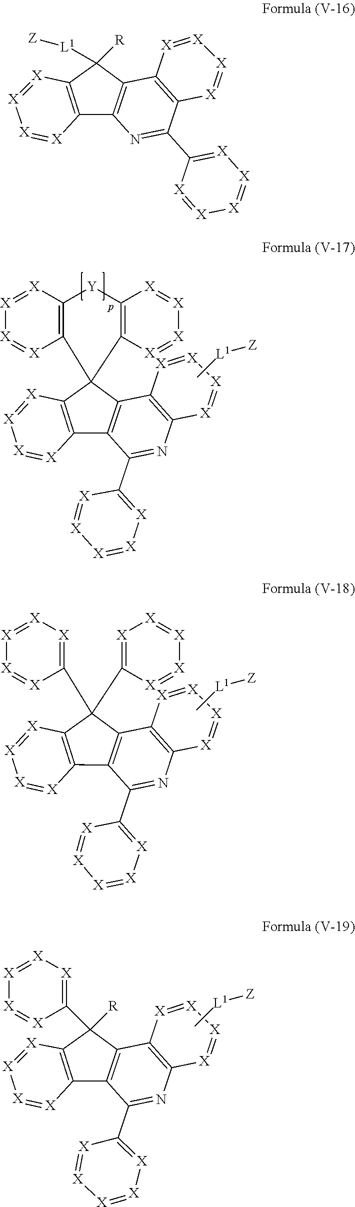

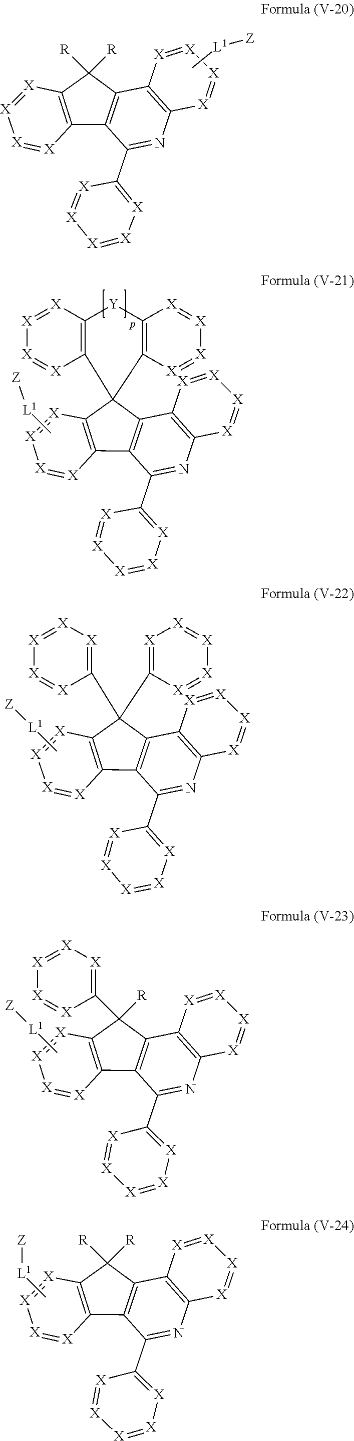

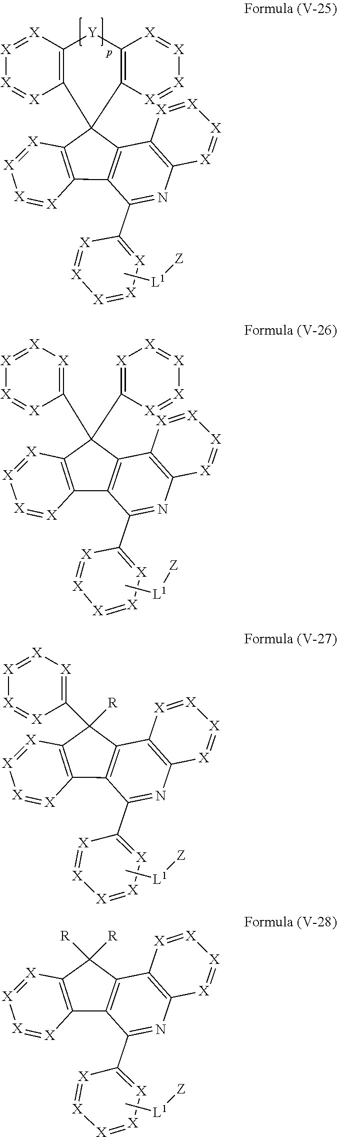

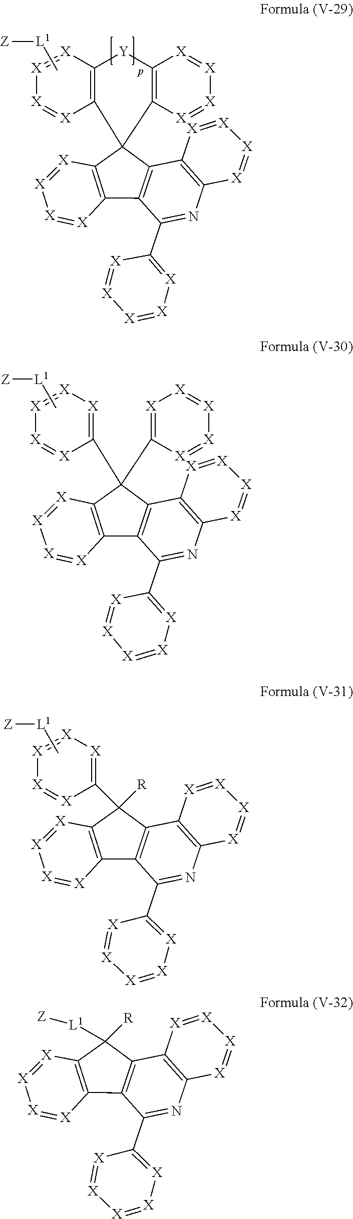

where the symbol X represents N, CR or C if the -L¹-Z group is bonded to X, and the symbols R, Y, p, L¹ and Z used have the definition given above, especially for formulae (Ia), (Ib), (II-1) to (II-16) and/or (L¹-Z).

In addition, particular preference is given to structures (preferably compounds) of the formulae (V-17), (V-21), (V-25) and (V-29):

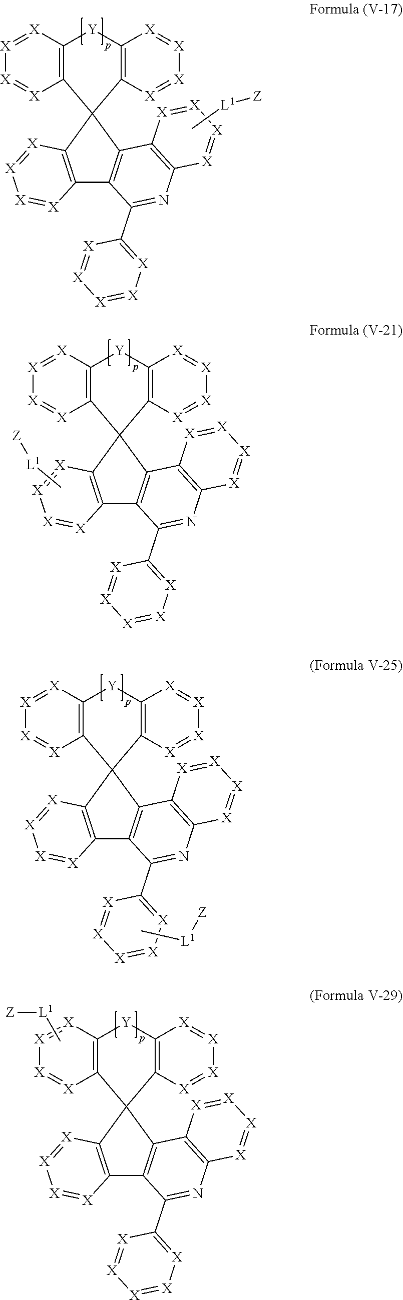

where the symbol X represents N, CR or C if the -L¹-Z group is bonded to X, and the symbols R, Y, p, L¹ and Z used have the definition given above, especially for formulae (Ia), (Ib), (II-1) to (II-16) and/or (L¹-Z). In relation to the formulae (V-17), (V-21), (V-25) and (V-29), preference is given to structures (preferably compounds) in which p=0, such that a bond is formed between the two rings.

In a preferred configuration, it may be the case that the compounds of the invention comprise at least one structure of the formula (VI-1), (VI-2), (VI-3), (VI-4), (VI-5), (VI-6), (VI-7) and/or (VI-8), where the compounds can preferably be represented by structures of the formula (VI-1), (VI-2), (VI-3), (VI-4), (VI-5), (VI-6), (VI-7) and/or (VI-8):

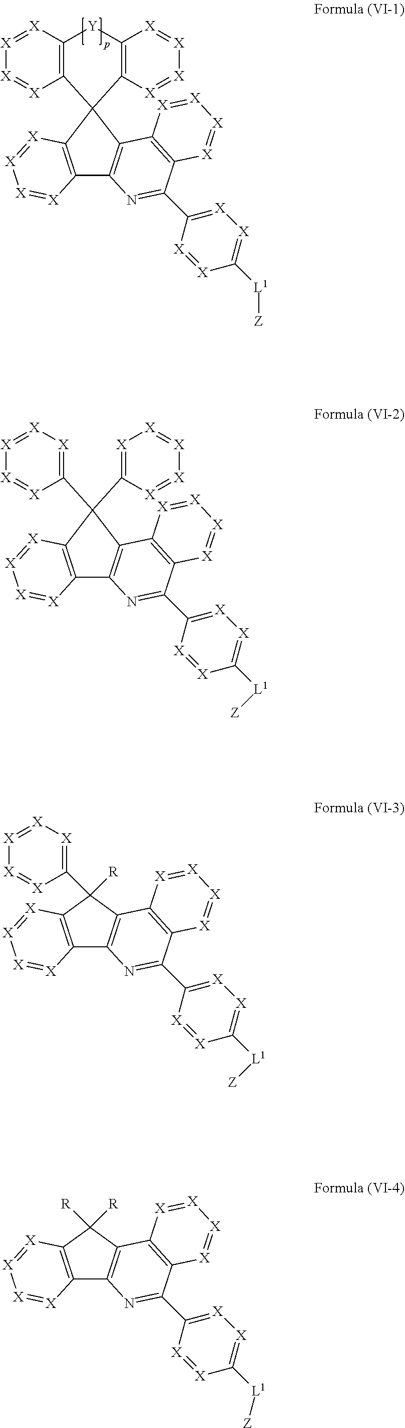

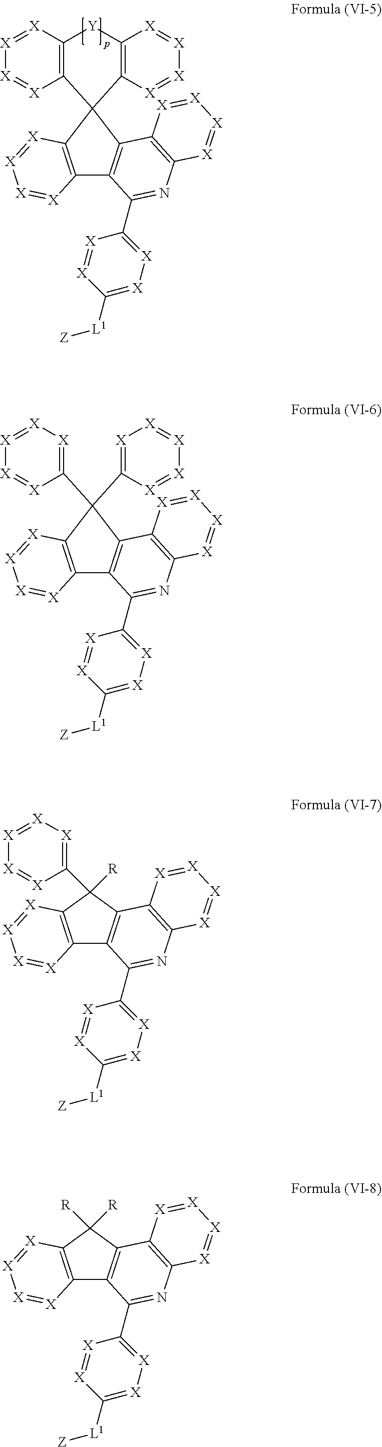

where the symbol X represents N, CR or C if the -L¹-Z group is bonded to X, and the symbols R, Y, p, L¹ and Z used have the definition given above, especially for formulae (Ia), (Ib), (II-1) to (II-16) and/or (L¹-Z). Preference is given here to structures (preferably compounds) of the formulae (VI-1), (VI-2), (VI-4), (VI-5), (VI-6) and (VI-8), particular preference to structures (preferably compounds) of the formulae (VI-5), (VI-6), (VI-8). Particular preference is given to formula (VI-5), preference being given to structures (preferably compounds) in which p=0, such that a bond is formed between the two rings.

The Y radical, especially in a structure of the formulae (I1-1) to (II-16), (III-1) to (III-16), (IV-1) to (IV-24), (V-1) to (V-32) and/or (VI-1) to (VI-8), is preferably O or NR.

More preferably, it may be the case that the compounds of the invention comprise at least one structure of the formula (VII-1), (VII-2), (VII-3), (VII-4), (VII-5), (VII-6), (VII-7) and/or (VII-8), where the compounds can preferably be represented by structures of the formula (VII-1), (VII-2), (VII-3), (VII-4), (VII-5), (VII-6), (VII-7) and/or (VII-8):

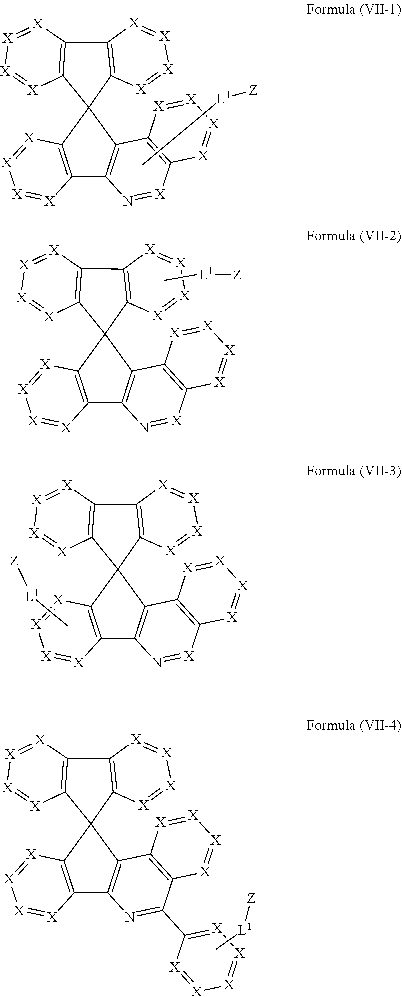

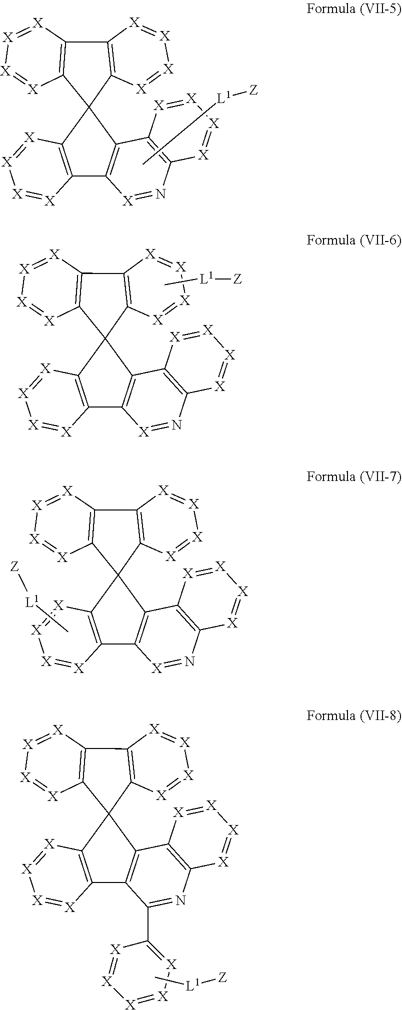

where the symbol X represents N, CR or C if the -L¹-Z group is bonded to X, and the symbols R, L¹ and Z used have the definition given above, especially for formulae (Ia), (Ib), (II-1) to (II-16) and/or (L¹-Z).

In a further preferred embodiment, it may be the case that the compounds of the invention comprise at least one structure of the formula (VIII-1), (VIII-2), (VIII-3), (VIII-4), (VIII-5), (VIII-6), (VIII-7), (VIII-8), (VIII-9) and/or (VIII-10), where the compounds can preferably be represented by structures of the formula (VIII-1), (VIII-2), (VIII-3), (VIII-4), (VIII-5), (VIII-6), (VIII-7), (VIII-8), (VIII-9) and/or (VIII-10):

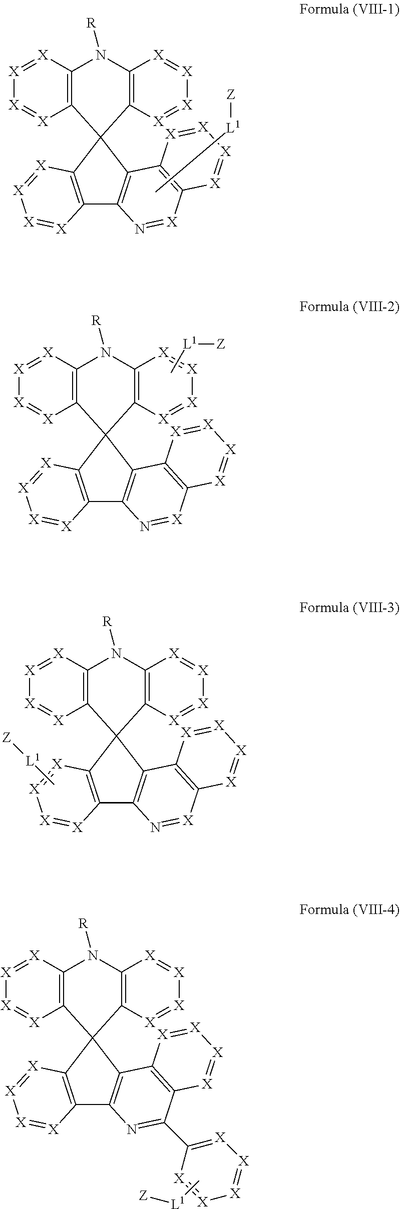

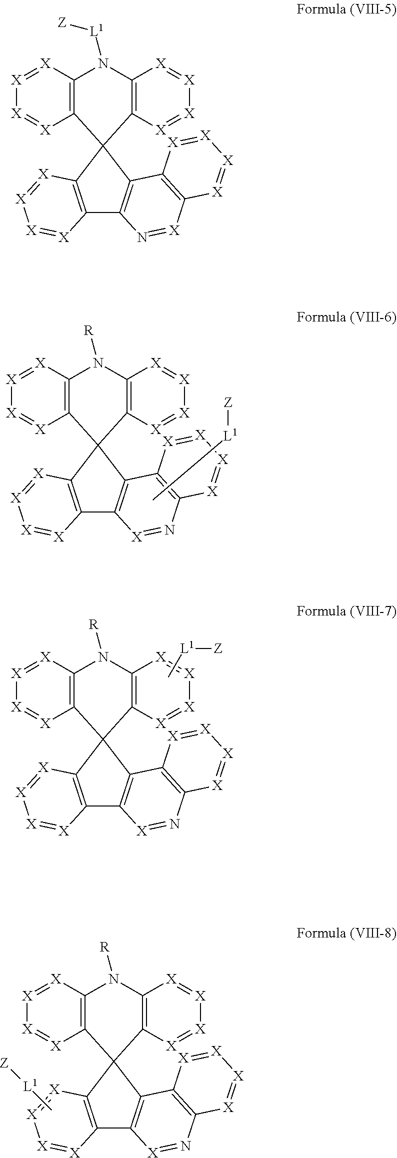

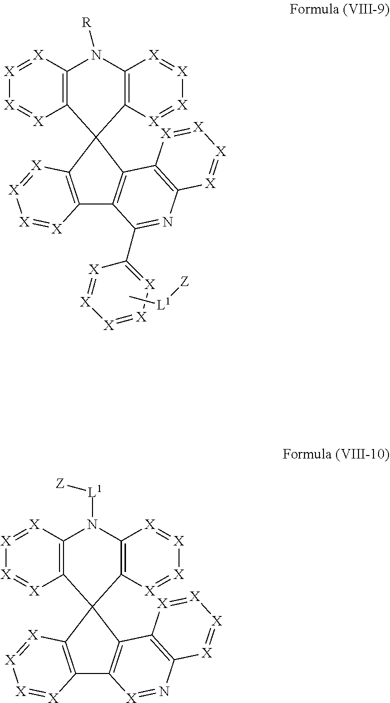

where the symbol X represents N, CR or C if the -L¹-Z group is bonded to X, and the symbols R, L¹ and Z used have the definition given above, especially for formulae (Ia), (Ib), (II-1) to (II-16) and/or (L¹-Z).

It may preferably further be the case that the compounds of the invention comprise at least one structure of the formula (IX-1), (IX-2), (IX-3), (IX-4), (IX-5), (IX-6), (IX-7) and/or (IX-8), where the compounds can preferably be represented by structures of the formula (IX-1), (IX-2), (IX-3), (IX-4), (IX-5), (IX-6), (IX-7) and/or (IX-8):

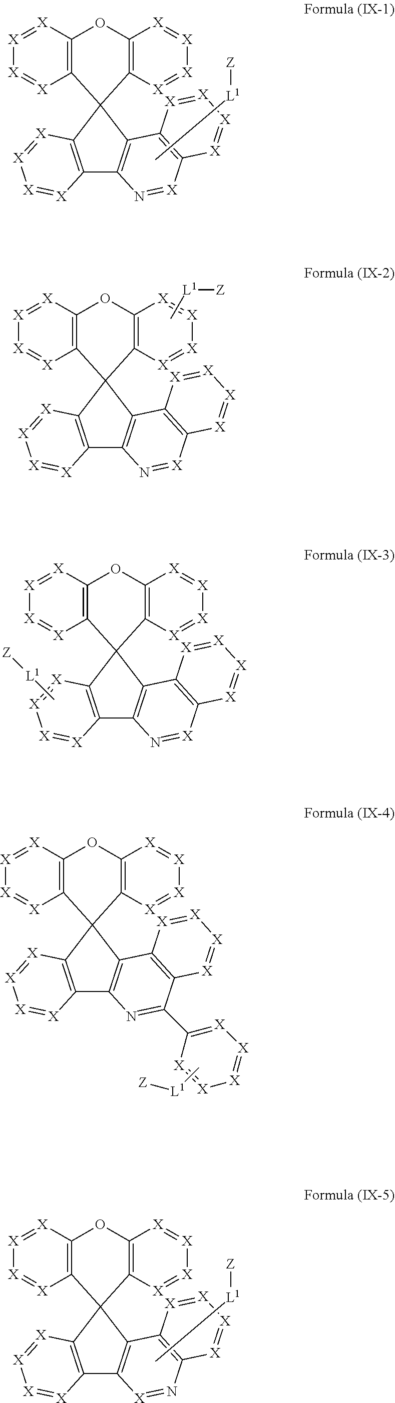

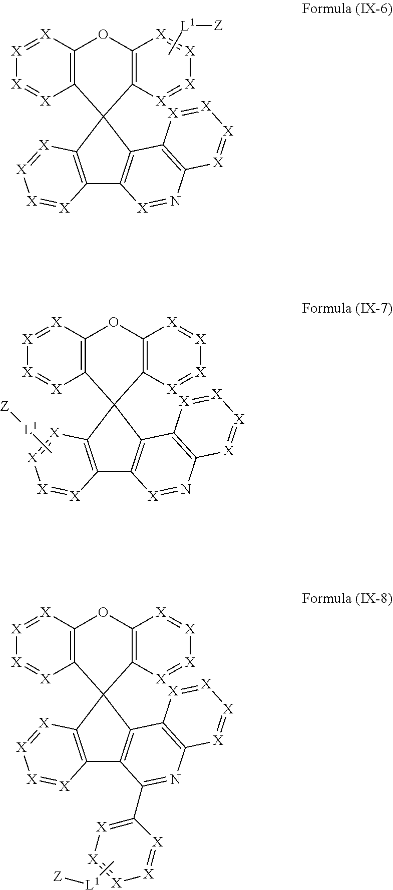

where the symbol X represents N, CR or C if the -L¹-Z group is bonded to X, and the symbols R, L¹ and Z used have the definition given above, especially for formulae (Ia), (Ib), (II-1) to (II-16) and/or (L¹-Z).

It may further be the case that the compounds of the invention comprise at least one structure of the formula (X-1), (X-2), (X-3), (X-4), (X-5), (X-6), (X-7) and/or (X-8), where the compounds can preferably be represented by structures of the formula (X-1), (X-2), (X-3), (X-4), (X-5), (X-6), (X-7) and/or (X-8):

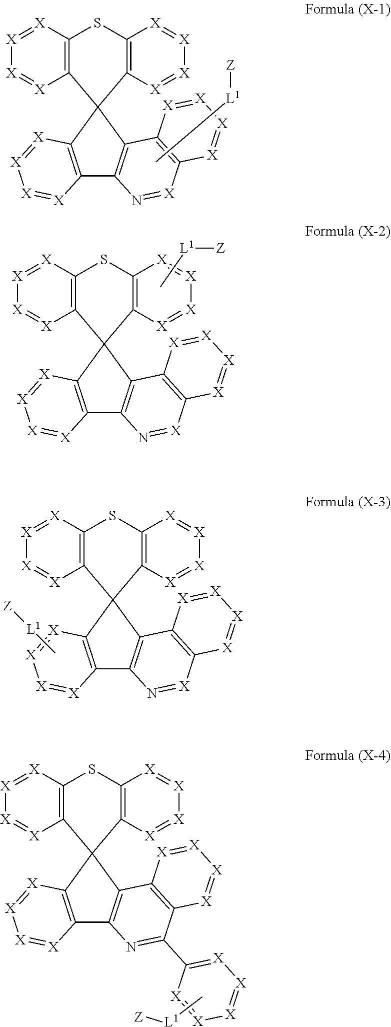

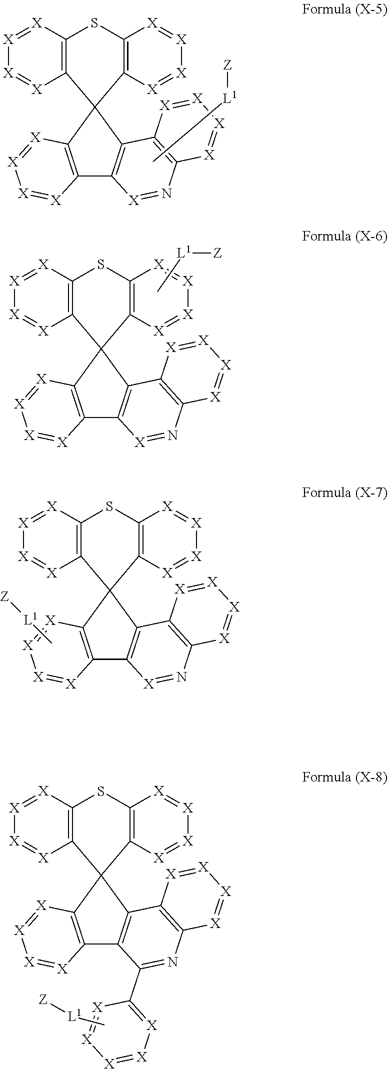

where the symbol X represents N, CR or C if the -L¹-Z group is bonded to X, and the symbols R, L¹ and Z used have the definition given above, especially for formulae (Ia), (Ib), (II-1) to (II-16) and/or (L¹-Z).

It may preferably further be the case that the compounds of the invention comprise at least one structure of the formula (XI-1), (XI-2), (XI-3), (XI-4), (XI-5), (XI-6), (XI-7), (XI-8), (XI-9) and/or (XI-10), where the compounds can preferably be represented by structures of the formula (XI-1), (XI-2), (XI-3), (XI-4), (XI-5), (XI-6), (XI-7), (XI-8), (XI-9) and/or (XI-10):

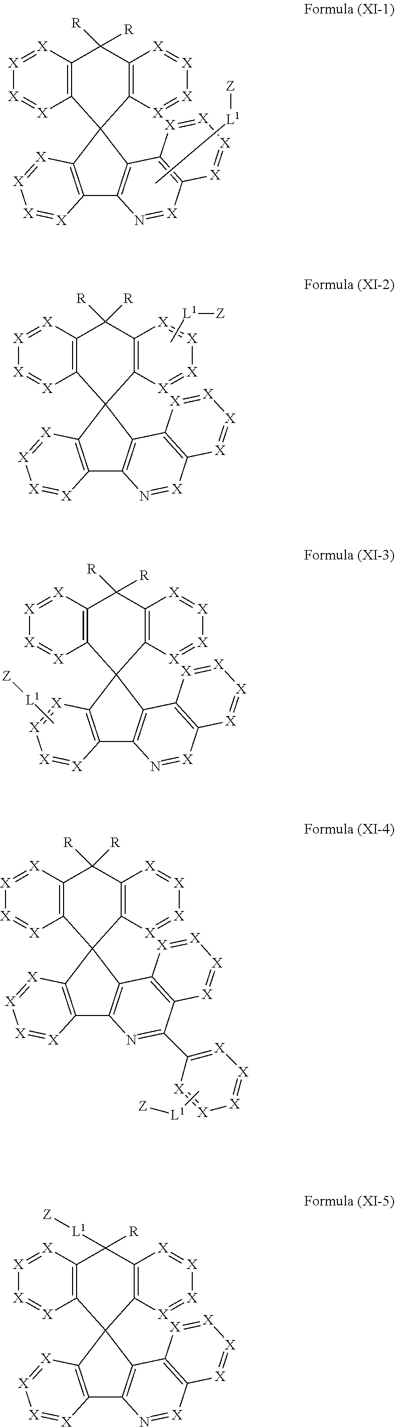

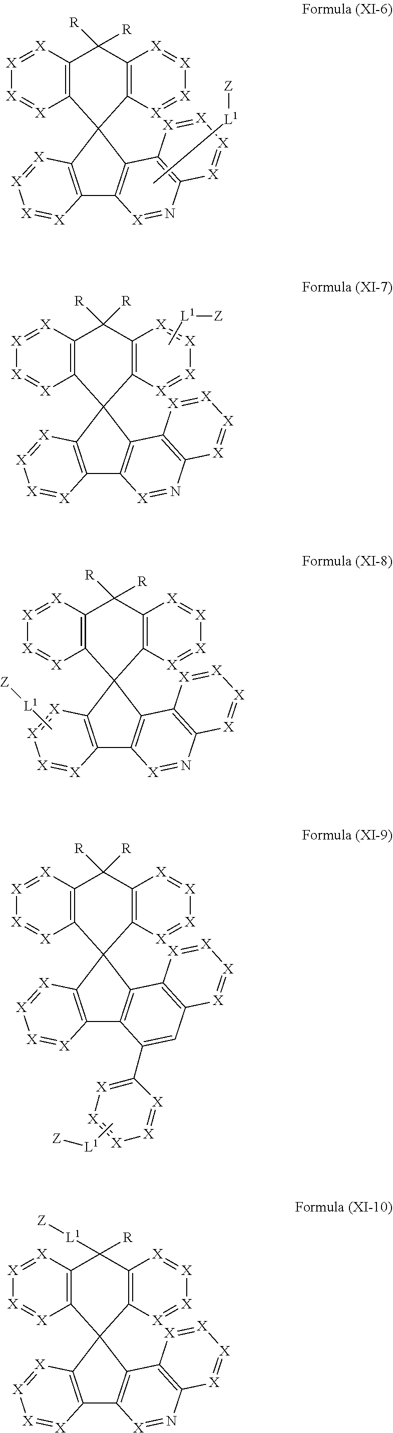

where the symbol X represents N, CR or C if the -L¹-Z group is bonded to X, and the symbols R, L¹ and Z used have the definition given above, especially for formulae (Ia), (Ib), (II-1) to (II-16) and/or (L¹-Z).

It may preferably be the case that, in formulae (Ia), (Ib), (II-1) to (II-16), (III-1) to (III-16), (IV-1) to (IV-24), (V-1) to (V-32), (VI-1) to (VI-8), (VII-1) to (VII-8), (VIII-1) to (VIII-10), (IX-1) to (IX-8), (X-1) to (X-8) and/or (XI-1) to (XI-10), not more than two X groups per ring are N; preferably at least one and more preferably at least two of the X groups per ring is/are selected from C-H and C-D.

Preferably, in formulae (Ia), (Ib), (II-1) to (II-16), (III-1) to (III-16), (IV-1) to (IV-24), (V-1) to (V-32), (VI-1) to (VI-8), (VII-1) to (VII-8), (VIII-1) to (VIII-10), (IX-1) to (IX-8), (X-1) to (X-8) and/or (XI-1) to (XI-10), not more than four and preferably not more than two X groups are N; more preferably, all X groups are CR, where preferably not more than 4, more preferably not more than 3 and especially preferably not more than 2 of the CR groups that X represents are not the CH group.

It may further the case that the compounds of the invention comprise at least one structure of the formula (XII-1), (XII-2), (XII-3), (XII-4), (XII-5), (XII-6), (XII-7), (XII-8), (XII-9), (XII-10), (XII-11), (XII-12), (XII-13), (XII-14), (XII-15), (XII-16), (XII-17), (XII-18), (XII-19), (XII-20), (XII-21), (XII-22), (XII-23) and/or (XII-24), where the compounds can preferably be represented by structures of the formula (XII-1), (XII-2), (XII-3), (XII-4), (XII-5), (XII-6), (XII-7), (XII-8), (XII-9), (XII-10), (XII-11), (XII-12), (XII-13), (XII-14), (XII-15), (XII-16), (XII-17), (XII-18), (XII-19), (XII-20), (XII-21), (XII-22), (XII-23) and/or (XII-24):

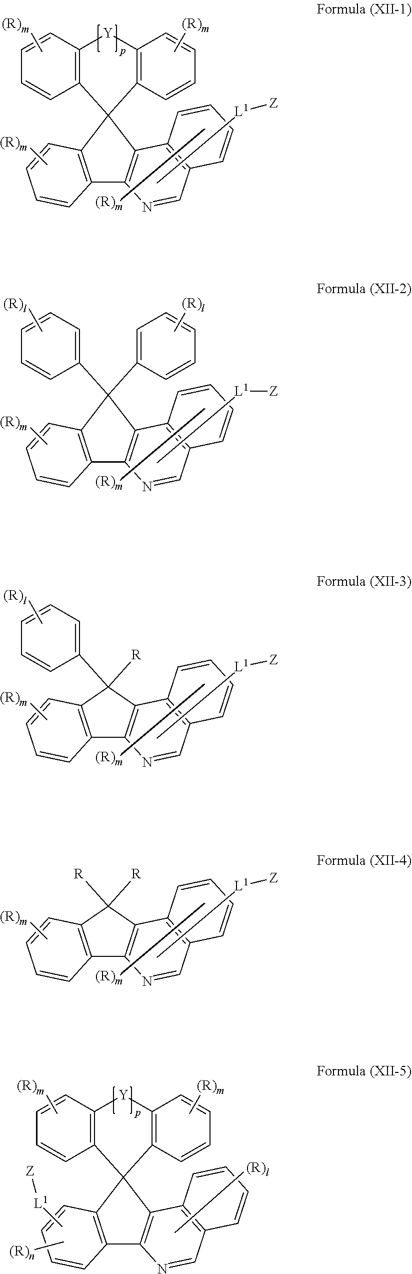

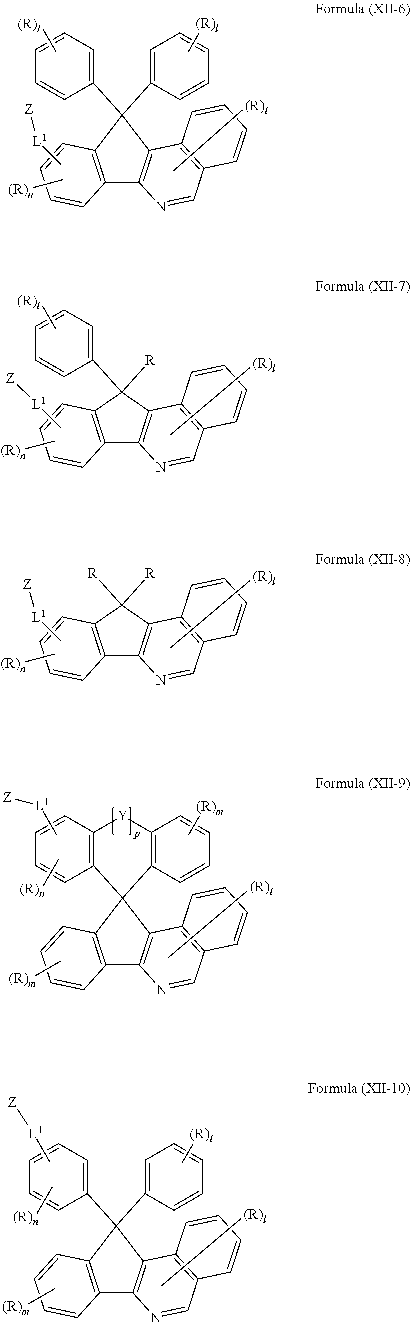

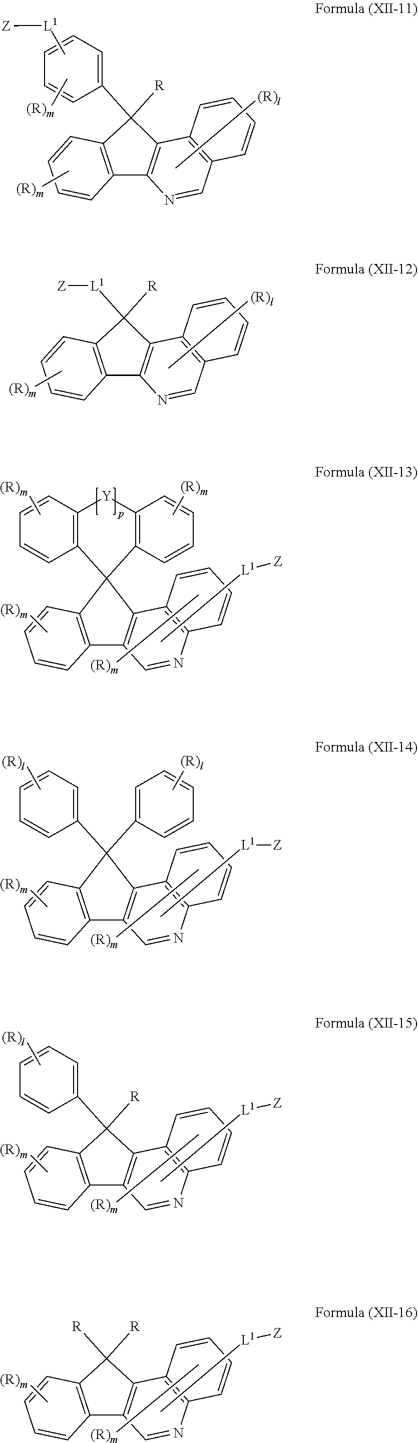

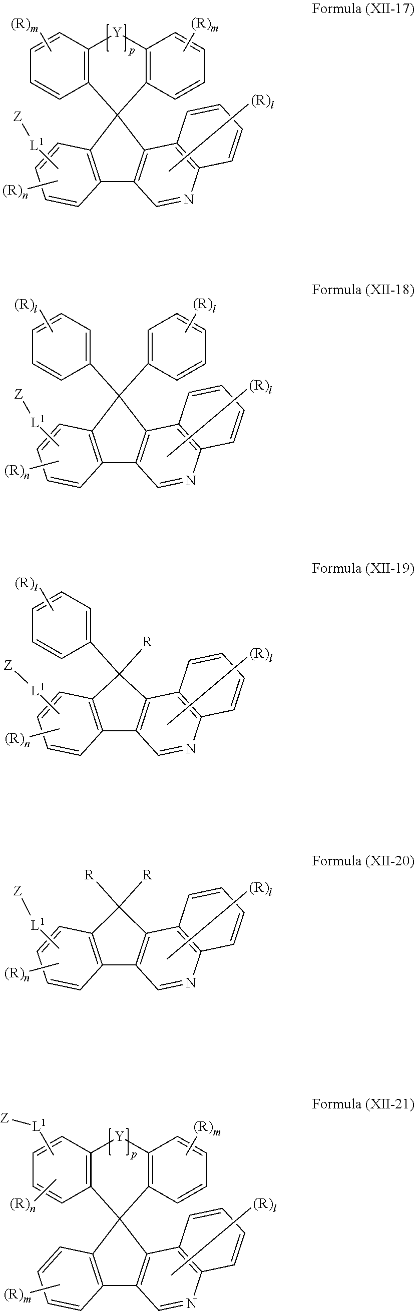

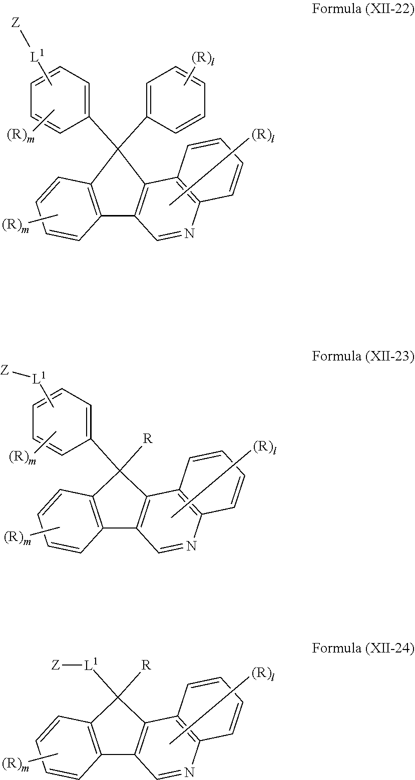

where the symbols R, Y, p, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1. Preference is given here to compounds having groups of the formulae (XII-1) to (XII-9) and (XII-13) to (XII-21).

In addition, particular preference is given to structures (preferably compounds) of the formulae (XII-13), (XII-17) and (XII-21):

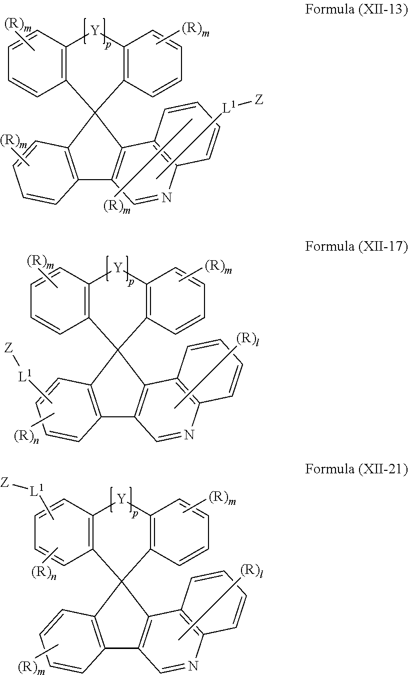

where the symbols R, Y, p, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1. In relation to the formulae (XII-13), (XII-17) and (XII-21), preference is given to structures (preferably compounds) in which p=0, such that a bond is formed between the two rings.

It may preferably be the case that the compounds of the invention comprise at least one structure of the formula (XIII-1), (XIII-2), (XIII-3), (XIII-4), (XIII-5), (XIII-6), (XIII-7), (XIII-8), (XIII-9), (XIII-10), (XIII-11), (XIII-12), (XIII-13), (XIII-14), (XIII-15), (XIII-16), (XIII-17), (XIII-18), (XIII-19), (XIII-20), (XIII-21), (XIII-22), (XIII-23), (XIII-24), (XIII-25), (XIII-26), (XIII-27), (XIII-28), (XIII-29), (XIII-30), (XIII-31) and/or (XIII-32), where the compounds can preferably be represented by structures of the formula (XIII-1), (XIII-2), (XIII-3), (XIII-4), (XIII-5), (XIII-6), (XIII-7), (XIII-8), (XIII-9), (XIII-10), (XIII-11), (XIII-12), (XIII-13), (XIII-14), (XIII-15), (XIII-16), (XIII-17), (XIII-18), (XIII-19), (XIII-20), (XIII-21), (XIII-22), (XIII-23), (XIII-24), (XIII-25), (XIII-26), (XIII-27), (XIII-28), (XIII-29), (XIII-30), (XIII-31) and/or (XIII-32):

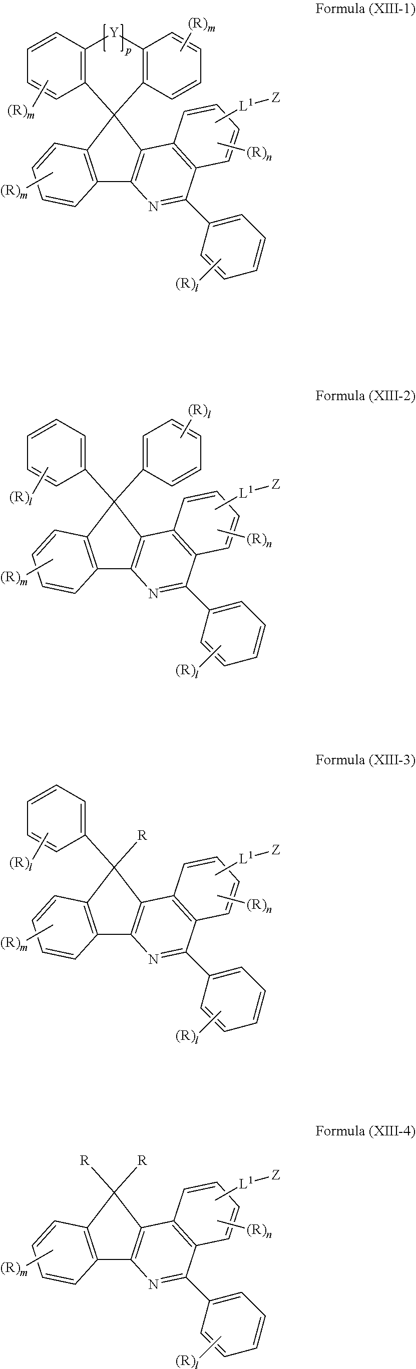

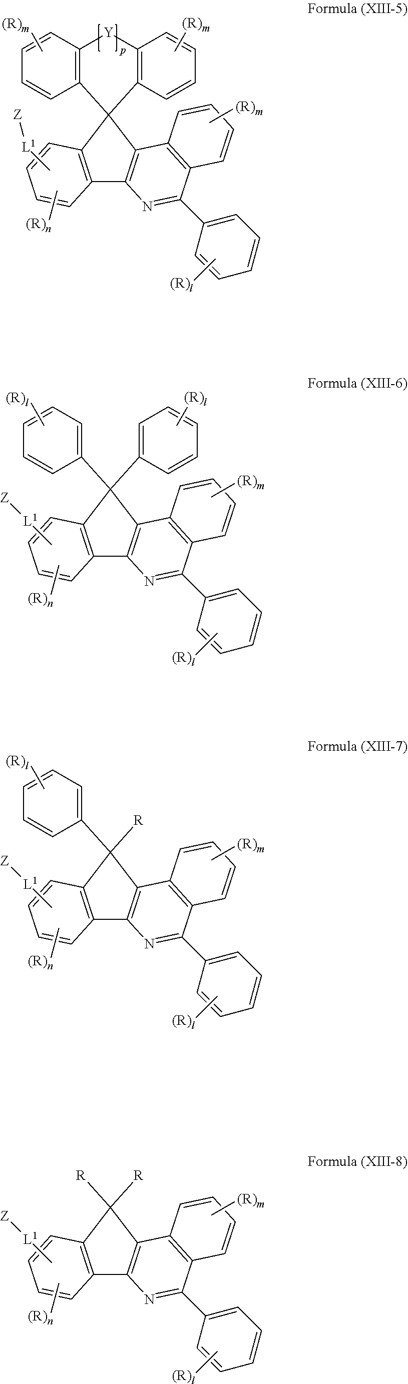

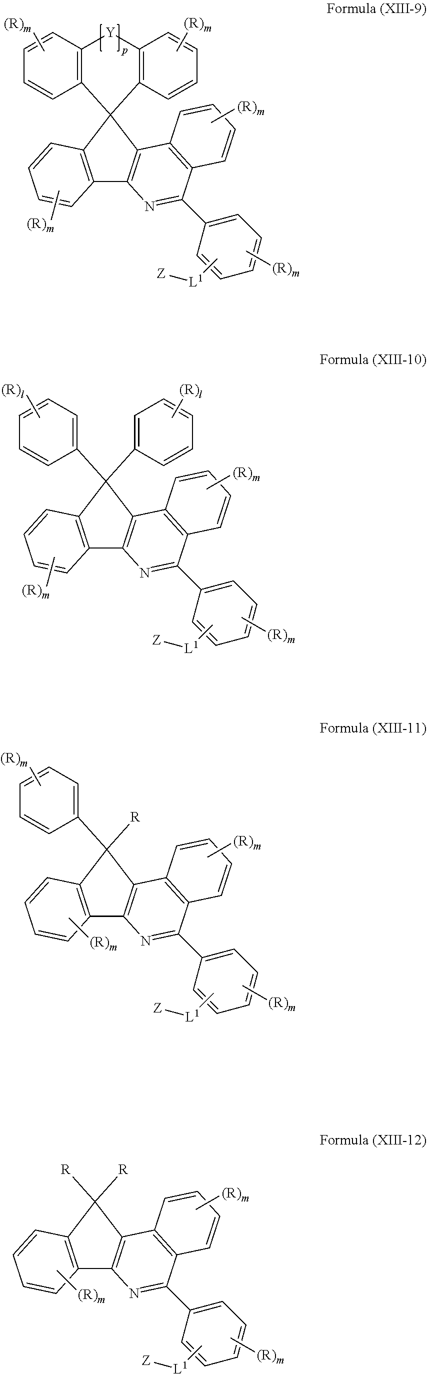

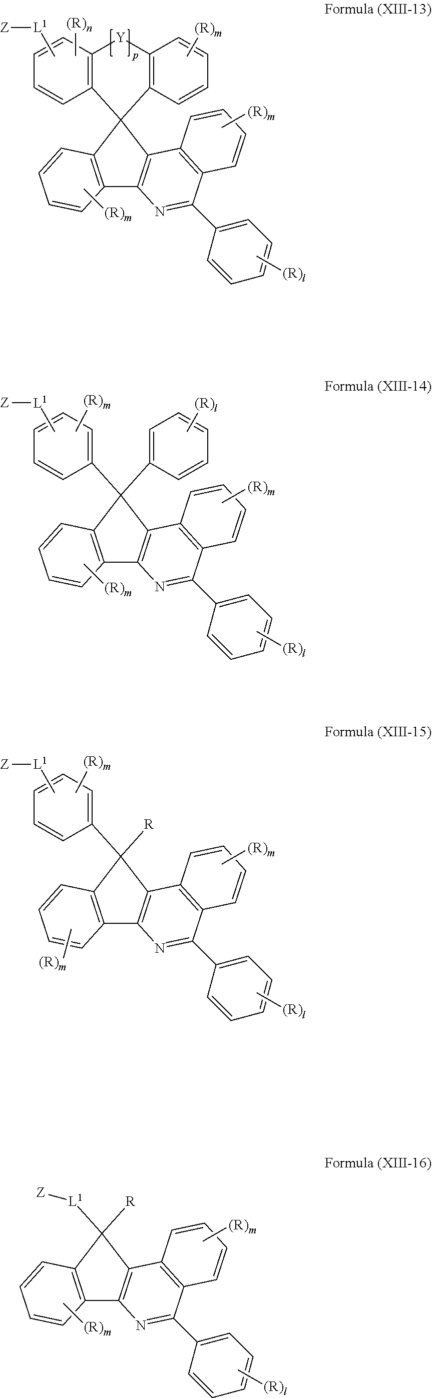

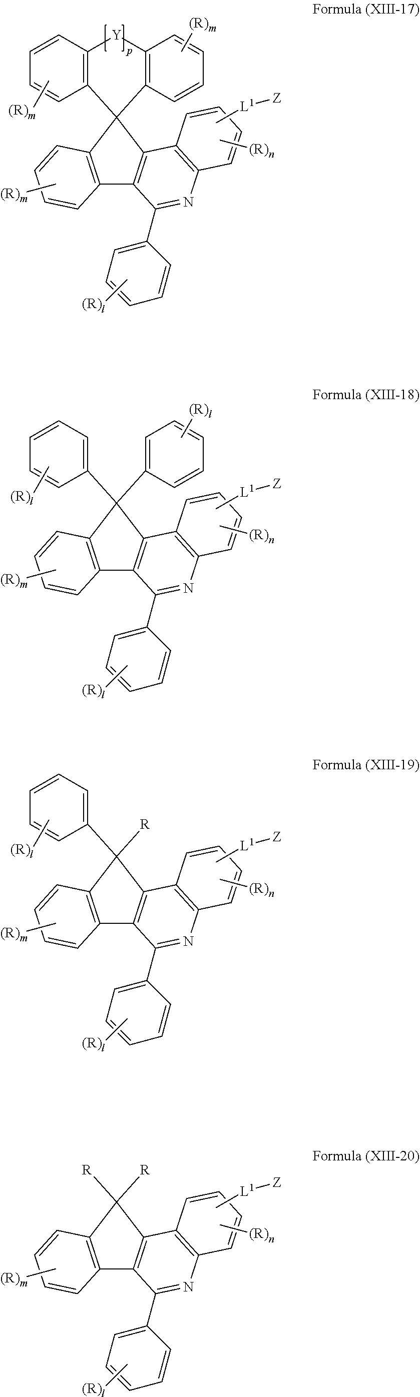

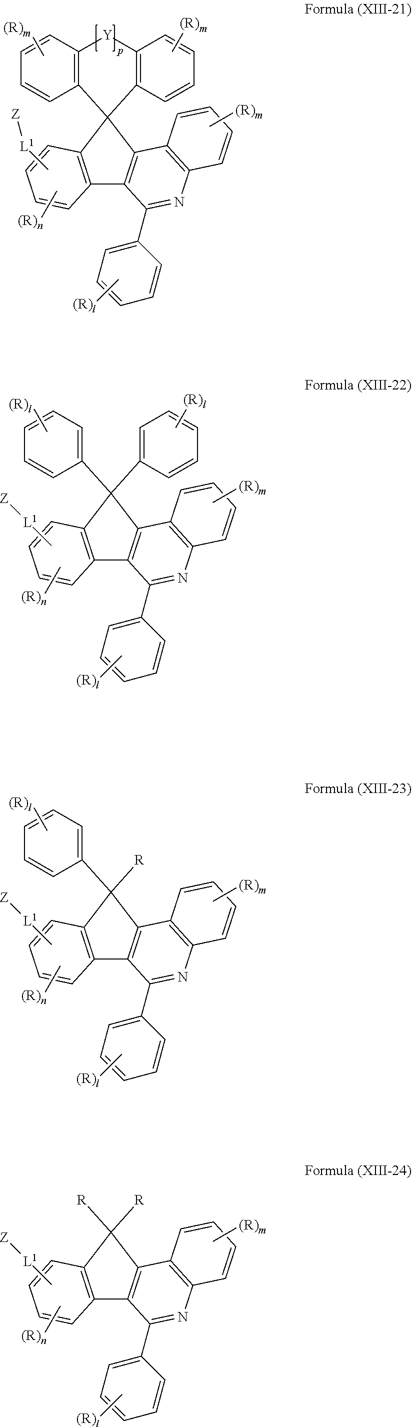

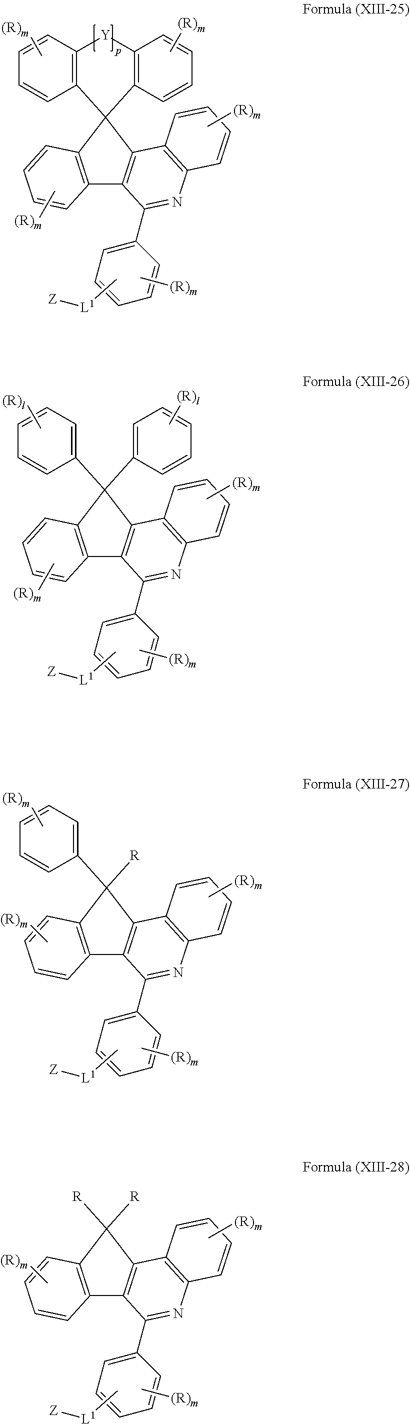

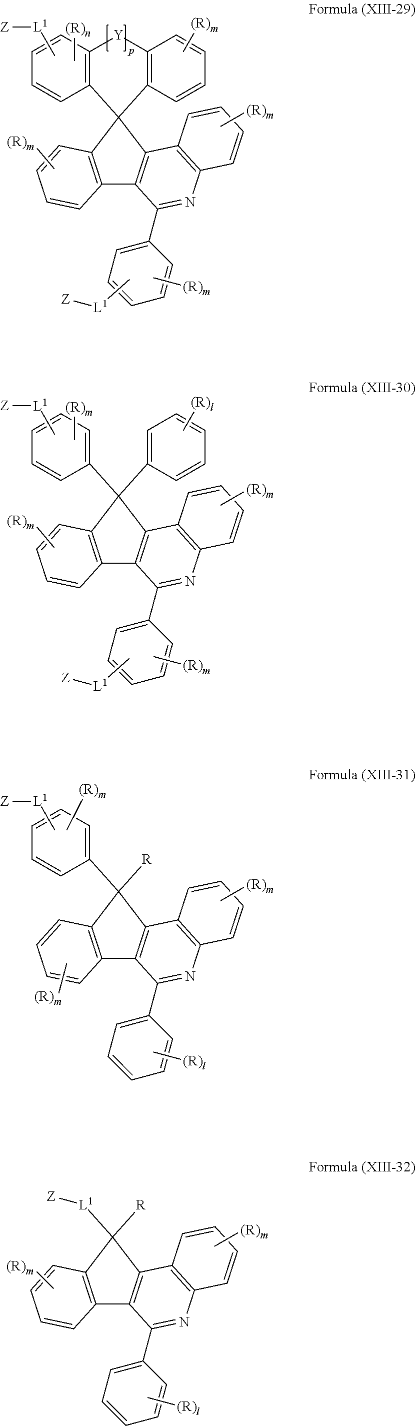

where the symbols R, Y, p, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1.

In addition, particular preference is given to structures (preferably compounds) of the formulae (XIII-13), (XIII-17), (XIII-21), (XIII-25) and (XIII-29):

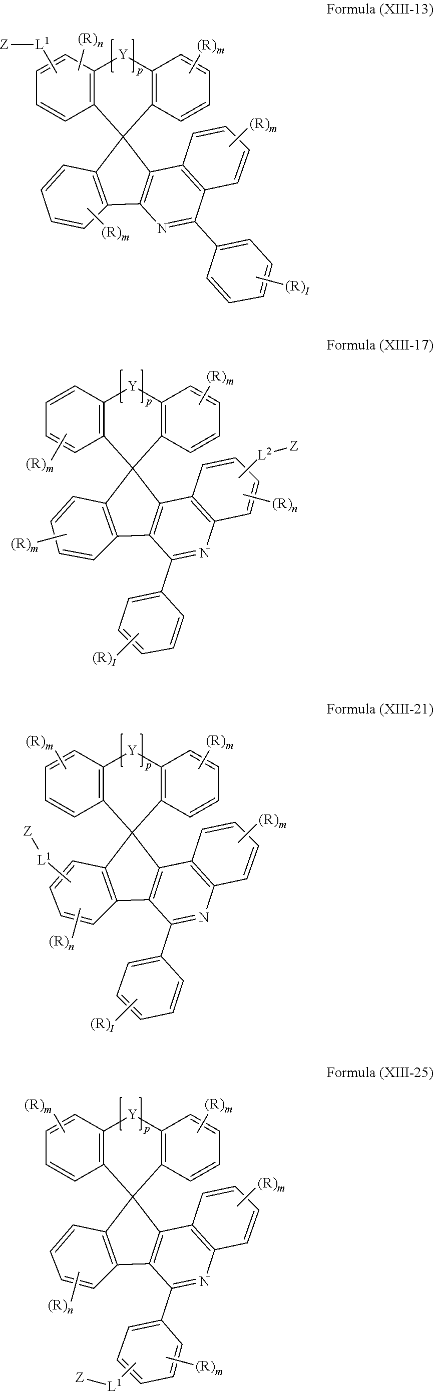

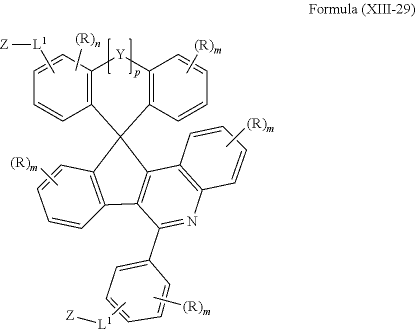

where the symbols R, Y, p, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1. In relation to the formulae (XIII-13), (XIII-17), (XIII-21), (XIII-25) and (XIII-29), preference is given to structures (preferably compounds) in which p=0, such that a bond is formed between the two rings.

More preferably, it may be the case that the compounds of the invention comprise at least one structure of the formula (XIV-1), (XIV-2), (XIV-3), (XIV-4), (XIV-5), (XIV-6), (XIV-7) and/or (XIV-8), where the compounds can preferably be represented by structures of the formula (XIV-1), (XIV-2), (XIV-3), (XIV-4), (XIV-5), (XIV-6), (XIV-7) and/or (XIV-8):

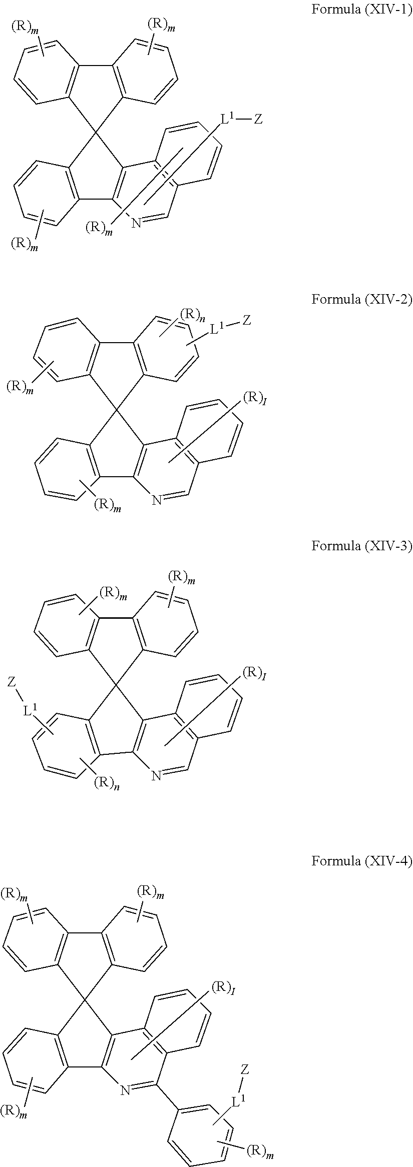

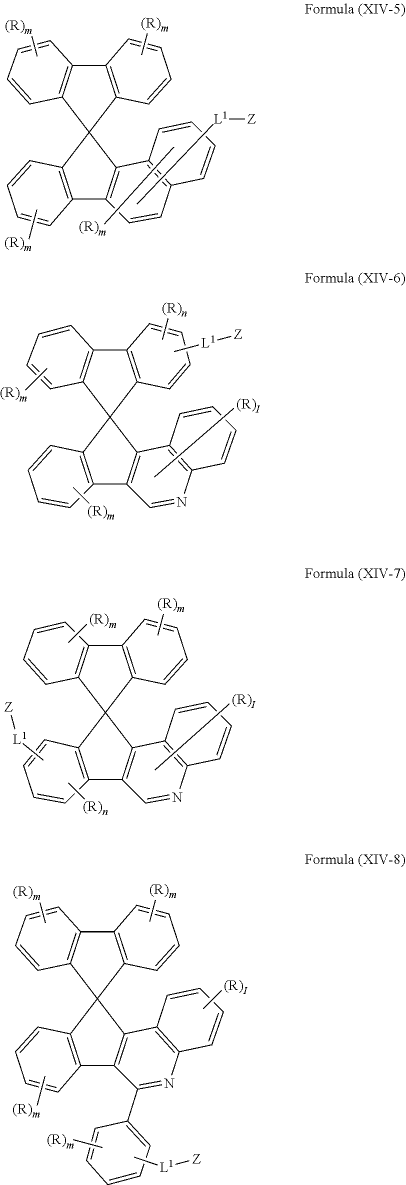

where the symbols R, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1.

It may preferably further be the case that the compounds of the invention comprise at least one structure of the formula (XV-1), (XV-2), (XV-3), (XV-4), (XV-5), (XV-6), (XV-7), (XV-8), (XV-9) and/or (XV-10), where the compounds can preferably be represented by structures of the formula (XV-1), (XV-2), (XV-3), (XV-4), (XV-5), (XV-6), (XV-7), (XV-8), (XV-9) and/or (XV-10):

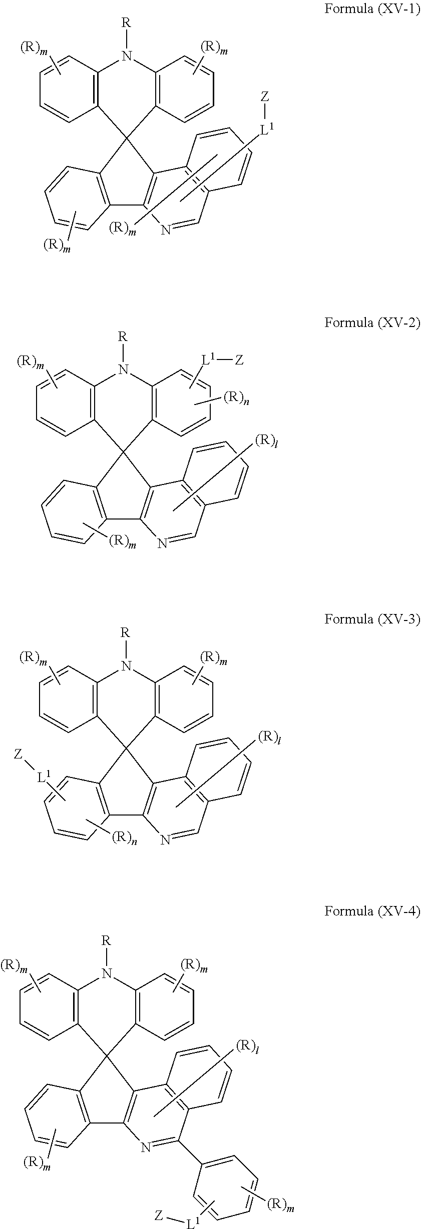

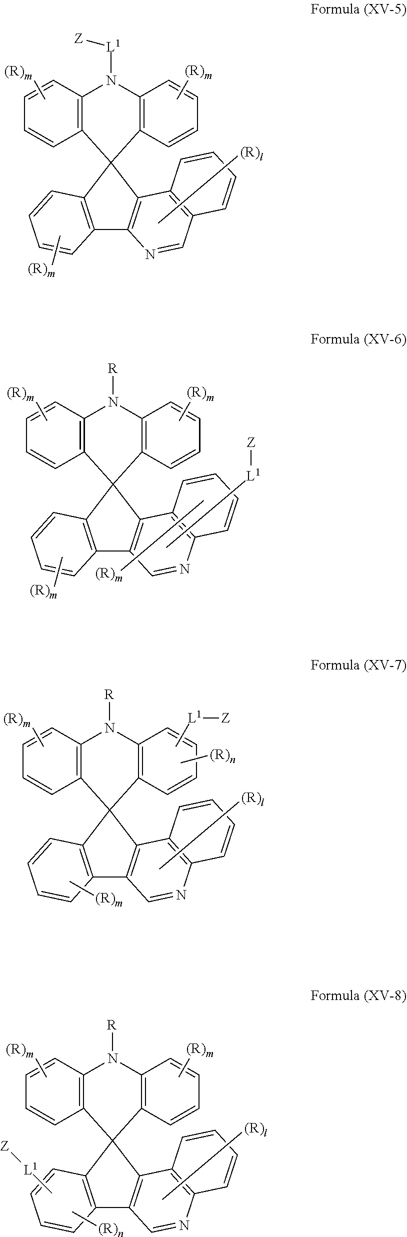

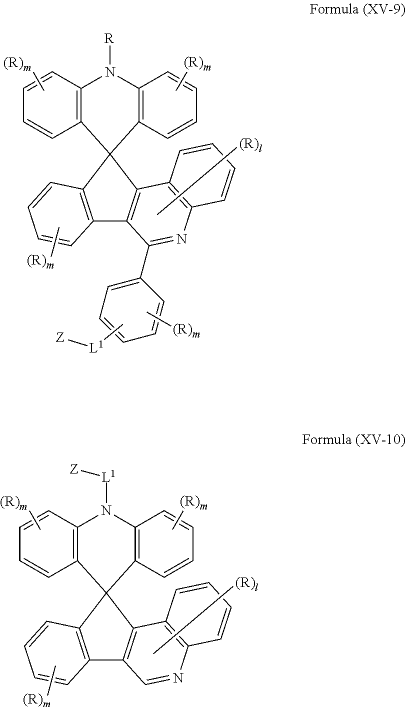

where the symbols R, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1.

In a further-preferred embodiment, it may be the case that the compounds of the invention comprise at least one structure of the formula (XVI-1), (XVI-2), (XVI-3), (XVI-4), (XVI-5), (XVI-6), (XVI-7) and/or (XVI-8), where the compounds can preferably be represented by structures of the formula (XVI-1), (XVI-2), (XVI-3), (XVI-4), (XVI-5), (XVI-6), (XVI-7) and/or (XVI-8):

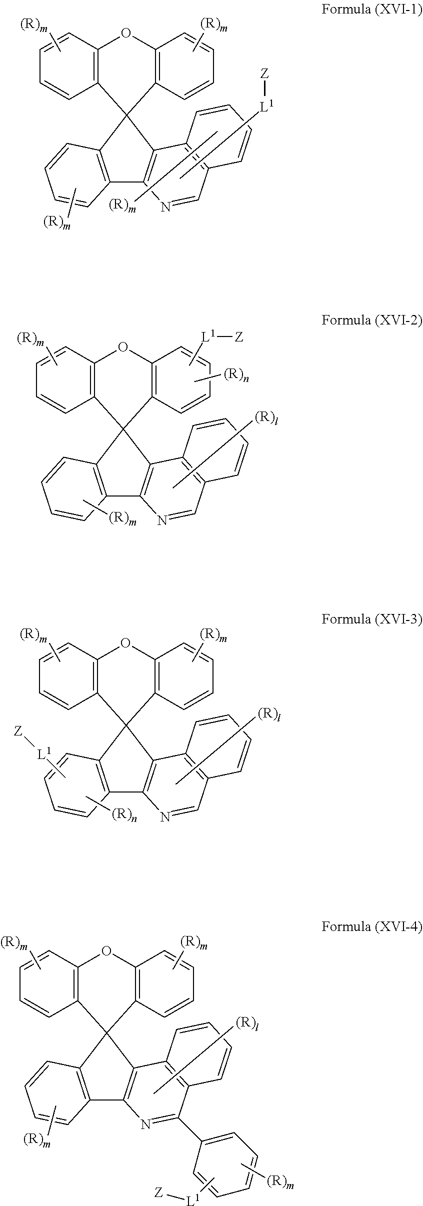

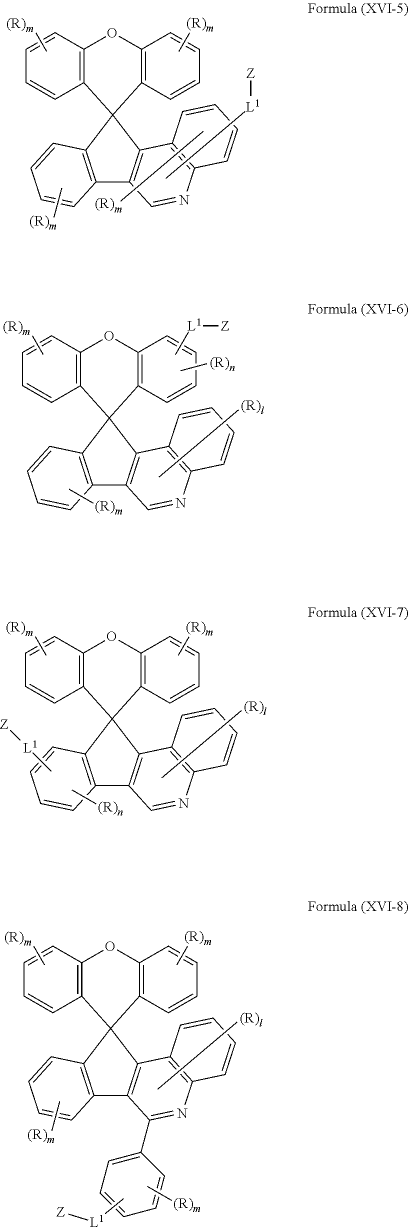

where the symbols R, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1.

It may further be the case that the compounds of the invention comprise at least one structure of the formula (XVII-1), (XVII-2), (XVII-3), (XVII-4), (XVII-5), (XVII-6), (XVII-7) and/or (XVII-8), where the compounds can preferably be represented by structures of the formula (XVII-1), (XVII-2), (XVII-3), (XVII-4), (XVII-5), (XVII-6), (XVII-7) and/or (XVII-8):

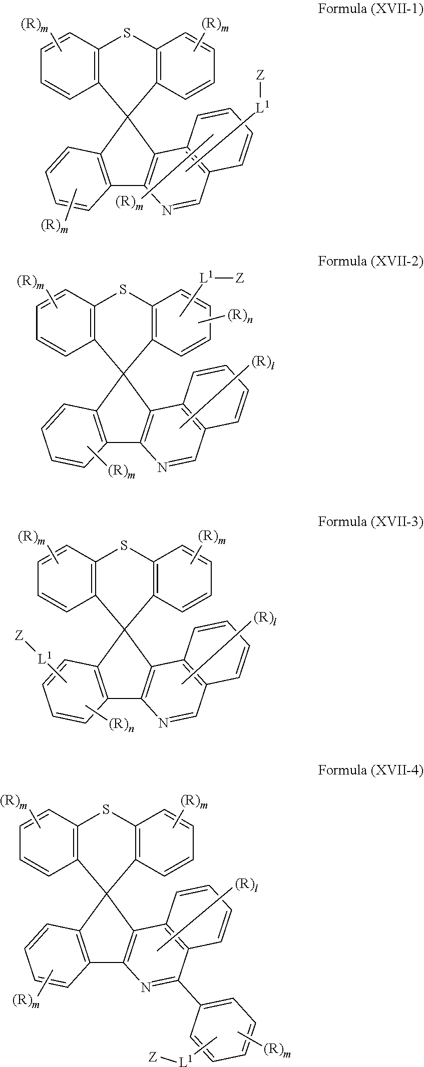

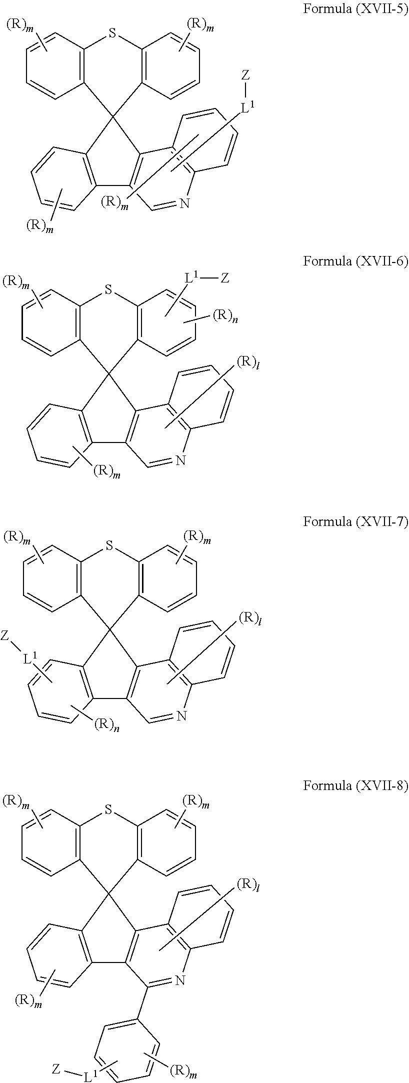

where the symbols R, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1.

It may preferably be the case that the compounds of the invention comprise at least one structure of the formula (XVIII-1), (XVIII-2), (XVIII-3), (XVIII-4), (XVIII-5), (XVIII-6), (XVIII-7), (XVIII-8), (XVIII-9) and/or (XVIII-10), where the compounds can preferably be represented by structures of the formula (XVIII-1), (XVIII-2), (XVIII-3), (XVIII-4), (XVIII-5), (XVIII-6), (XVIII-7), (XVIII-8), (XVIII-9) and/or (XVIII-10):

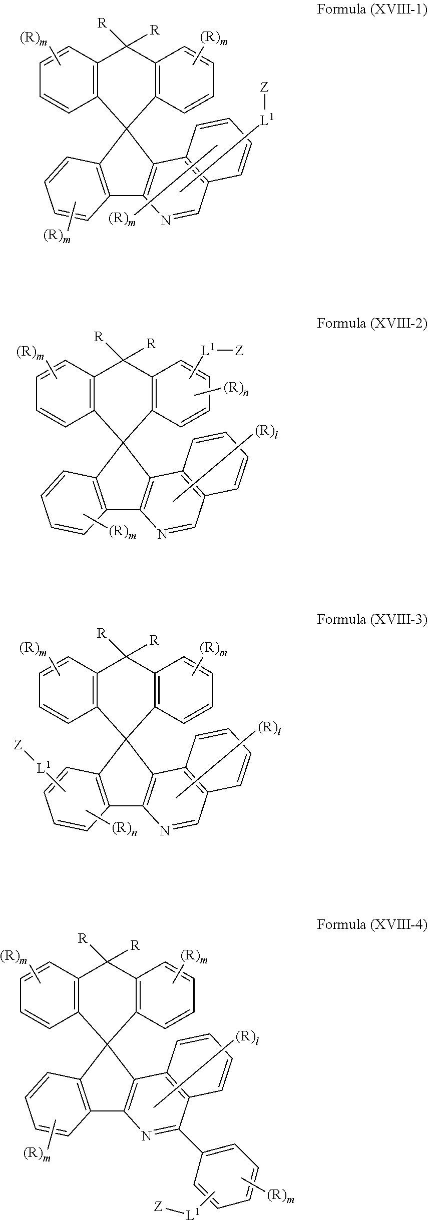

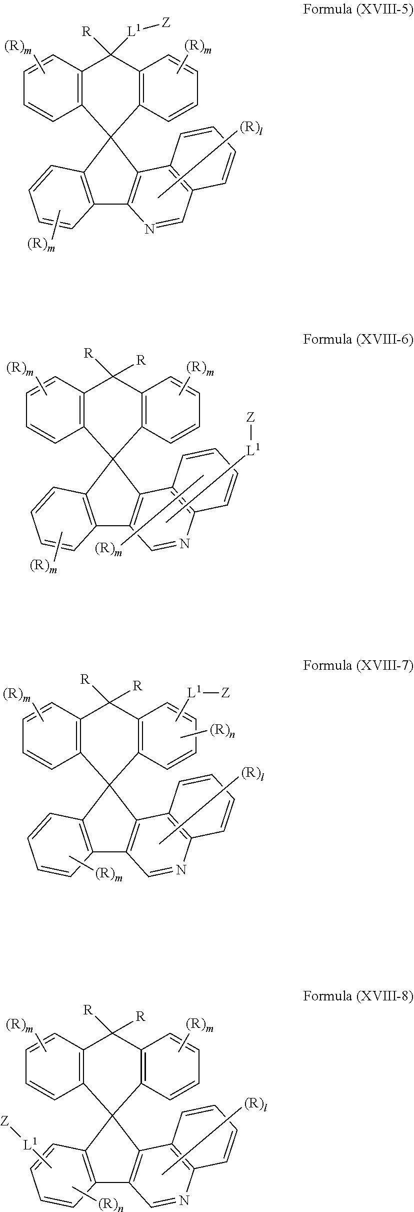

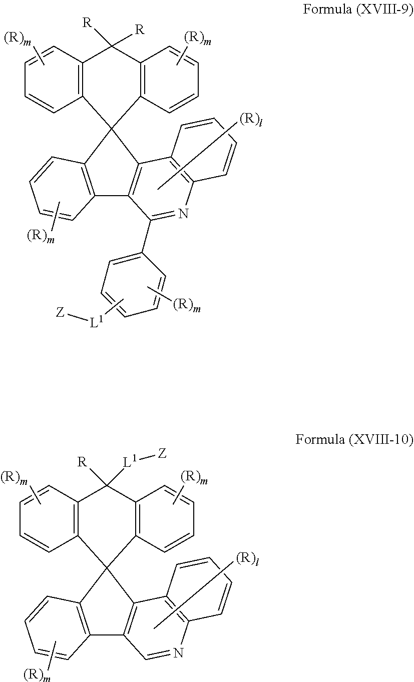

where the symbols R, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1.

In a further preferred embodiment, it may be the case that the compounds of the invention comprise at least one structure of the formula (XIX-1), (XIX-2), (XIX-3), (XIX-4), (XIX-5), (XIX-6), (XIX-7), (XIX-8), (XIX-9) and/or (XIX-10), where the compounds can preferably be represented by structures of the formula (XIX-1), (XIX-2), (XIX-3), (XIX-4), (XIX-5), (XIX-6), (XIX-7), (XIX-8), (XIX-9) and/or (XIX-10):

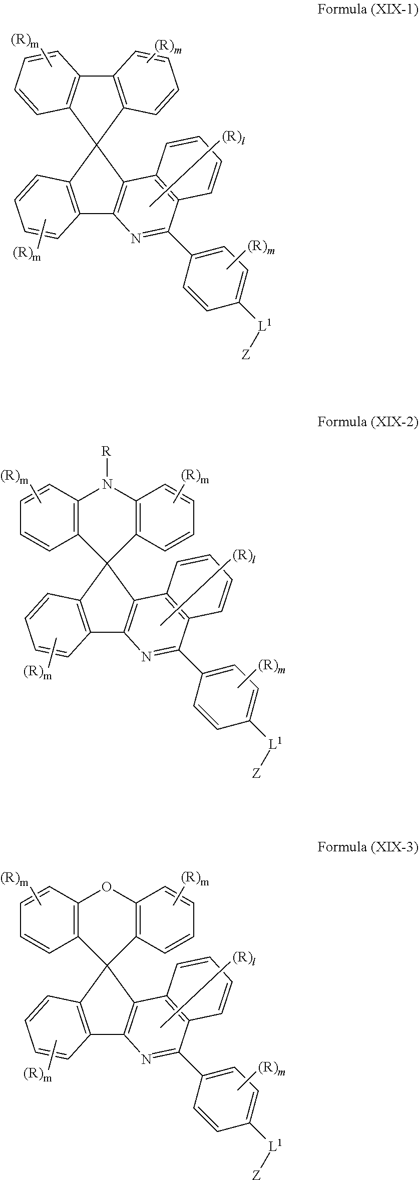

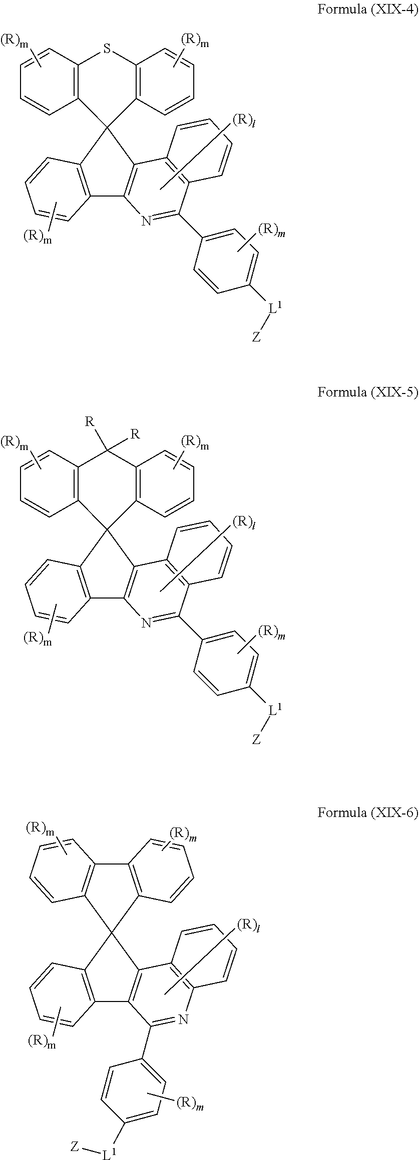

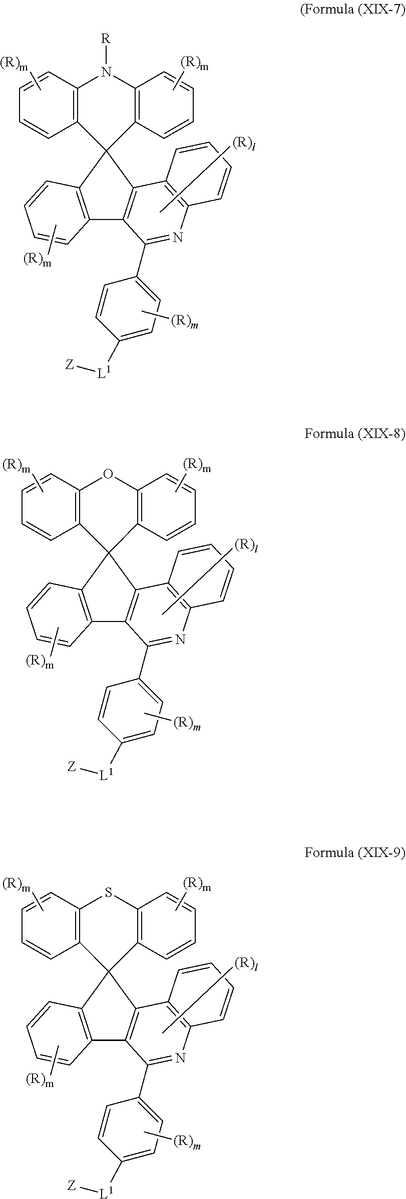

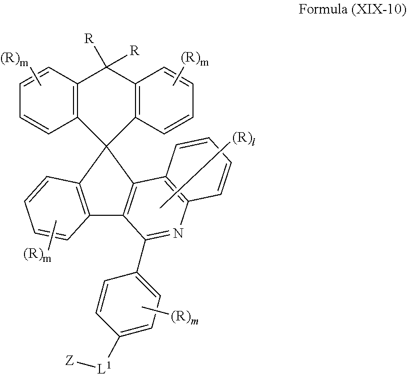

where the symbols R, L¹ and Z used have the definition given above, especially for formulae (Ia), (Ib), (II-1) to (II-16) and/or (L¹-Z), the index I is 1, 2, 3, 4 or 5, preferably 0, 1 or 2, the index m is 0, 1, 2, 3 or 4, preferably 0, 1, 2 or 3, more preferably 0, 1 or 2, and the index n is 0, 1, 2 or 3, preferably 0, 1 or 2, more preferably 0 or 1.

It may further be the case that, in the formulae (XII-1) to (XII-24), (XIII-1) to (XIII-32), (XVI-1) to (XIV-8), (XV-1) to (XV-10), (XVI-1) to (XVI-8), (XVII-1) to (XVII-8); (XVIII-1) to (XVIII-10) and/or (XIX-1) to (XIX-10), the sum total of the indices I, m and n is not more than 5, preferably not more than 3 and more preferably not more than 1.

It may further be the case that at least one R group, preferably all of the R groups, in the formulae (Ia), (Ib), (II-1) to (II-16), (III-1) to (III-16), (IV-1) to (IV-24), (V-1) to (V-32), (VI-1) to (VI-8), (VII-1) to (VII-8), (VIII-1) to (VIII-10), (IX-1) to (IX-8), (X-1) to (X-8), (XI-1) to (XI-10), (XII-1) to (XII-24), (XIII-1) to (XIII-32), (XVI-1) to (XIV-8), (XV-1) to (XV-10), (XVI-1) to (XVI-8), (XVII-1) to (XVII-8); (XVIII-1) to (XVIII-10) and/or (XIX-1) to (XIX-10) is/are a group that can be represented by the radicals of the formula R¹, as set out above and hereinafter, where R¹ has the definition given above, especially for formula (Ia) or (Ib).

The above-detailed hole transport groups of the formulae (H-1) to (H-26) and/or electron transport group-comprising radicals, preferably electron transport group-comprising radicals of formula (QL), are preferred R¹ radicals, in which case the R¹ groups in the formulae (H-1) to (H-26), (QL), and/or (Q-1) to (Q-44) should be replaced by R² radicals.

It may further be the case that the L¹-Z group in the above formulae, inter alia in the formulae (IV-1) to (IV-24), (V-1) to (V-32), (VI-1) to (VI-8), (VII-1) to (VII-8), (VIII-1) to (VIII-10), (IX-1) to (IX-8), (X-1) to (X-8), (XI-1) to (XI-10), (XII-1) to (XII-24), (XIII-1) to (XIII-32), (XVI-1) to (XIV-8), (XV-1) to (XV-10), (XVI-1) to (XVI-8), (XVII-1) to (XVII-8), (XVIII-1) to (XVIII-10) and/or (XIX-1) to (XIX-10), comprises a hole transport group and/or an electron transport group or is a hole transport group and/or an electron transport group.

It may further be the case that the symbol Z in formula L¹-Z or in a structure/compound of the formulae (IV-1) to (IV-24), (V-1) to (V-32), (VI-1) to (VI-8), (VII-1) to (VII-8), (VIII-1) to (VIII-10), (IX-1) to (IX-8), (X-1) to (X-8), (XI-1) to (XI-10), (XII-1) to (XII-24), (XIII-1) to (XIII-32), (XVI-1) to (XIV-8), (XV-1) to (XV-10), (XVI-1) to (XVI-8), (XVII-1) to (XVII-8), (XVIII-1) to (XVIII-10) and/or (XIX-1) to (XIX-10) is a group selected from the formulae (Z-1) to (Z¹-91):

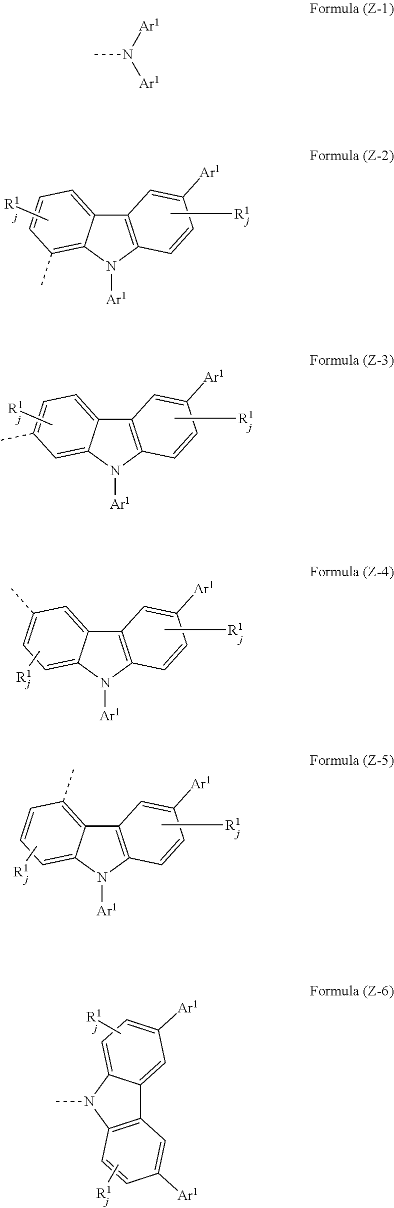

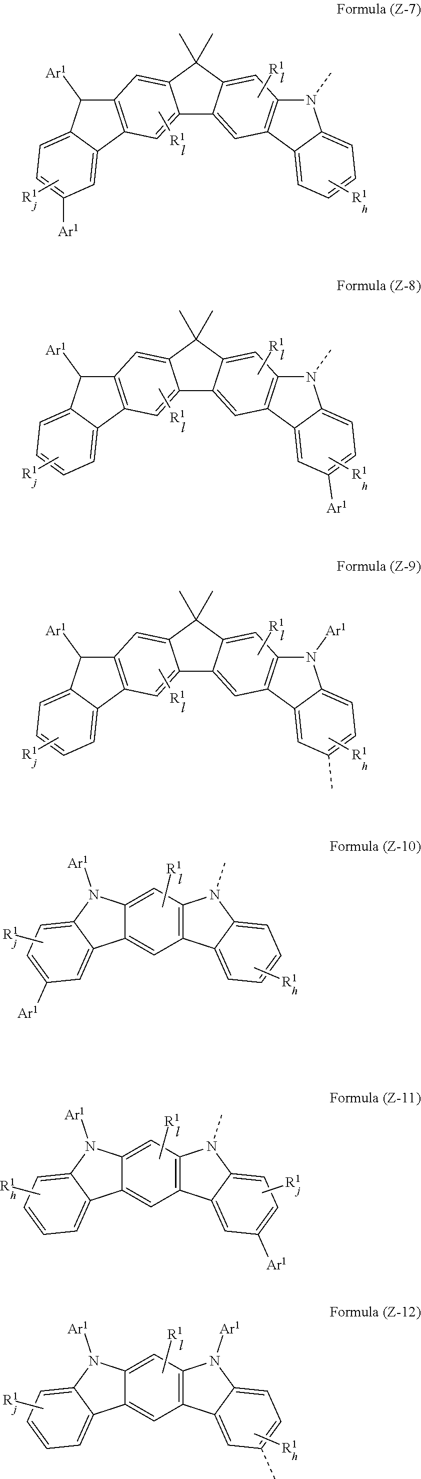

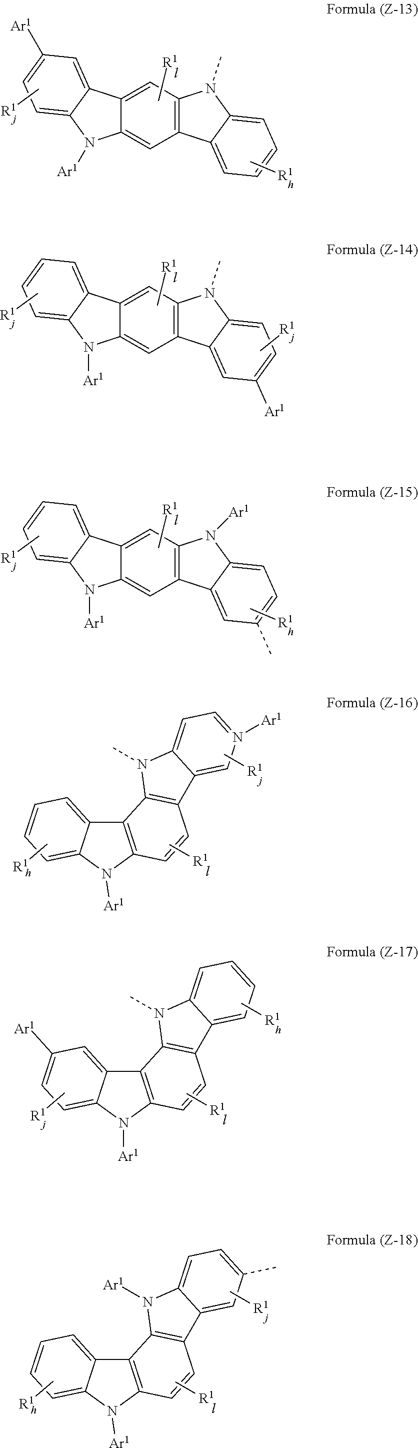

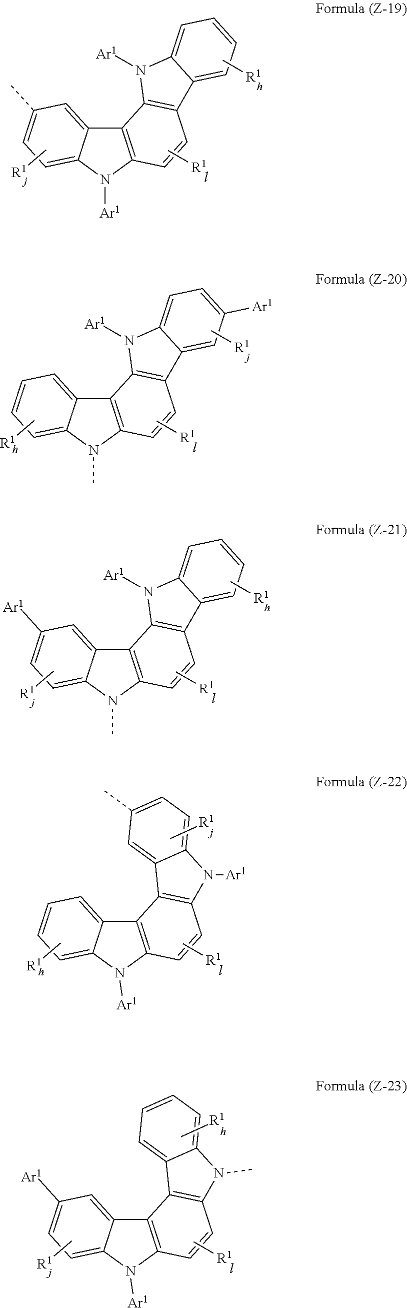

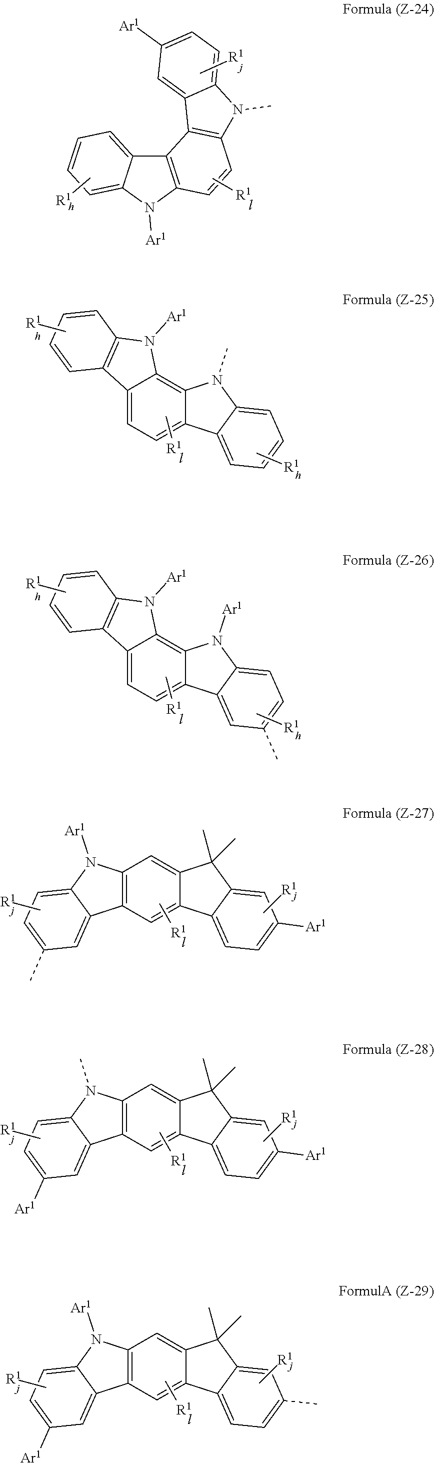

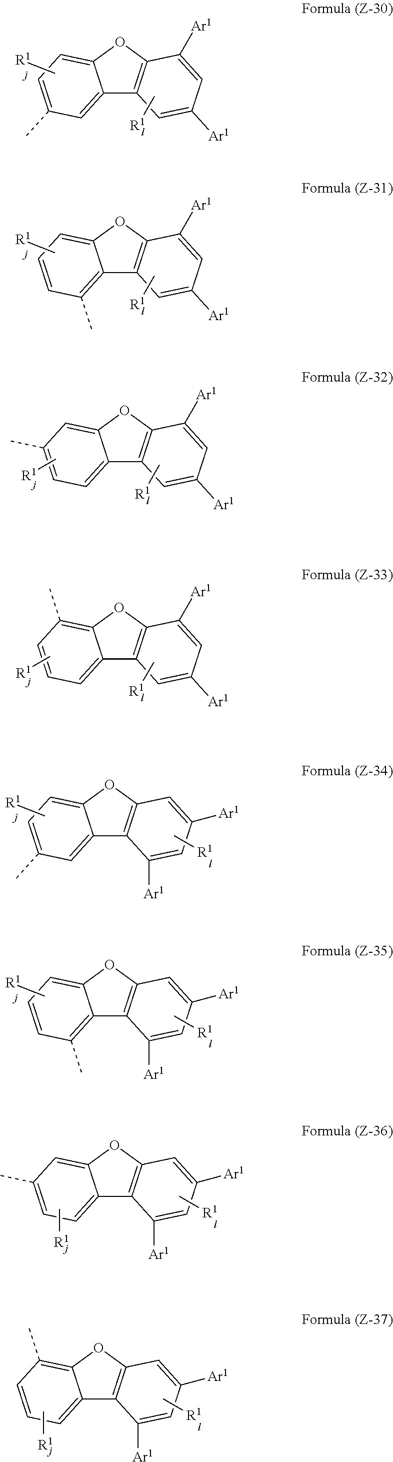

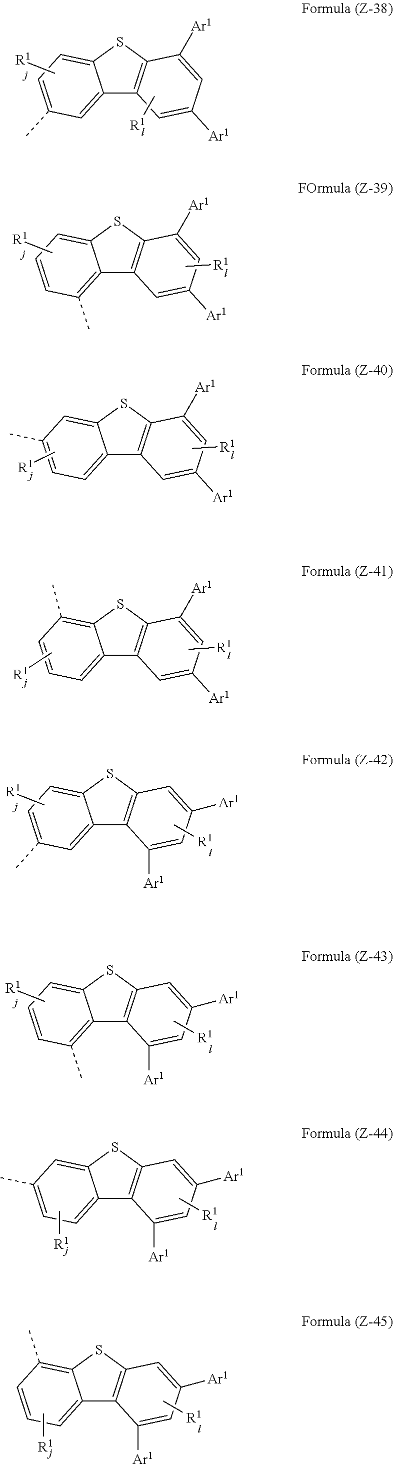

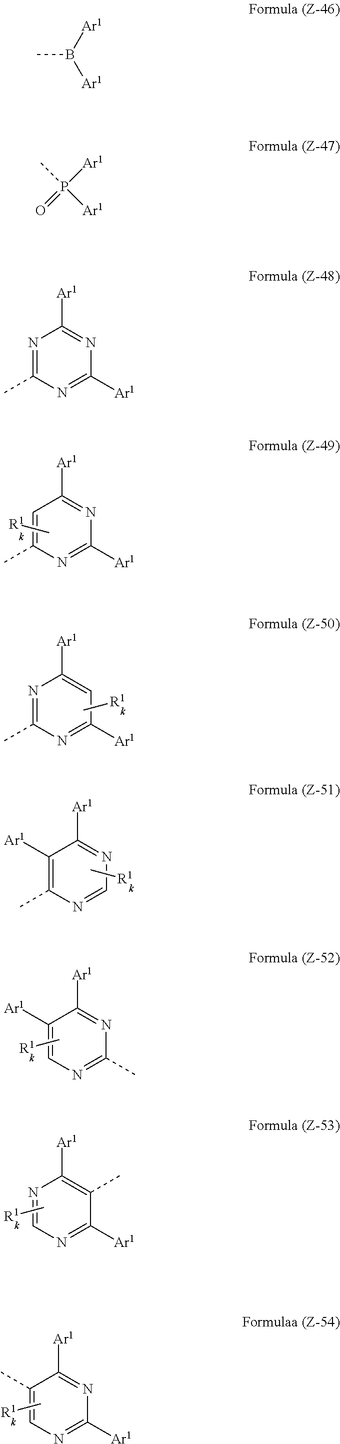

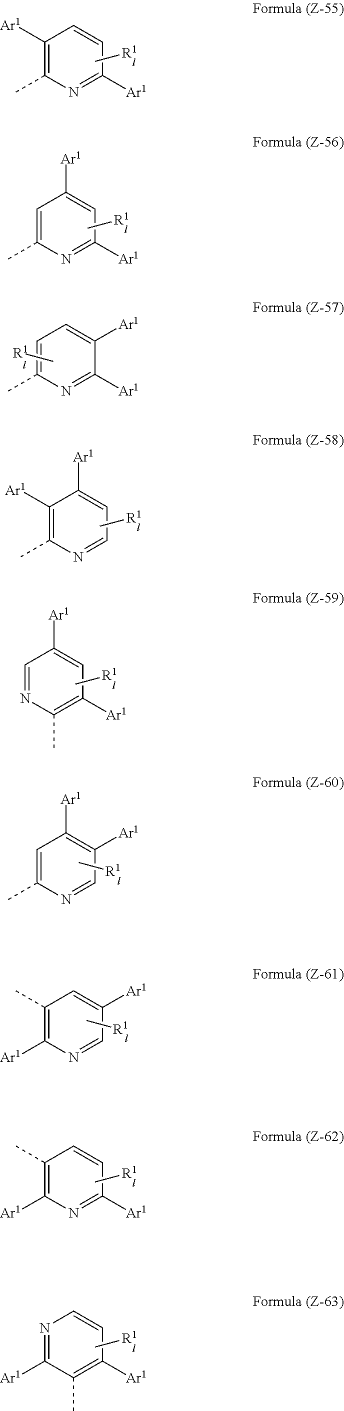

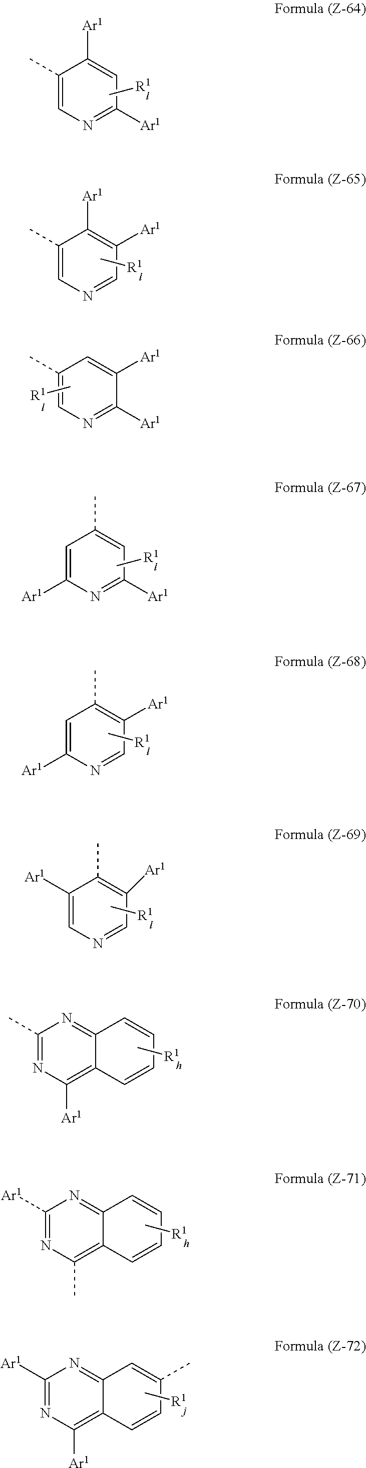

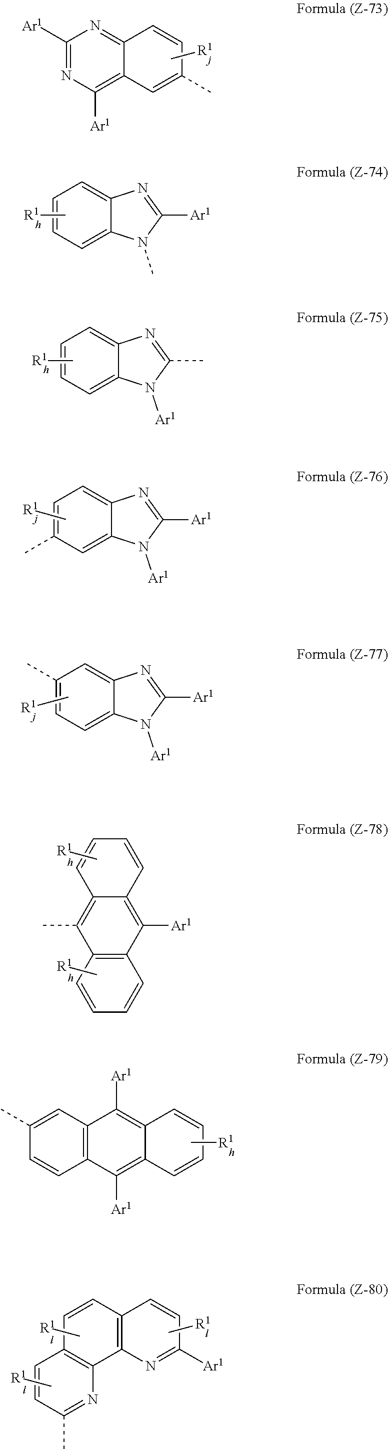

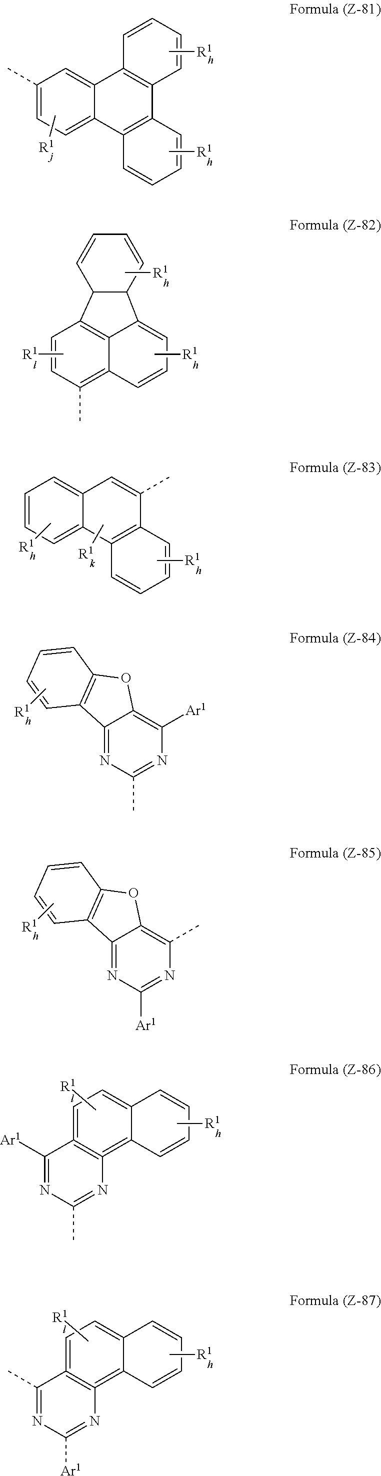

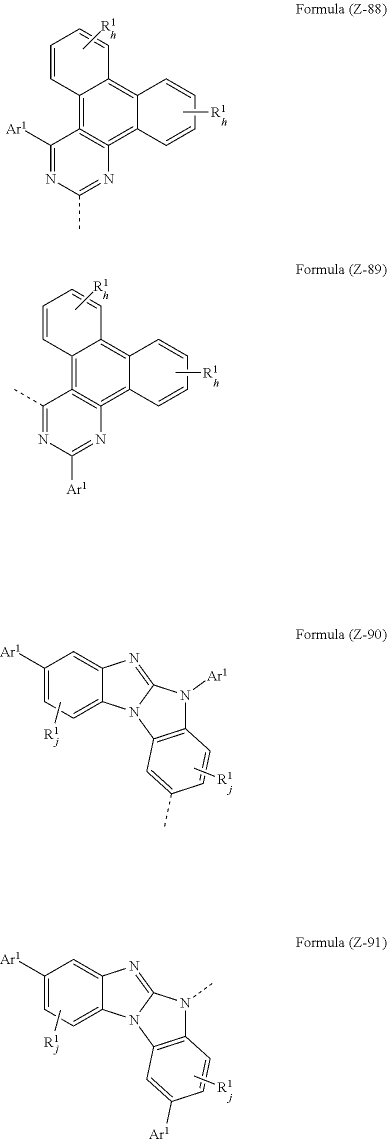

where the symbols used are as follows:

k at each instance is independently 0 or 1;

i at each instance is independently 0, 1 or 2;

j at each instance is independently 0, 1, 2 or 3;

h at each instance is independently 0, 1, 2, 3 or 4;

the dotted bond marks the position of attachment; and

Ar¹, R¹ have the definition given above, especially for formula (Ia) or (Ib).

Preferably, the L′ group may form through-conjugation with the Z group and the atom to which the L′ group or according to formula (L′-Z) is bonded. Through-conjugation of the aromatic or heteroaromatic systems is formed as soon as direct bonds are formed between adjacent aromatic or heteroaromatic rings. A further bond between the aforementioned conjugated groups, for example via a sulfur, nitrogen or oxygen atom or a carbonyl group, is not detrimental to conjugation.

In a further preferred embodiment of the invention, L′ is a bond or an aromatic or heteroaromatic ring system which has 5 to 14 aromatic or heteroaromatic ring atoms, preferably an aromatic ring system which has 6 to 12 carbon atoms, and which may be substituted by one or more R¹ radicals, but is preferably unsubstituted, where R¹ may have the definition given above, especially for formulae (Ia) and/or (Ib). More preferably, L′ is an aromatic ring system having 6 to 10 aromatic ring atoms or a heteroaromatic ring system having 6 to 13 heteroaromatic ring atoms, each of which may be substituted by one or more R¹ radicals, but is preferably unsubstituted, where R¹ may have the definition given above, especially for formula (Ia) and/or (Ib).

Further preferably, the symbol L¹ shown in formula (L¹-Z) inter alia is the same or different at each instance and is a bond or an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded to the respective atom of the further group directly, i.e. via an atom of the aromatic or heteroaromatic group.

It may additionally be the case that the L¹ group shown in formula (L¹-Z) comprises an aromatic ring system having not more than two fused aromatic and/or heteroaromatic 6-membered rings, preferably does not comprise any fused aromatic or heteroaromatic ring system. Accordingly, naphthyl structures are preferred over anthracene structures. In addition, fluorenyl, spirobifluorenyl, dibenzofuranyl and/or dibenzothienyl structures are preferred over naphthyl structures.

Particular preference is given to structures having no fusion, for example phenyl, biphenyl, terphenyl and/or quaterphenyl structures.

Examples of suitable aromatic or heteroaromatic ring systems L¹ are selected from the group consisting of ortho-, meta- or para-phenylene, ortho-, meta- or para-biphenylene, terphenylene, especially branched terphenylene, quaterphenylene, especially branched quaterphenylene, fluorenylene, spirobifluorenylene, dibenzofuranylene, dibenzothienylene and carbazolylene, each of which may be substituted by one or more R¹ radicals, but are preferably unsubstituted.

It may further be the case that the L¹ group shown in formula (L¹-Z) inter alia has not more than 1 nitrogen atom, preferably not more than 2 heteroatoms, especially preferably not more than one heteroatom and more preferably no heteroatom.

It may preferably be the case that the L¹ group in the structural element L¹-Z of formulae (IV-1) to (IV-24), (V-1) to (V-32), (VI-1) to (VI-8), (VII-1) to (VII-8), (VIII-1) to (VIII-10), (IX-1) to (IX-8), (X-1) to (X-8), (XI-1) to (XI-10), (XII-1) to (XII-24), (XIII-1) to (XIII-32), (XIV-1) to (XIV-8), (XV-1) to (XV-10), (XVI-1) to (XVI-8), (XVII-1) to (XVII-8), (XVIII-1) to (XVIII-10) and/or (XIX-1) to (XIX-10), and/or, in formula QL, the L¹ group or the Are group of formulae H-1 to H-44, is a bond or a group selected from the formulae (L¹-1) to (L¹-167):

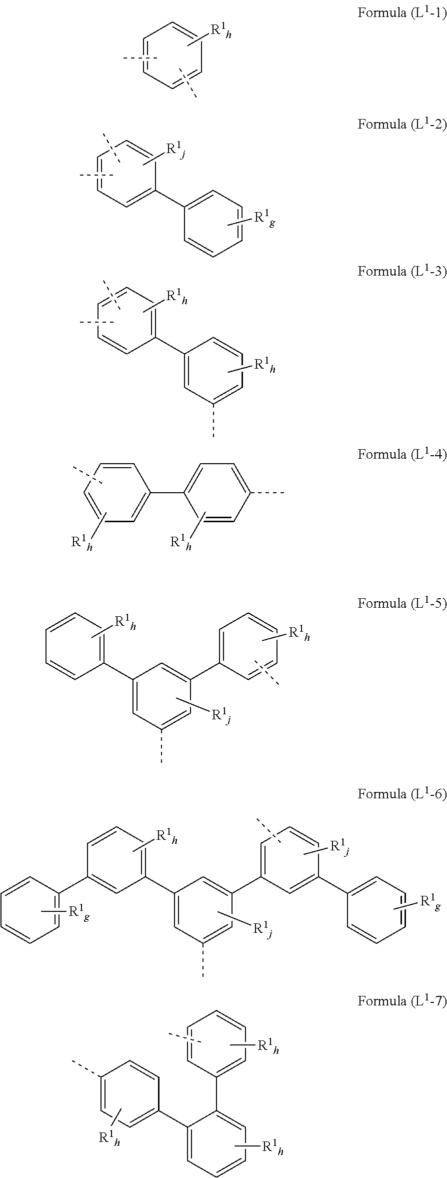

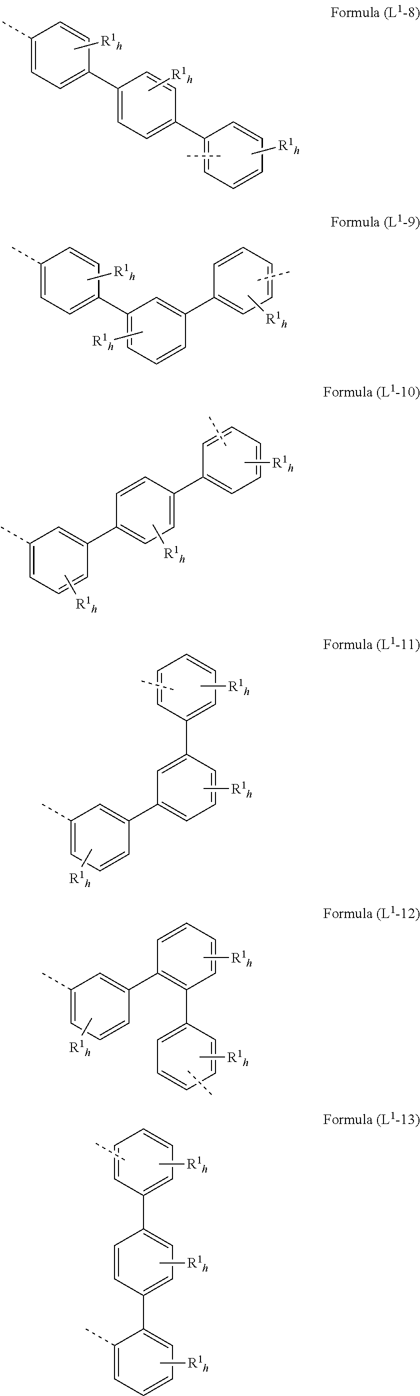

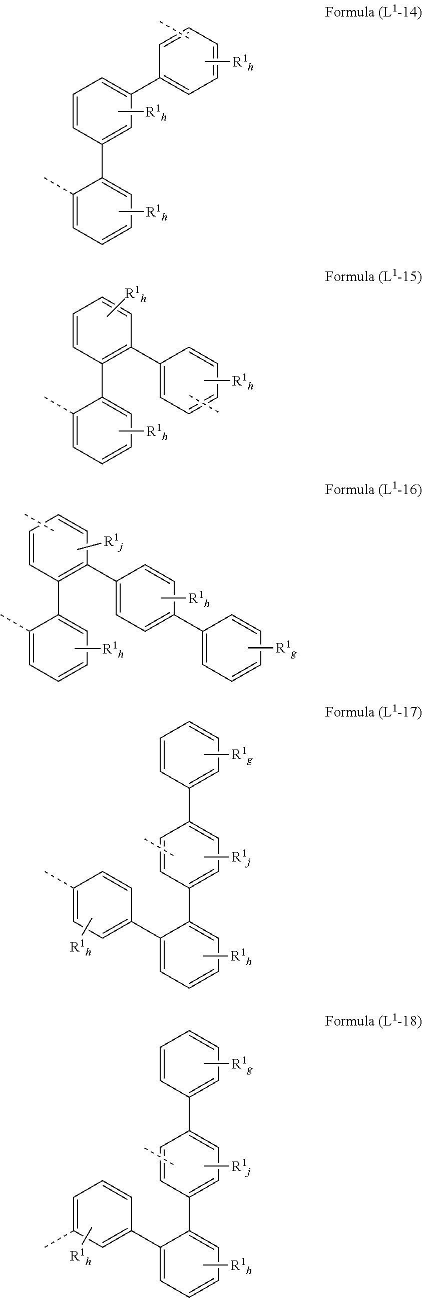

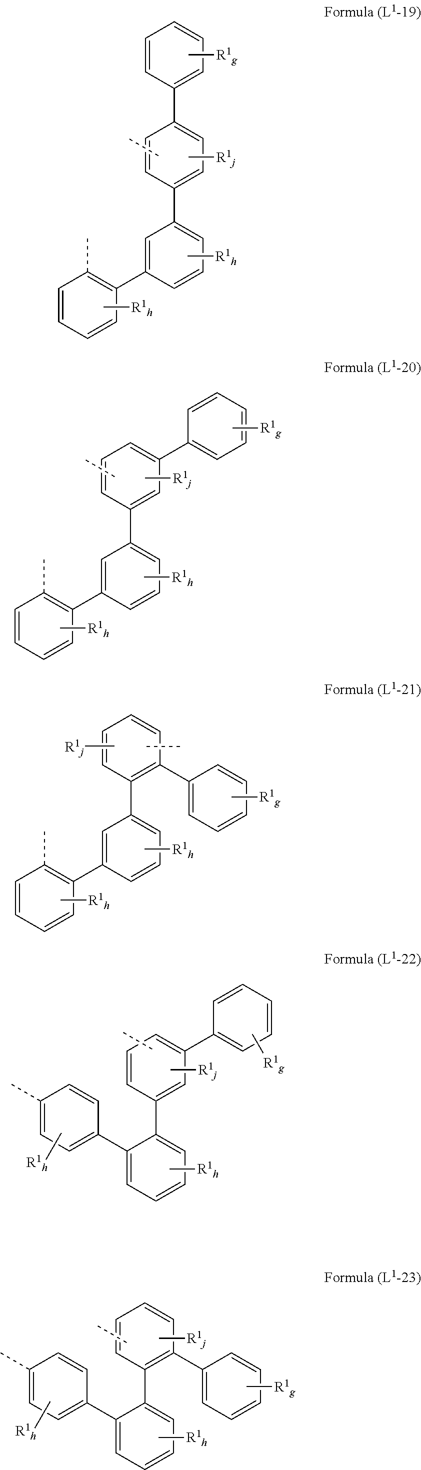

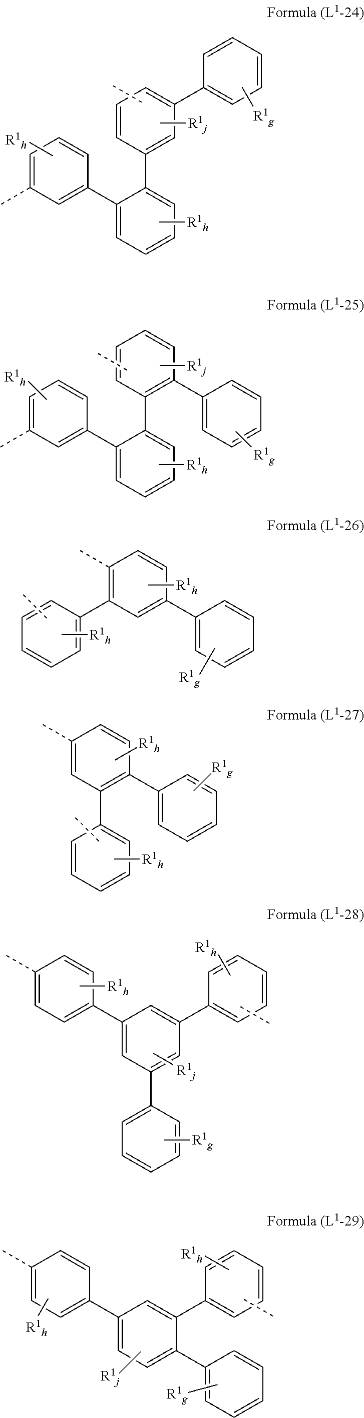

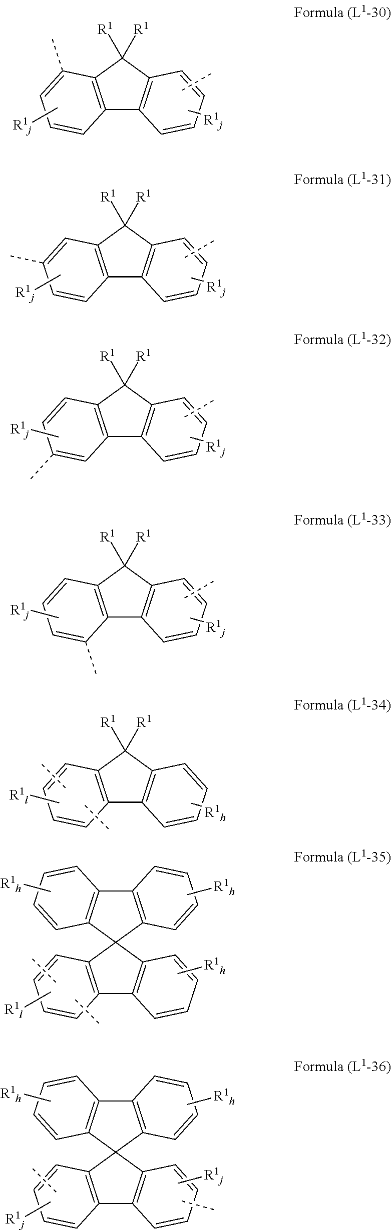

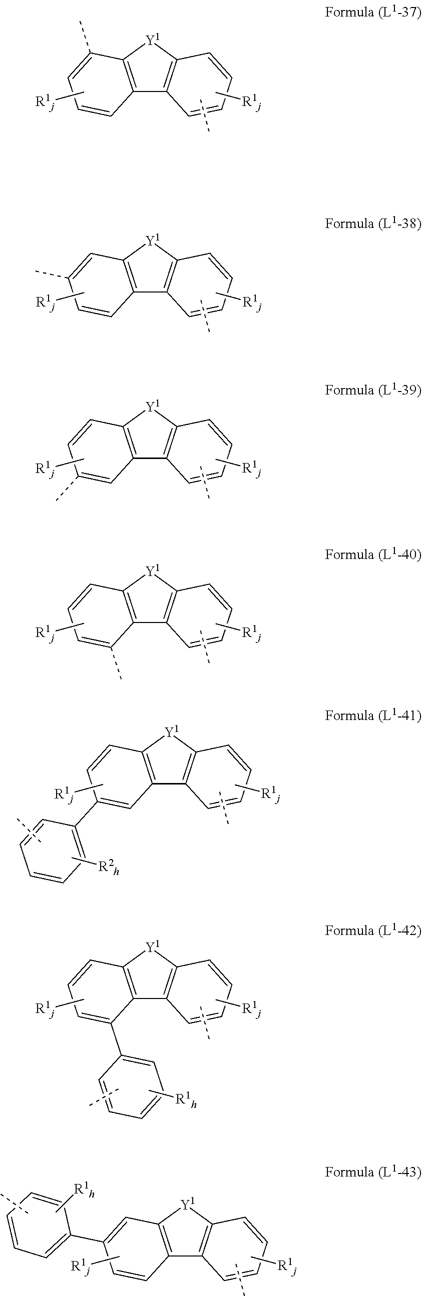

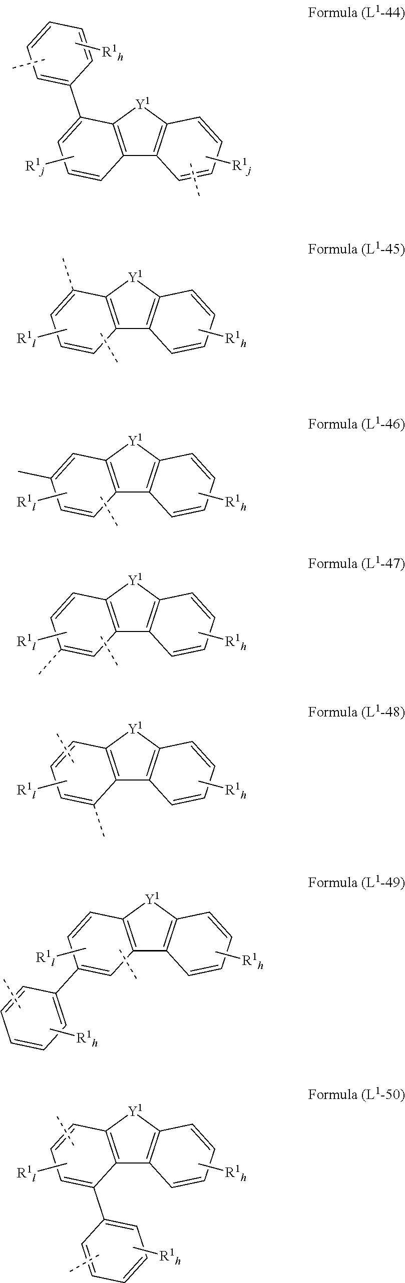

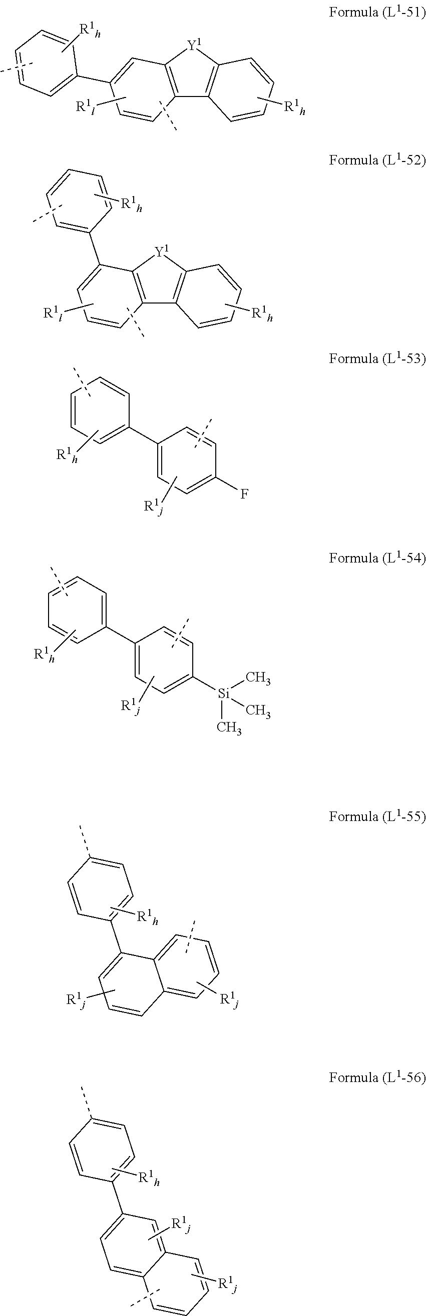

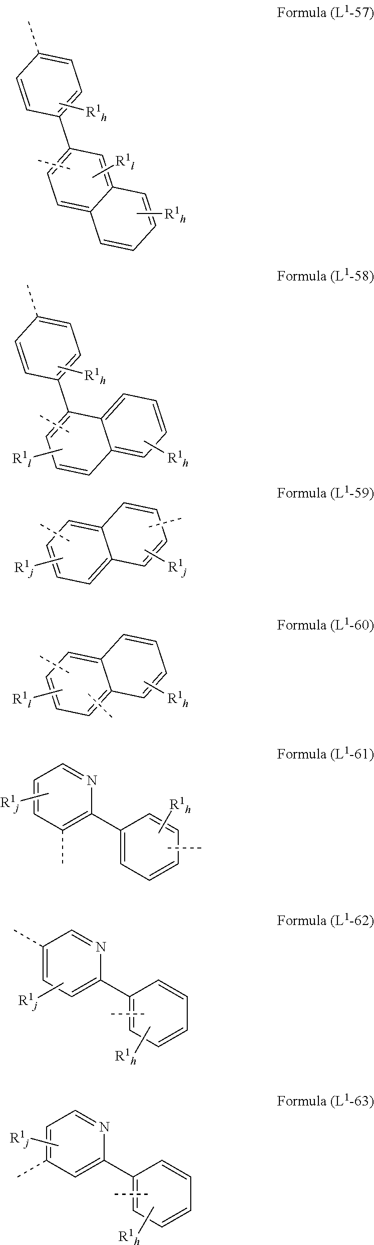

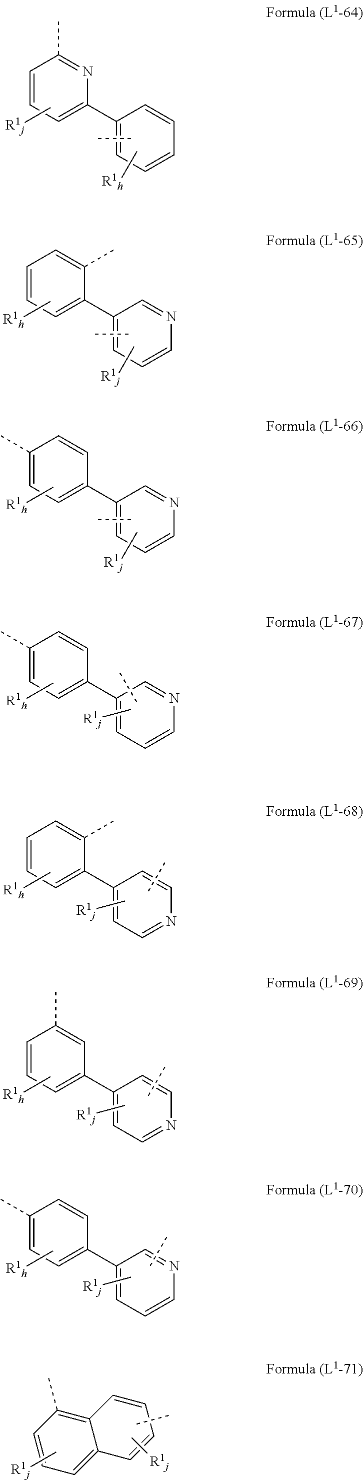

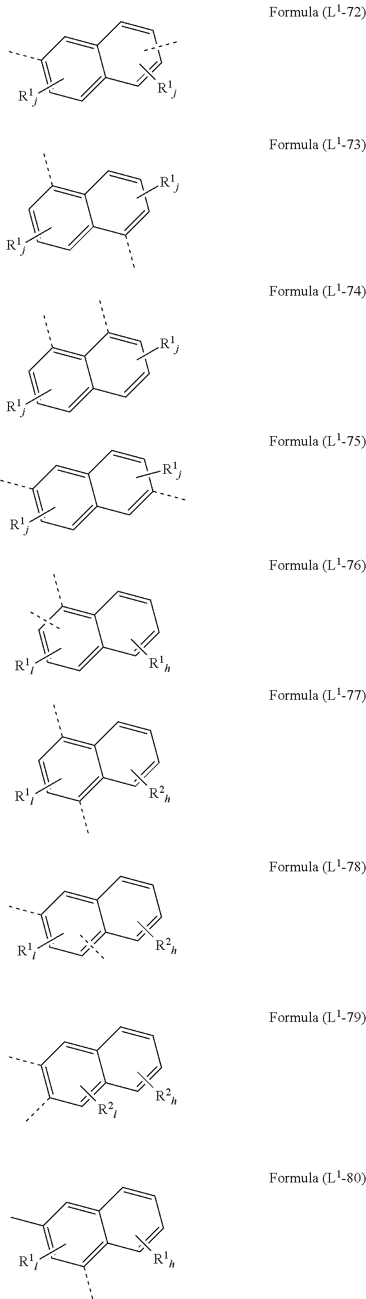

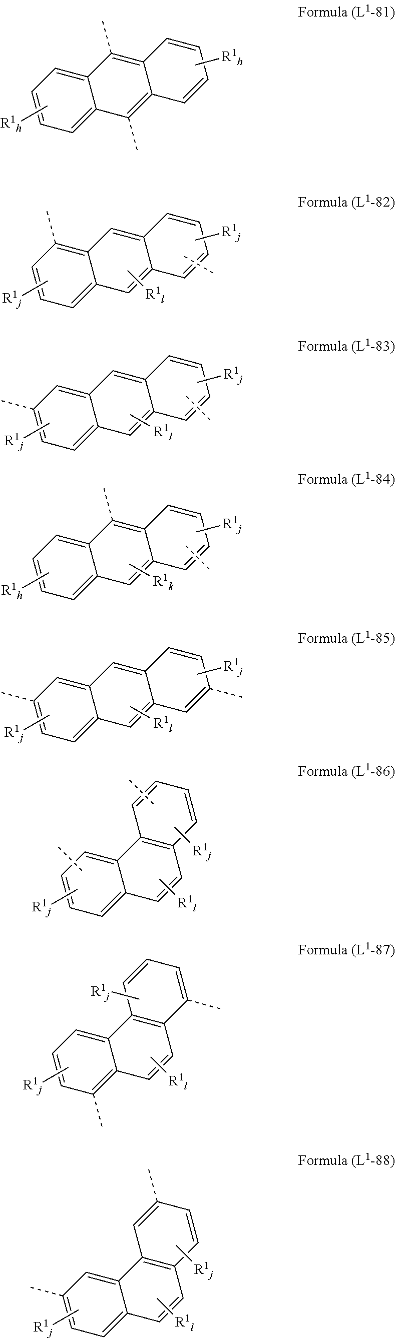

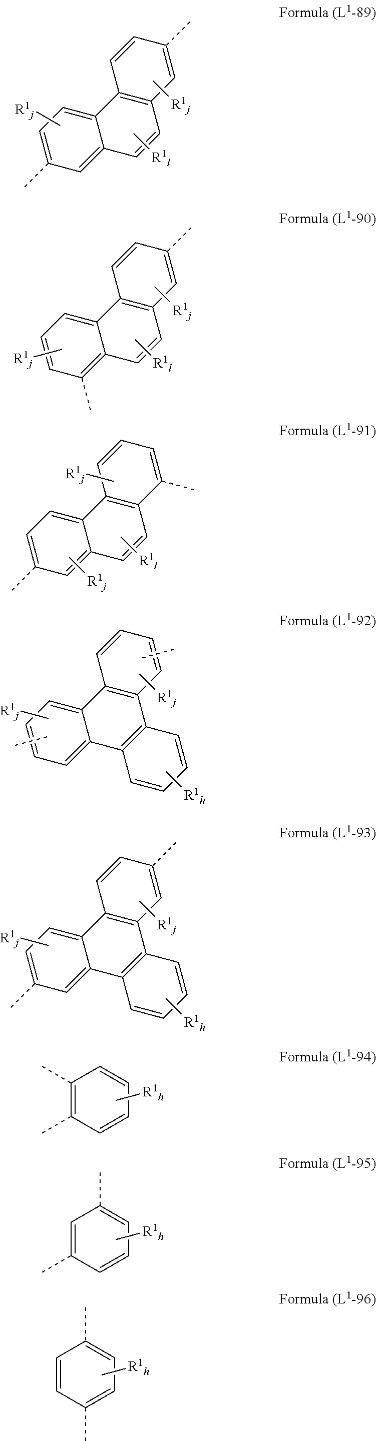

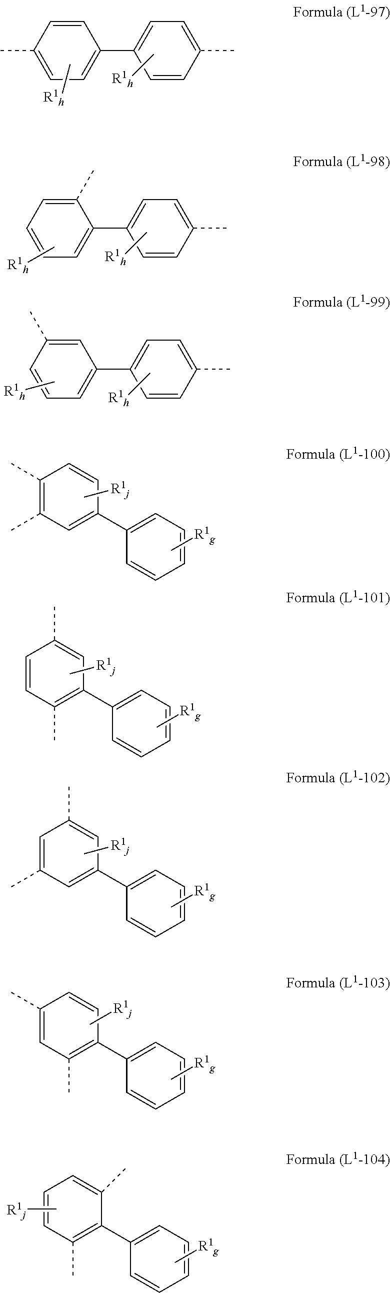

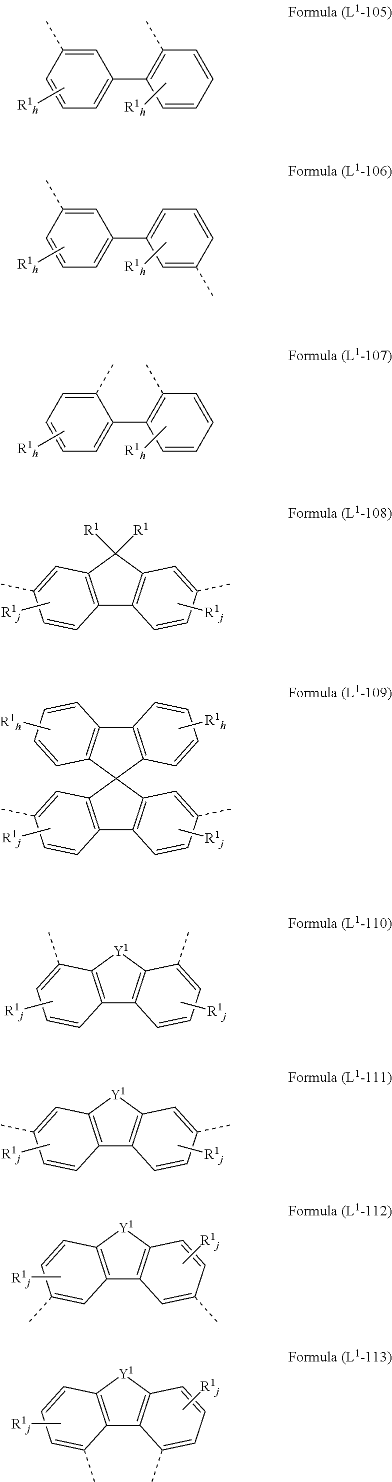

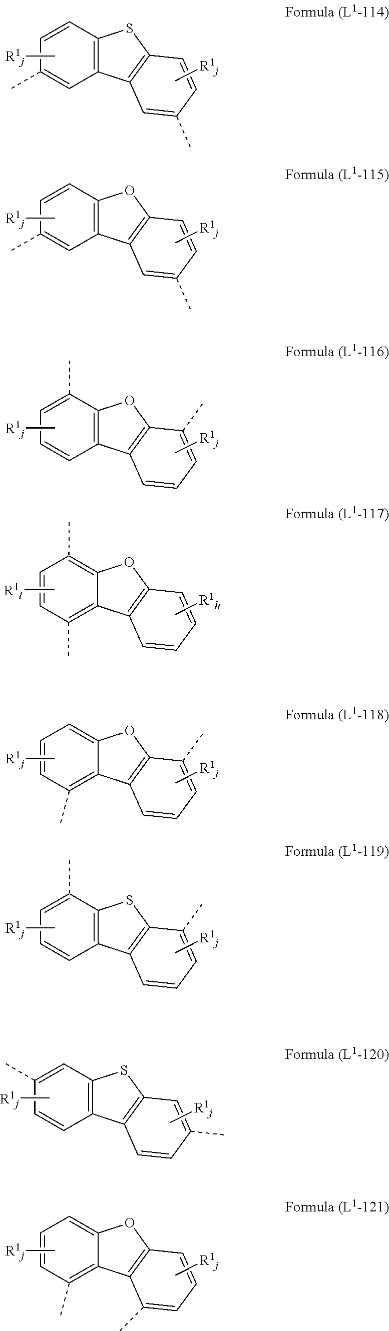

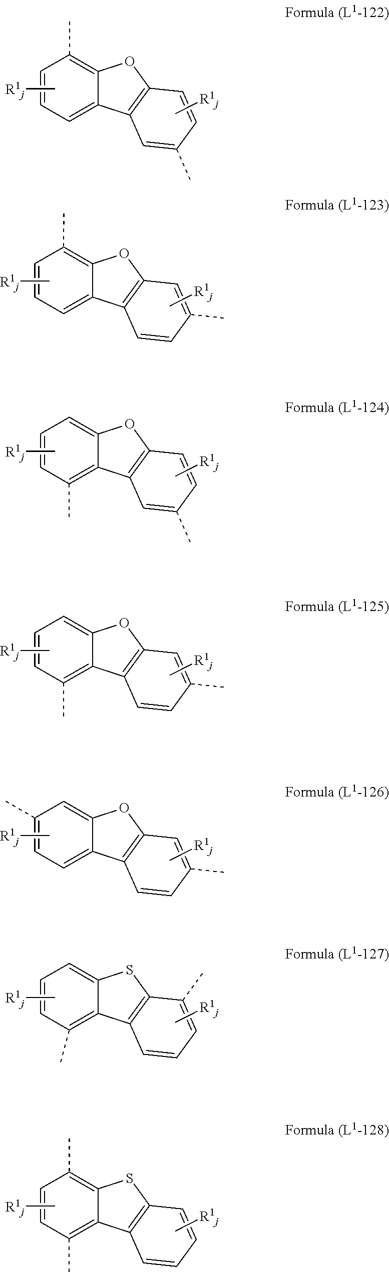

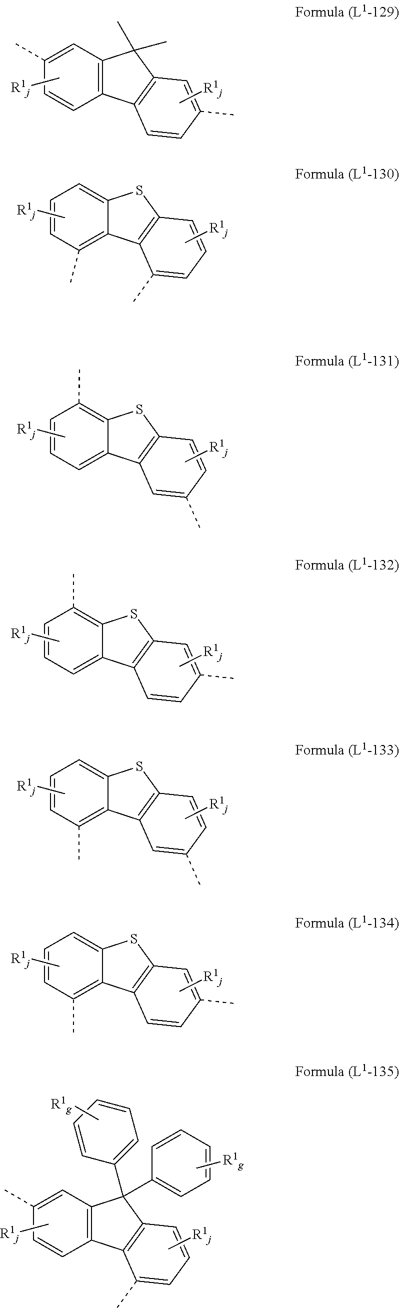

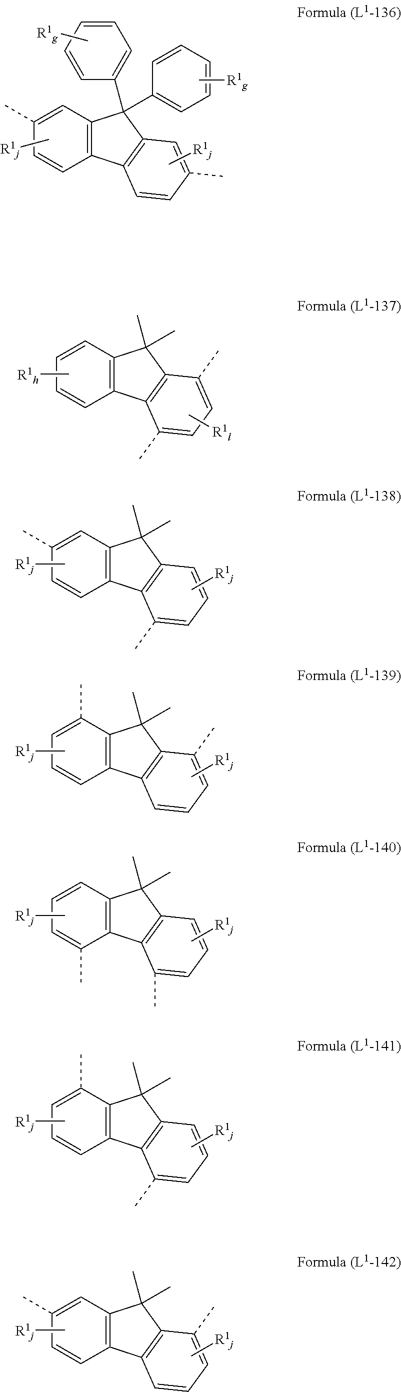

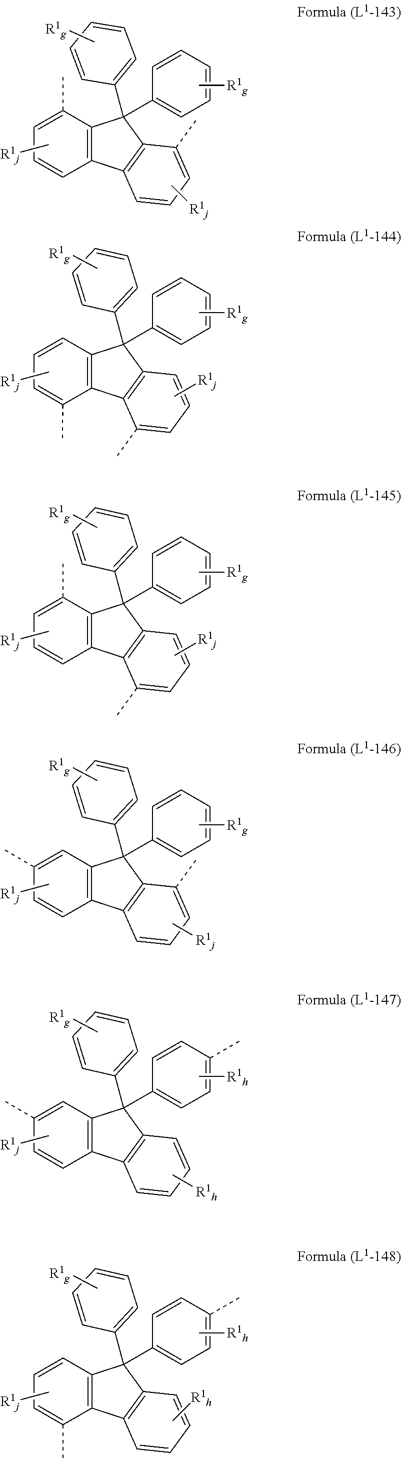

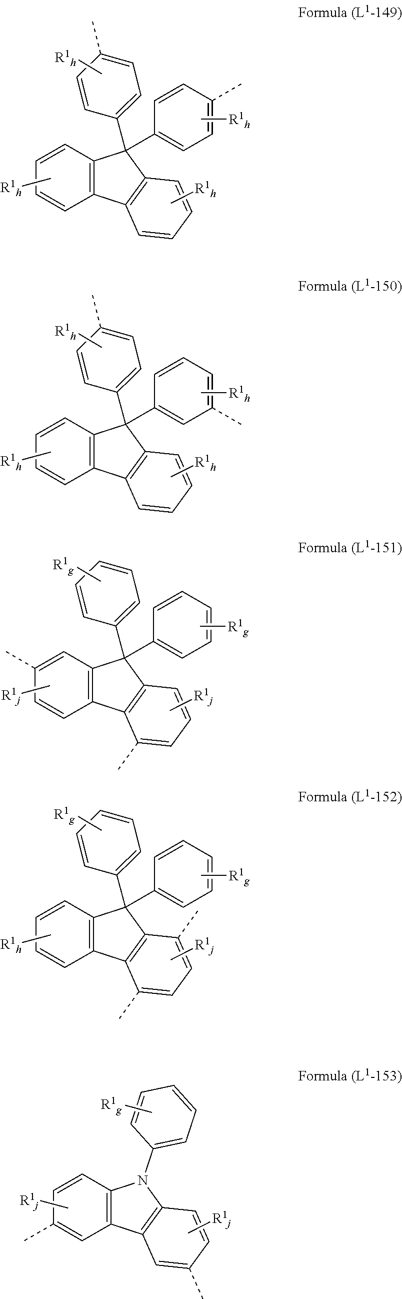

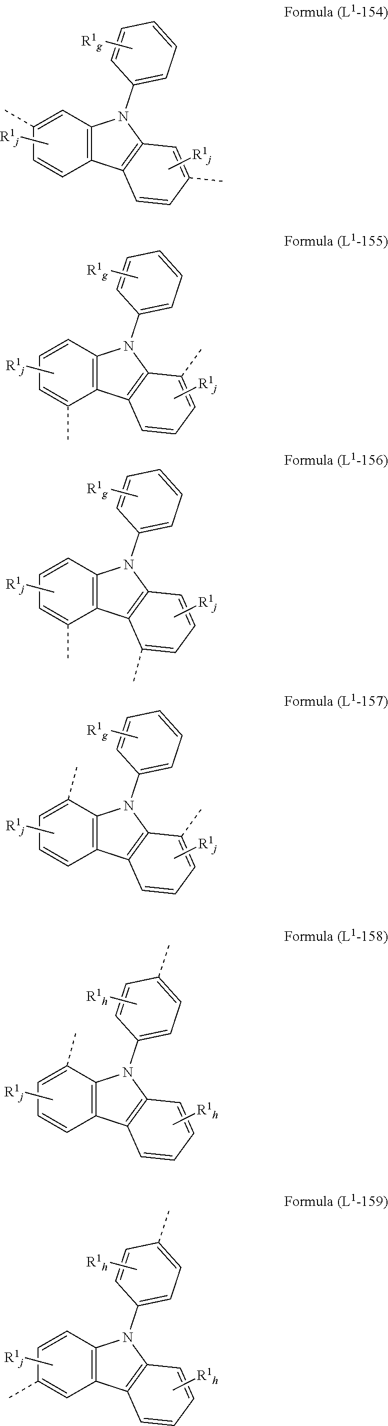

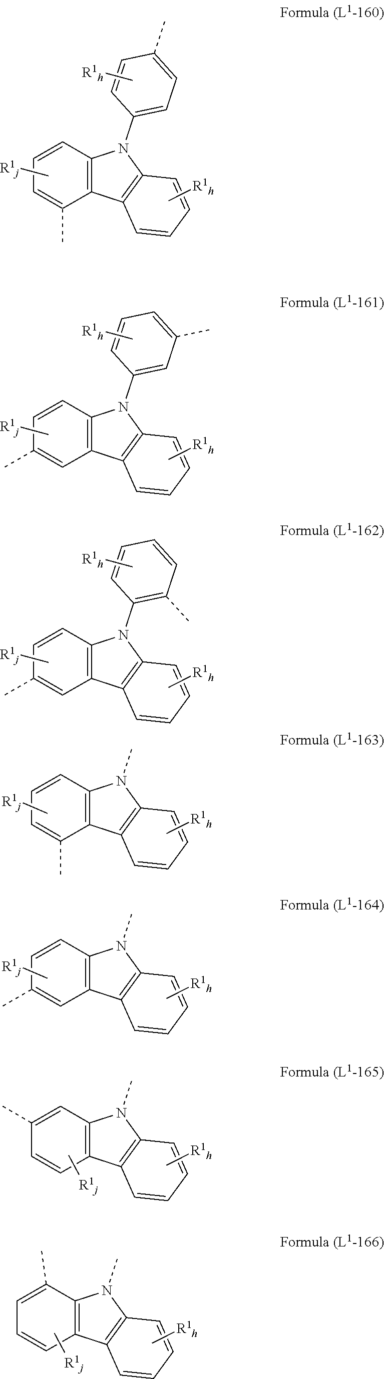

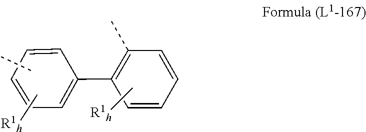

where the dotted bonds in each case mark the positions of attachment, the index k is 0 or 1, the index I is 0, 1 or 2, the index j at each instance is independently 0, 1, 2 or 3; the index h at each instance is independently 0, 1, 2, 3 or 4, the index g is 0, 1, 2, 3, 4 or 5; the symbol Y¹ is 0, S or NR¹, preferably O or S; and the symbol R¹ has the definition given above, especially for formulae (Ia) or (Ib).

It may preferably be the case that the sum total of the indices k, l, g, h and j in the structures of the formula (L¹-1) to (L¹-167) is at most 3 in each case, preferably at most 2 and more preferably at most 1.

Preferred compounds of the invention having a group of the formula (L¹-Z) and/or (QL) comprise an L¹ group which represents a bond or which is selected from one of the formulae (L¹-1) to (L¹-78) and/or (L¹-92) to (L¹-167), preferably of the formula (L¹-1) to (L¹-54) and/or (L¹-92) to (L¹-167), especially preferably of the formula (L¹-1) to (L¹-29) and/or (L¹-92) to (L¹-167). Advantageously, the sum total of the indices k, l, g, h and j in the structures of the formulae (L¹-1) to (L¹-78) and/or (L¹-92) to (L¹-167), preferably of the formula (L¹-1) to (L¹-54) and/or (L¹-92) to (L¹-167), especially preferably of the formula (L¹-1) to (L¹-29) and/or (L¹-92) to (L¹-167), may in each case be not more than 3, preferably not more than 2 and more preferably not more than 1.

Preferably, the R¹ radicals in the formulae (L¹-1) to (L¹-167) do not form a fused aromatic or heteroaromatic ring system, and preferably do not form any fused ring system, with the ring atoms of the aryl group or heteroaryl group to which the R¹ radicals are bonded. This includes the formation of a fused ring system with possible substituents R², R³ which may be bonded to the R¹ or R² radicals.

It may also be the case that the Ar¹ and/or R¹ group is selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, pyrenyl, triazinyl, imidazolyl, benzimidazolyl, benzoxazolyl, benzothiazolyl, 1-, 2-, 3- or 4-carbazolyl, 1- or 2-naphthyl, anthracenyl, preferably 9-anthracenyl, phenanthrenyl and/or triphenylenyl, each of which may be substituted by one or more R² radicals, but are preferably unsubstituted, particular preference being given to phenyl, spirobifluorene, fluorene, dibenzofuran, dibenzothiophene, anthracene, phenanthrene, triphenylene groups.

It may further be the case that, in a structure of formula (Ia), (Ib), (II-1) to (II-16), (III-1) to (III-16), (IV-1) to (IV-24), (V-1) to (V-32), (VI-1) to (VI-8), (VII-1) to (VII-8), (VIII-1) to (VIII-10), (IX-1) to (IX-8), (X-1) to (X-8), (XI-1) to (XI-10), (XII-1) to (XII-24), (XIII-1) to (XIII-32), (XIV-1) to (XIV-8), (XV-1) to (XV-10), (XVI-1) to (XVI-8), (XVII-1) to (XVII-8), (XVIII-1) to (XVIII-10) and/or (XIX-1) to (XIX-10), at least one R¹ or Ar¹ radical is a group selected from the formulae (R¹-1) to (R¹-179), or, in a structure of formula (H-1) to (H-26), (Q-1) to (Q-44), (Z-1) to (Z-91), (L¹-1) to (L¹-167), at least one Ar¹ or R¹ radical is a group selected from the formulae (R¹-1) to (R¹-179):

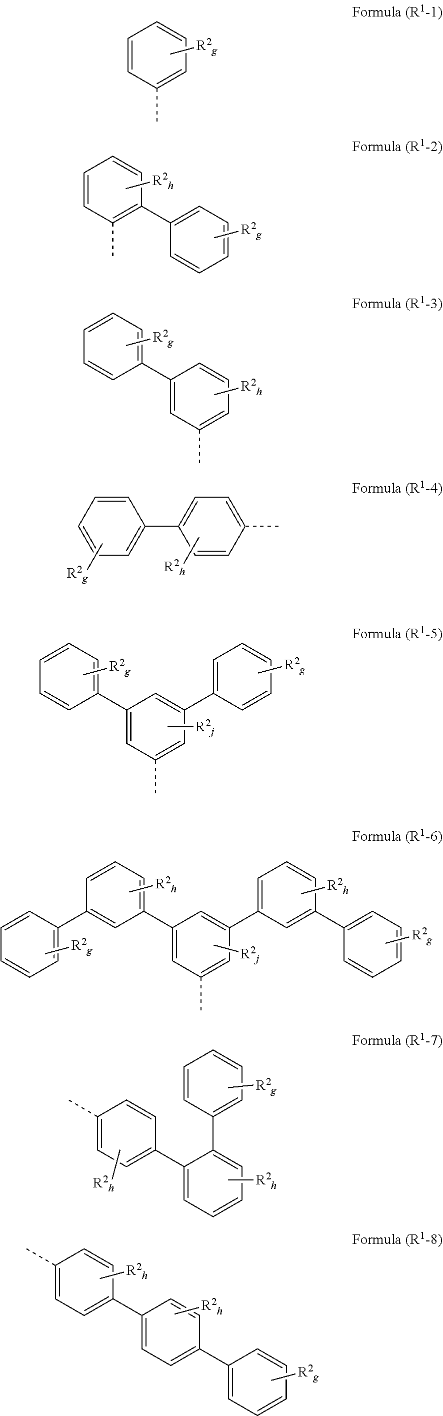

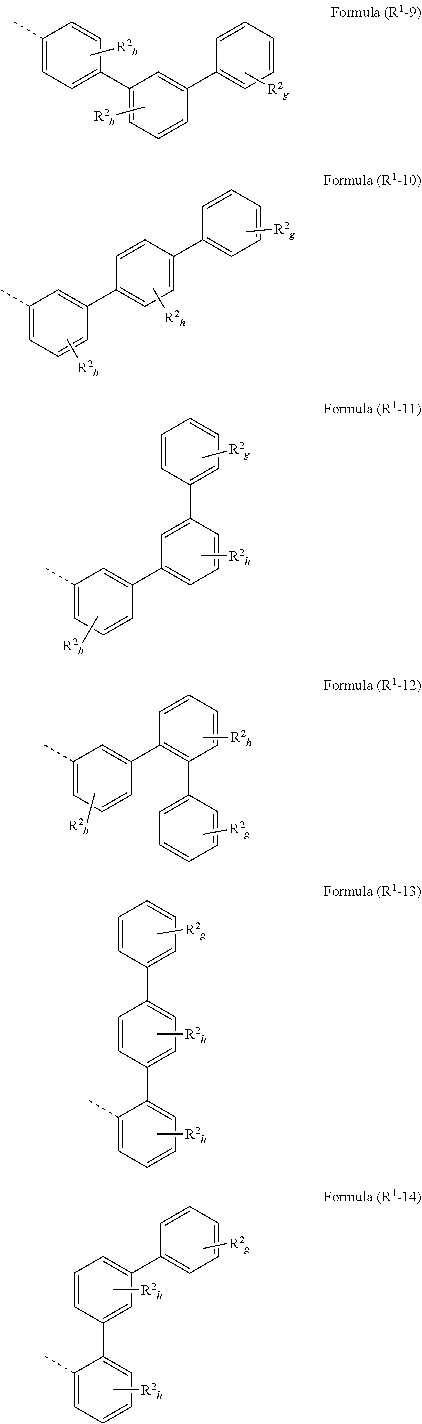

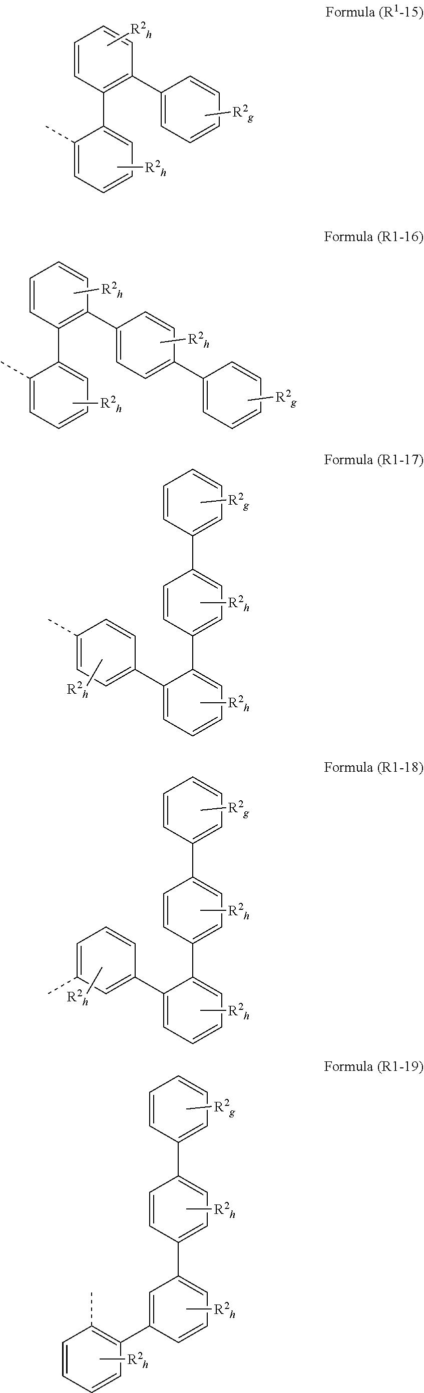

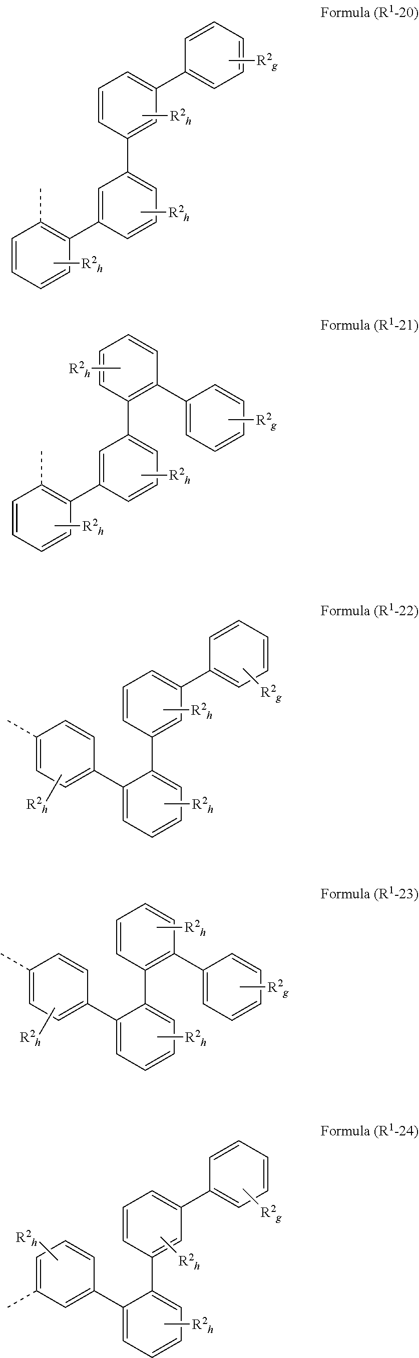

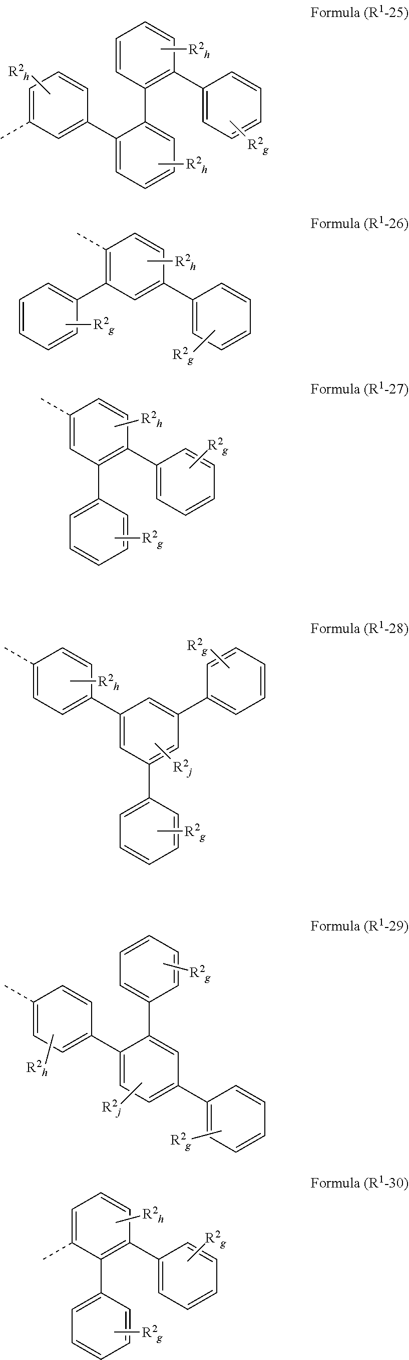

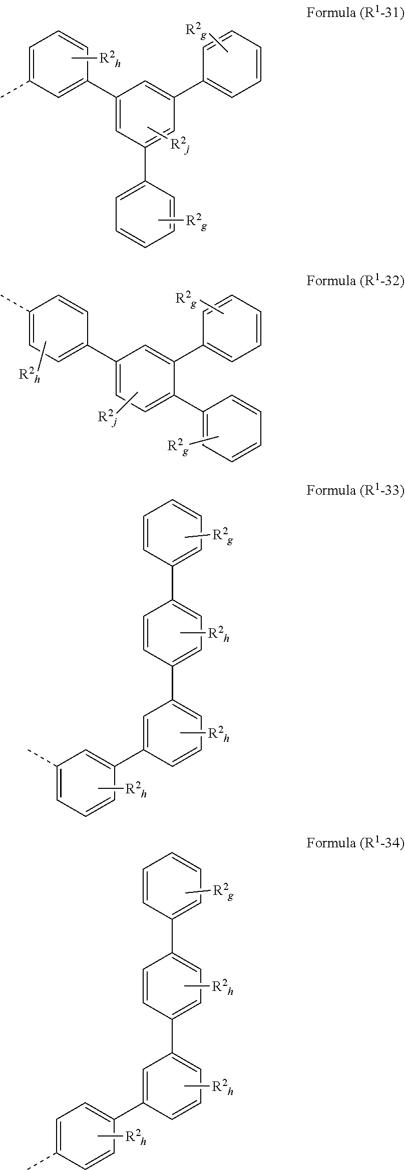

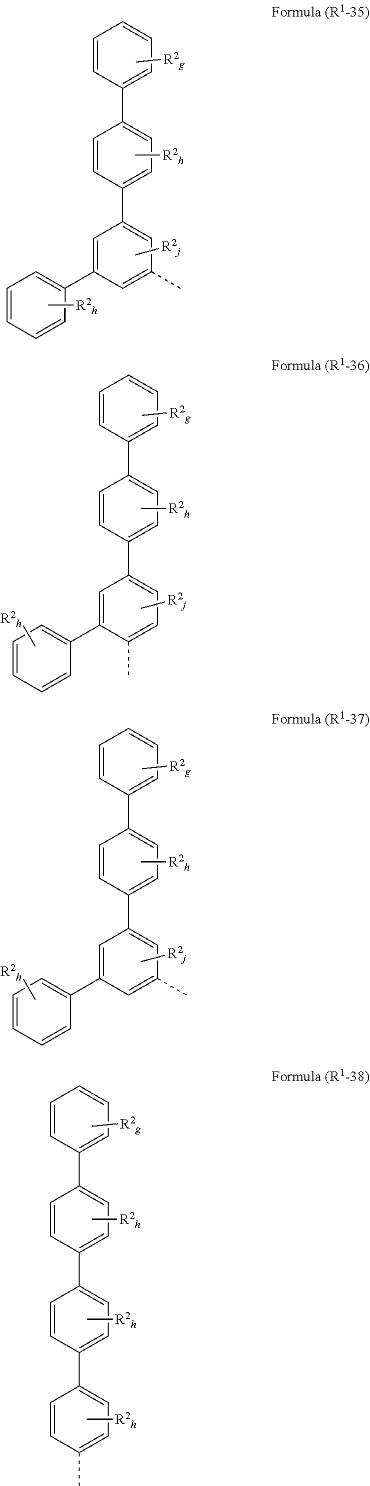

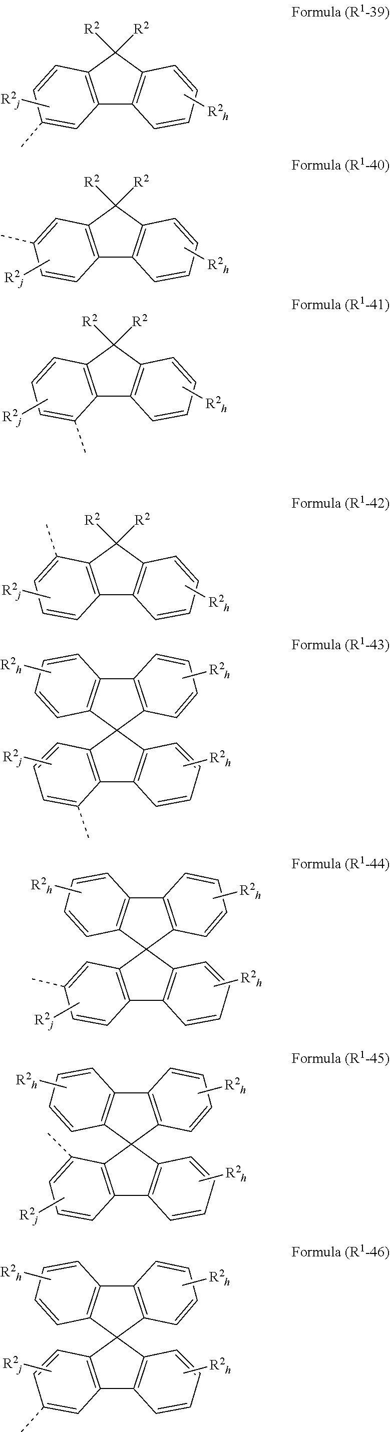

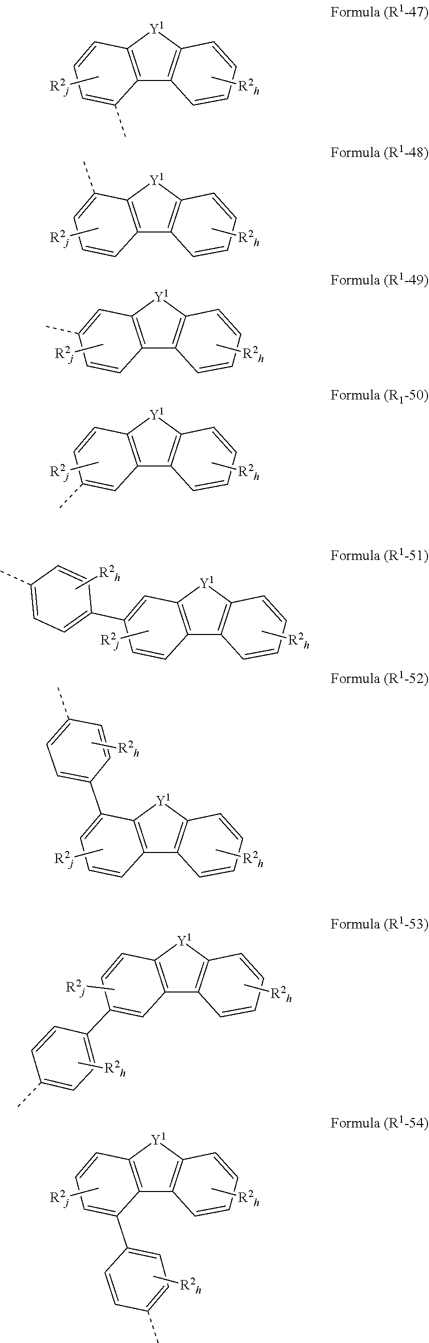

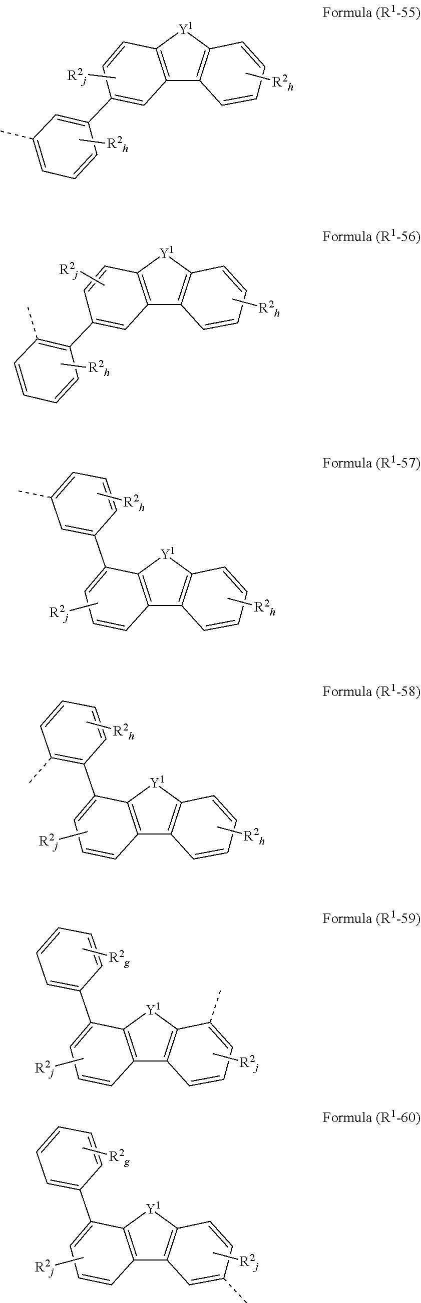

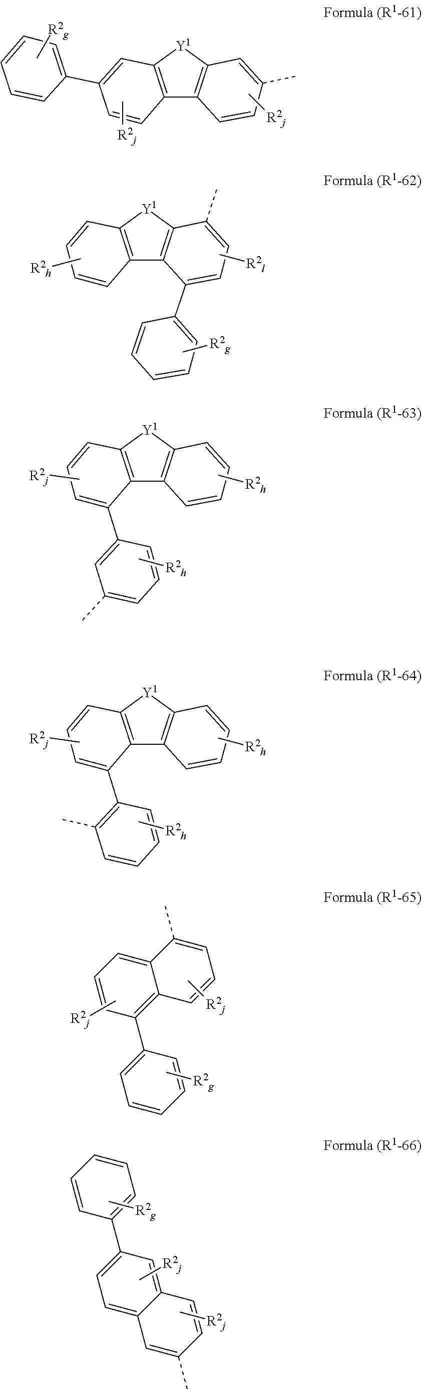

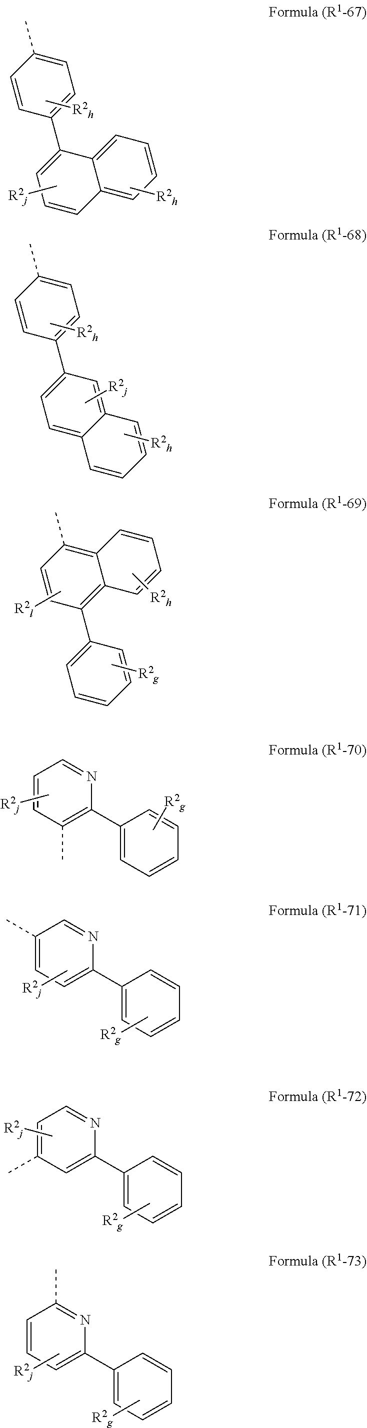

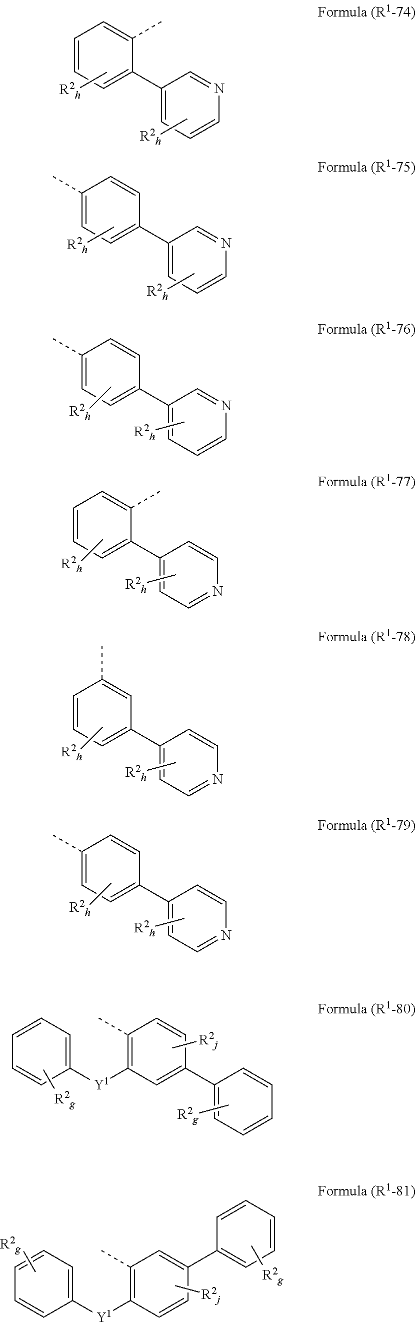

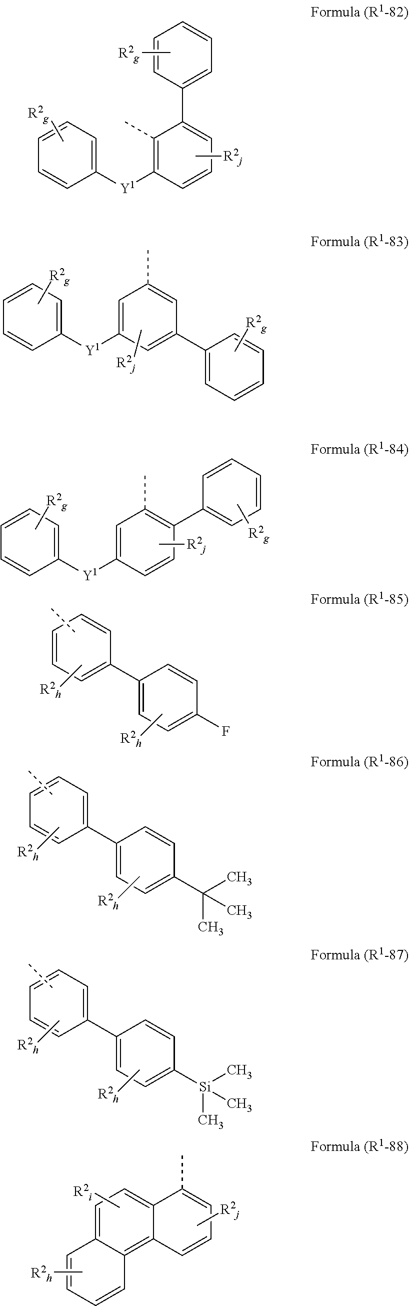

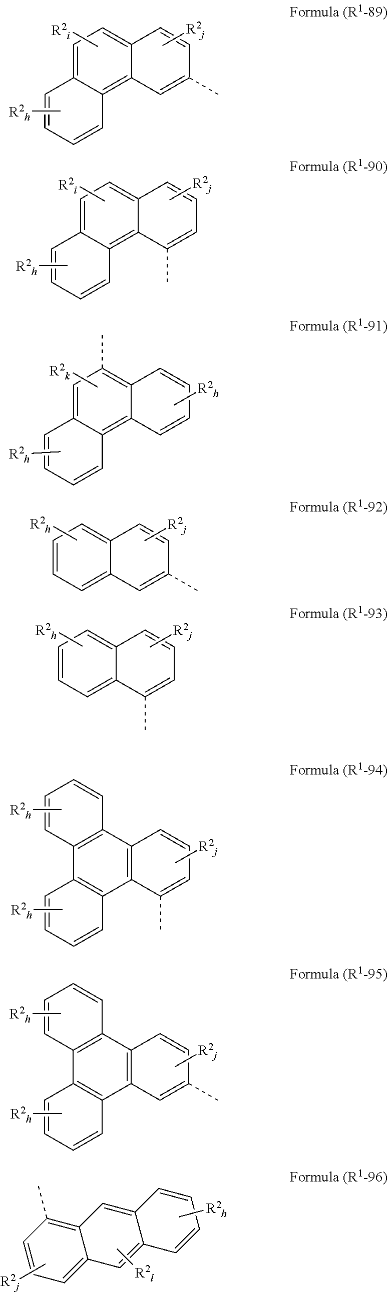

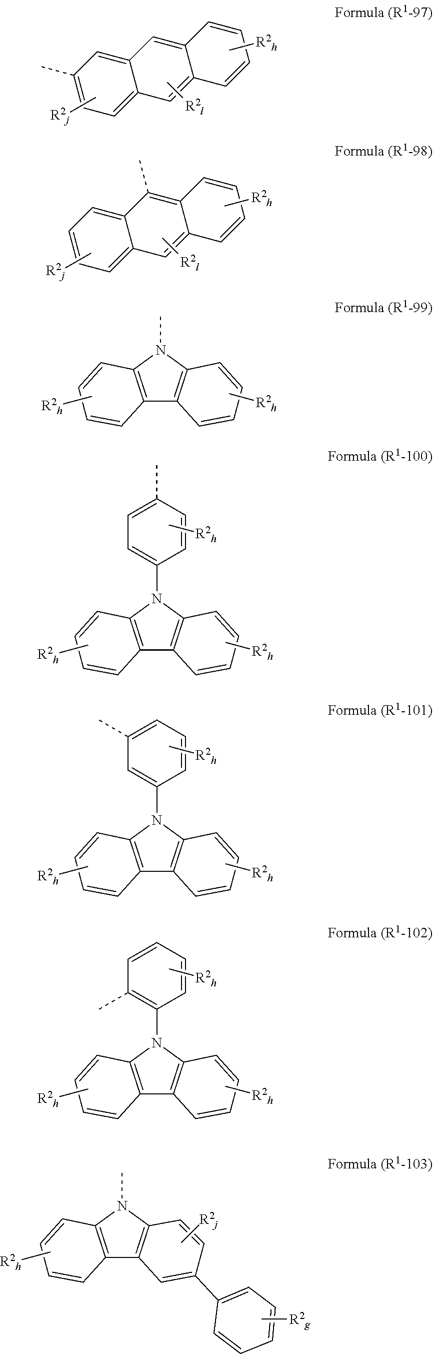

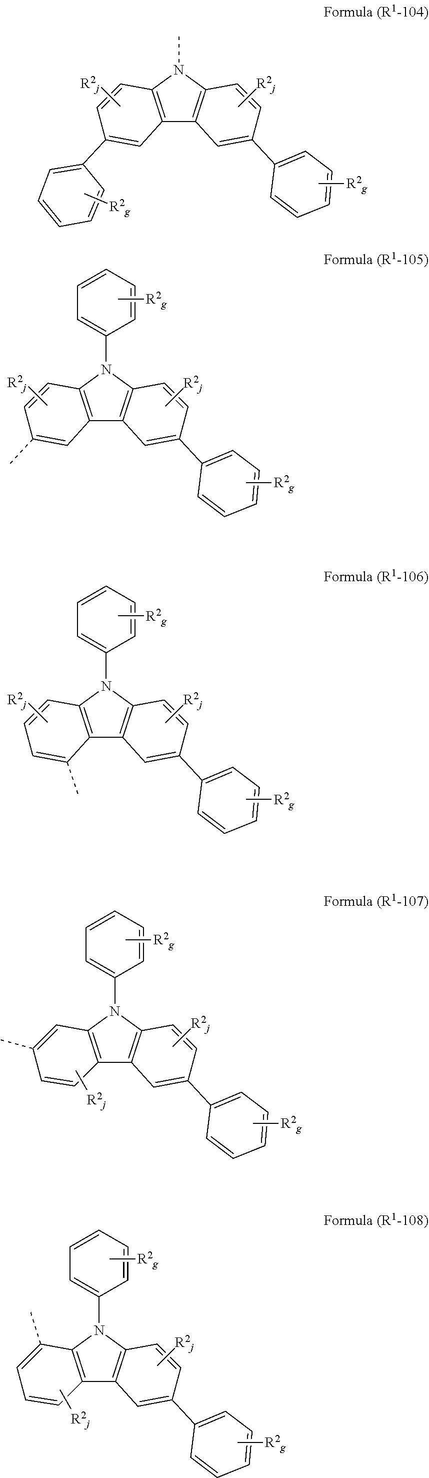

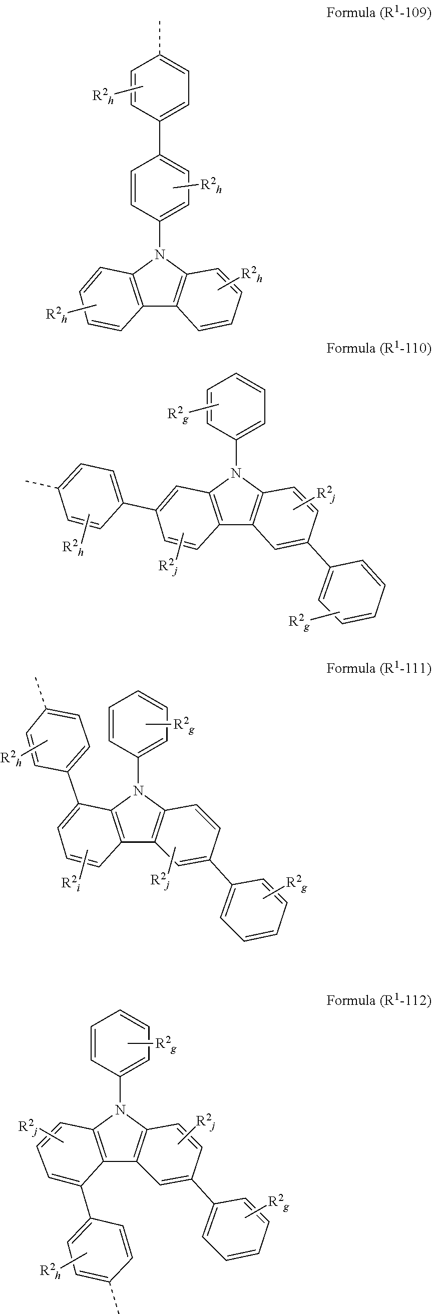

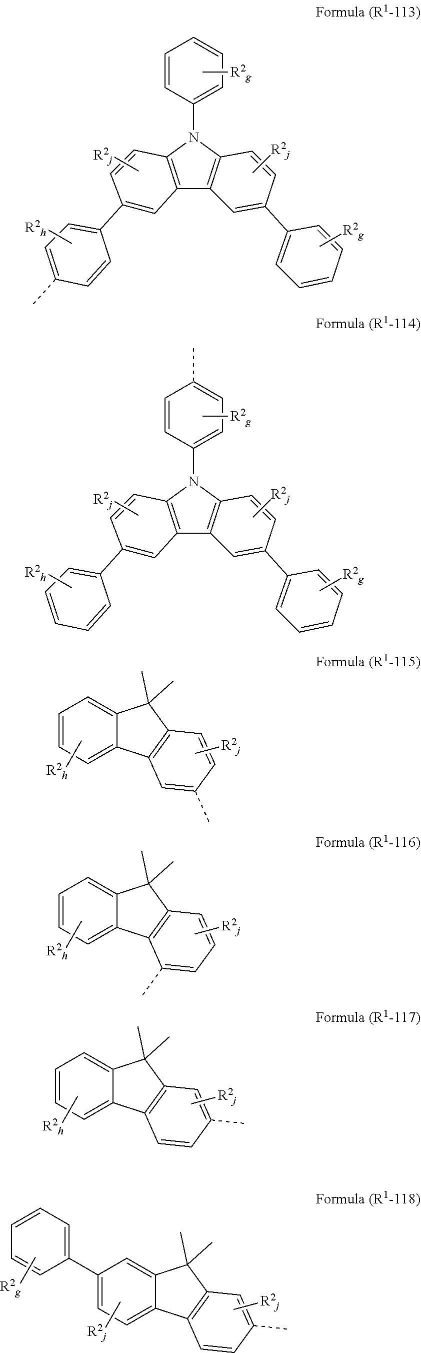

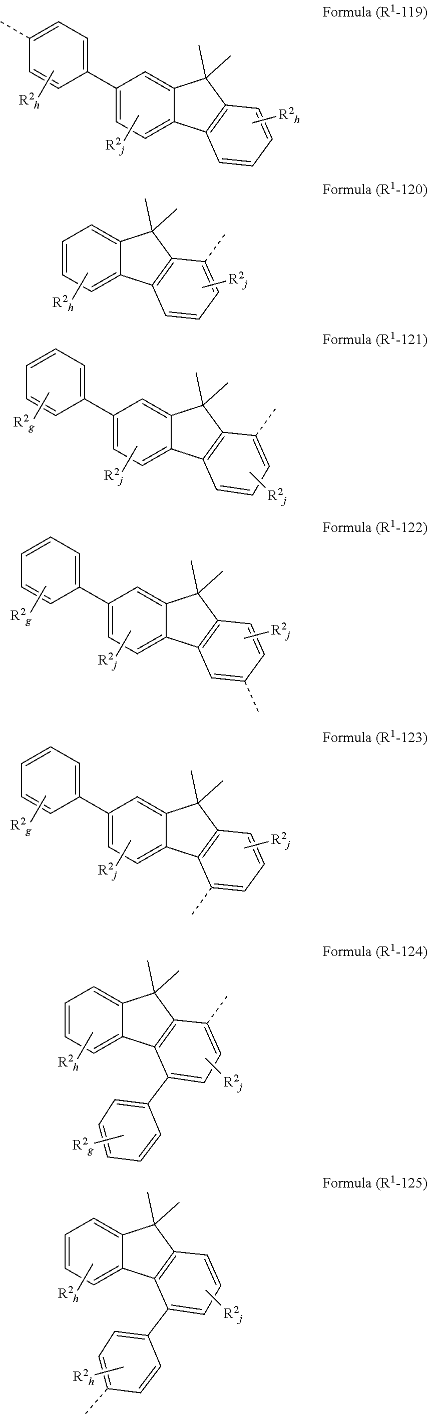

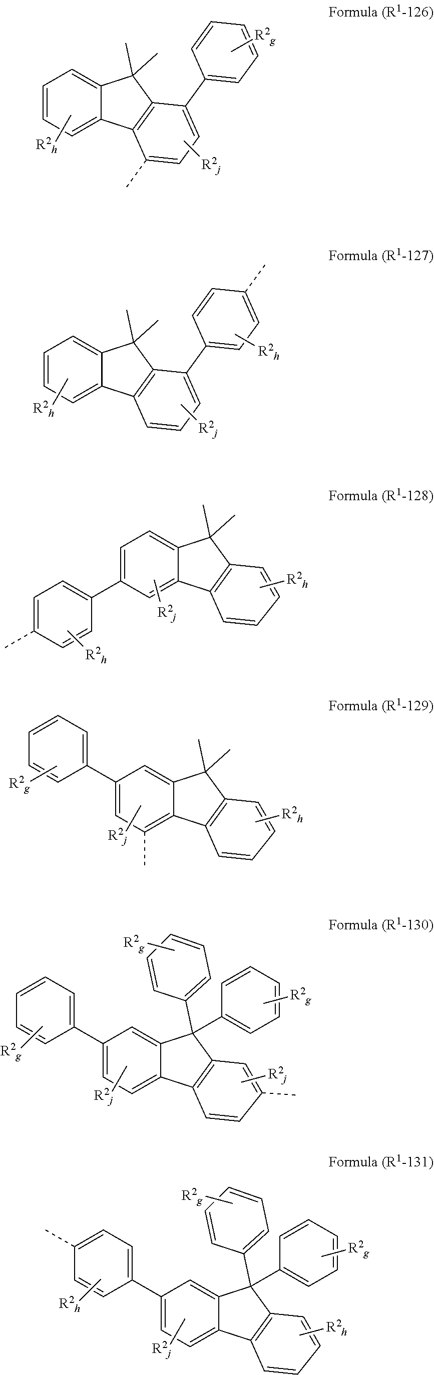

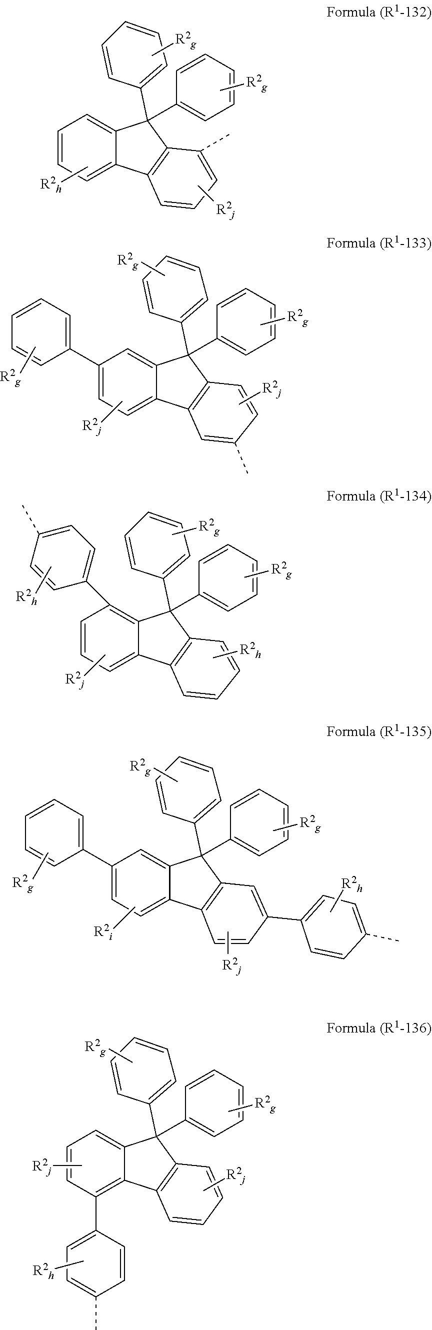

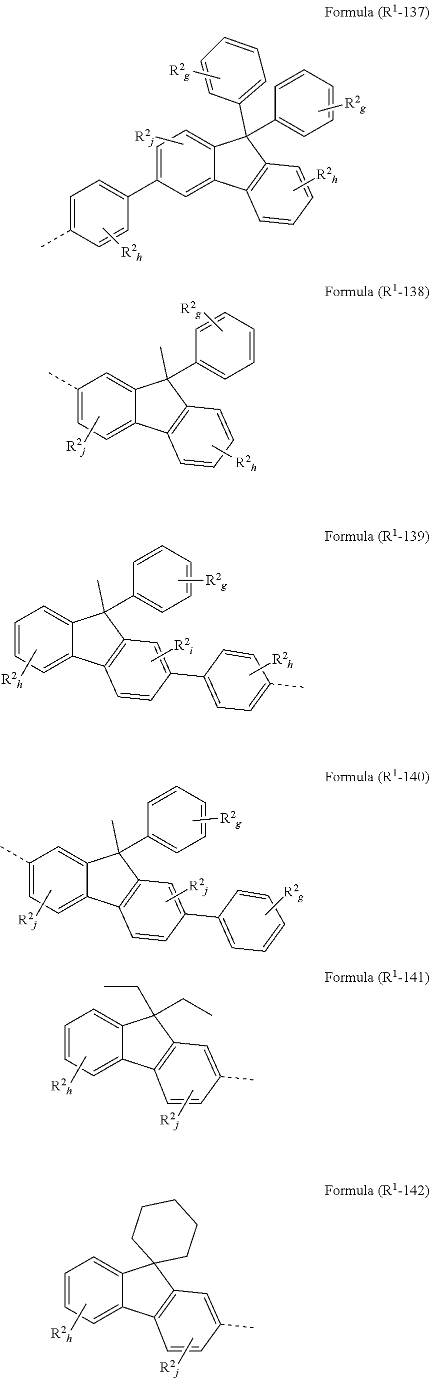

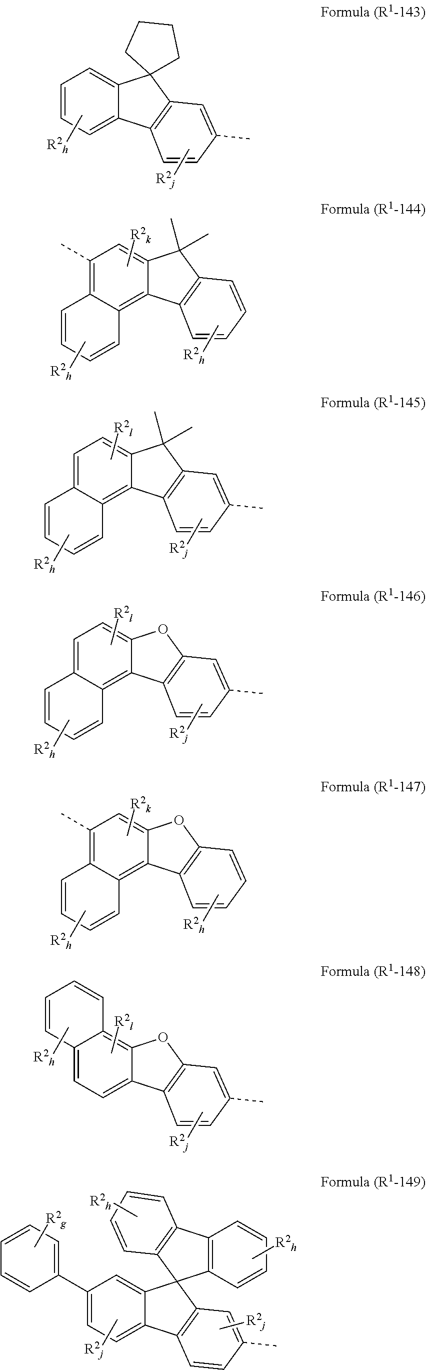

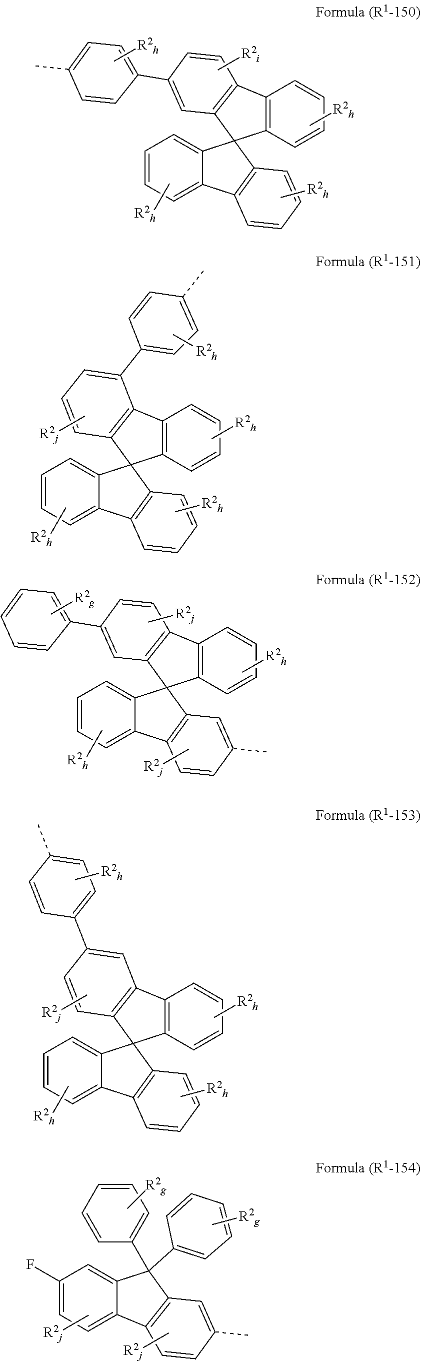

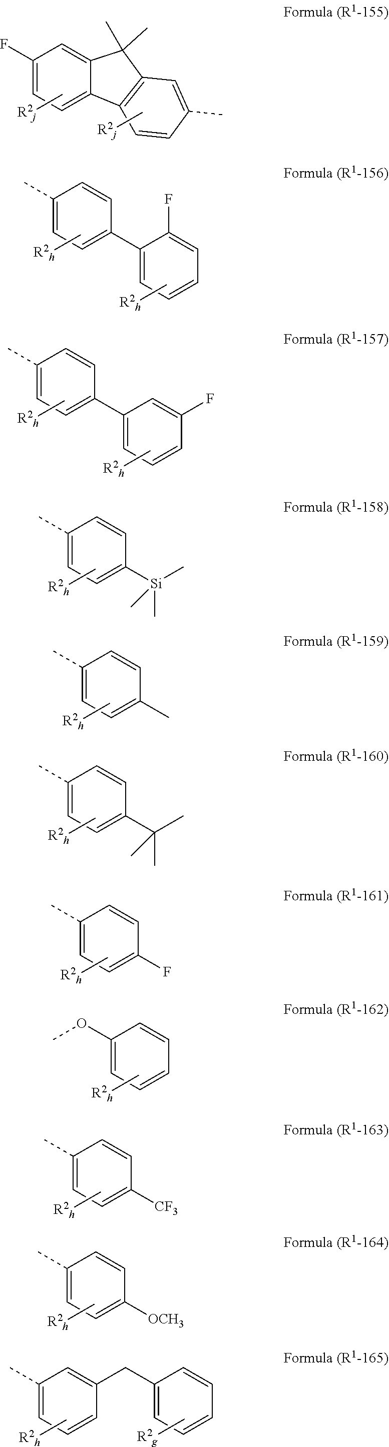

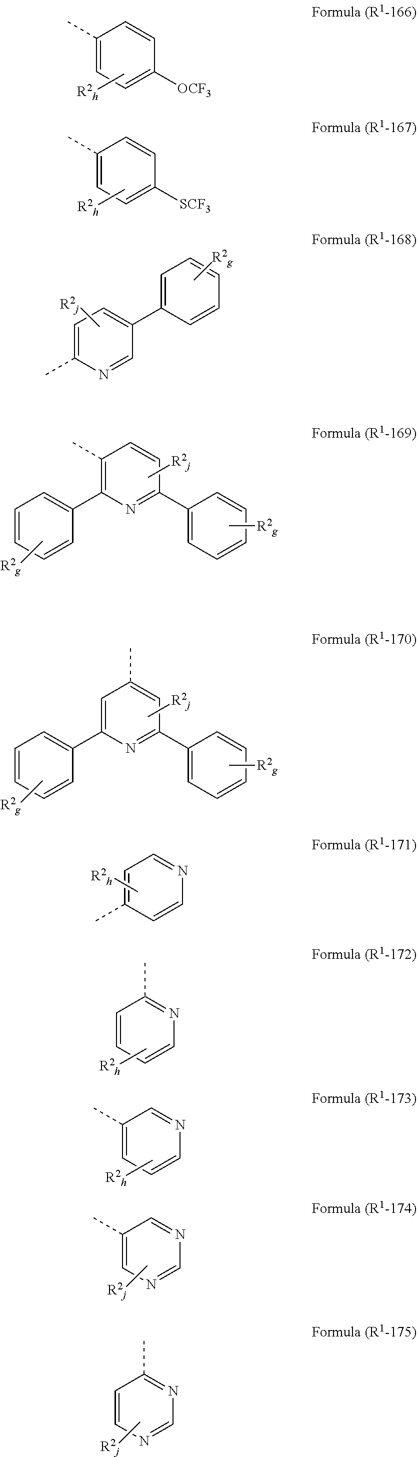

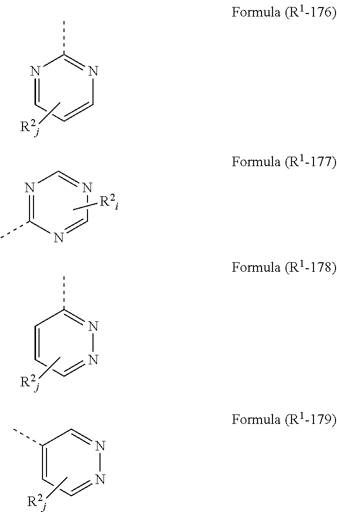

where the symbols used are as follows:

Y¹ is O, S or NR², preferably O or S,

k at each instance is independently 0 or 1;

i at each instance is independently 0, 1 or 2;

at each instance is independently 0, 1, 2 or 3;

h at each instance is independently 0, 1, 2, 3 or 4;

g at each instance is independently 0, 1, 2, 3, 4 or 5;

R² has the definition given above, especially for formula (Ia) or (Ib),
and

the dotted bond marks the position of attachment.

Of the aforementioned structures of the formulae (R¹-1) to (R¹-179), preference is given to the groups of the formulae (R¹-1) to (R¹-177), particular preference being given to groups of the formulae (R¹-1) to (R¹-64) and (R¹-94) to (R¹-177), and very particular preference to the groups of the formulae (R¹-1) to (R¹-64) and (R¹-115) to (R¹-177).

It may preferably be the case that the sum total of the indices k, i, j, h and g in the structures of the formula (R¹-1) to (R¹-179) in each case is not more than 3, preferably not more than 2 and more preferably not more than 1.

Preferably, the R² radicals in the formulae (R¹-1) to (R¹-179) do not form a fused aromatic or heteroaromatic ring system, and preferably do not form any fused ring system, with the ring atoms of the aryl group or heteroaryl group to which the R² radicals are bonded. This includes the formation of a fused ring system with possible substituents R³ which may be bonded to the R² radicals.

When the compound of the invention is substituted by aromatic or heteroaromatic R¹ or R² groups, especially in the case of configuration thereof as host material, electron transport material or hole transport material, it is preferable when they do not have any aryl or heteroaryl groups having more than two aromatic six-membered rings fused directly to one another. More preferably, the substituents do not have any aryl or heteroaryl groups having six-membered rings fused directly to one another at all. The reason for this preference is the low triplet energy of such structures. Fused aryl groups which have more than two aromatic six-membered rings fused directly to one another but are nevertheless also suitable in accordance with the invention are phenanthrene and triphenylene, since these also have a high triplet level.

In a further preferred embodiment of the invention, R², for example in a structure of formula (Ia) and/or (Ib) and preferred embodiments of this structure or the structures where reference is made to these formulae, is the same or different at each instance and is selected from the group consisting of H, D, an aliphatic hydrocarbyl radical having 1 to 10 carbon atoms, preferably having 1, 2, 3 or 4 carbon atoms, or an aromatic or heteroaromatic ring system which has 5 to 30 aromatic ring atoms, preferably 5 to 24 aromatic ring atoms, more preferably 5 to 13 aromatic ring atoms, and may be substituted by one or more alkyl groups each having 1 to 4 carbon atoms, but is preferably unsubstituted.

In a further preferred embodiment of the invention, R³, for example in a structure of formula (Ia) and/or (Ib) and preferred embodiments of this structure or the structures where reference is made to these formulae, is the same or different at each instance and is selected from the group consisting of H, D, F, CN, an aliphatic hydrocarbyl radical having 1 to 10 carbon atoms, preferably having 1, 2, 3 or 4 carbon atoms, or an aromatic or heteroaromatic ring system which has 5 to 30 aromatic ring atoms, preferably 5 to 24 aromatic ring atoms, more preferably 5 to 13 aromatic ring atoms, and may be substituted by one or more alkyl groups each having 1 to 4 carbon atoms, but is preferably unsubstituted.

Particular preference is given to compounds of the invention having structures of the formulae (Ia), (Ib), (II-1) to (II-16), (IV-1) to (IV-24), (V-1) to (V-32), (VI-1) to (VI-8), (VII-1) to (VII-8), (VIII-1) to (VIII-10), (IX-1) to (IX-8), (X-1) to (X-8), (XI-1) to (XI-10), where a total of not more than 4, preferably not more than 2, radicals of the formula X are not CH or CD, where at least one R radical comprises an electron transport group, preferably a triazine group, more preferably a group of the formula (L¹-Z) in which Z represents a group of the formula (Z-48), (Z-49), (Z-50) or (Z-51), more preferably a group of the formula (Z-48), having the following properties:

| | | | |
|---|---|---|---|
| R, not H or D, or | preferably | in formula (L¹— | preferably |
| Ar¹ | | Z): L¹ | |
| R¹-1 to R¹-177 | R¹-1 to R¹-5 | a bond or L¹-1 | a bond or (L¹- |
| | and (R¹-39) to | to L¹-167 | 94) to (L¹-134) |
| | (R¹-50) | | |
| R¹-1 to R¹-177 | R¹-1 to R¹-5 | a bond or (L¹- | a bond |
| | and (R¹-39) to | 94) to (L¹-134) | |
| | (R¹-50) | | |
| R¹-1 to R¹-4 | R¹-1 | a bond or L¹-1 | a bond or (L¹- |
| | | to L¹-167 | 94) to (L¹-134) |
| R¹-1 to R¹-4 | R¹-1 | a bond or (L¹- | a bond |
| | | 94) to (L¹-134) | |

Particular preference is given to compounds of the invention having structures of the formulae (Ia), (Ib), (II-1) to (II-16), (IV-1) to (IV-24), (V-1) to (V-32), (VI-1) to (VI-8), (VII-1) to (VII-8), (VIII-1) to (VIII-10), (IX-1) to (IX-8), (X-1) to (X-8), (XI-1) to (XI-10), where a total of not more than 4, preferably not more than 2, radicals of the formula X are not CH or CD, where at least one R radical comprises a fused aromatic ring system, preferably an anthracene, phenanthrene, triphenylene group, more preferably a group of the formula (L¹-Z) in which Z represents a group of the formula (Z-78), (Z-79), (Z-80) having the following properties:

| R, not H or D, or Ar¹ | preferably | in formula | preferably |
|---|---|---|---|
| | | (L¹—Z): L¹ | |
| R¹-1 to R¹-177 | R¹-1 to R¹-5 | a bond or L¹- | a bond or (L¹- |
| | and (R¹-39) | 1 to L¹-167 | 94) to (L¹-134) |
| | to (R¹-50) | | |
| R¹-1 to R¹-177 | R¹-1 to R¹-5 | a bond or (L¹- | a bond |
| | and (R¹-39) | 94) to (L¹- | |
| | to (R¹-50) | 134) | |
| R¹-1 to R¹-4 | R¹-1 | a bond or L¹- | a bond or (L¹- |
| | | 1 to L¹-167 | 94) to (L¹-134) |
| R¹-1 to R¹-4 | R¹-1 | a bond or (L¹- | a bond |
| | | 94) to (L¹- | |
| | | 134) | |

Examples of suitable compounds of the invention are the structures of the following formulae 1 to 432 shown below:

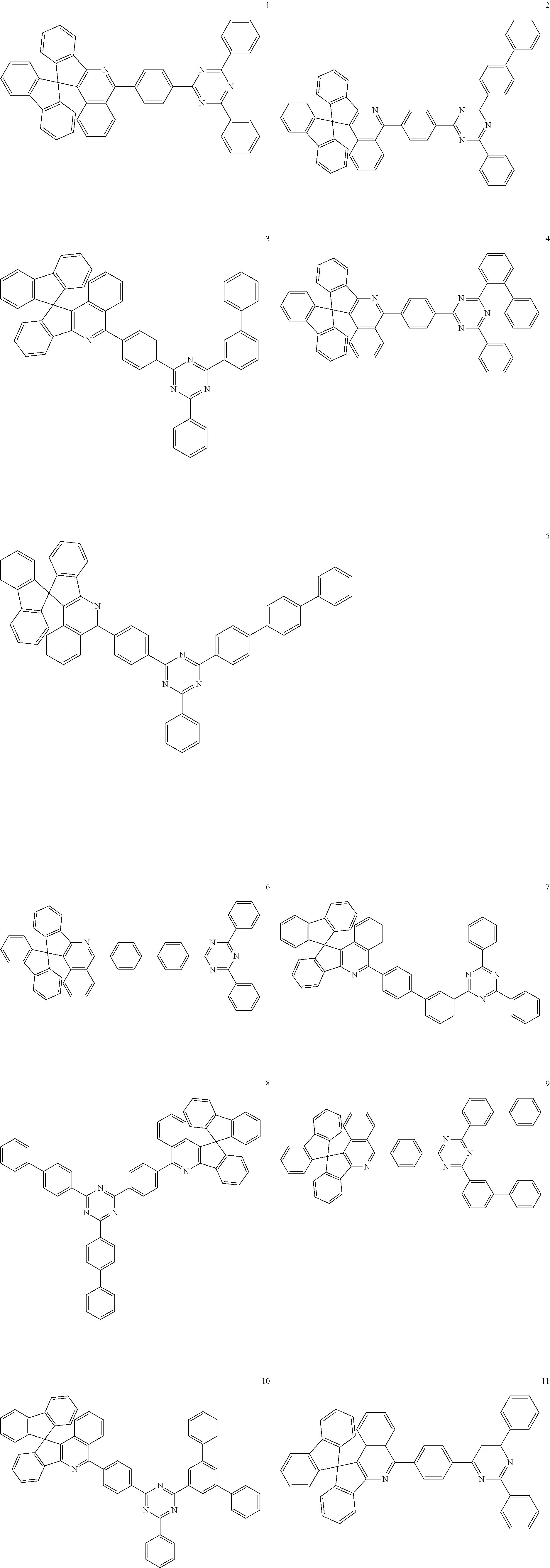

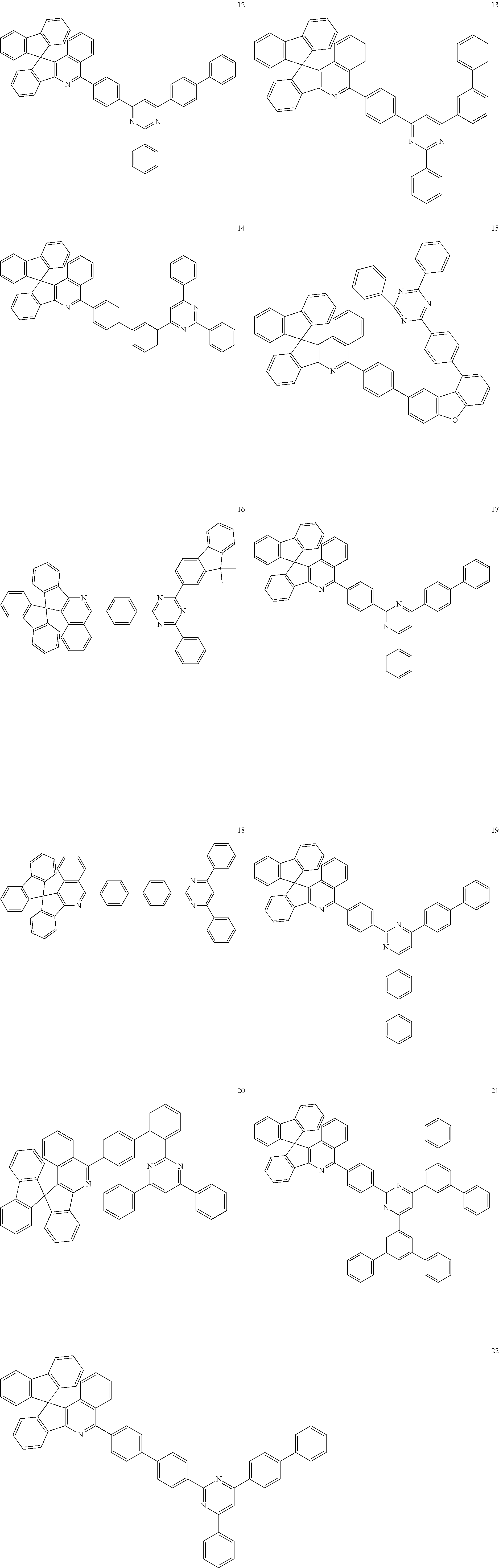

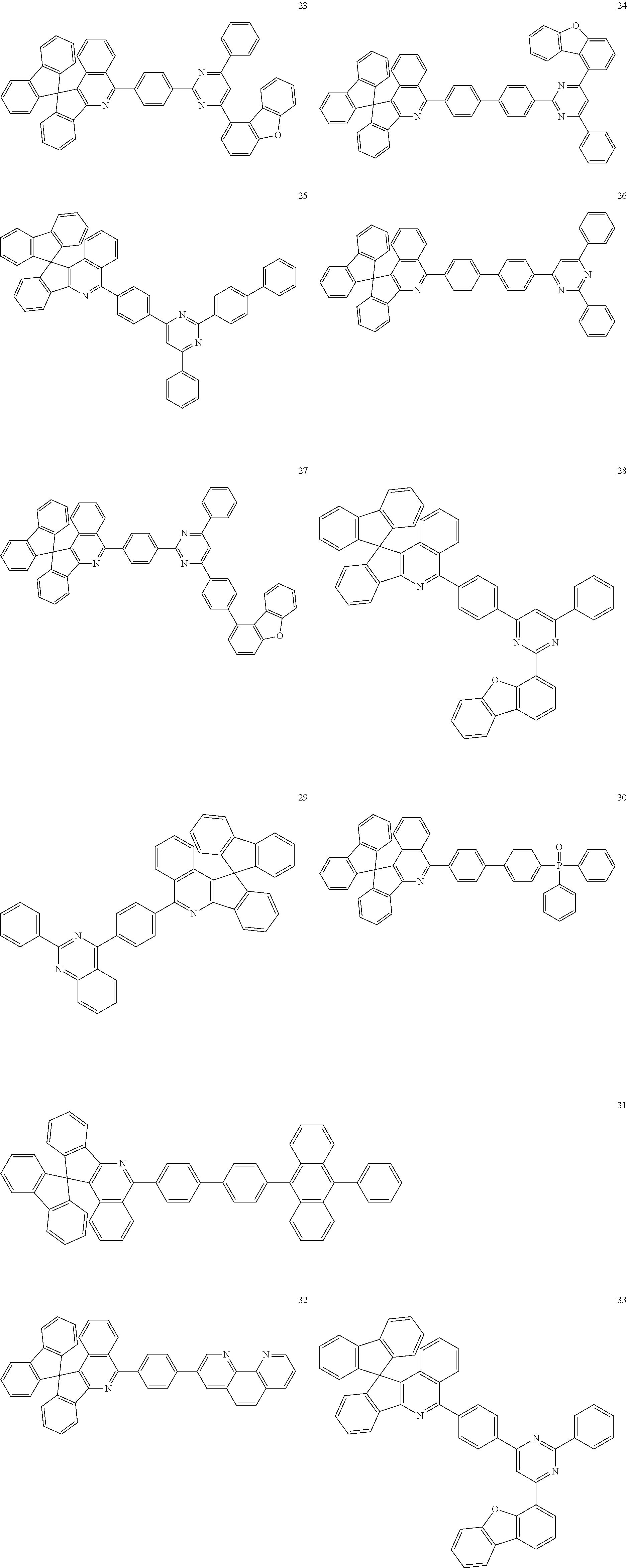

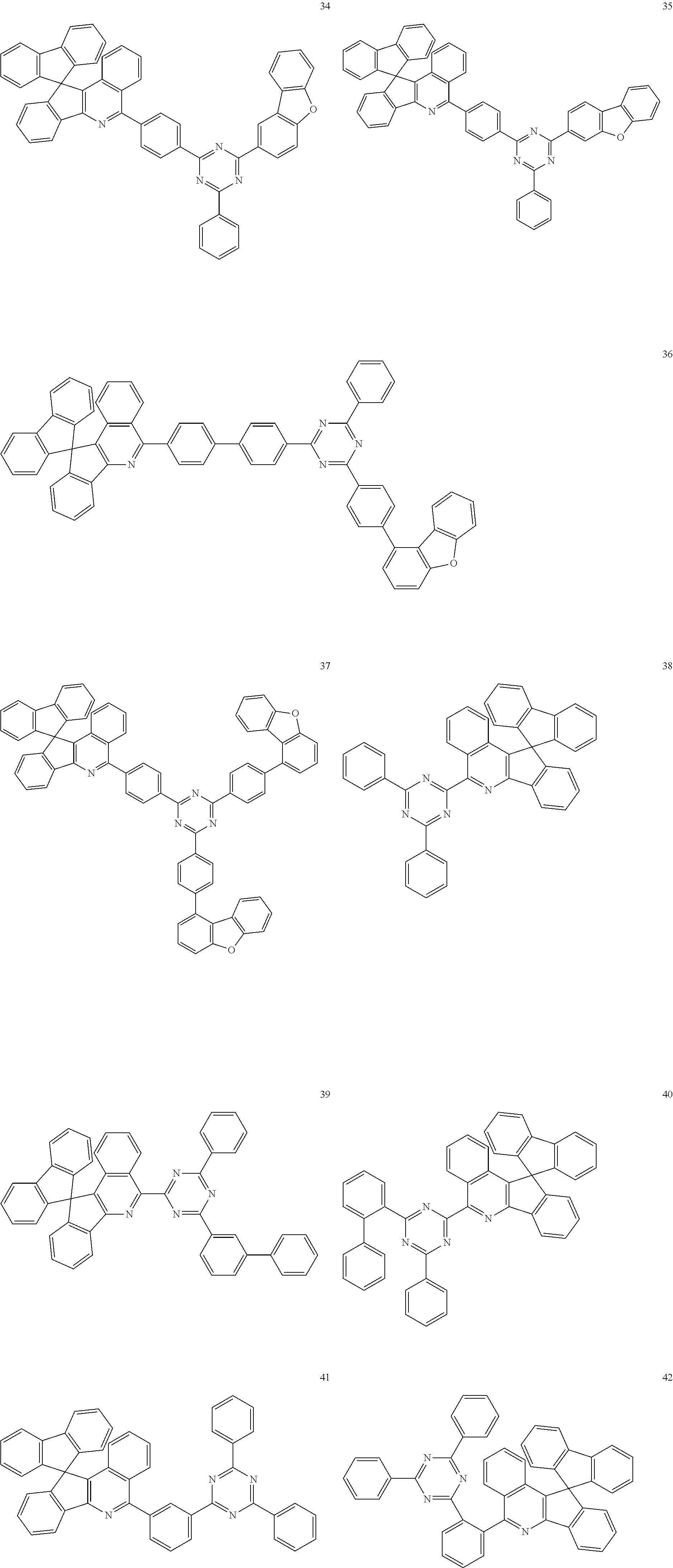

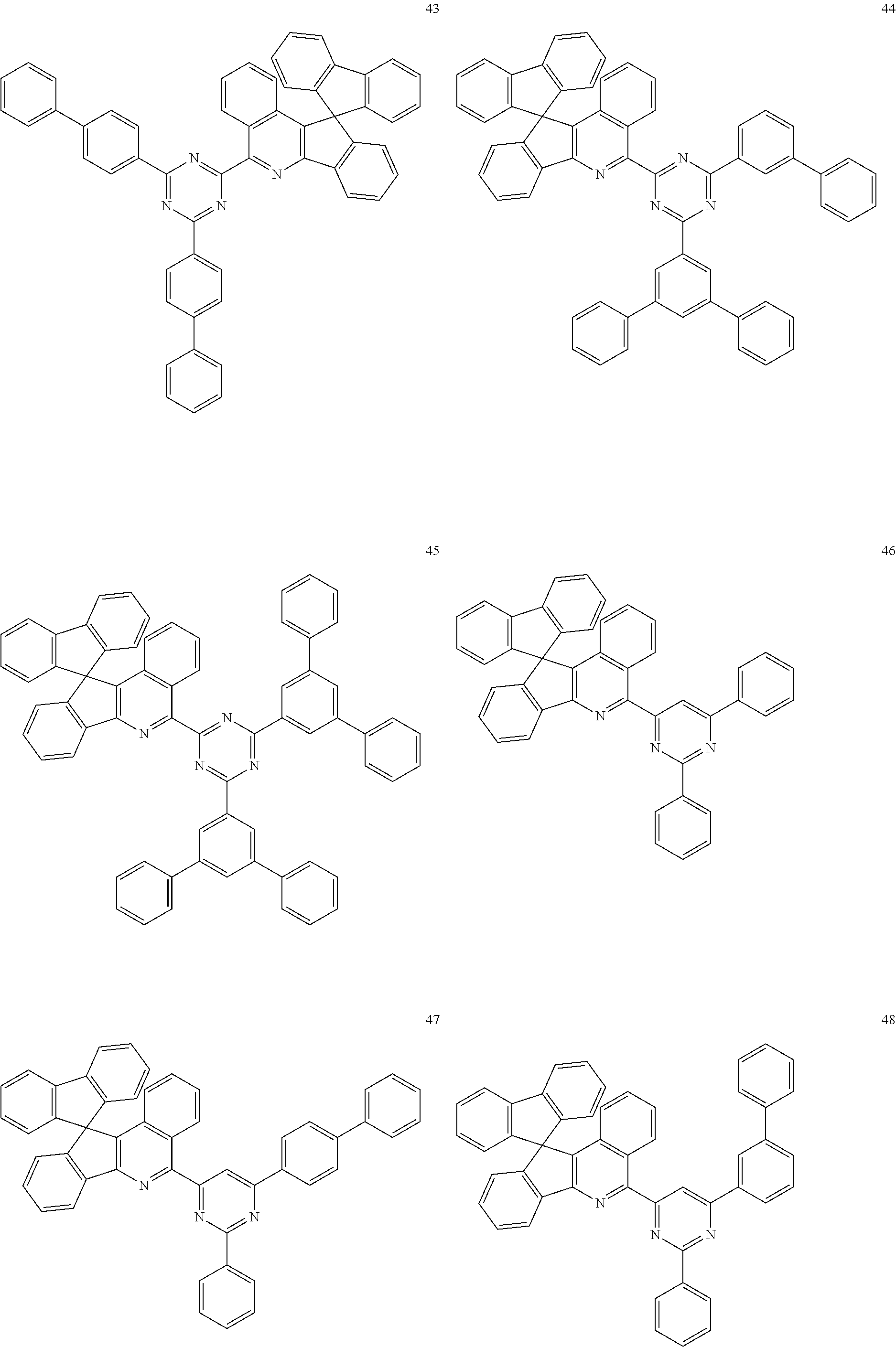

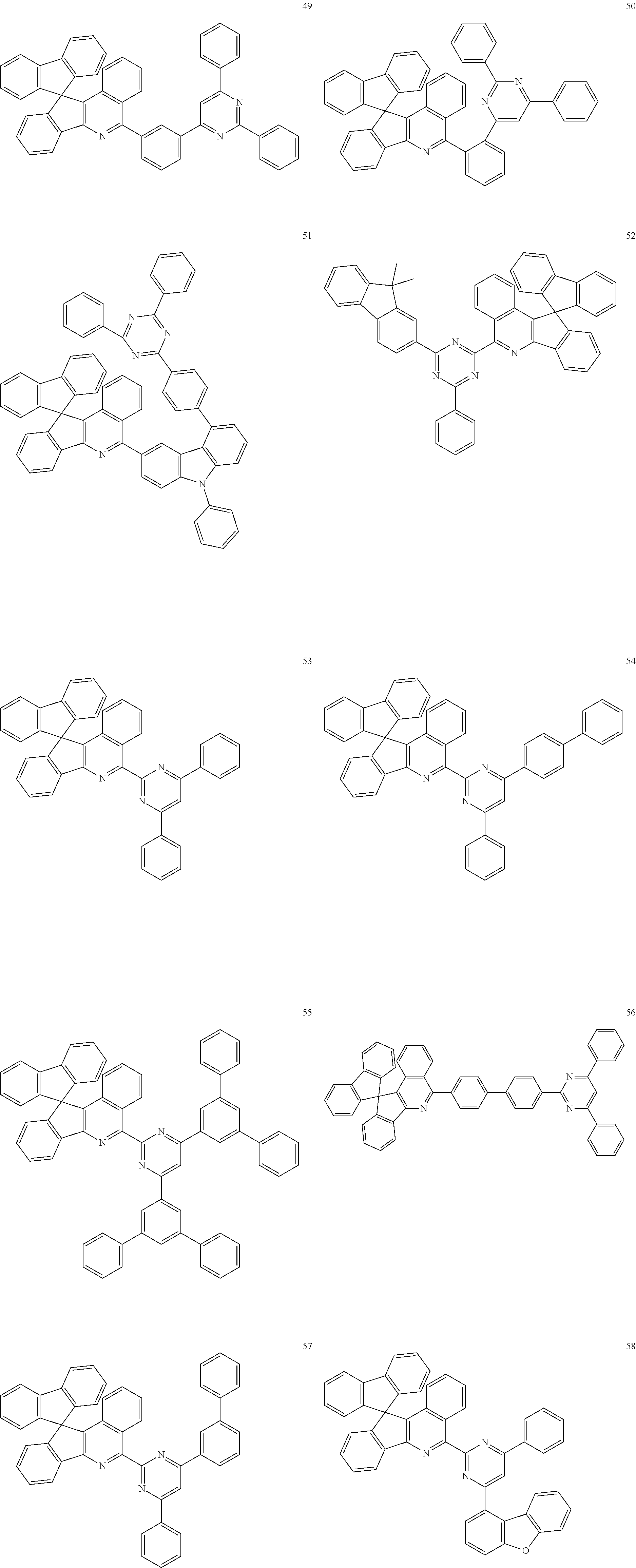

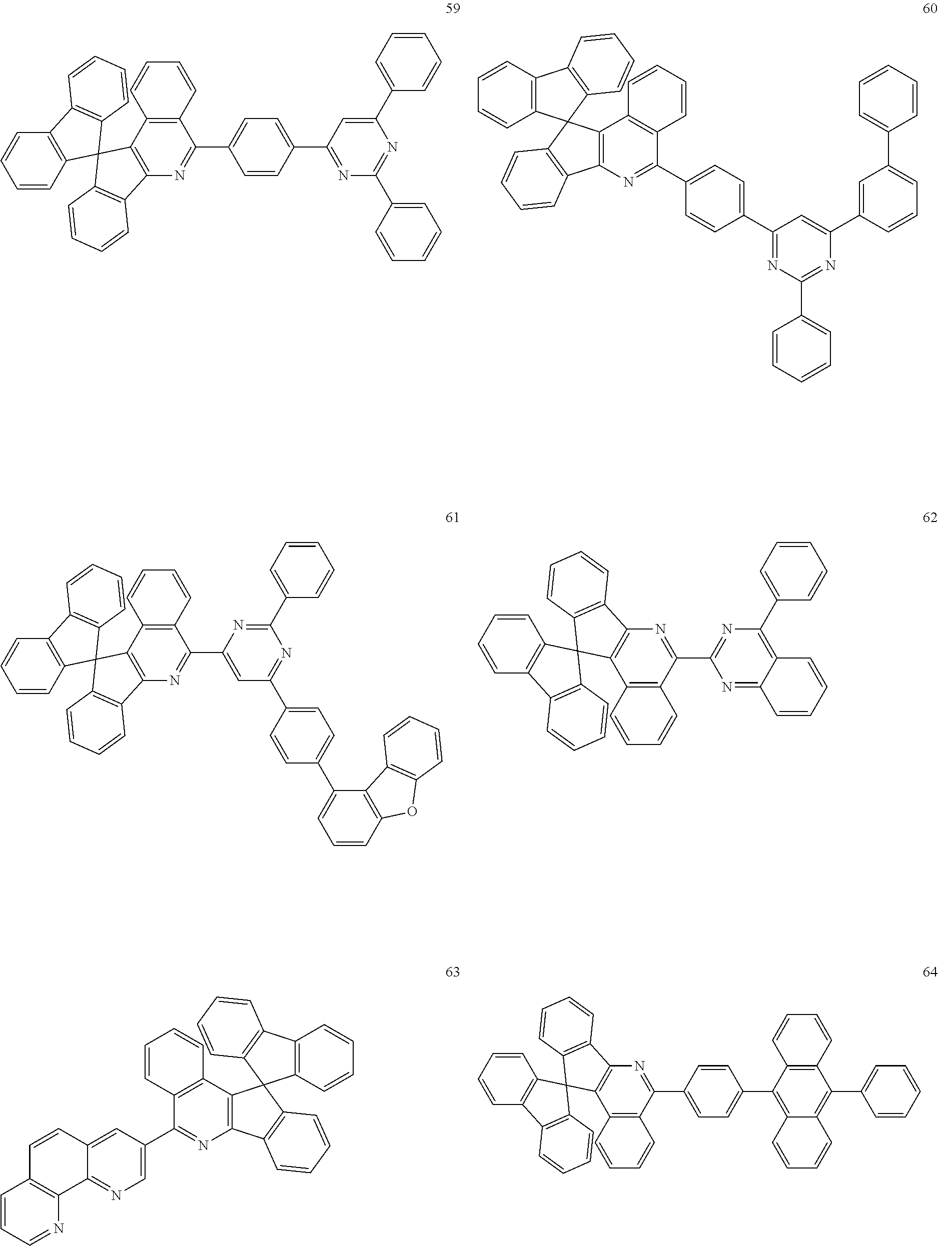

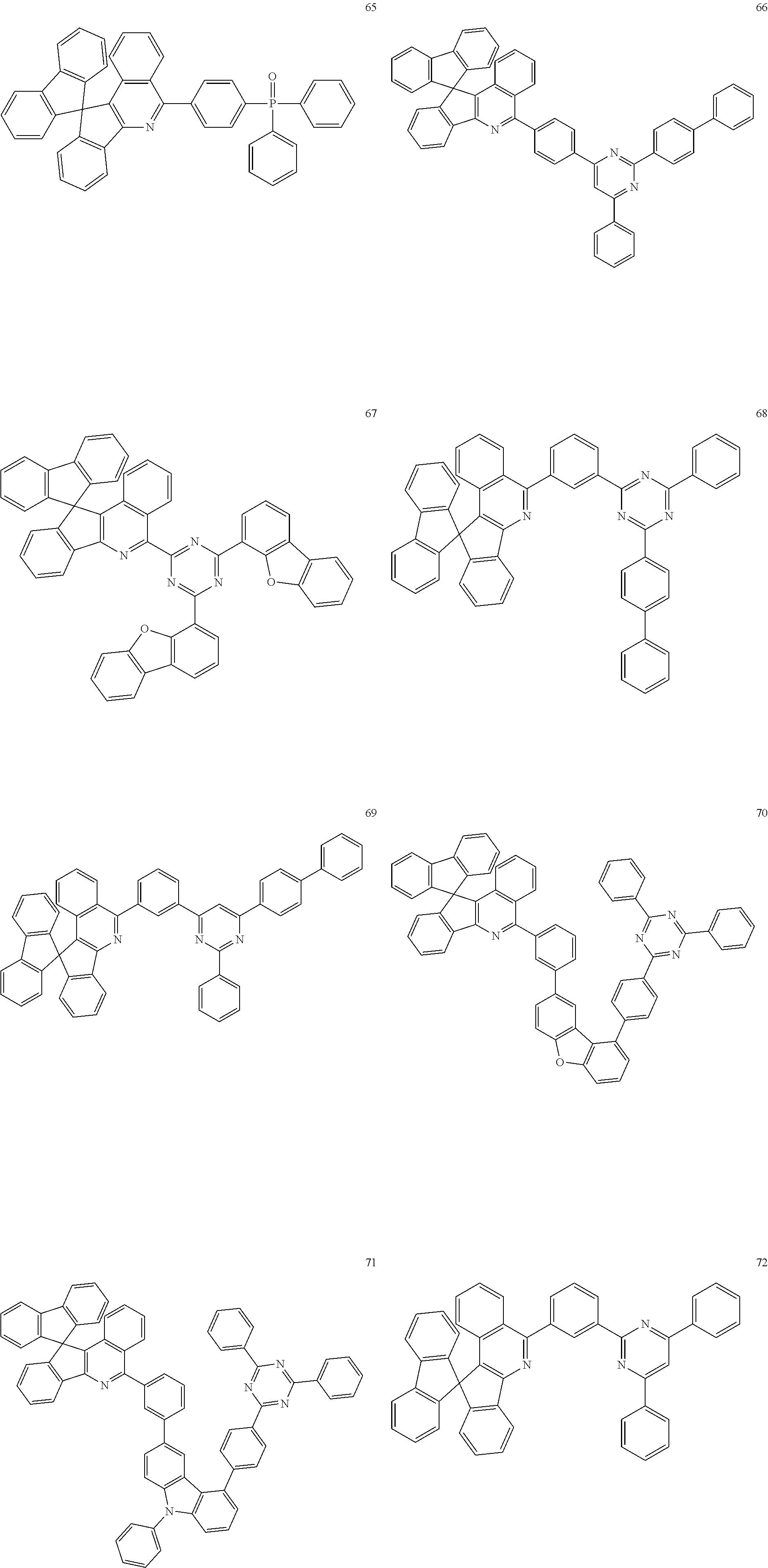

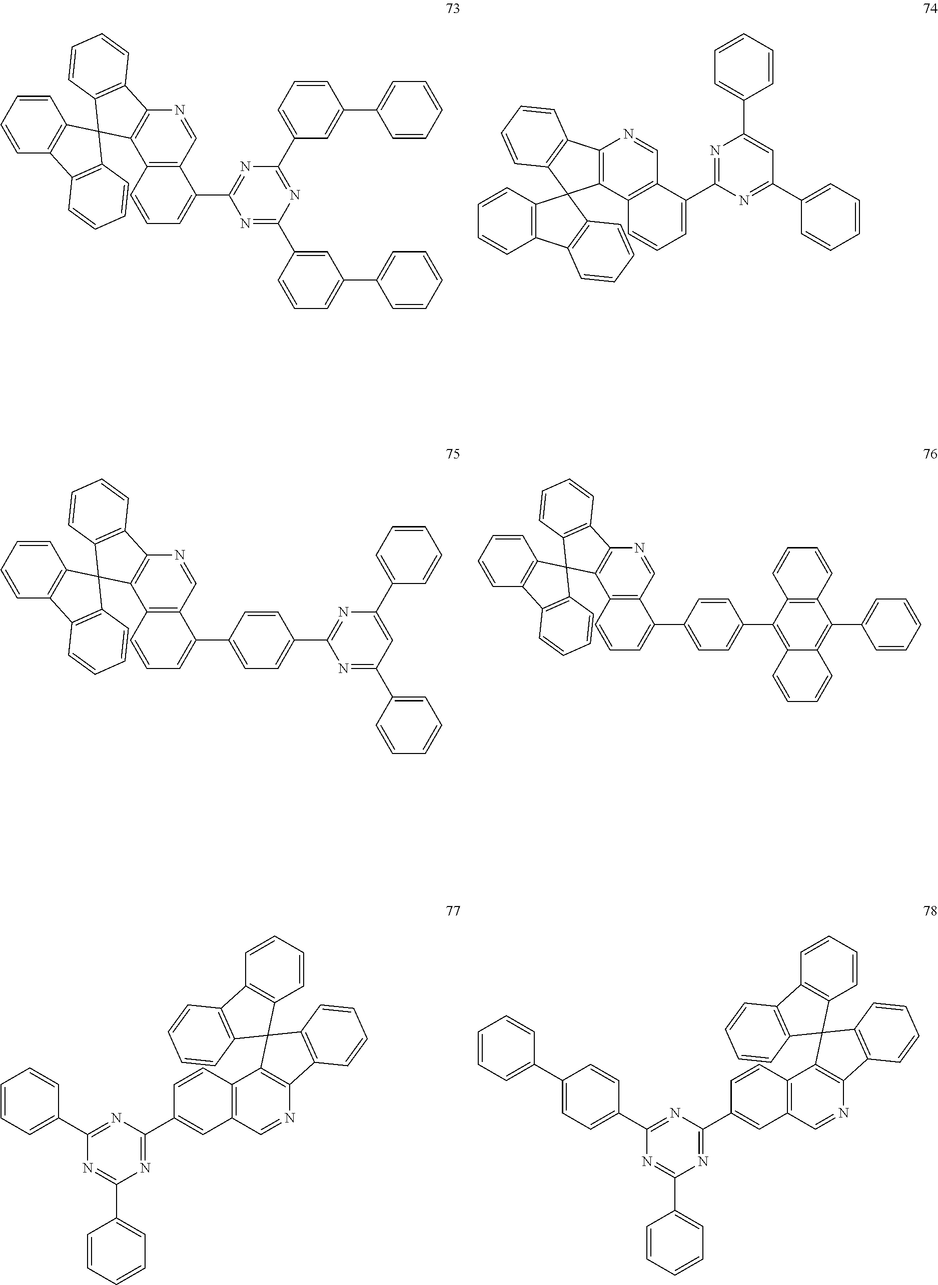

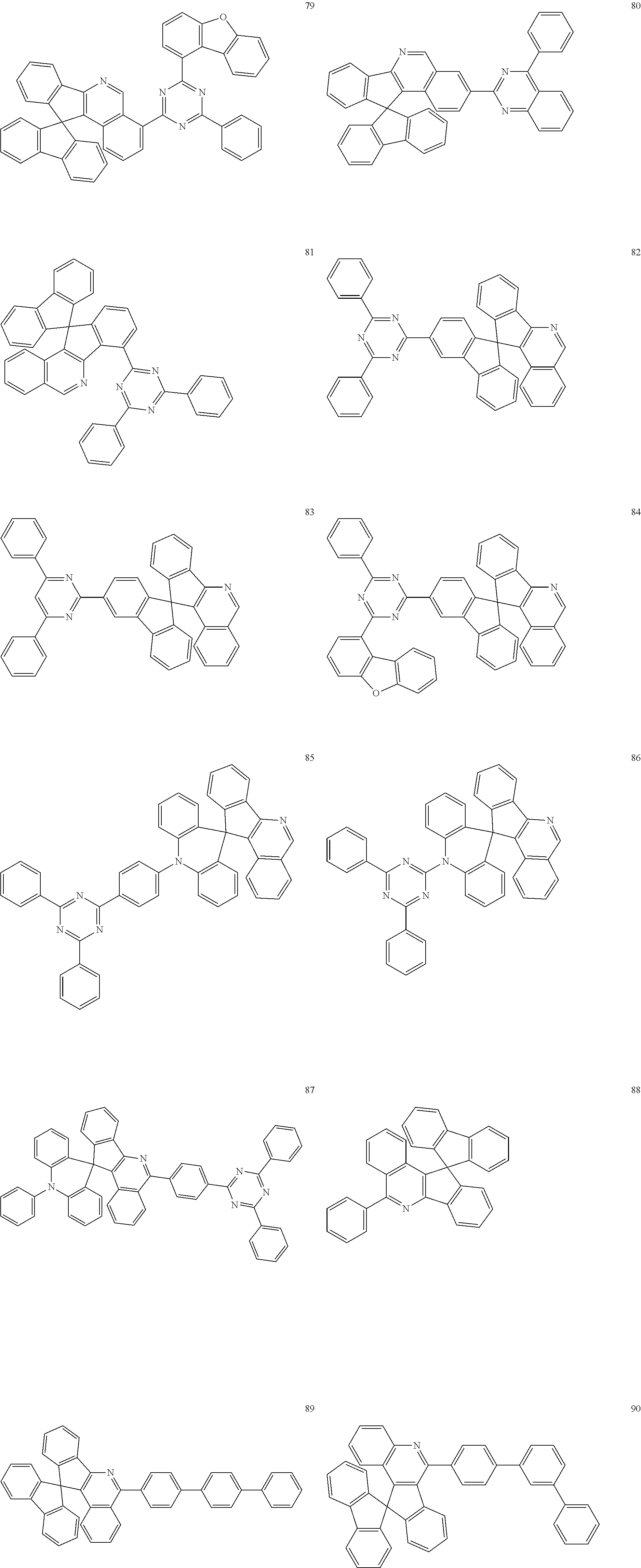

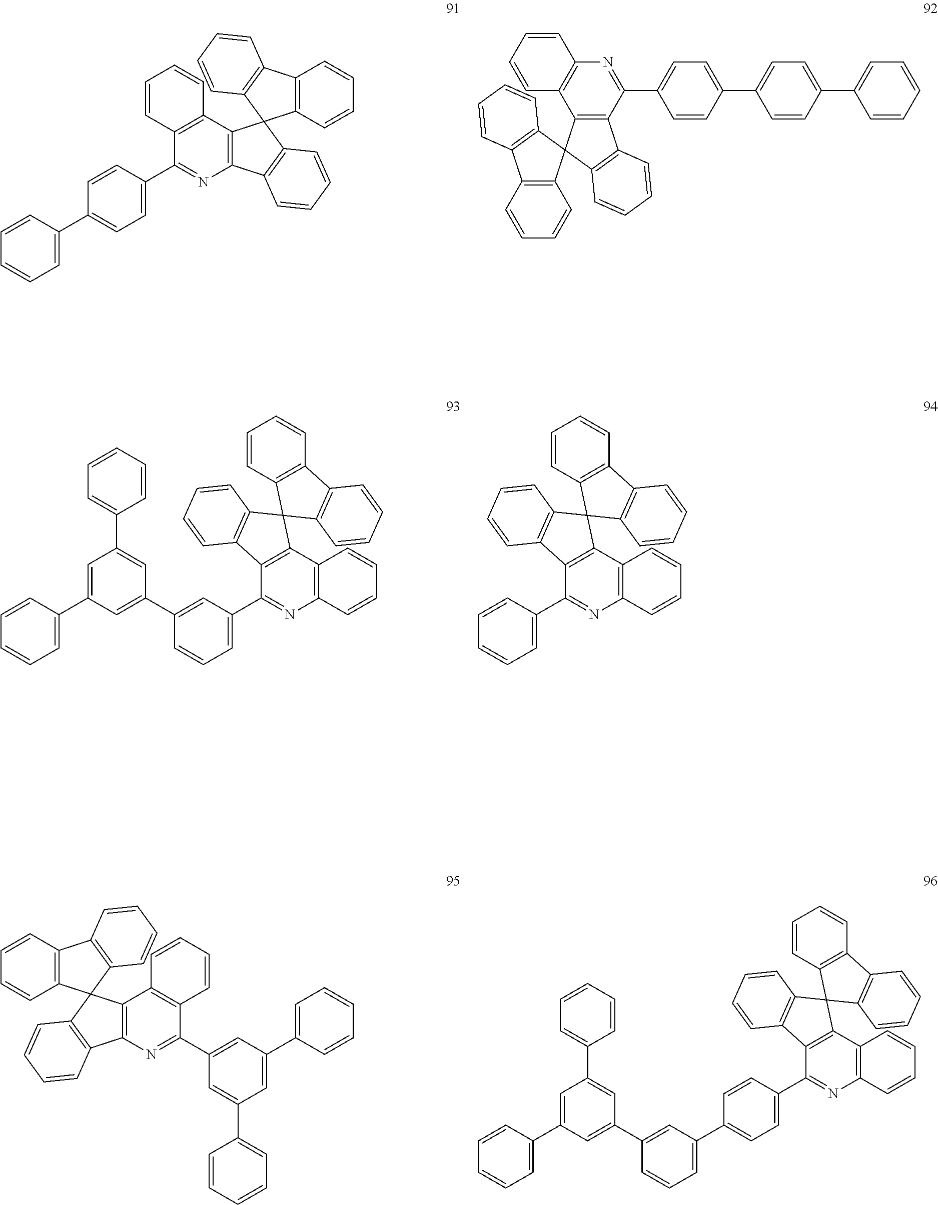 [SMILES: c1ccc(-c2ccc(-c3nc4c(c5ccccc35)C3(c5ccccc5-c5ccccc53)c3ccccc3-4)cc2)cc1 ; c1ccc(-c2ccc(-c3ccc(-c4nc5ccccc5c5c4-c4ccccc4C54c5ccccc5-c5ccccc54)cc3)cc2)cc1 ; c1ccc(-c2cc(-c3ccccc3)cc(-c3cccc(-c4nc5ccccc5c5c4-c4ccccc4C54c5ccccc5-c5ccccc54)c3)c2)cc1 ; c1ccc(-c2nc3ccccc3c3c2-c2ccccc2C32c3ccccc3-c3ccccc32)cc1 ; c1ccc(-c2cc(-c3ccccc3)cc(-c3nc4c(c5ccccc35)C3(c5ccccc5-c5ccccc53)c3ccccc3-4)c2)cc1 ; c1ccc(-c2cc(-c3ccccc3)cc(-c3cccc(-c4ccc(-c5nc6ccccc6c6c5-c5ccccc5C65c6ccccc6-c6ccccc65)cc4)c3)c2)cc1]

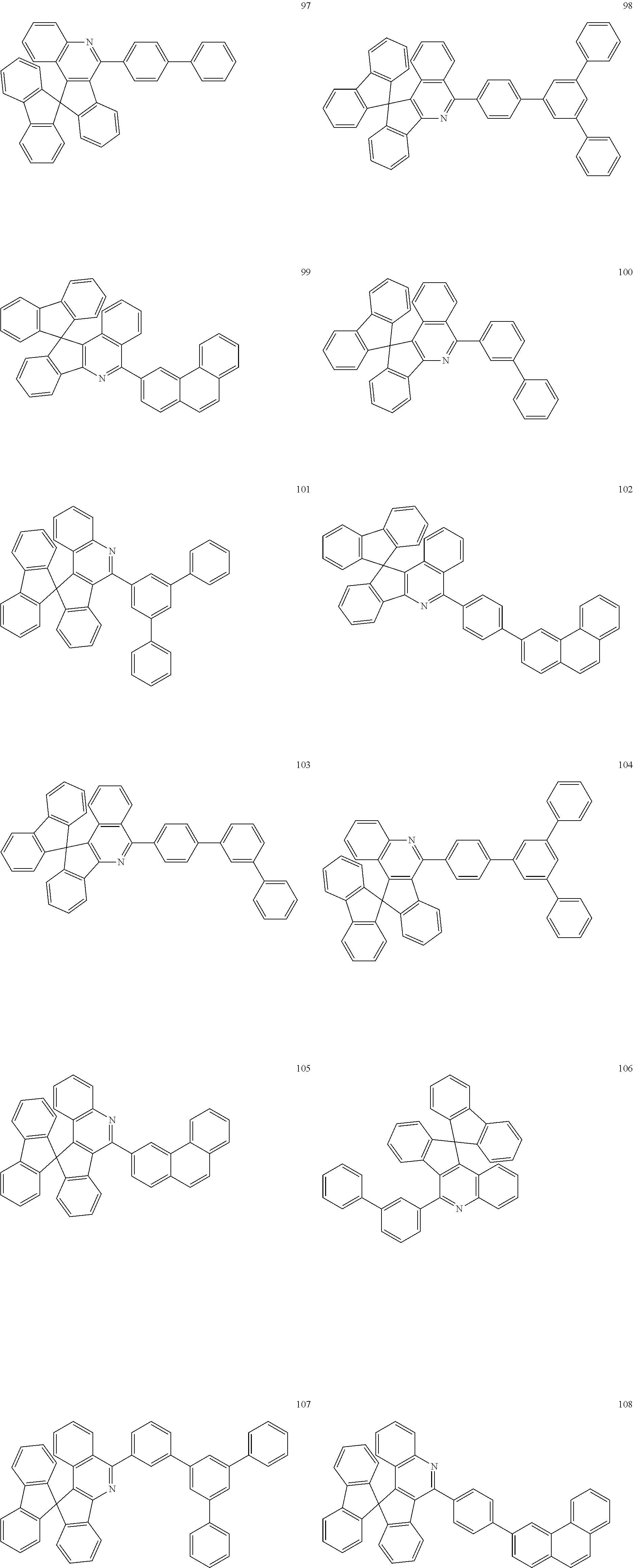

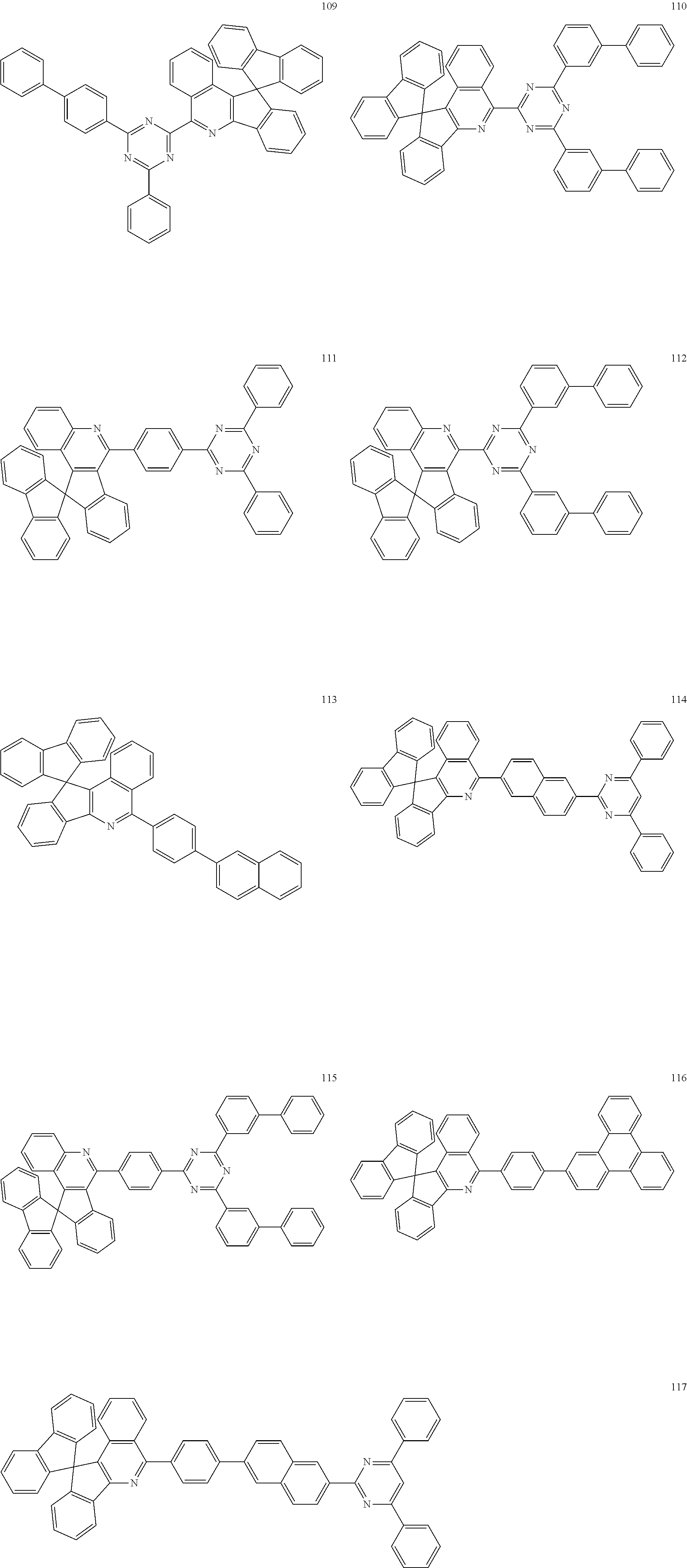

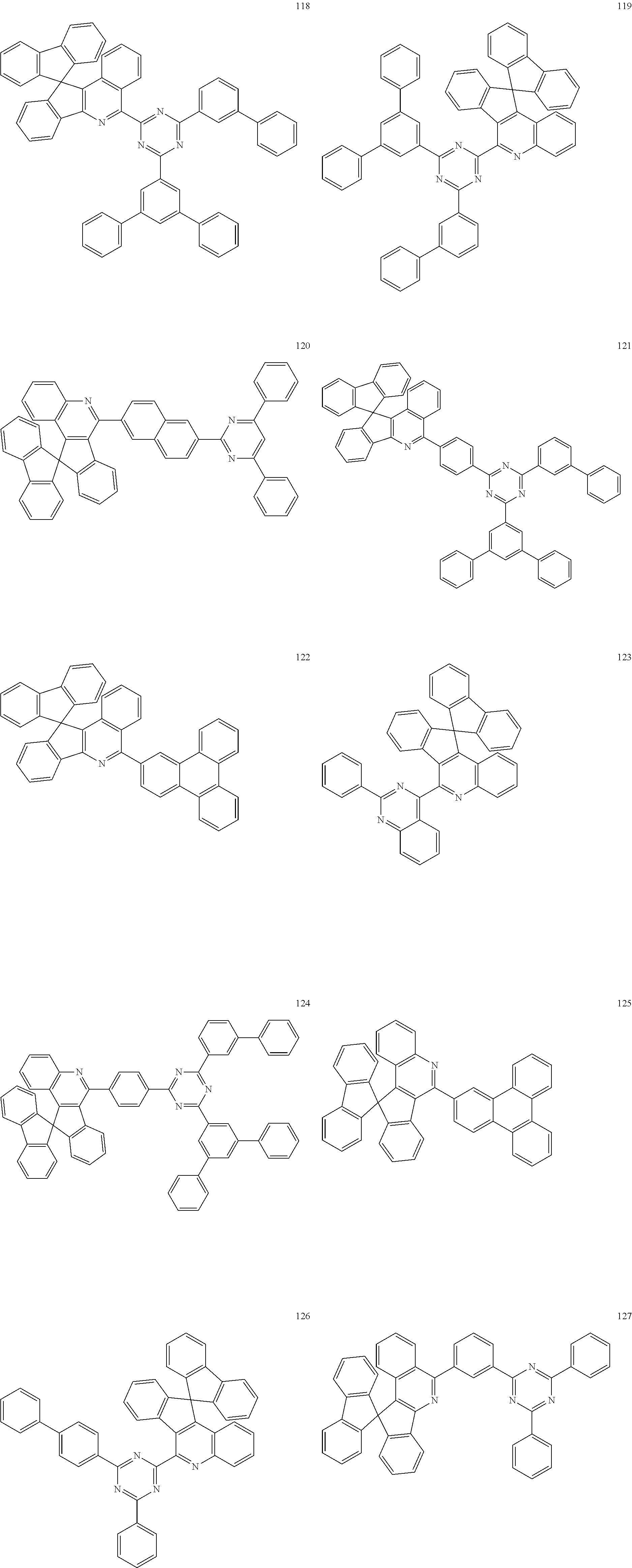

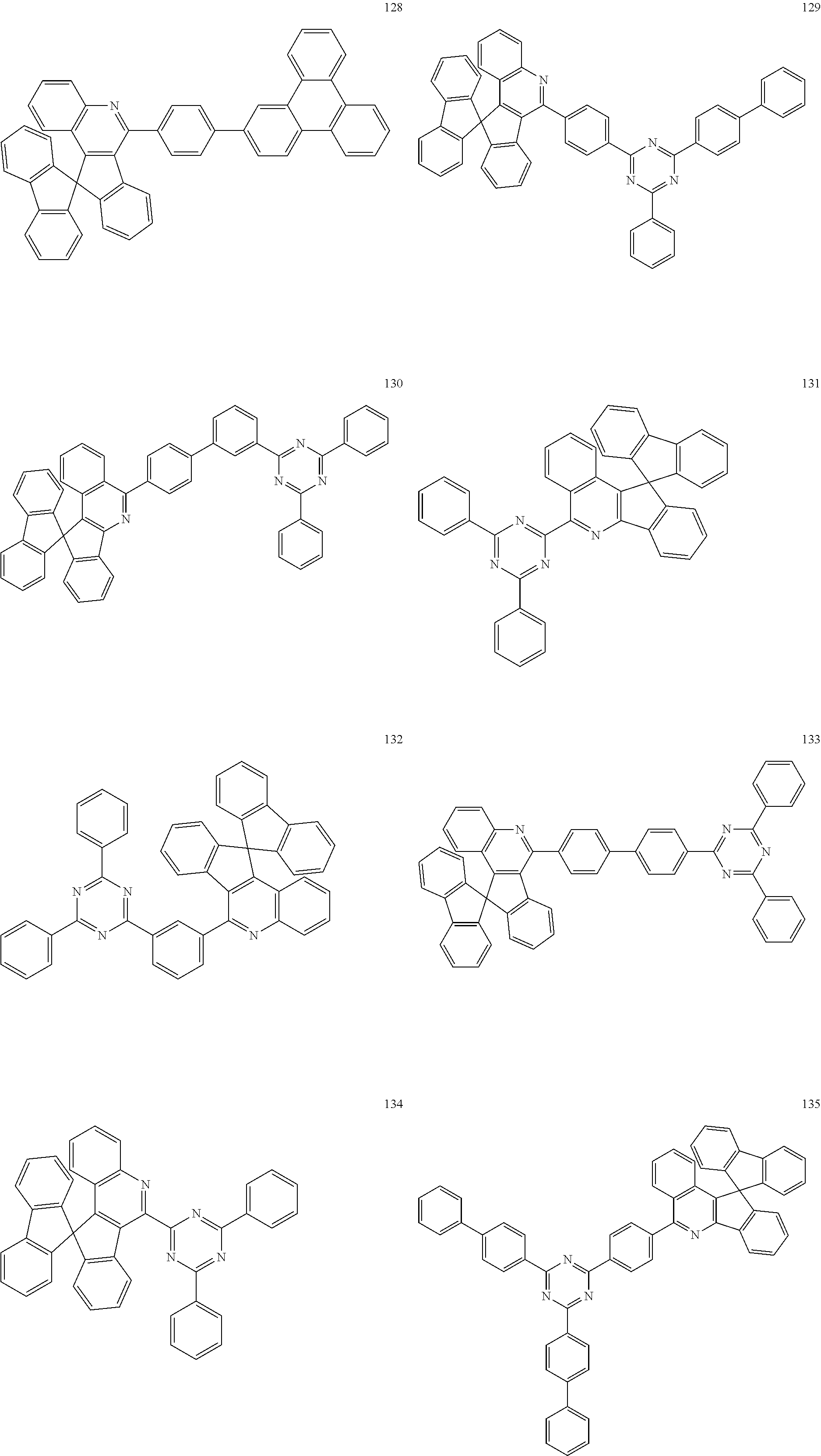

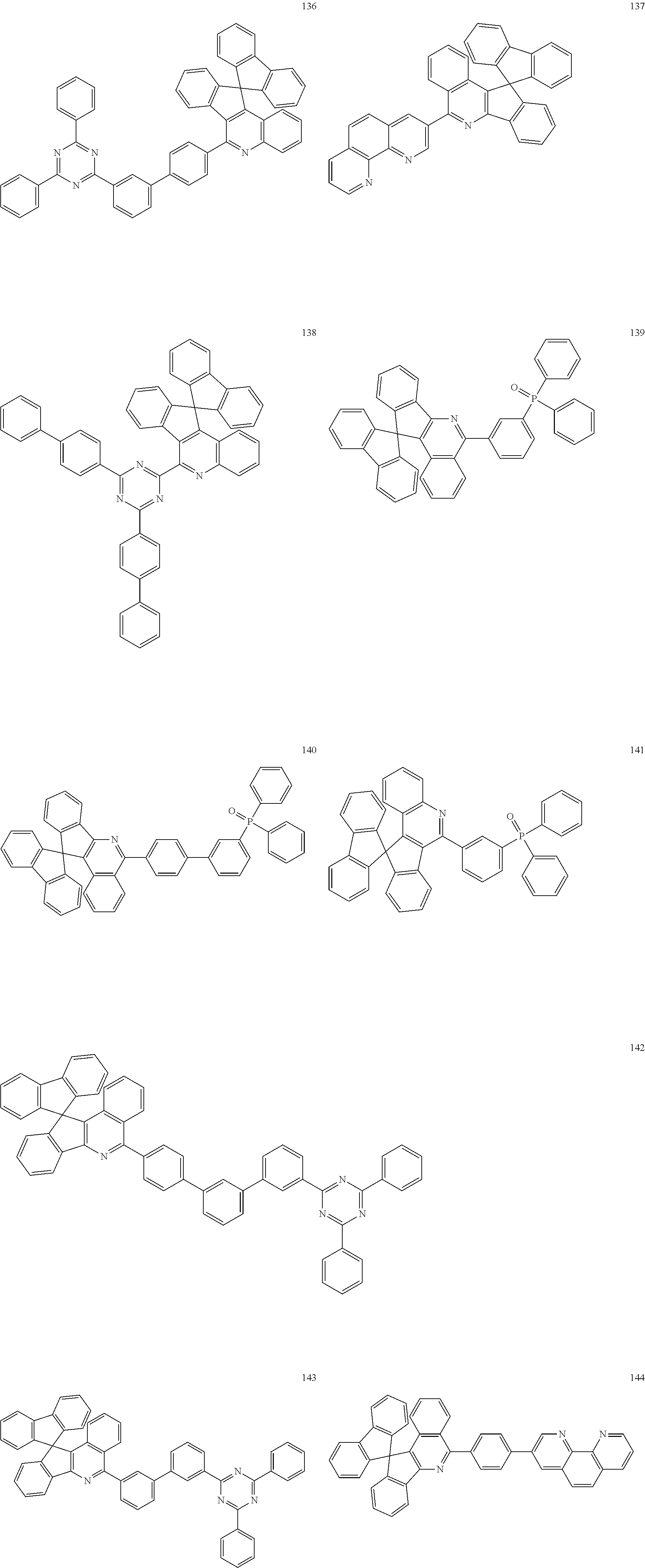

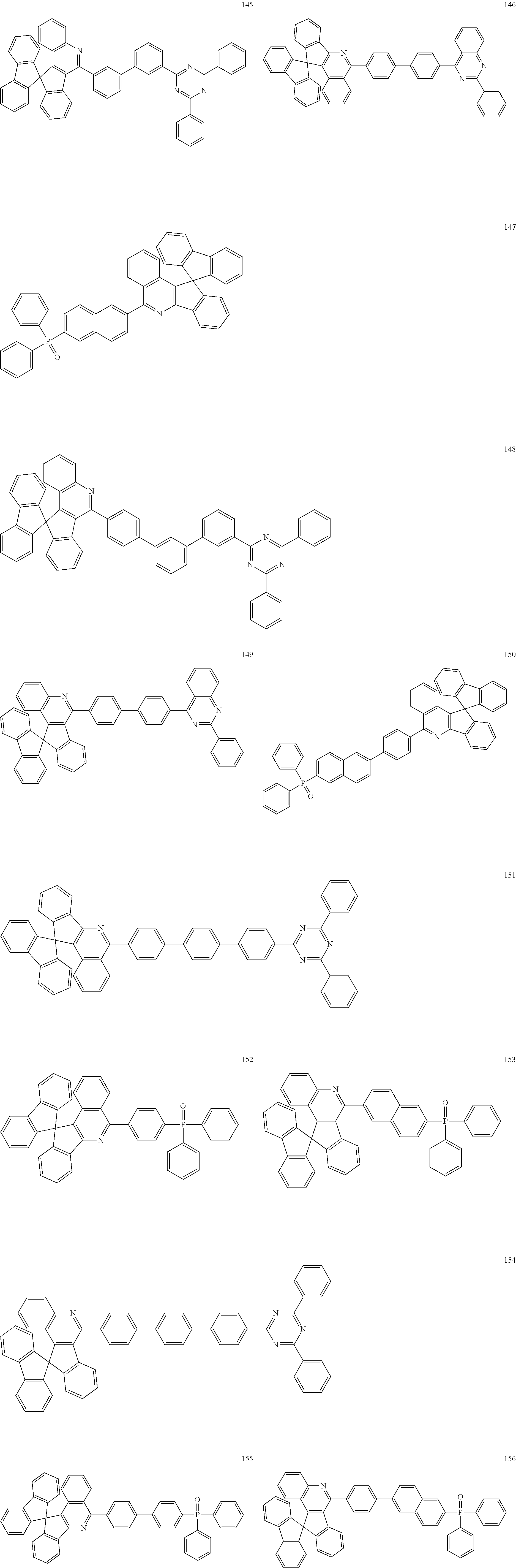

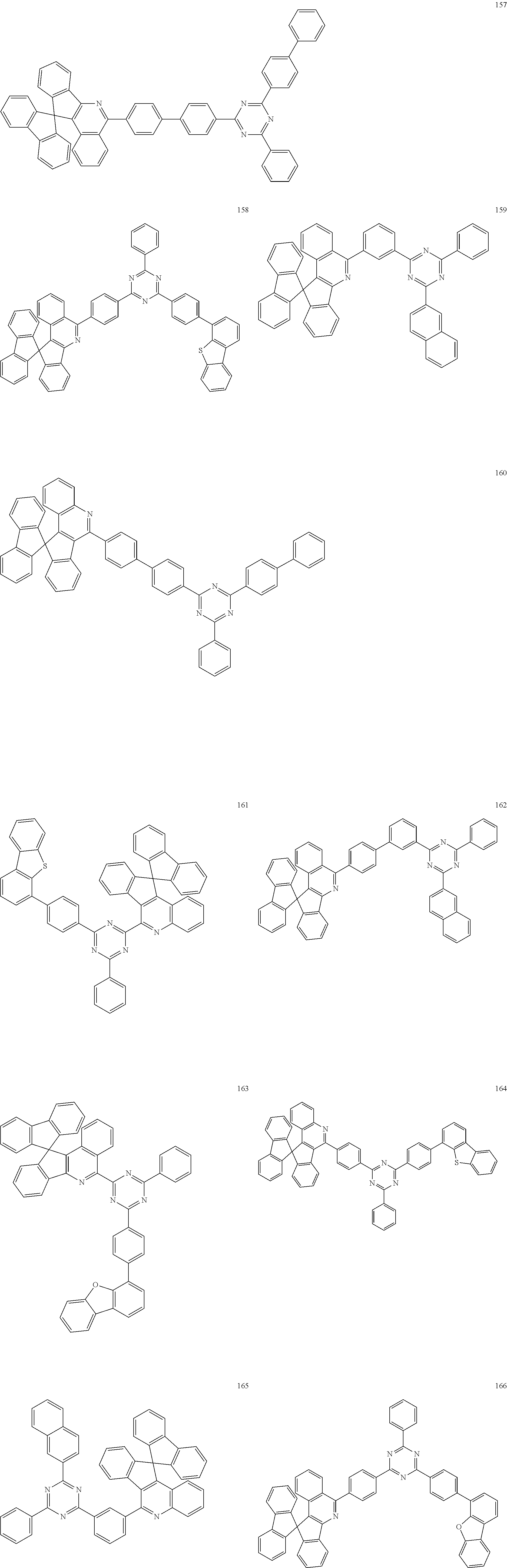

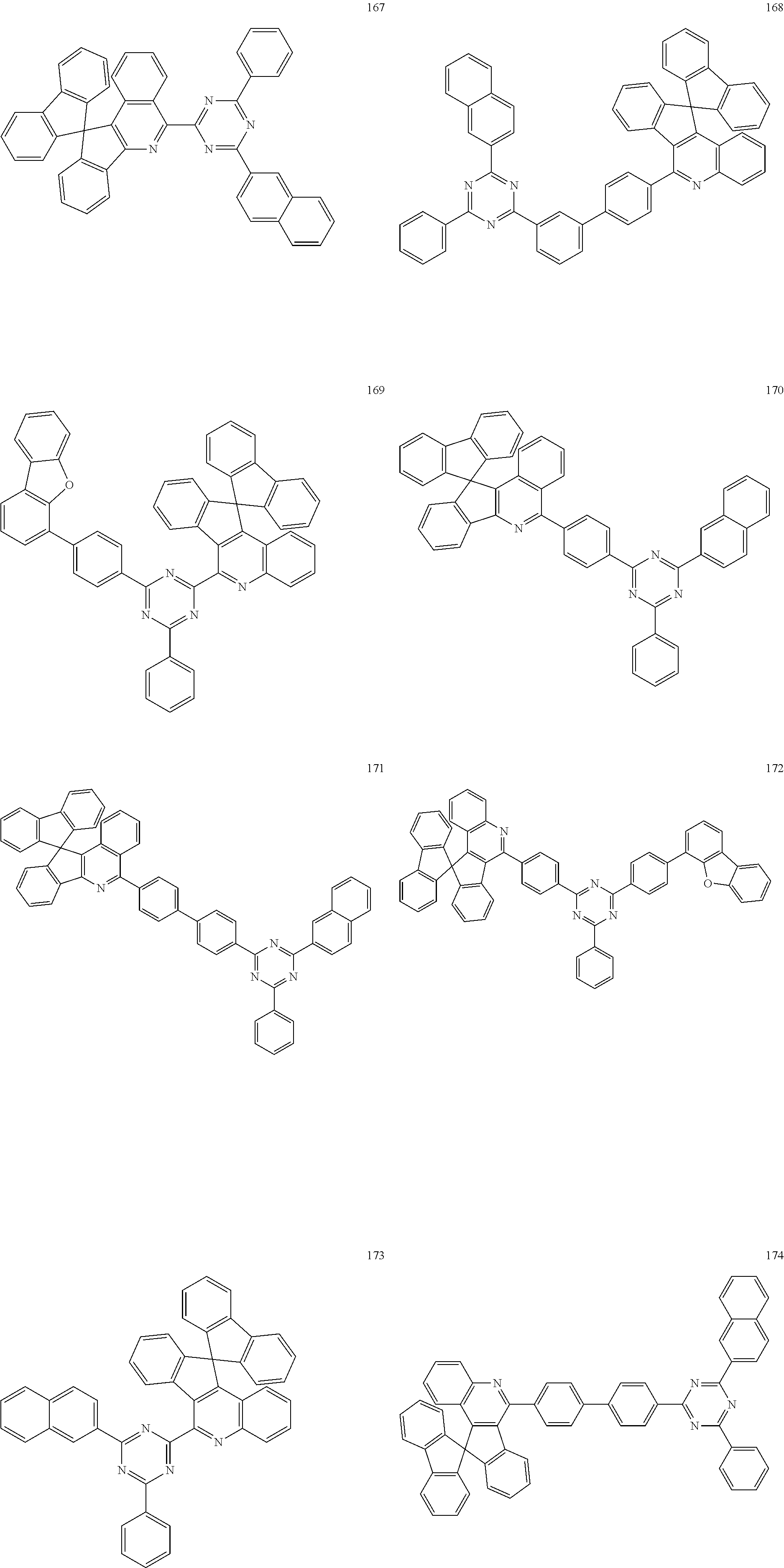

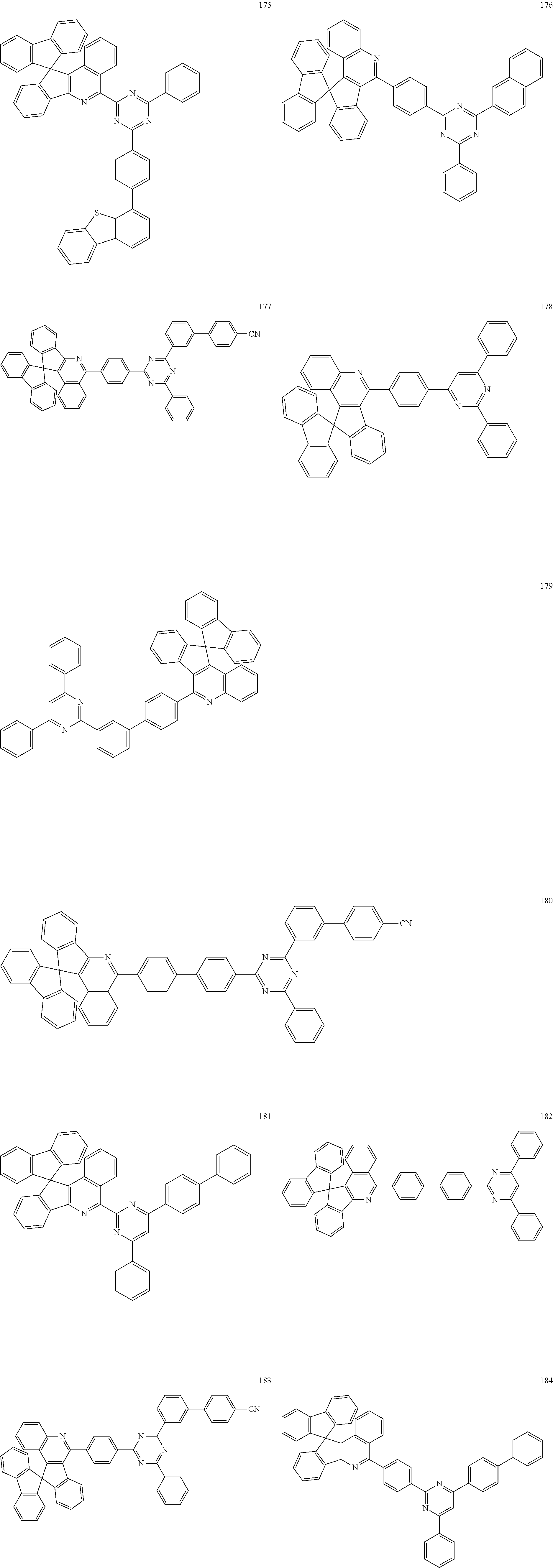

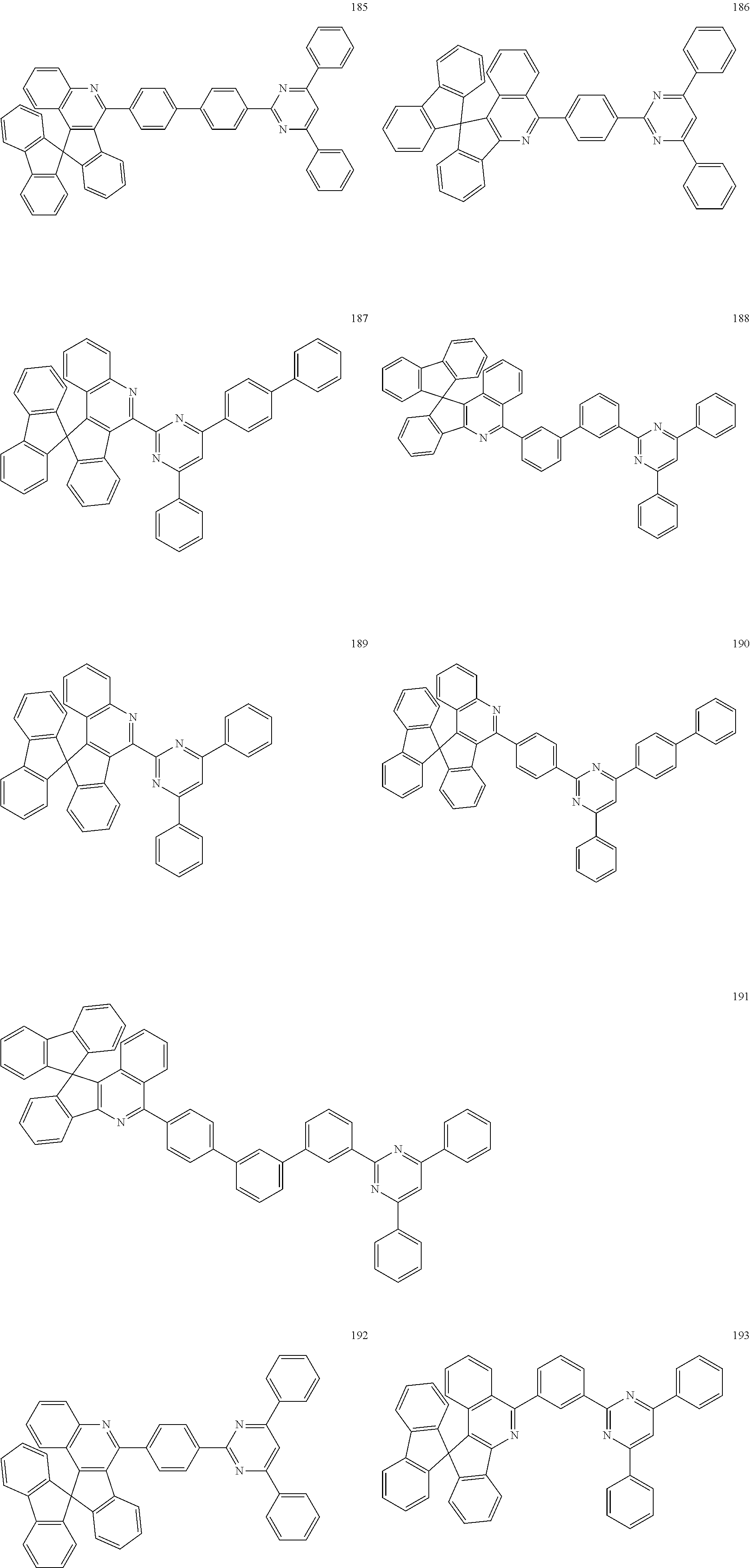

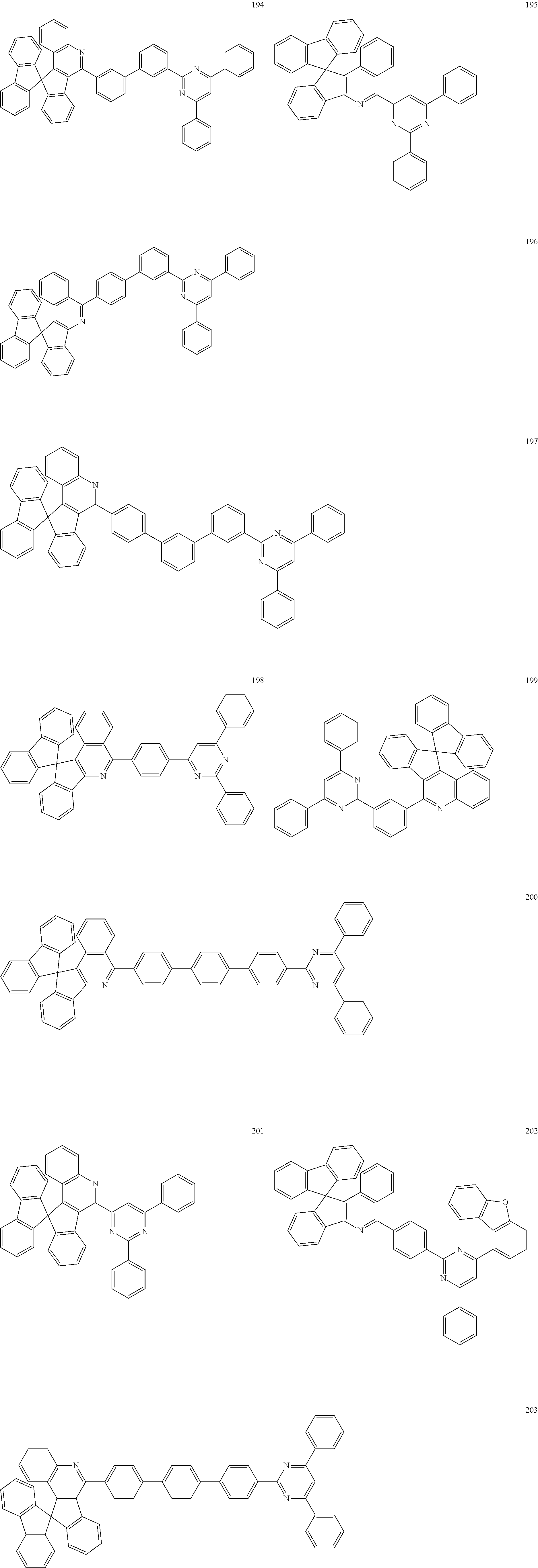

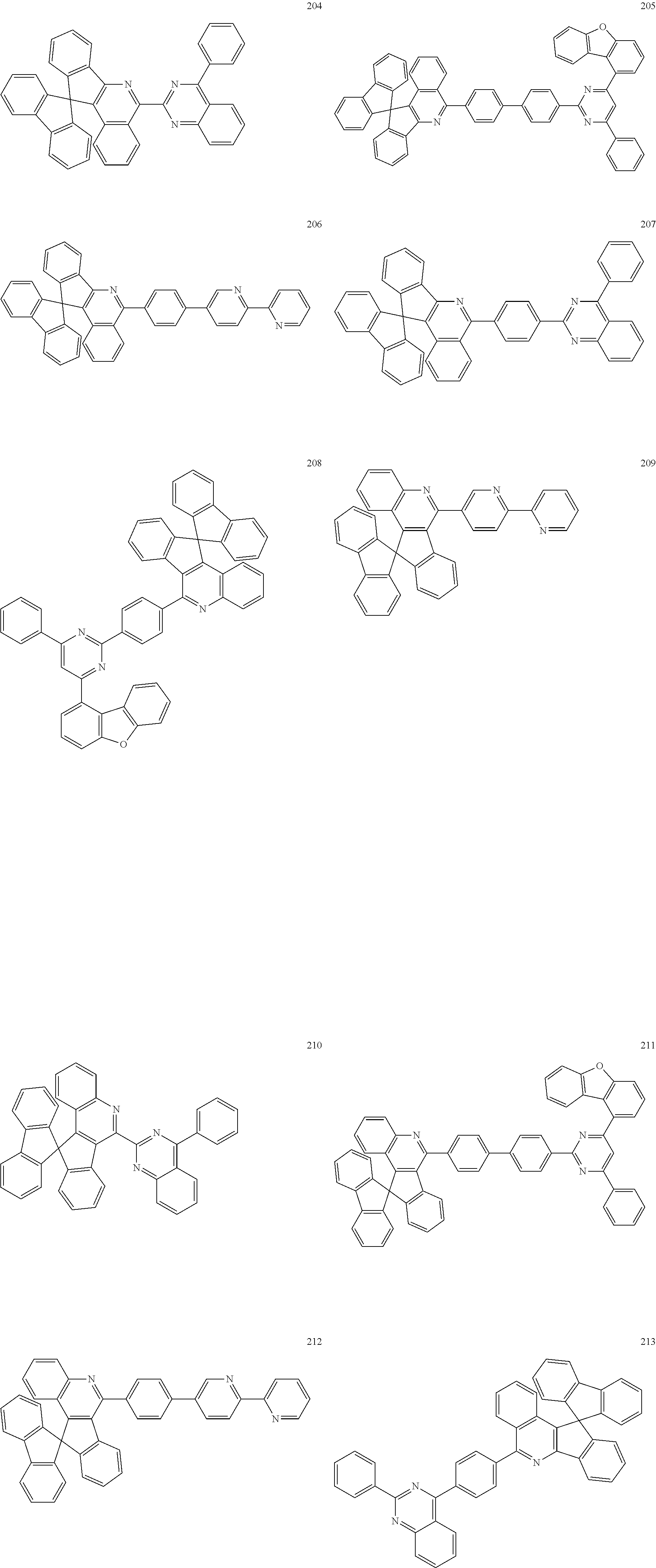

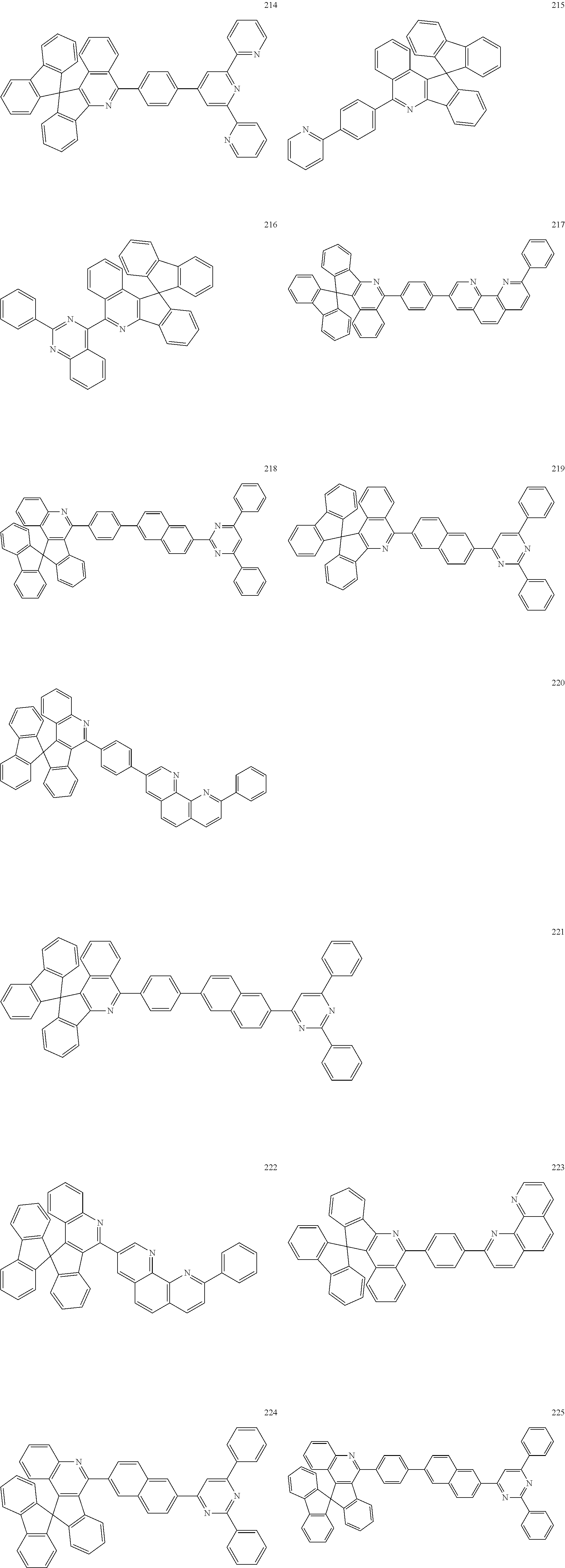

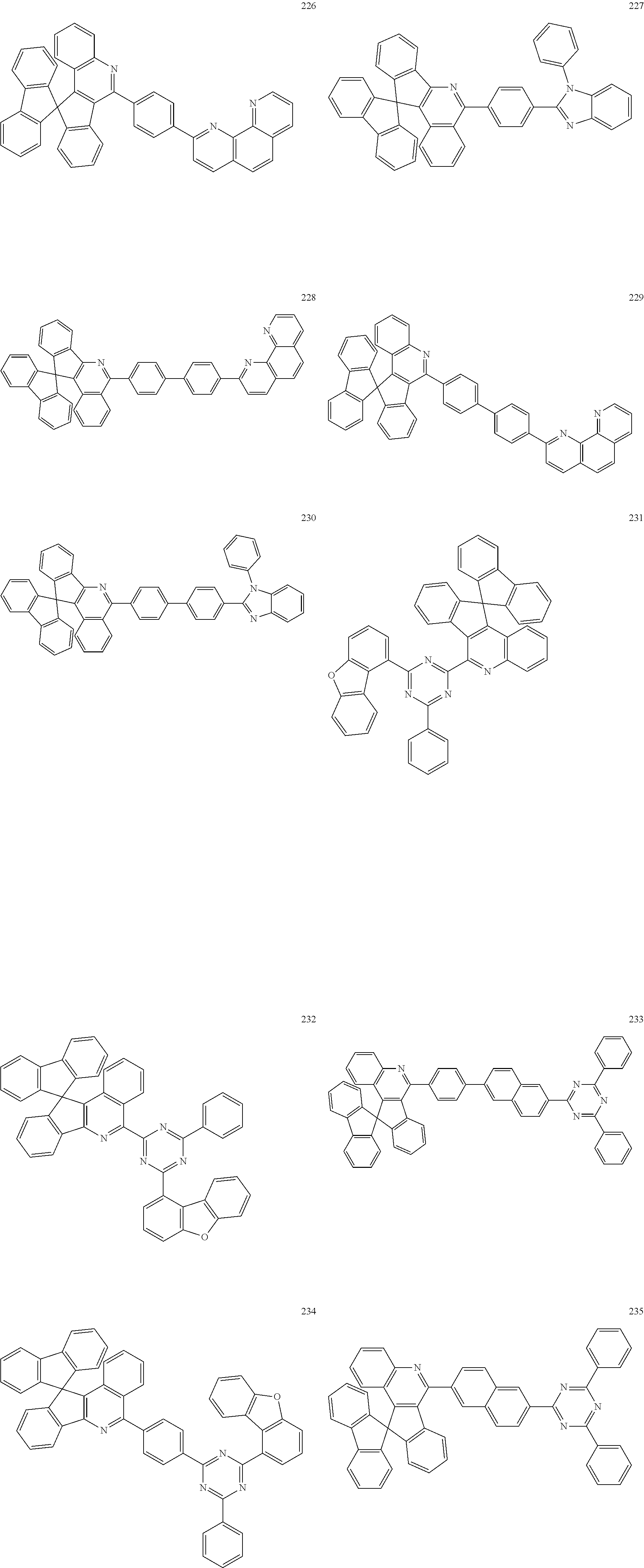

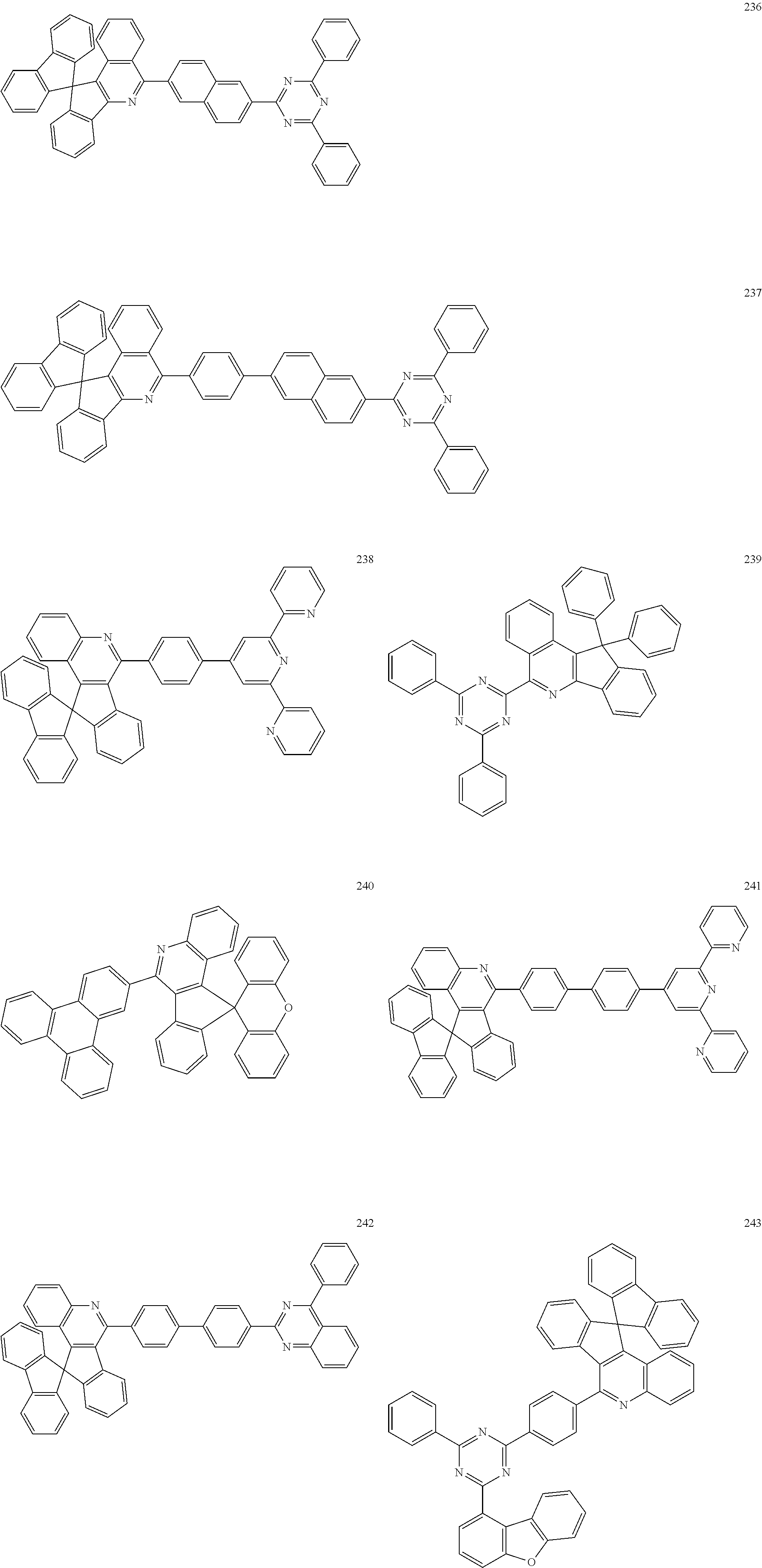

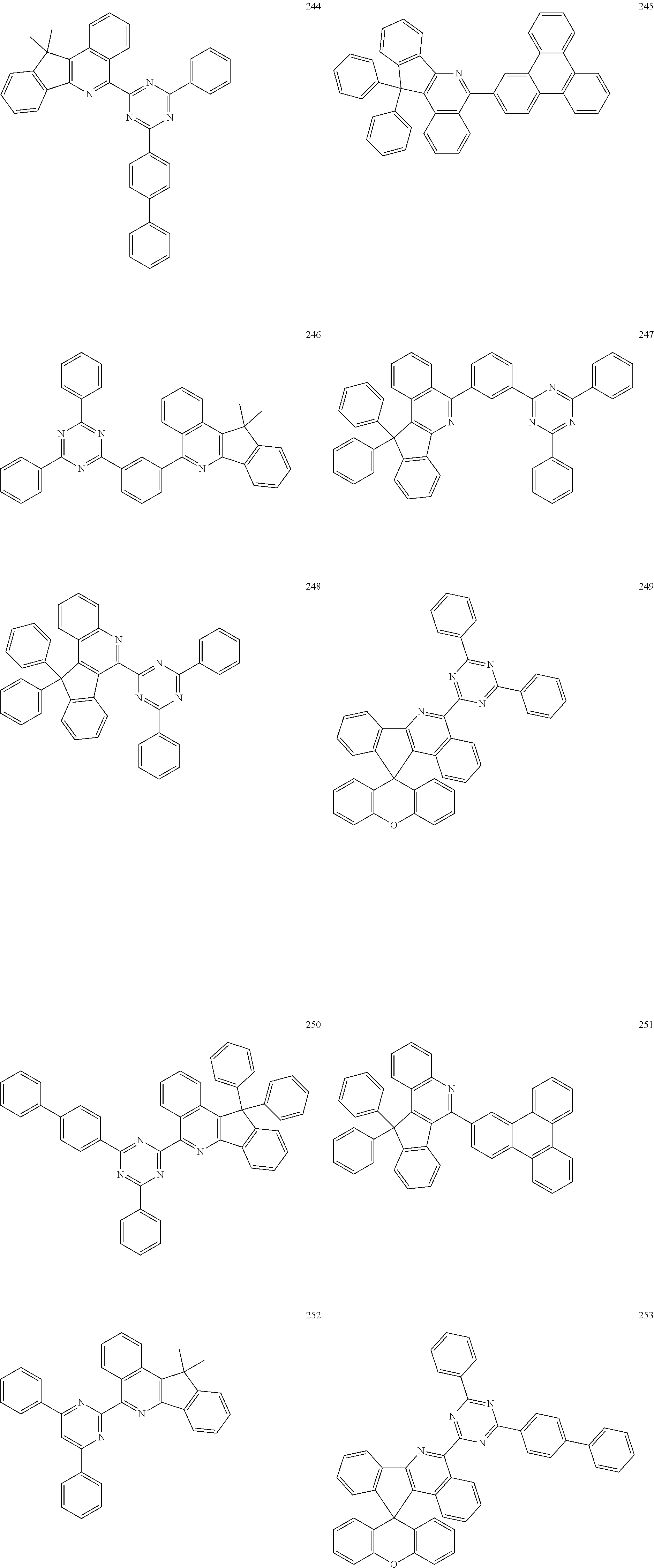

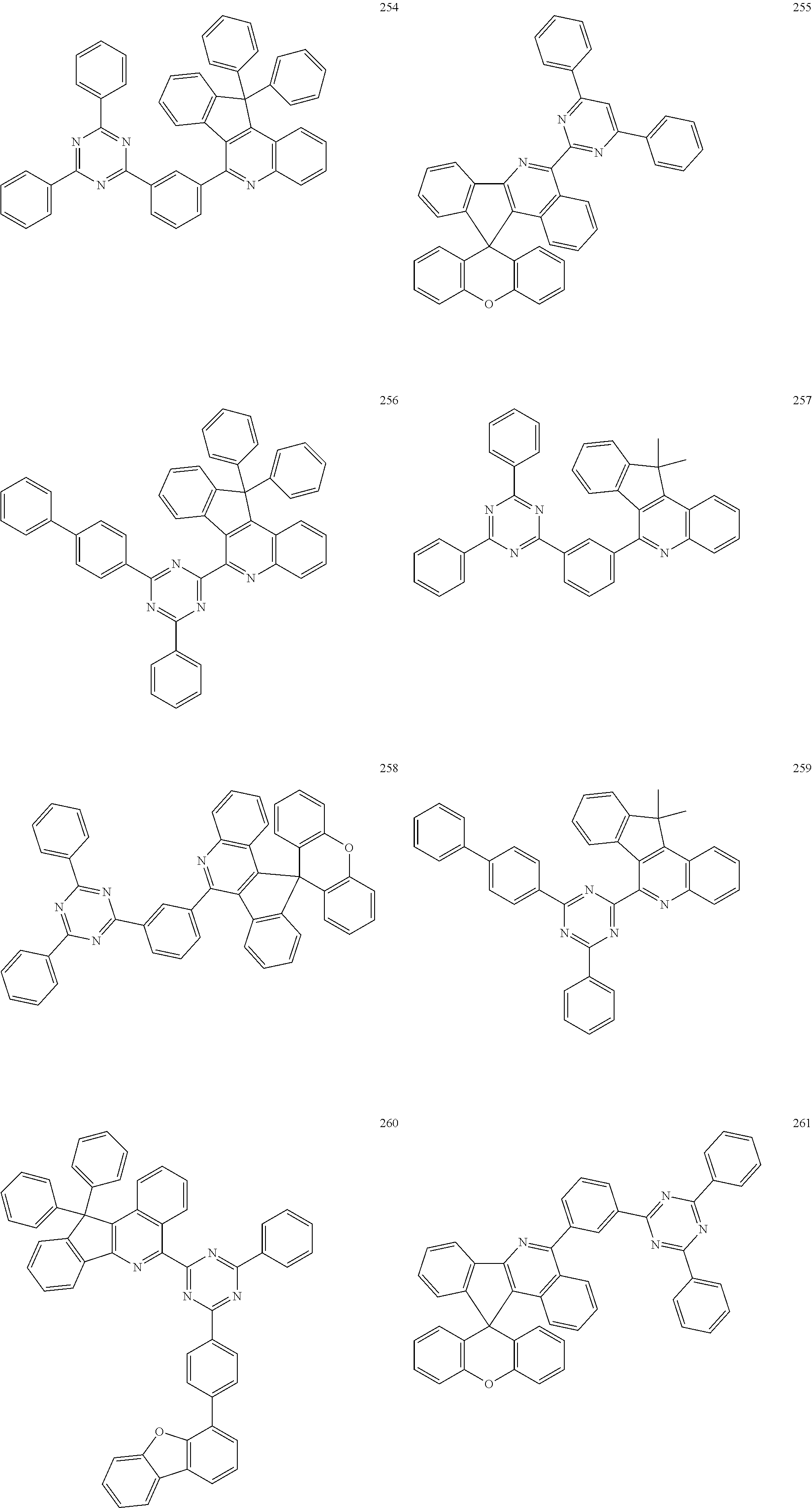

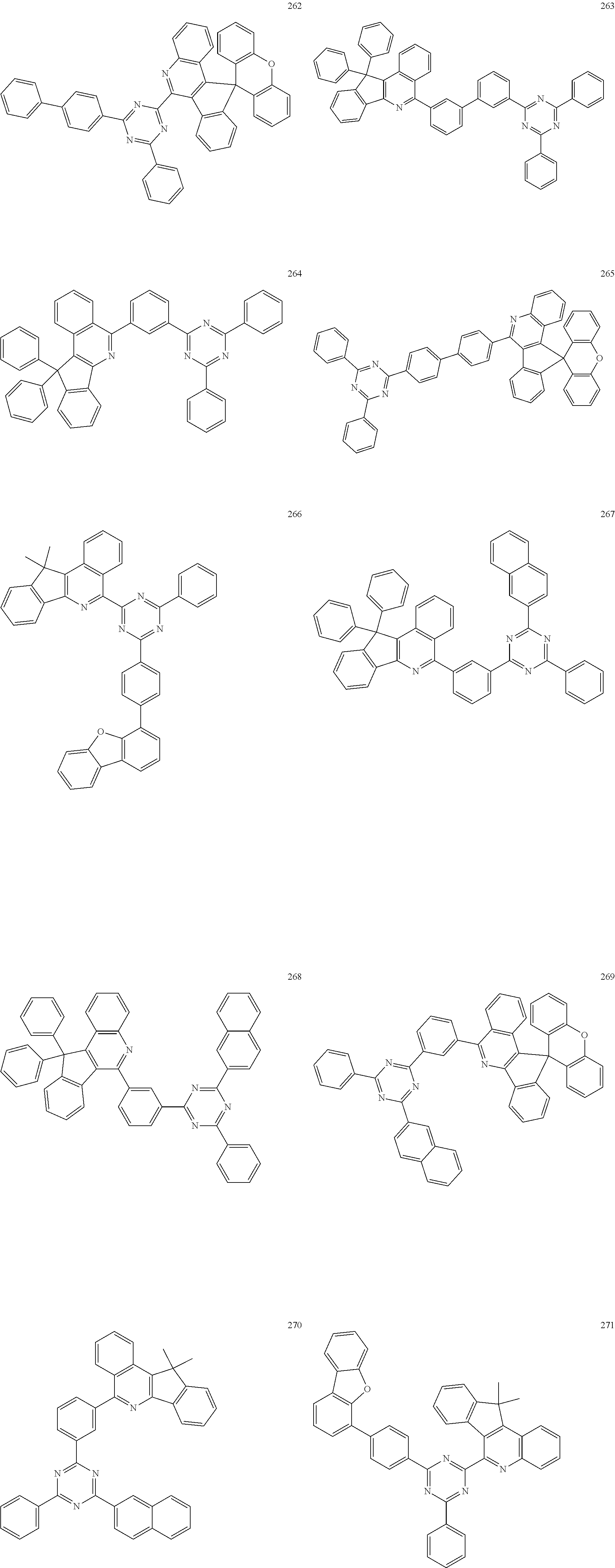

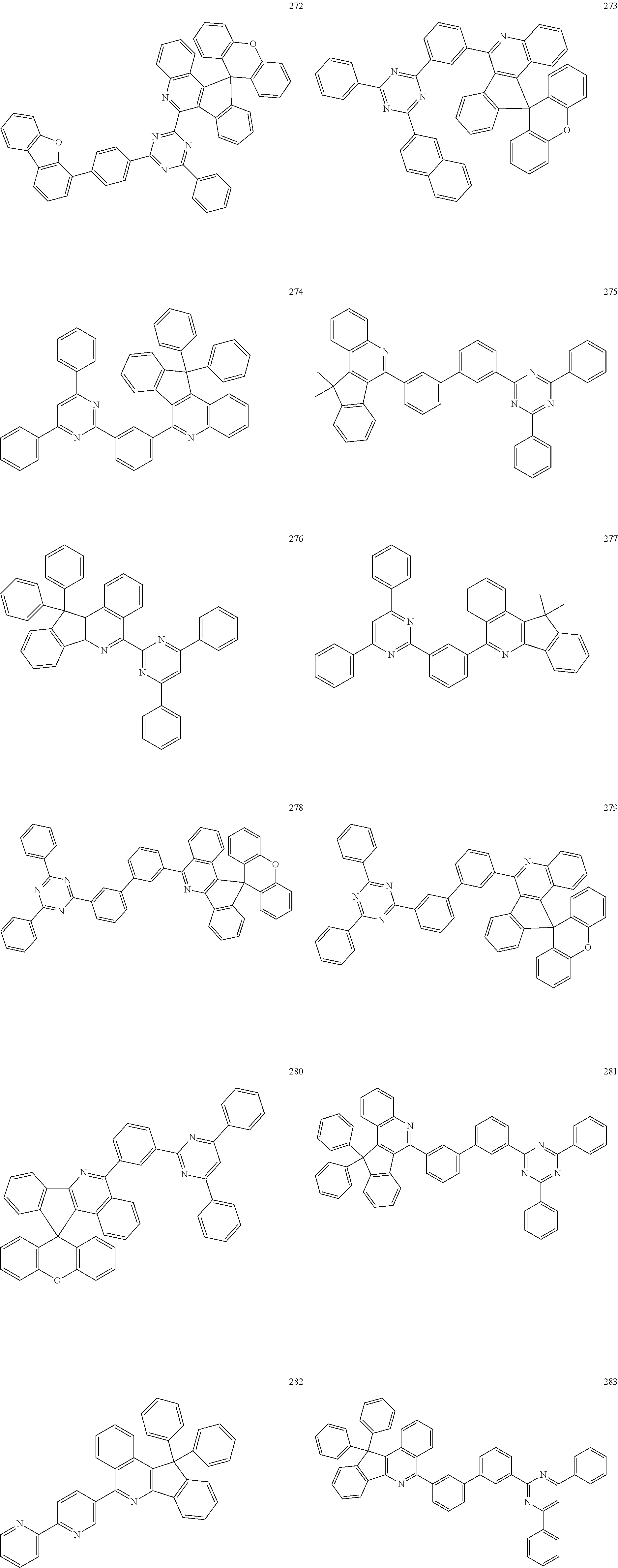

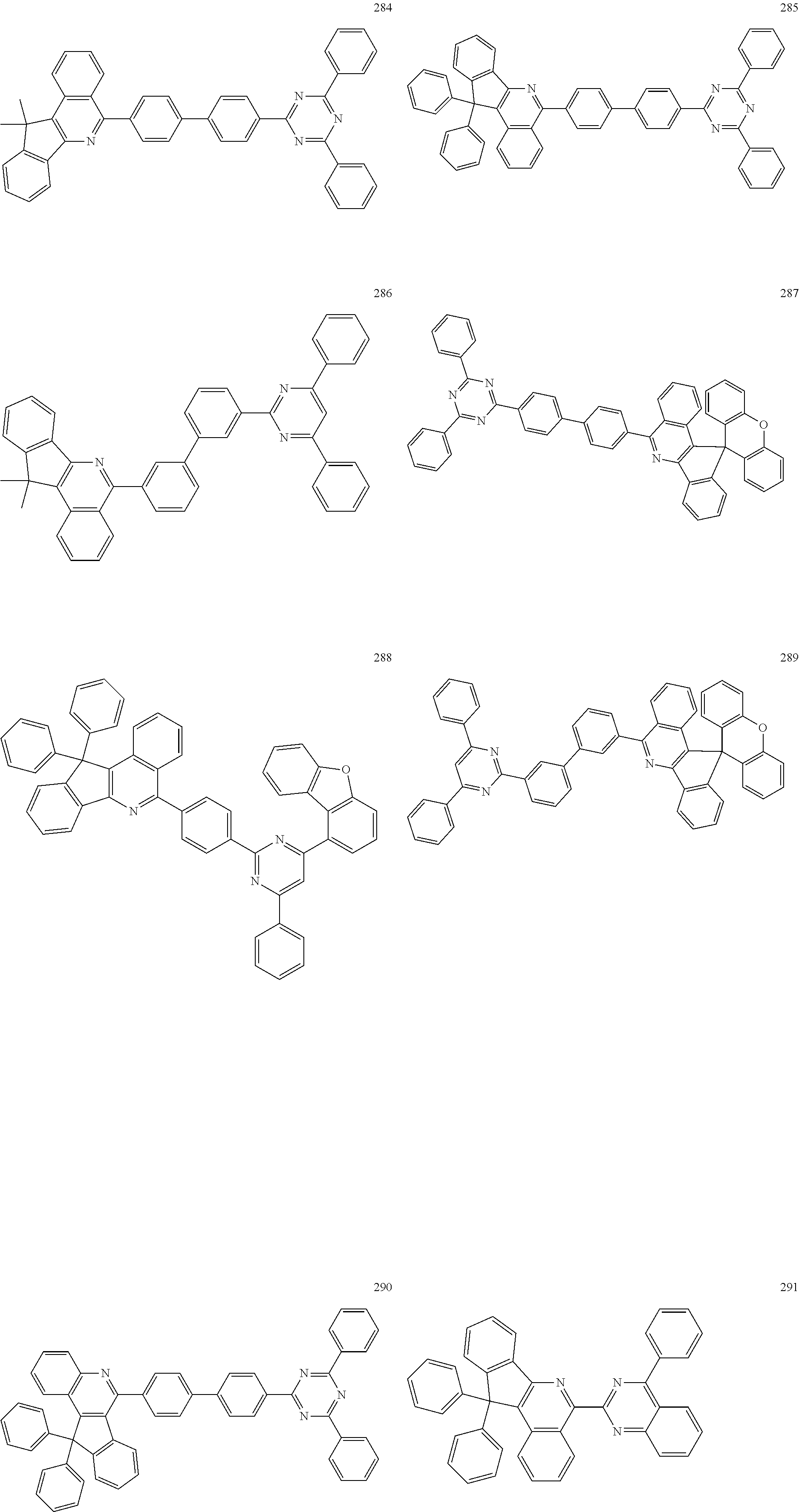

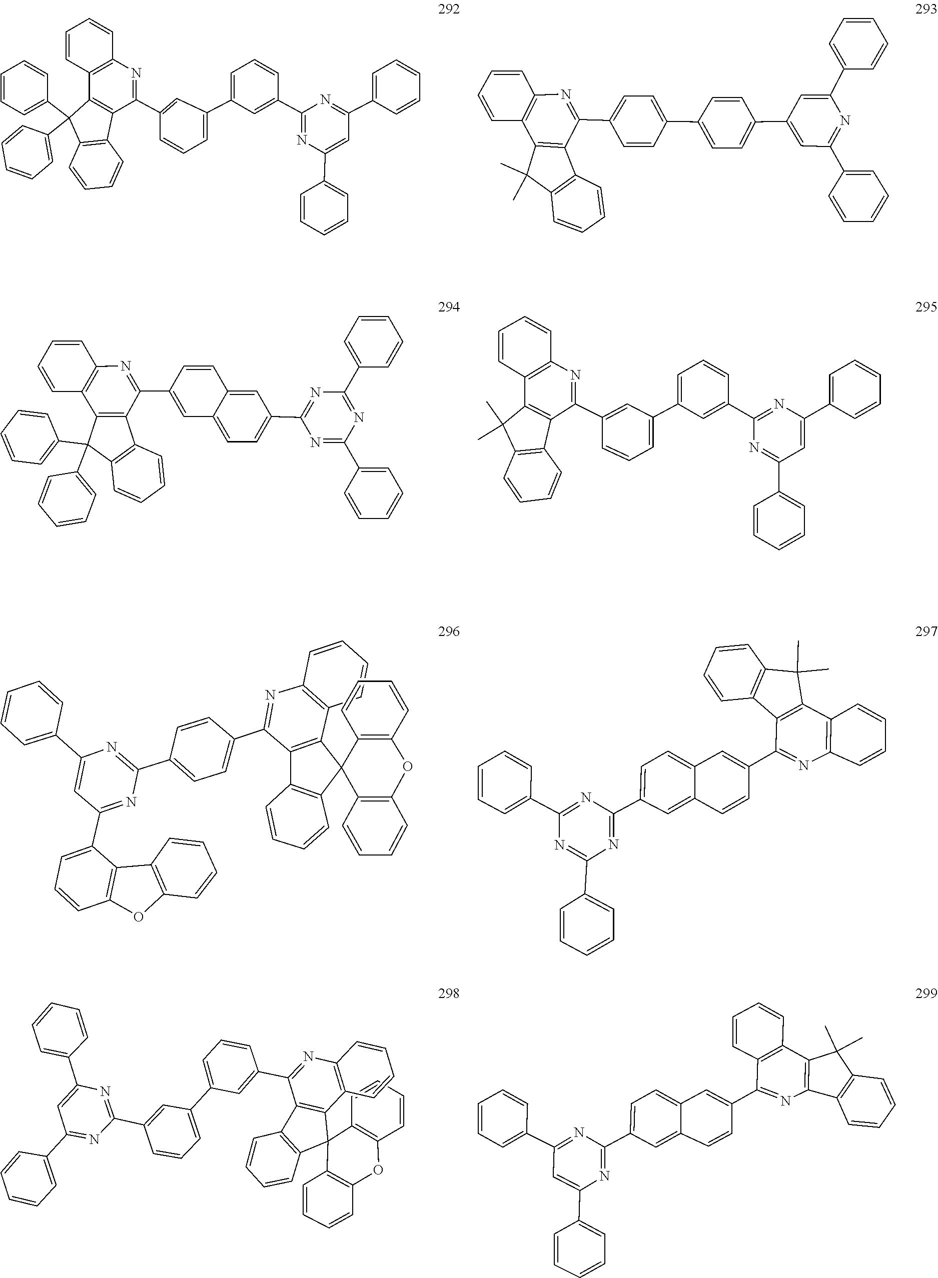

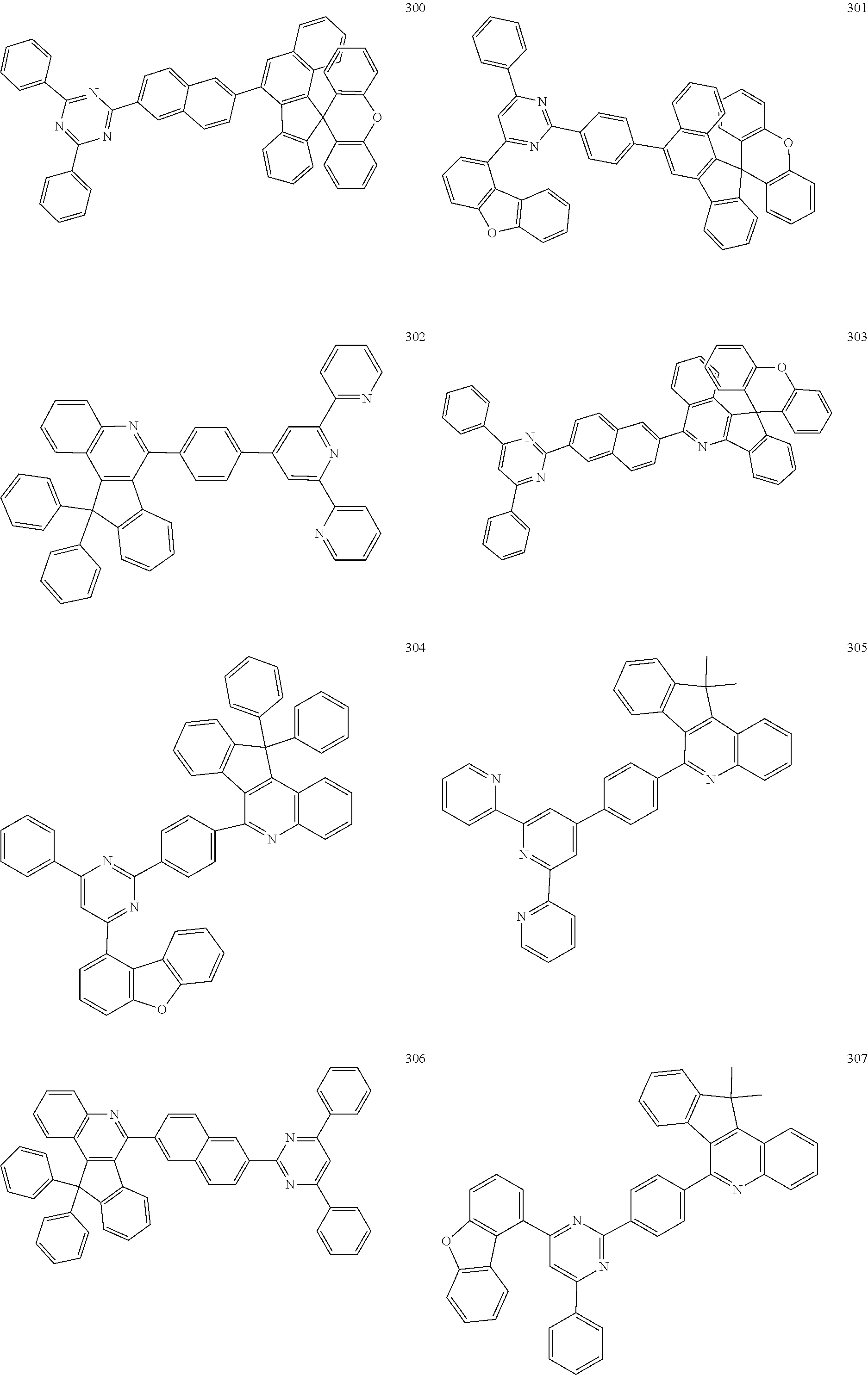

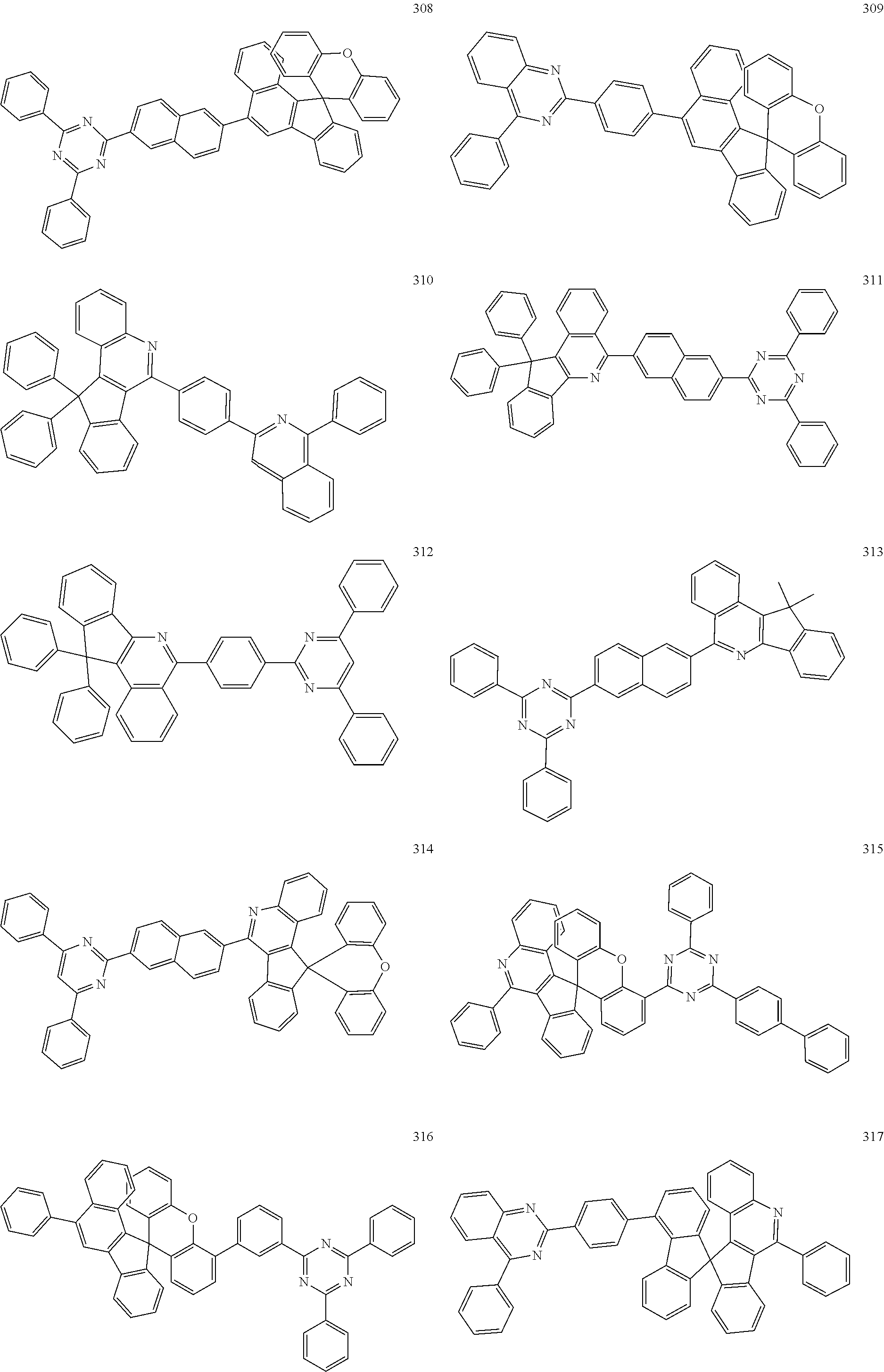

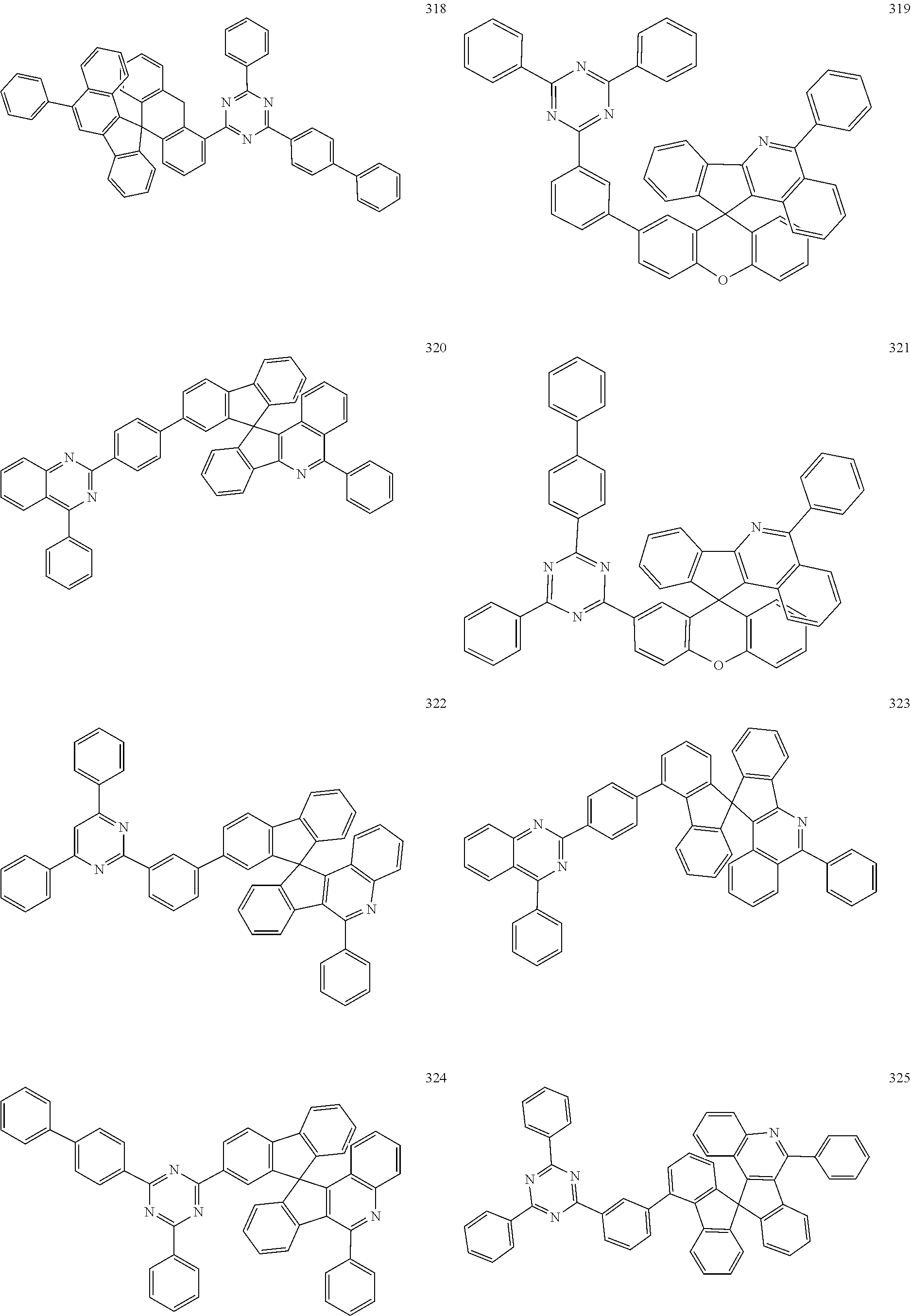

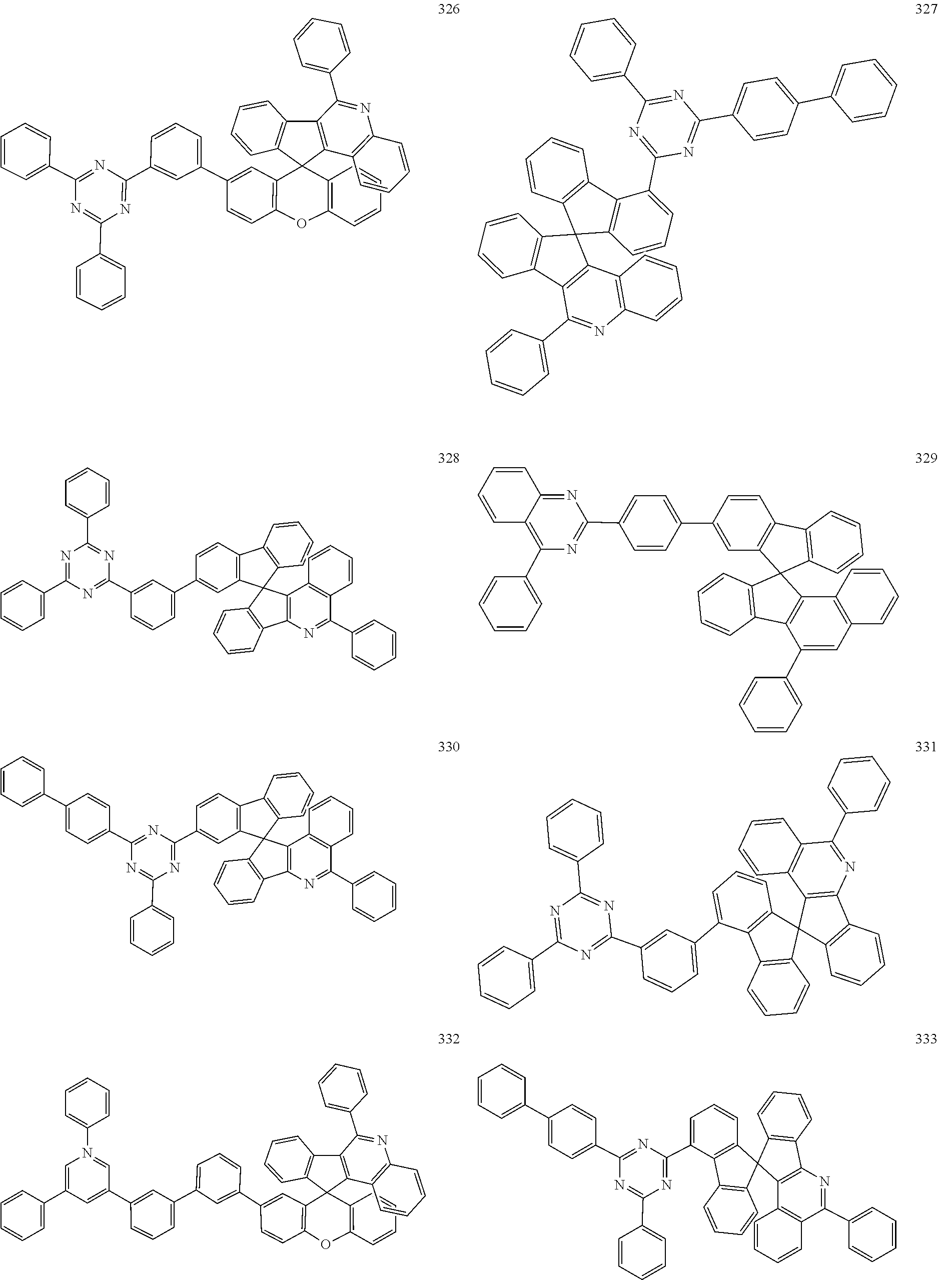

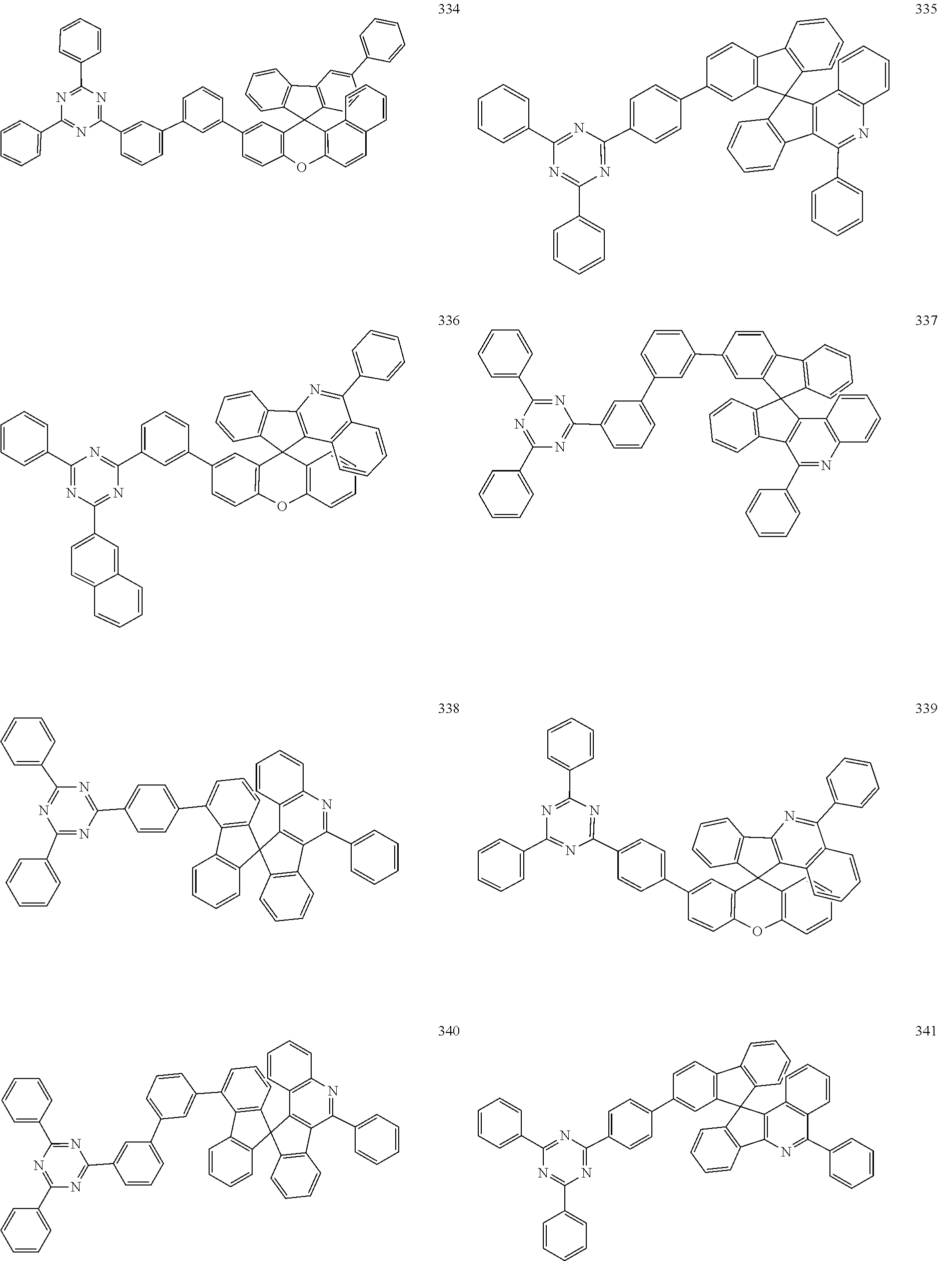

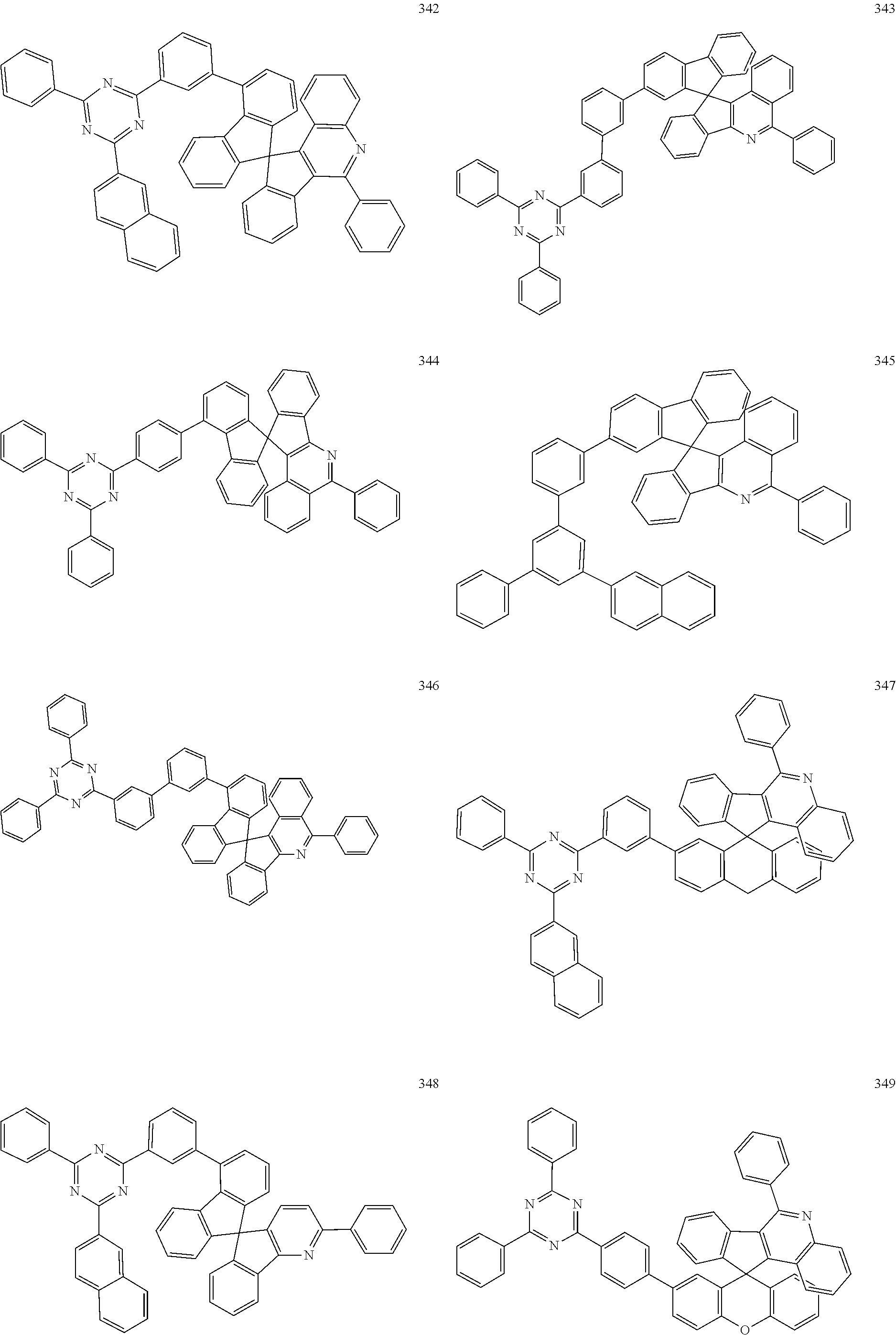

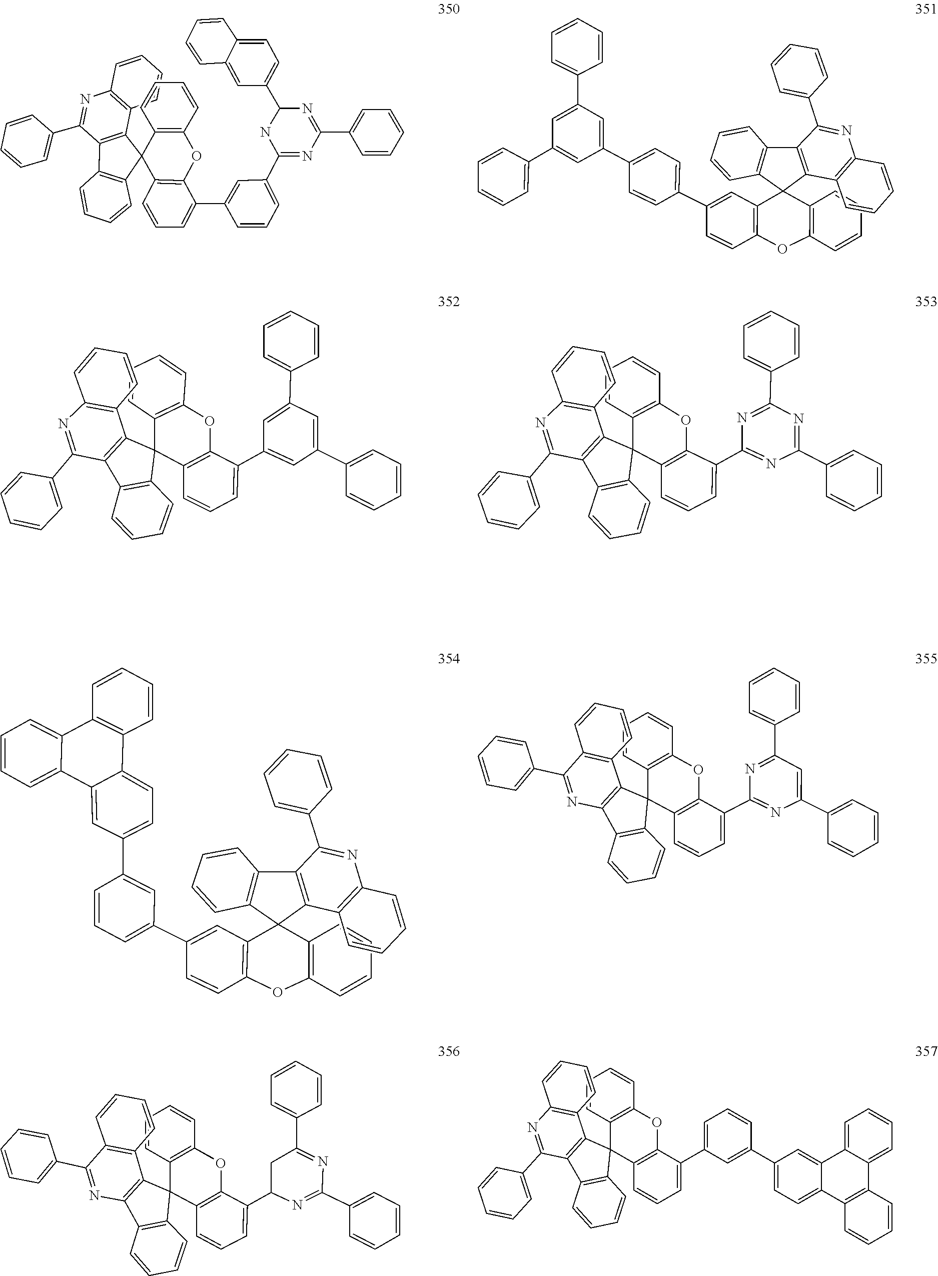

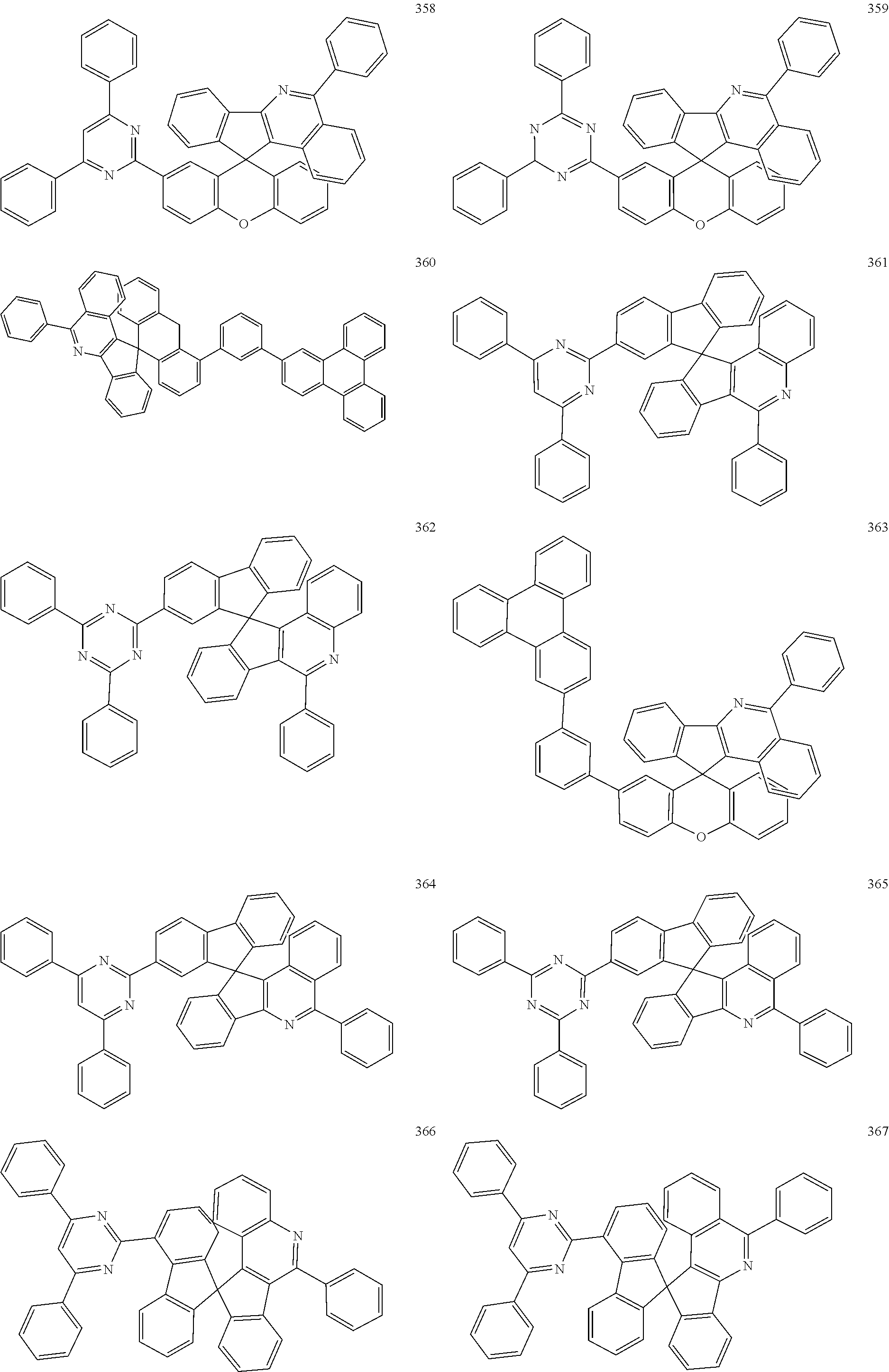

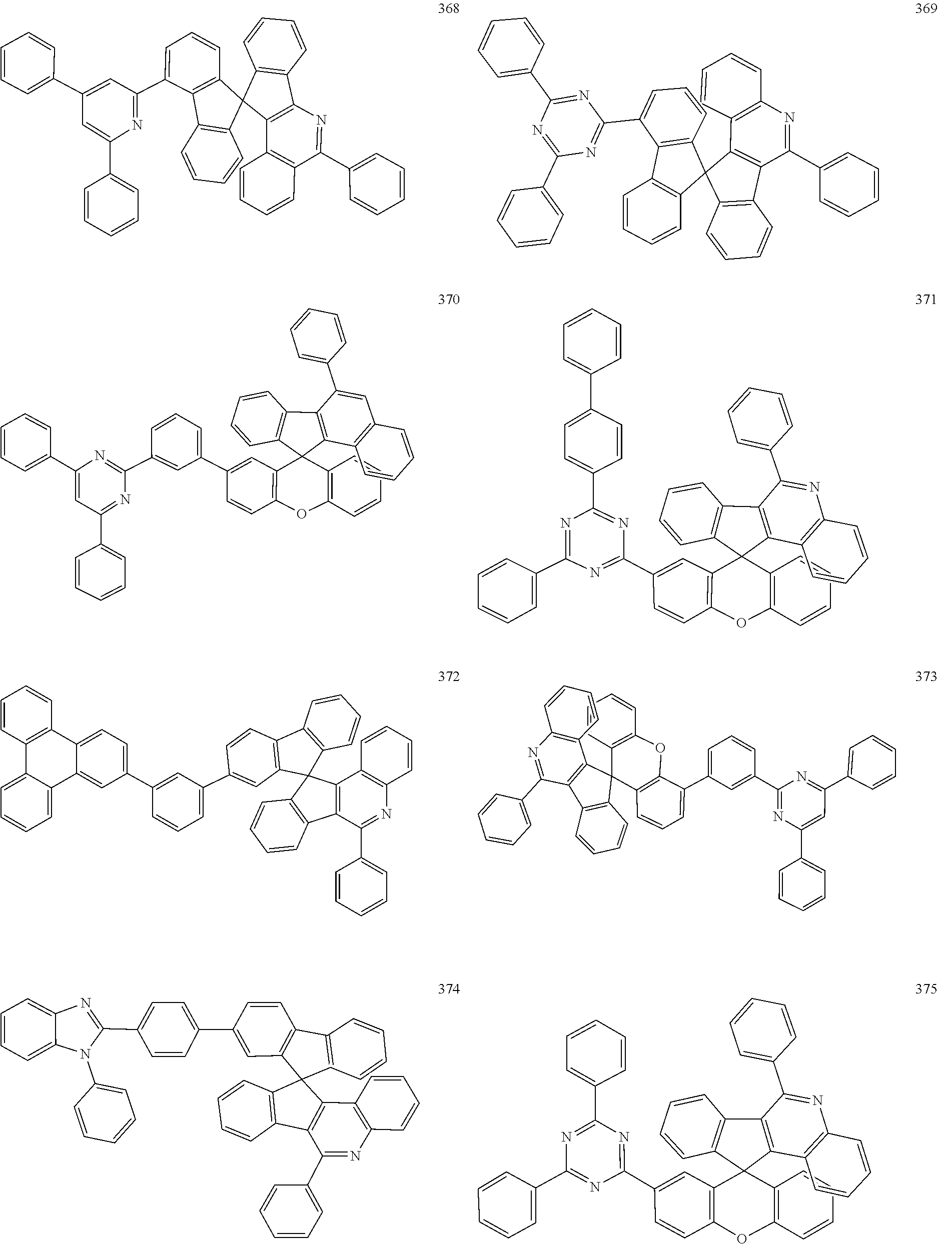

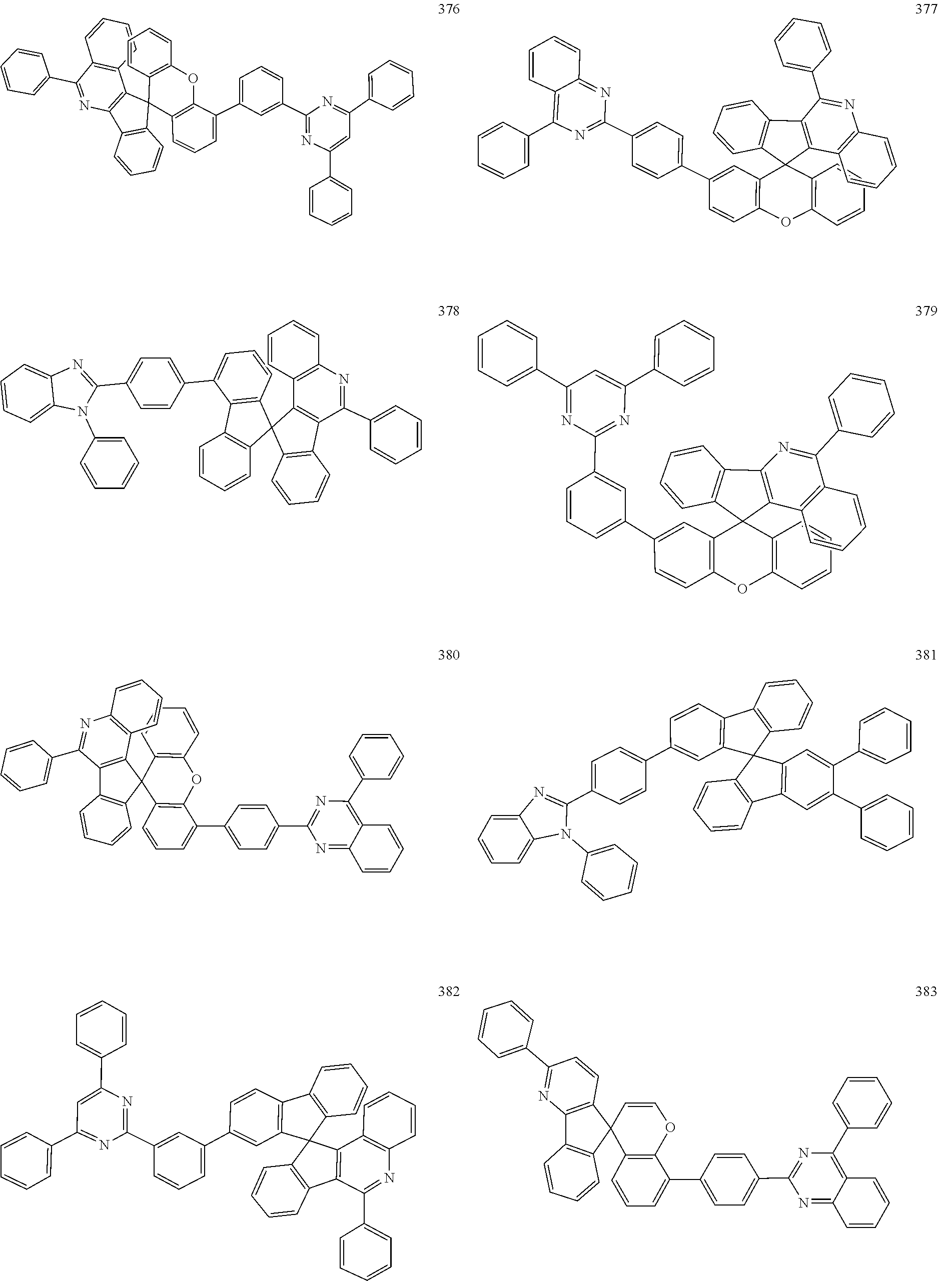

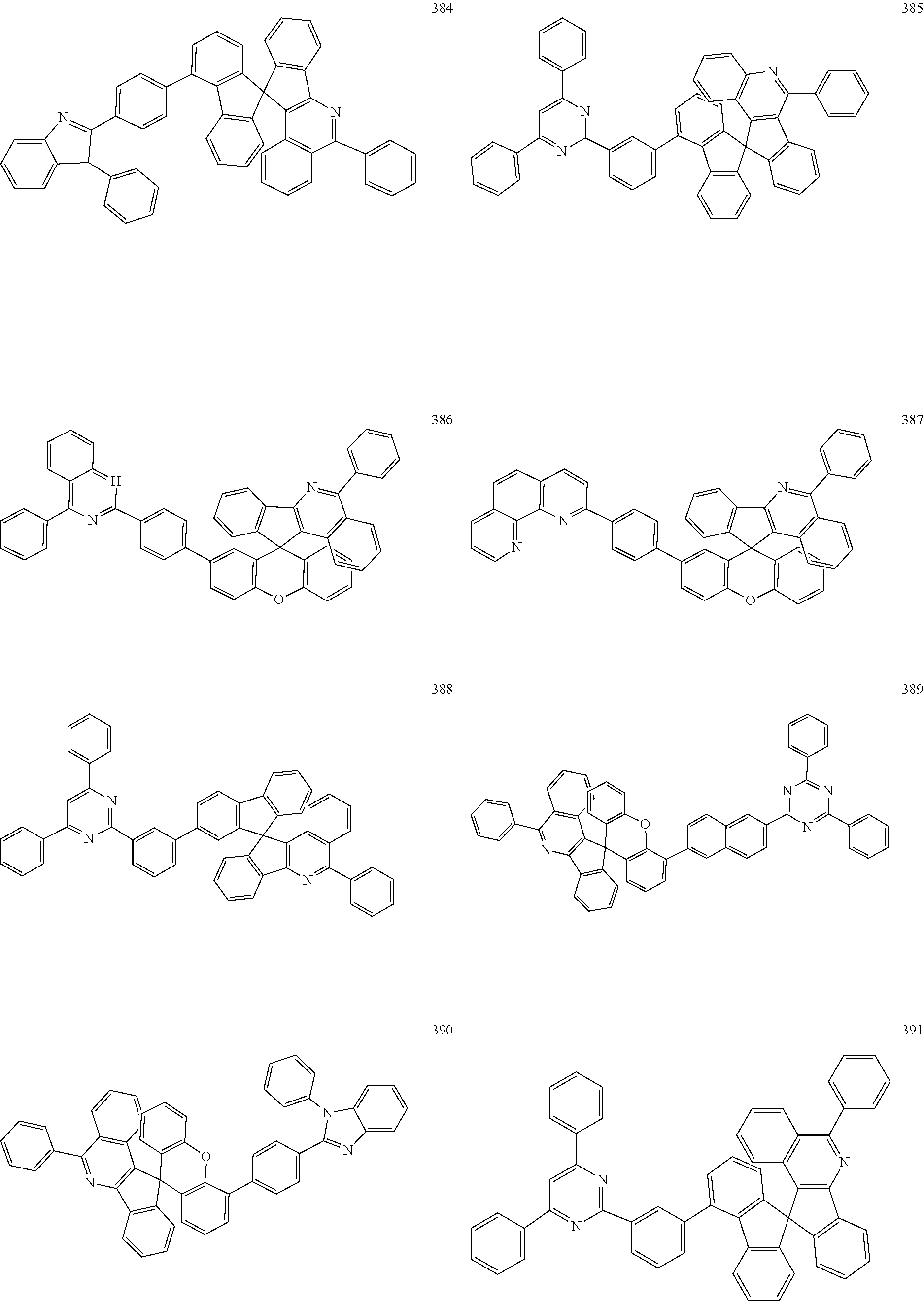

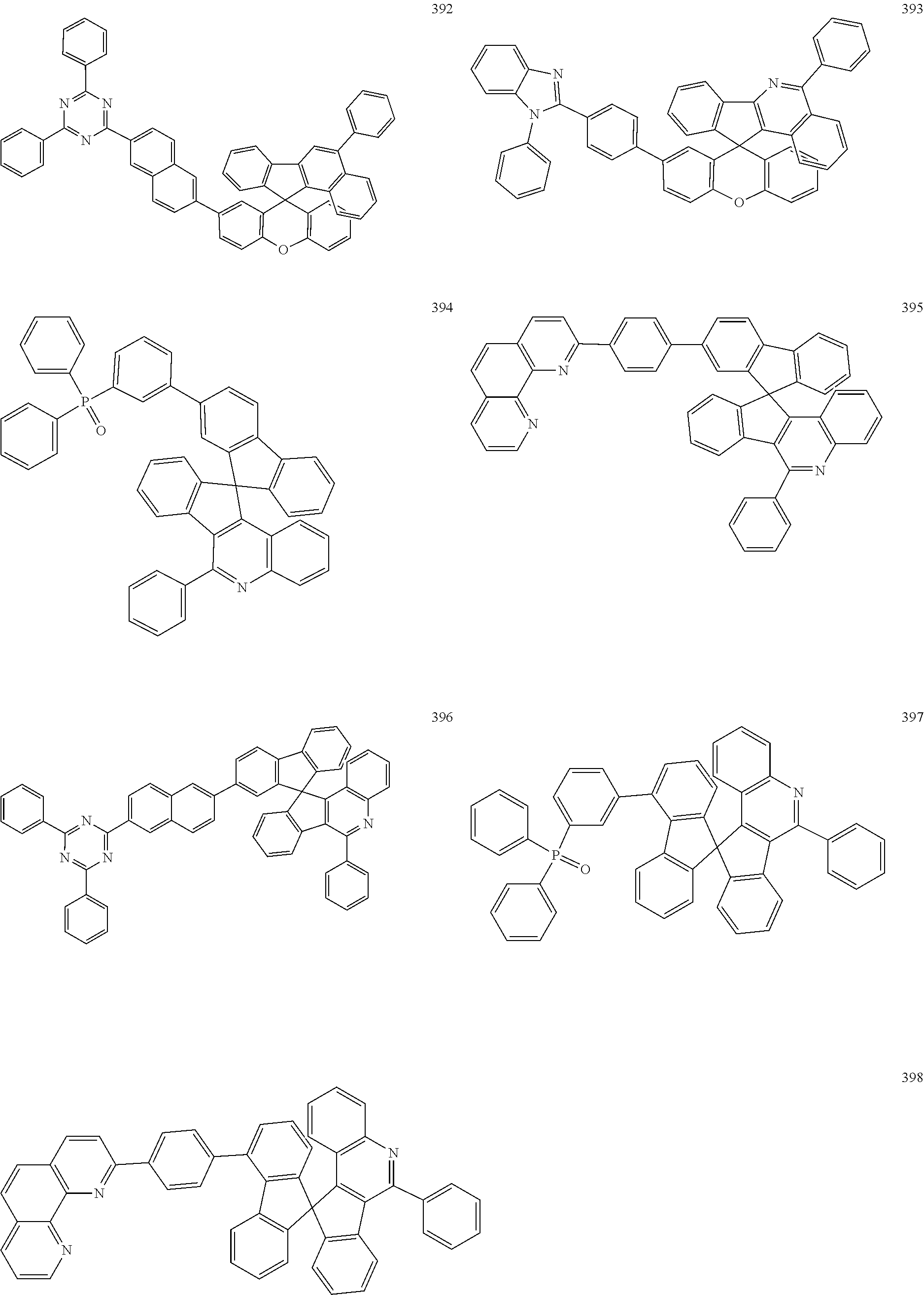

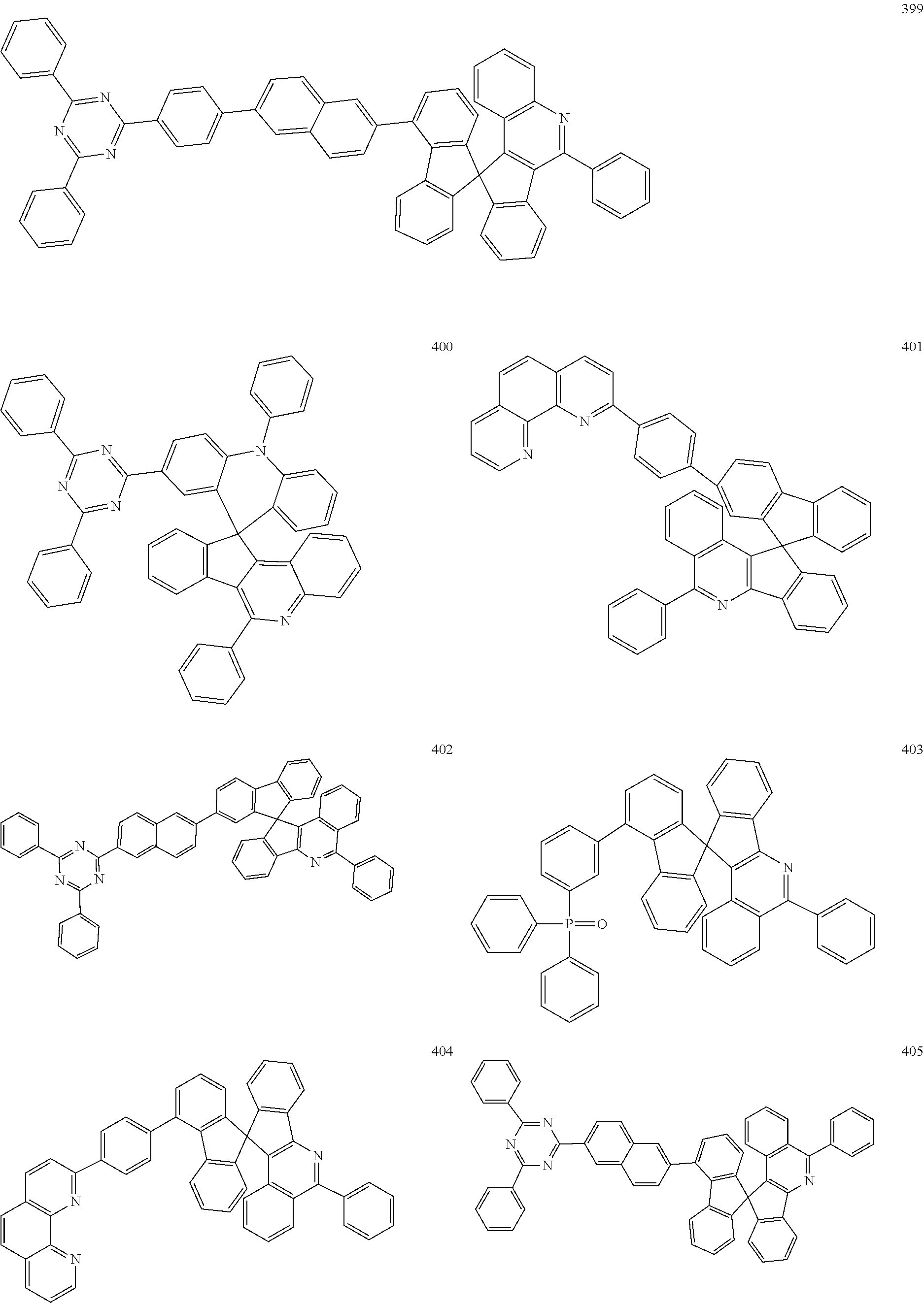

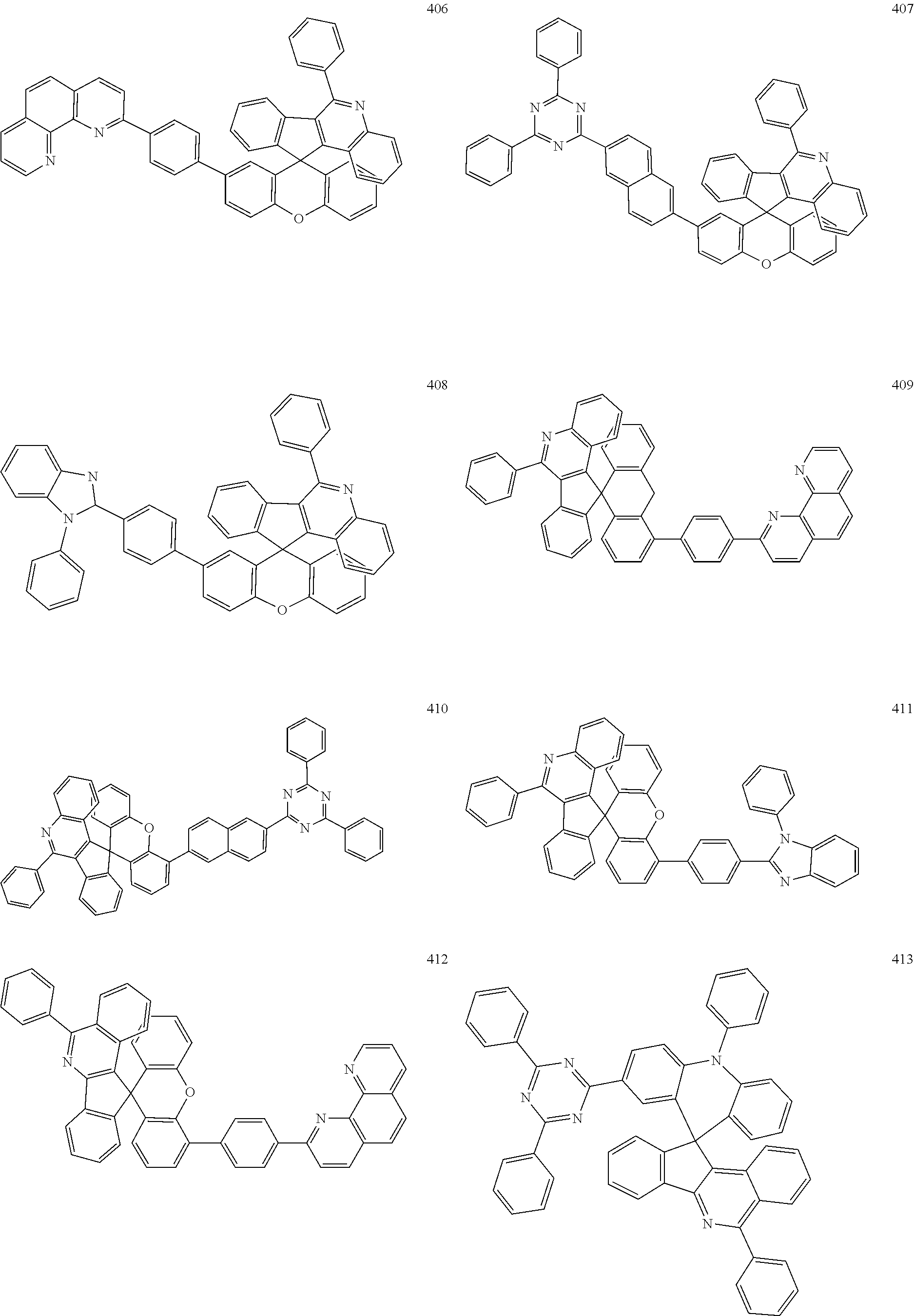

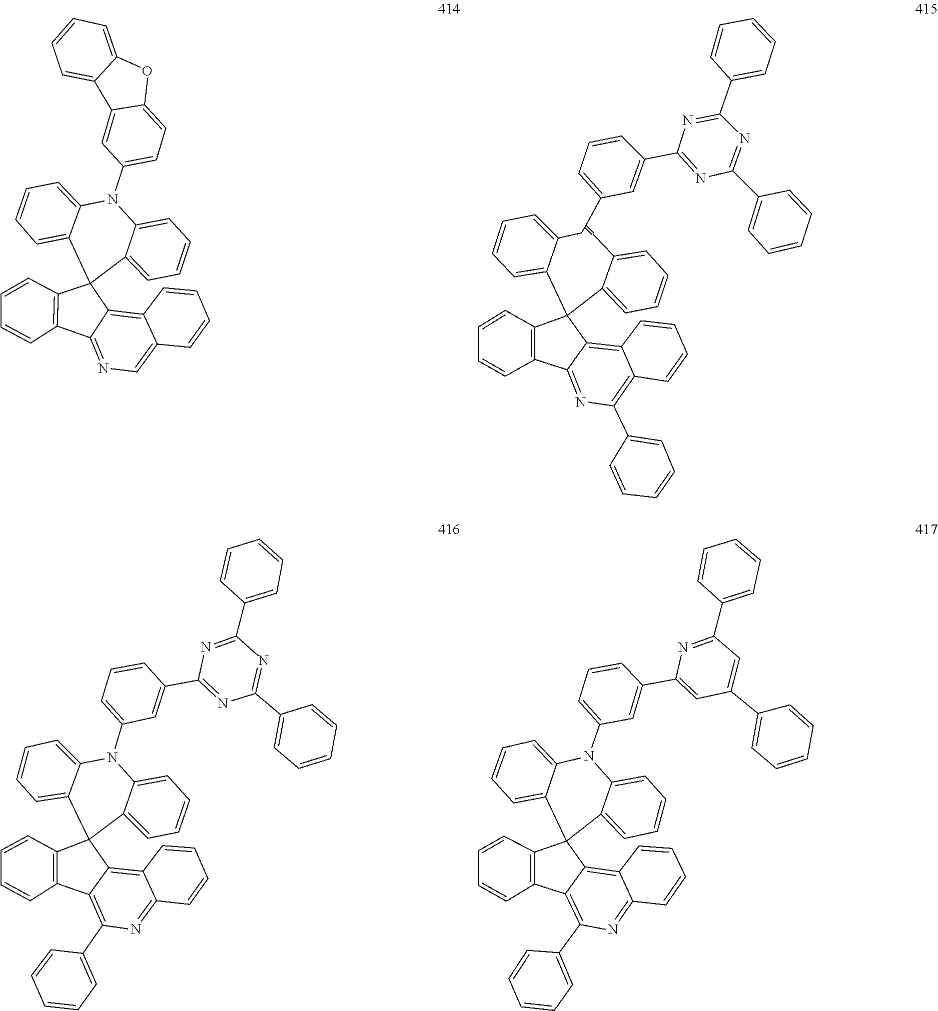

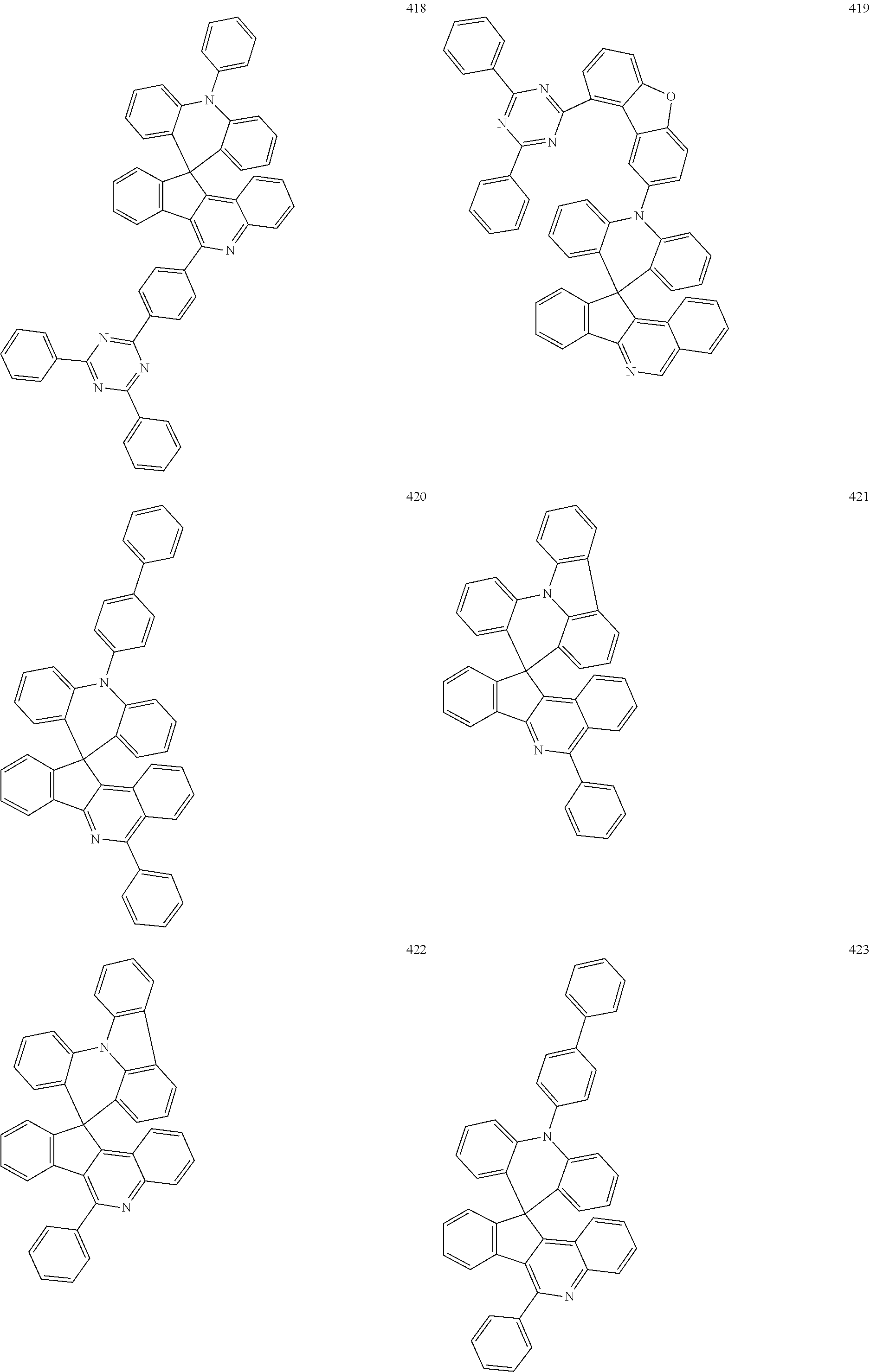

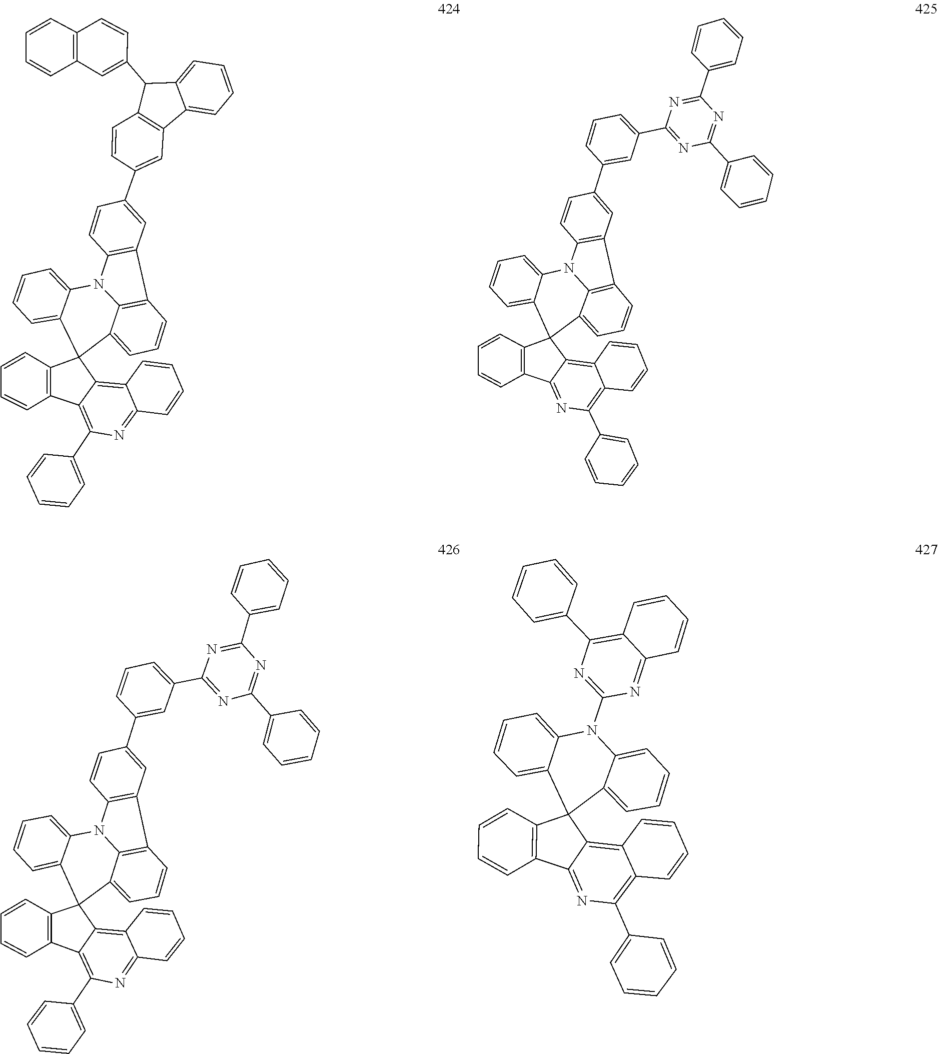 [SMILES: c1ccc(-c2nc3ccccc3c3c2-c2ccccc2C32c3ccccc3-n3c4ccc(-c5ccc6c(c5)-c5ccccc5C6c5ccc6ccccc6c5)cc4c4cccc2c43)cc1 ; c1ccc(-c2nc(-c3ccccc3)nc(-c3cccc(-c4ccc5c(c4)c4cccc6c4n5-c4ccccc4C64c5ccccc5-c5nc(-c6ccccc6)c6ccccc6c54)c3)n2)cc1 ; c1ccc(-c2nc(-c3ccccc3)nc(-c3cccc(-c4ccc5c(c4)c4cccc6c4n5-c4ccccc4C64c5ccccc5-c5c(-c6ccccc6)nc6ccccc6c54)c3)n2)cc1 ; c1ccc(-c2nc(N3c4ccccc4C4(c5ccccc5-c5nc(-c6ccccc6)c6ccccc6c54)c4ccccc43)nc3ccccc23)cc1]

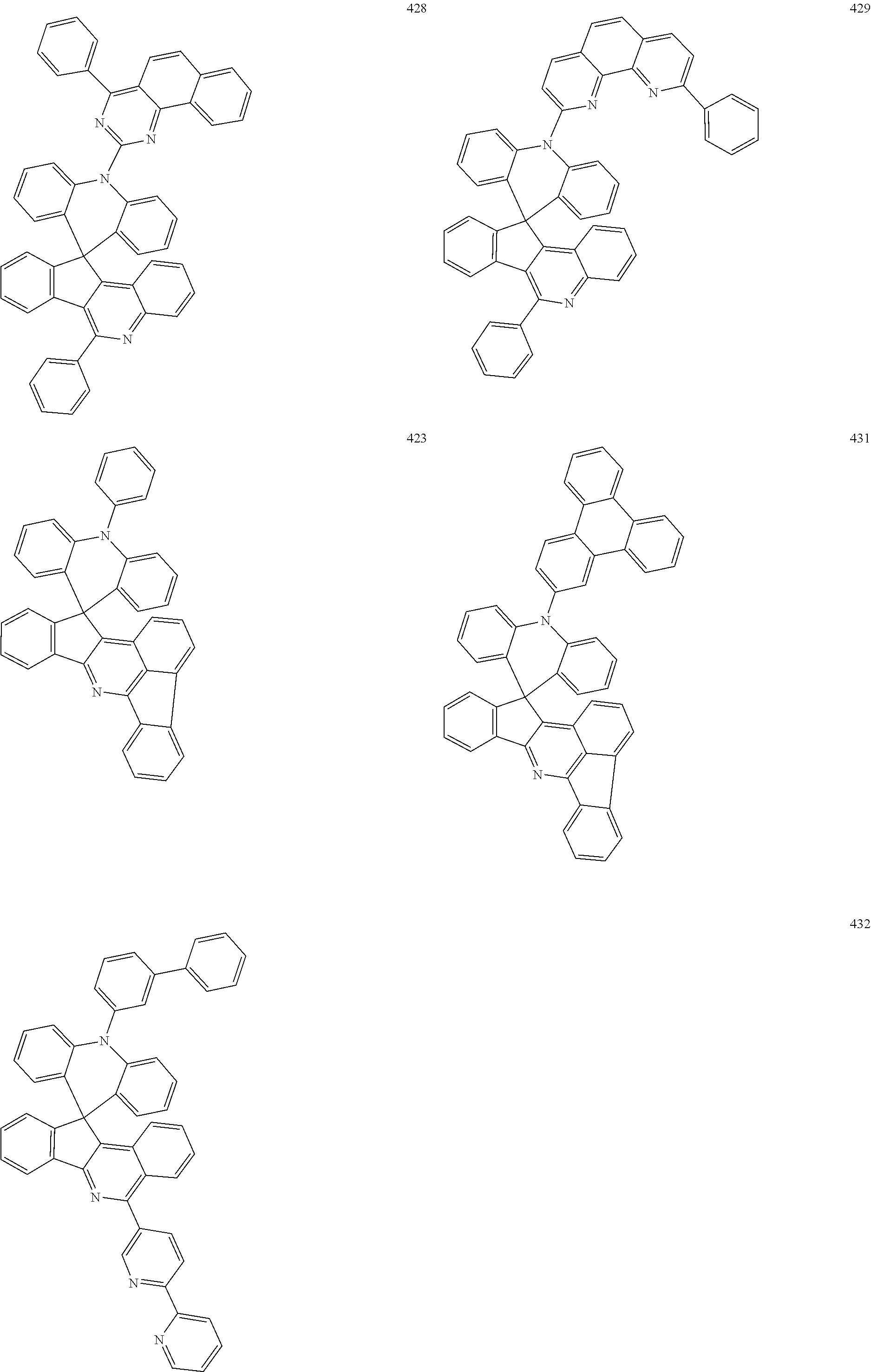 [SMILES: c1ccc(-c2nc3ccccc3c3c2-c2ccccc2C32c3ccccc3N(c3nc(-c4ccccc4)c4ccc5ccccc5c4n3)c3ccccc32)cc1 ; c1ccc(-c2ccc3ccc4ccc(N5c6ccccc6C6(c7ccccc7-c7c(-c8ccccc8)nc8ccccc8c76)c6ccccc65)nc4c3n2)cc1 ; c1ccc(N2c3ccccc3C3(c4ccccc4-c4nc5c6c(cccc6c43)-c3ccccc3-5)c3ccccc32)cc1 ; c1ccc2c(c1)-c1cccc3c4c(nc-2c13)-c1ccccc1C41c2ccccc2N(c2ccc3c4ccccc4c4ccccc4c3c2)c2ccccc21 ; c1ccc(-c2cccc(N3c4ccccc4C4(c5ccccc5-c5nc(-c6ccc(-c7ccccn7)nc6)c6ccccc6c54)c4ccccc43)c2)cc1]

Preferred embodiments of compounds of the invention are recited in detail in the examples, these compounds being usable alone or in combination with further compounds for all purposes of the invention.

Provided that the conditions specified in claim 1 are met, the abovementioned preferred embodiments can be combined with one another as desired. In a particularly preferred embodiment of the invention, the abovementioned preferred embodiments apply simultaneously.

The compounds of the invention are preparable in principle by various processes. However, the processes described hereinafter have been found to be particularly suitable.

Therefore, the present invention further provides a process for preparing the compounds comprising structures of formula (Ia) and/or (Ib) in which, in a coupling reaction, a compound comprising at least one nitrogen-containing heterocyclic group is joined to a compound comprising at least one aromatic or heteroaromatic group.

Suitable compounds comprising at least one nitrogen-containing heterocyclic group are in many cases commercially available, and the starting compounds detailed in the examples are obtainable by known processes, and so reference is made thereto.

Compounds comprising at least one nitrogen-containing heterocyclic group can be reacted with further aryl or heteroaryl compounds by known coupling reactions, the necessary conditions for this purpose being known to the person skilled in the art, and detailed specifications in the examples give support to the person skilled in the art in conducting these reactions.

Particularly suitable and preferred coupling reactions which all lead to C—C bond formations and/or C—N bond formations are those according to BUCHWALD, SUZUKI, YAMAMOTO, STILLE, HECK, NEGISHI, SONOGASHIRA and HIYAMA. These reactions are widely known, and the examples will provide the person skilled in the art with further pointers.

The principles of the preparation processes detailed above are known in principle from the literature for similar compounds and can be adapted easily by the person skilled in the art for the preparation of the compounds of the invention. Further information can be found in the examples.

It is possible by these processes, if necessary followed by purification, for example recrystallization or sublimation, to obtain the compounds of the invention comprising structures of the formula (Ia) and/or (Ib) in high purity, preferably more than 99% (determined by means of ¹H NMR and/or HPLC).

The compounds of the invention may also have suitable substituents, for example be substituted by relatively long alkyl groups (about 4 to 20 carbon atoms), especially branched alkyl groups, or optionally substituted aryl groups, for example xylyl, mesityl or branched terphenyl or quaterphenyl groups, which bring about solubility in standard organic solvents, such that the compounds are soluble at room temperature in toluene or xylene, for example, in sufficient concentration to be able to process the compounds from solution. These soluble compounds are of particularly good suitability for processing from solution, for example by printing methods. In addition, it should be emphasized that the compounds of the invention comprising at least one structure of the formula (Ia) and/or (Ib) already have enhanced solubility in these solvents.

The compounds of the invention may also be mixed with a polymer. It is likewise possible to incorporate these compounds covalently into a polymer. This is especially possible with compounds substituted by reactive leaving groups such as bromine, iodine, chlorine, boronic acid or boronic ester, or by reactive polymerizable groups such as olefins or oxetanes. These may find use as monomers for production of corresponding oligomers, dendrimers or polymers. The oligomerization or polymerization is preferably effected via the halogen functionality or the boronic acid functionality or via the polymerizable group. It is additionally possible to crosslink the polymers via groups of this kind. The compounds and polymers of the invention may be used in the form of a crosslinked or uncrosslinked layer.

The invention therefore further provides oligomers, polymers or dendrimers containing one or more of the above-detailed structures of the formula (Ia) and/or (Ib) or compounds of the invention, wherein one or more bonds in the compounds of the invention or in the structures of the formula (Ia) and/or (Ib) to the polymer, oligomer or dendrimer are present. According to the linkage of the structures of the formula (Ia) and/or (Ib) or of the compounds, these therefore form a side chain of the oligomer or polymer or are bonded within the main chain. The polymers, oligomers or dendrimers may be conjugated, partly conjugated or nonconjugated. The oligomers or polymers may be linear, branched or dendritic. For the repeat units of the compounds of the invention in oligomers, dendrimers and polymers, the same preferences apply as described above.

For preparation of the oligomers or polymers, the monomers of the invention are homopolymerized or copolymerized with further monomers. Preference is given to copolymers wherein the units of formula (Ia) and/or (Ib) or the preferred embodiments recited above and hereinafter are present to an extent of 0.01 to 99.9 mol %, preferably 5 to 90 mol %, more preferably 20 to 80 mol %. Suitable and preferred comonomers which form the polymer base skeleton are chosen from fluorenes (for example according to EP 842208 or WO 2000/022026), spirobifluorenes (for example according to EP 707020, EP 894107 or WO 2006/061181), paraphenylenes (for example according to WO 92/18552), carbazoles (for example according to WO 2004/070772 or WO 2004/113468), thiophenes (for example according to EP 1028136), dihydrophenanthrenes (for example according to WO 2005/014689), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers may contain still further units, for example hole transport units, especially those based on triarylamines, and/or electron transport units.

Additionally of particular interest are compounds of the invention which feature a high glass transition temperature. In this connection, preference is given especially to compounds of the invention comprising structures of the general formula (Ia) and/or (Ib) or the preferred embodiments recited above and hereinafter which have a glass transition temperature of at least 70° C., more preferably of at least 110° C., even more preferably of at least 125° C. and especially preferably of at least 150° C., determined in accordance with DIN 51005 (2005-08 version).

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (-)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, hexamethylindane or mixtures of these solvents.

The present invention therefore further provides a formulation comprising a compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound, especially a phosphorescent dopant, and/or a further matrix material. This further compound may also be polymeric.

The present invention therefore still further provides a composition comprising a compound of the invention and at least one further organofunctional material. Functional materials are generally the organic or inorganic materials introduced between the anode and cathode. Preferably, the organically functional material is selected from the group consisting of fluorescent emitters, phosphorescent emitters, polypodal emitters, emitters that exhibit TADF (thermally activated delayed fluorescence), host materials, electron transport materials, electron injection materials, hole transport materials, hole injection materials, electron blocker materials, hole blocker materials, wide band gap materials and n-dopants.

In a particular aspect of the present invention, the compounds of the invention can be used as matrix material, especially for phosphorescent emitters, and matrix materials are in many cases used in combination with further matrix materials.

The present invention therefore also relates to a composition comprising at least one compound comprising structures of formula (Ia) and/or (Ib) or the preferred embodiments recited above and hereinafter and at least one further matrix material.

The present further provides a composition comprising at least one compound comprising at least one structure of formula (Ia) and/or (Ib) or the preferred embodiments recited above and hereinafter and at least one wide band gap material, a wide band gap material being understood to mean a material in the sense of the disclosure of U.S. Pat. No. 7,294,849. These systems exhibit exceptional advantageous performance data in electroluminescent devices.

Preferably, the additional compound may have a band gap of 2.5 eV or more, preferably 3.0 eV or more, very preferably of 3.5 eV or more. One way of calculating the band gap is via the energy levels of the highest occupied molecular orbital (HOMO) and the lowest unoccupied molecular orbital (LUMO).

Molecular orbitals, especially also the highest occupied molecular orbital (HOMO) and the lowest unoccupied molecular orbital (LUMO), the energy levels thereof and the energy of the lowest triplet state T₁ and that of the lowest excited singlet state S₁ of the materials are determined via quantum-chemical calculations. For calculation of organic substances, an optimization of geometry is first conducted by the “Ground State/Semi-empirical/Default Spin/AM1/Charge 0/Spin Singlet” method. Subsequently, an energy calculation is effected on the basis of the optimized geometry. This is done using the “TD-SCF/DFT/Default Spin/B3PW91” method with the “6-31G(d)” basis set (charge 0, spin singlet). The HOMO energy level HEh or LUMO energy level LEh is obtained from the energy calculation in Hartree units. This is used to determine the HOMO and LUMO energy levels in electron volts, calibrated by cyclic voltammetry measurements, as follows:

HOMO(eV)=((HEh*27.212)−0.9899)/1.1206

LUMO(eV)=((LEh*27.212)−2.0041)/1.385

These values are to be regarded as HOMO and LUMO energy levels of the materials in the context of this application.

The lowest triplet state T₁ is defined as the energy of the triplet state having the lowest energy, which is apparent from the quantum-chemical calculation described.

The lowest excited singlet state S₁ is defined as the energy of the excited singlet state having the lowest energy, which is apparent from the quantum-chemical calculation described.

The method described herein is independent of the software package used and always gives the same results. Examples of frequently utilized programs for this purpose are “Gaussian09 W” (Gaussian Inc.) and Q-Chem 4.1 (Q-Chem, Inc.).

The present invention also relates to a composition comprising at least one compound comprising structures of formula (Ia) and/or (Ib) or the preferred embodiments recited above and hereinafter and at least one phosphorescent emitter, the term “phosphorescent emitters” also being understood to mean phosphorescent dopants.

A dopant in a system comprising a matrix material and a dopant is understood to mean that component having the smaller proportion in the mixture. Correspondingly, a matrix material in a system comprising a matrix material and a dopant is understood to mean that component having the greater proportion in the mixture.

Preferred phosphorescent dopants for use in matrix systems, preferably mixed matrix systems, are the preferred phosphorescent dopants specified hereinafter.

The term “phosphorescent dopants” typically encompasses compounds where the emission of light is effected through a spin-forbidden transition, for example a transition from an excited triplet state or a state having a higher spin quantum number, for example a quintet state.

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum. In the context of the present invention, all luminescent compounds containing the abovementioned metals are regarded as phosphorescent compounds.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/000803, WO 2016/124304, WO 2016/015815, WO 2017/032439, WO 2018/019687, WO 2018/019688, WO 2018/041769, WO 2018/054798, WO 2018/069196, WO 2018/069197 and WO 2018/069273. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Explicit examples of phosphorescent dopants are adduced in the following table:

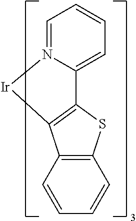

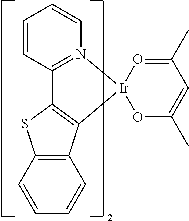

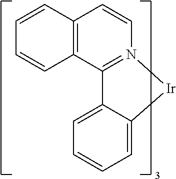

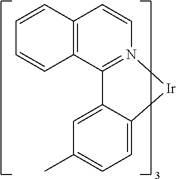

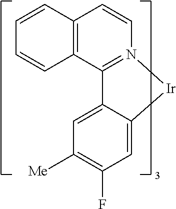

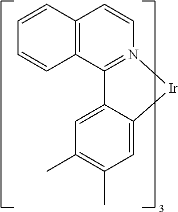

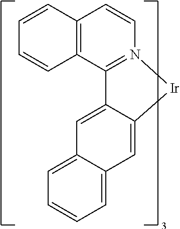

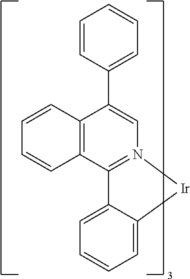

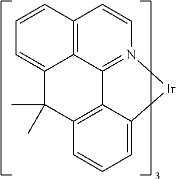

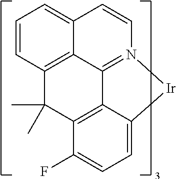

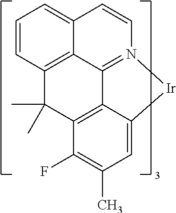

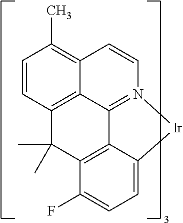

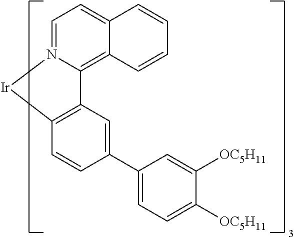

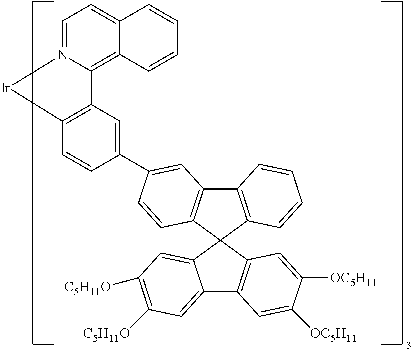

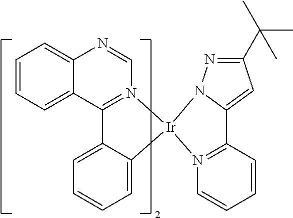

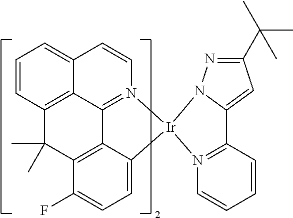

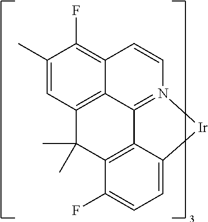

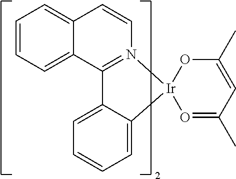

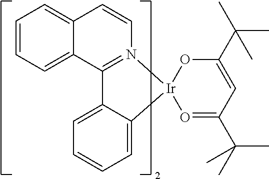

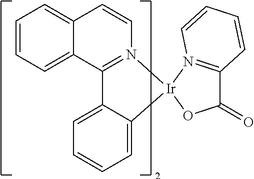

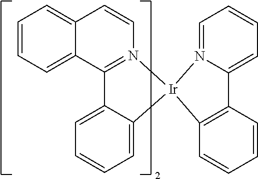

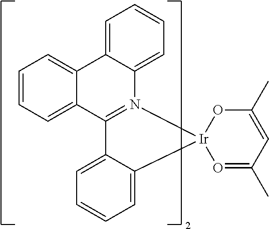

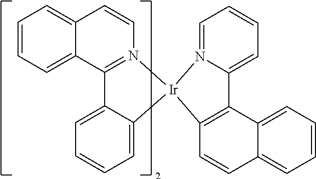

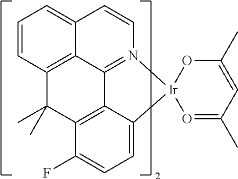

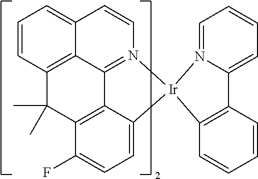

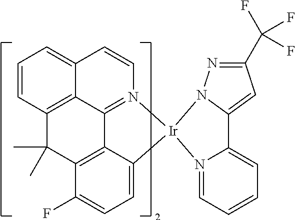

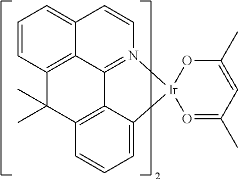

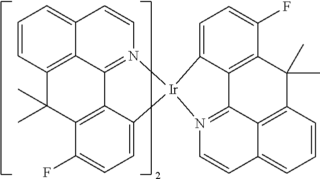

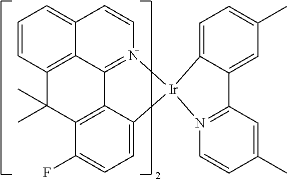

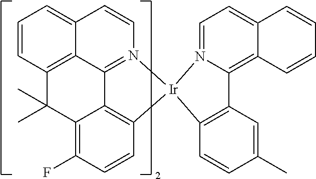

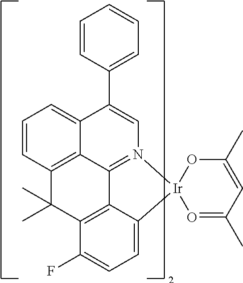

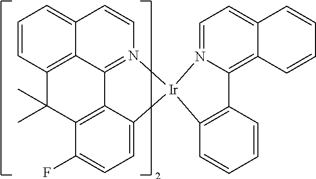

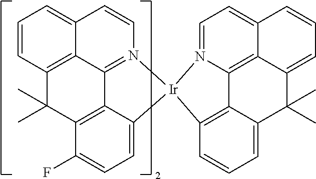

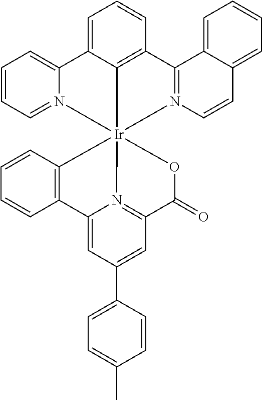

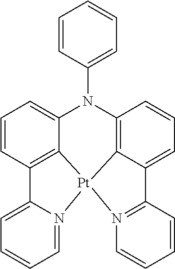

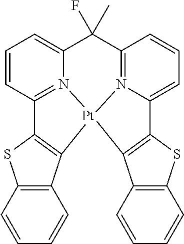

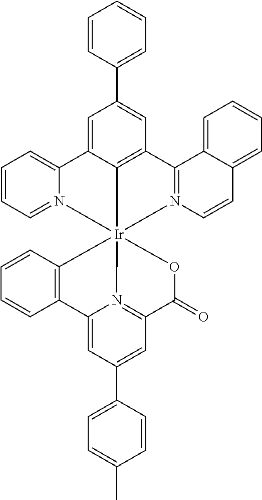

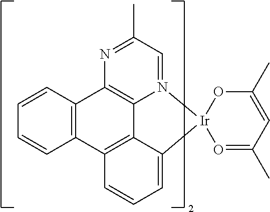

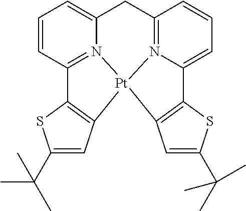

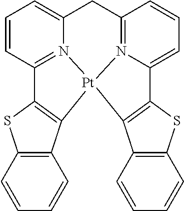

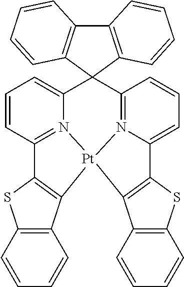

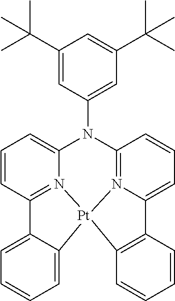

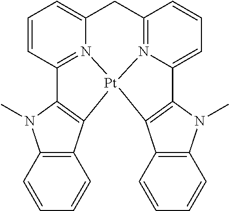

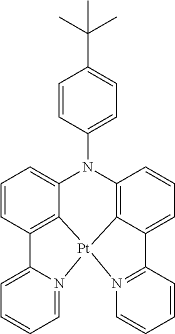

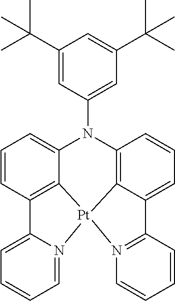

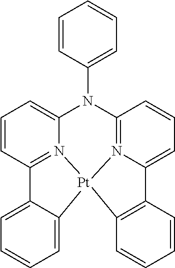

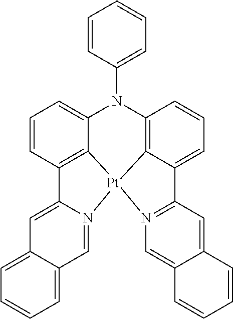

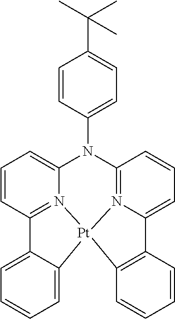

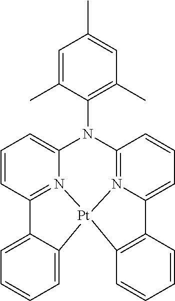

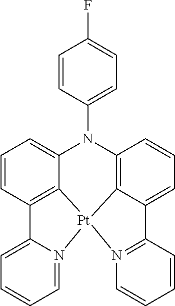

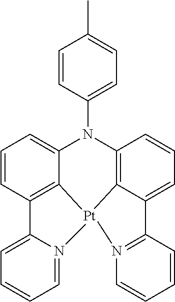

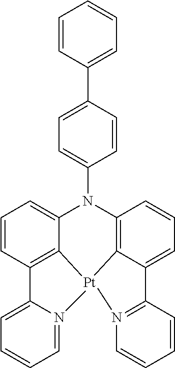

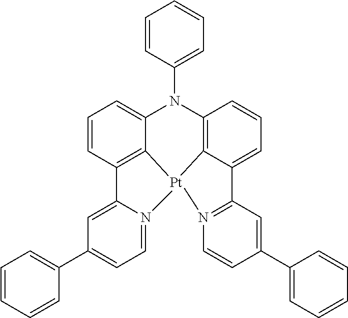

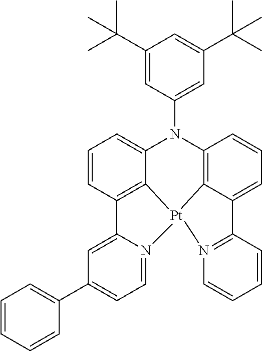

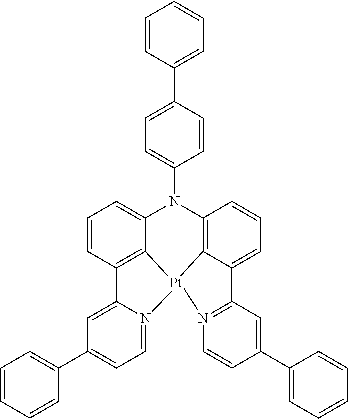

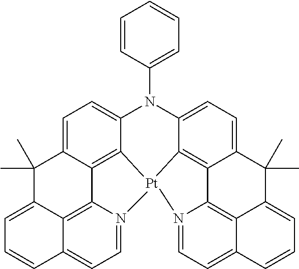

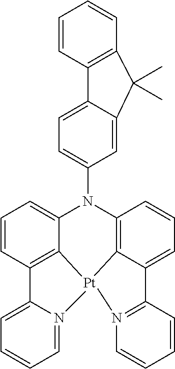

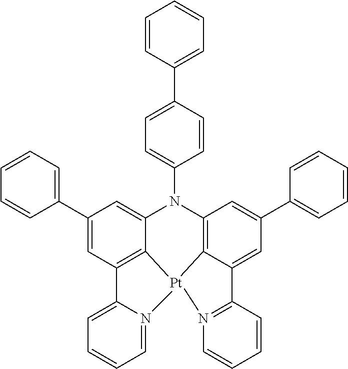

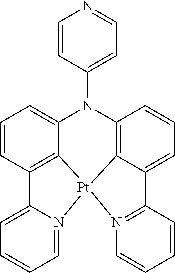

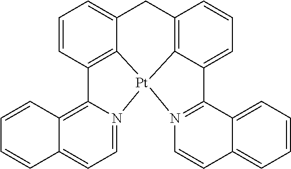

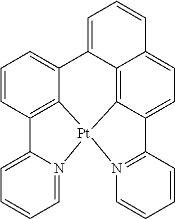

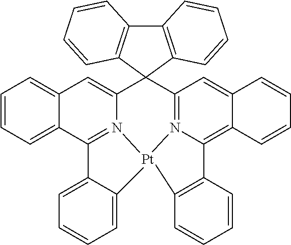

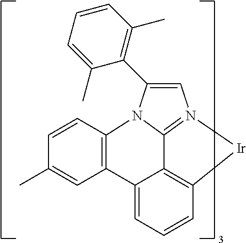

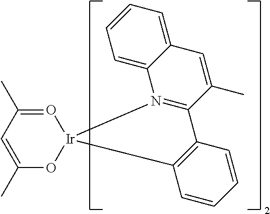

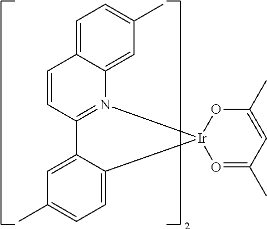

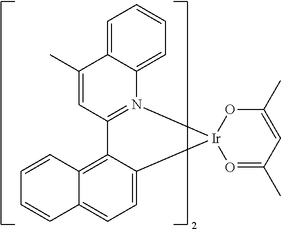

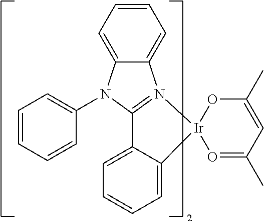

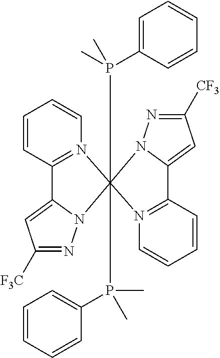

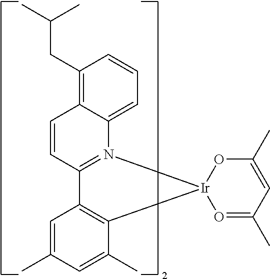

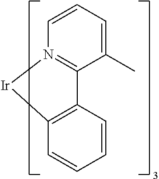

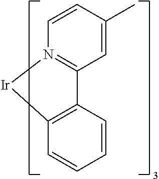

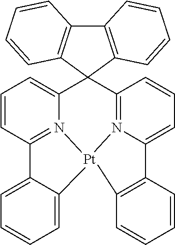

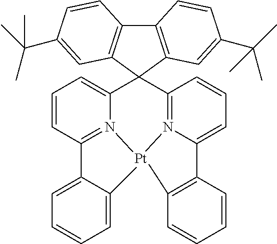

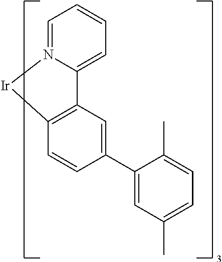

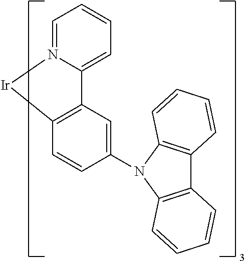

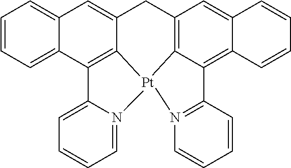

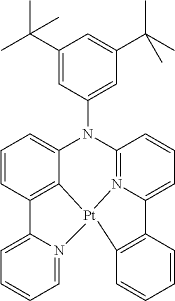

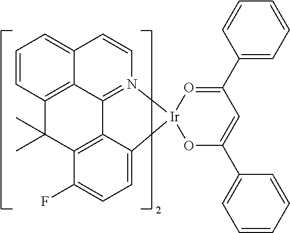

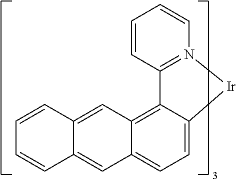

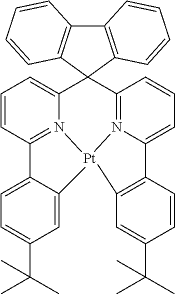

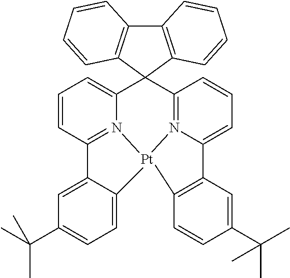

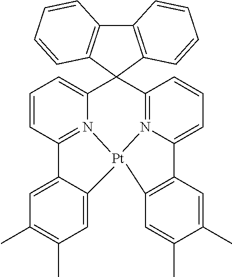

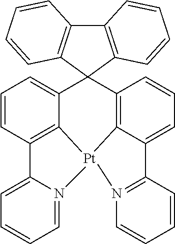

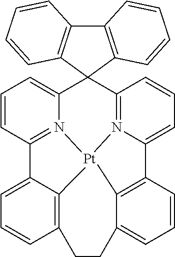

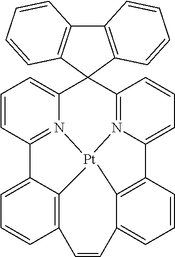

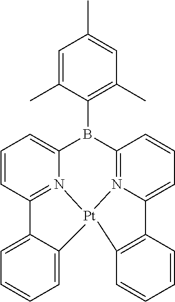

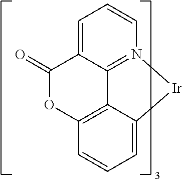

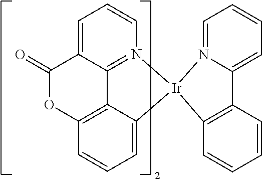

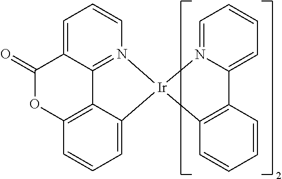

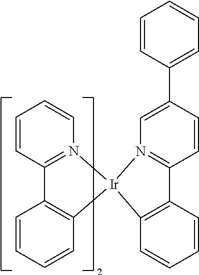

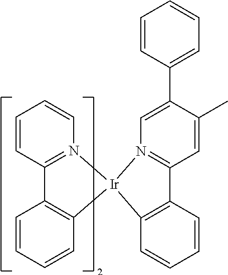

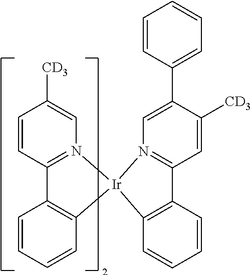

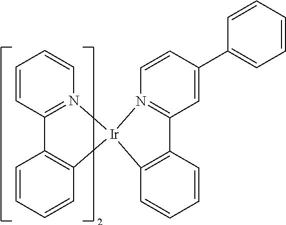

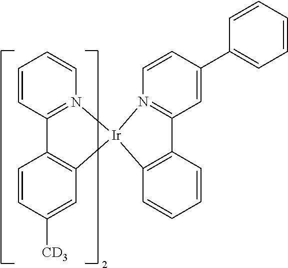

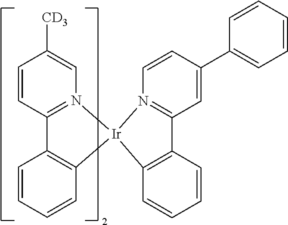

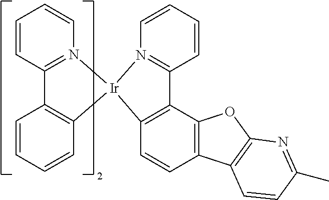

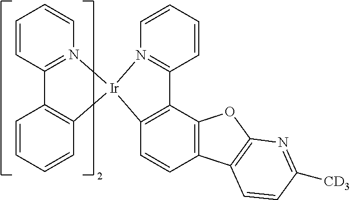

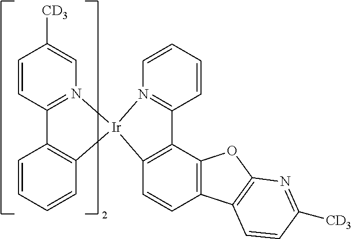

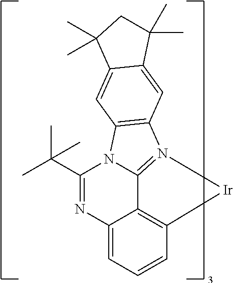

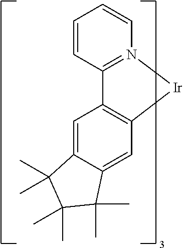

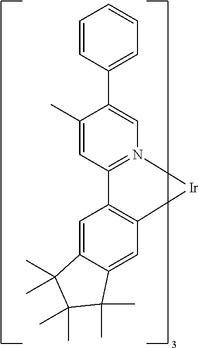

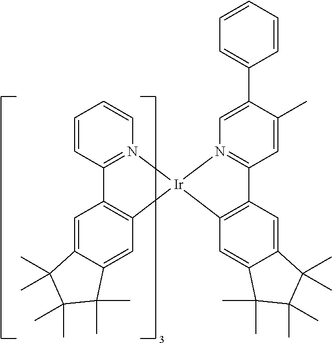

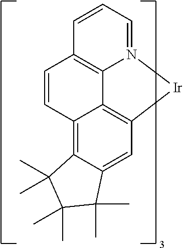

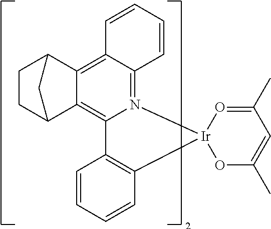

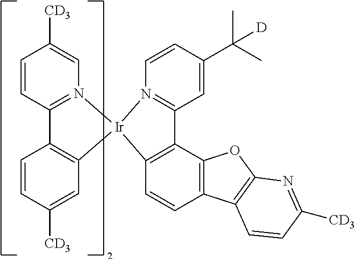

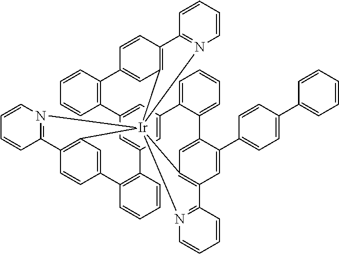

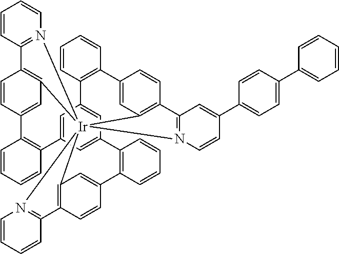

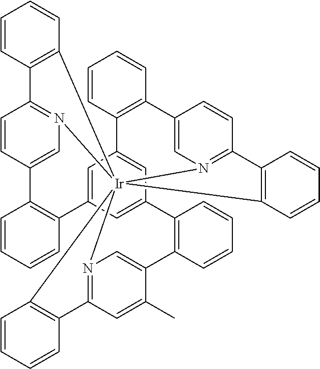

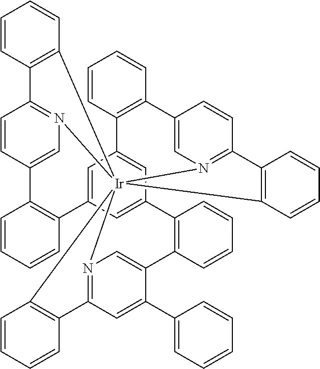

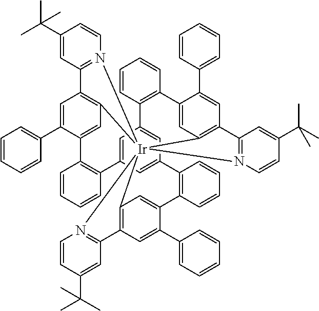

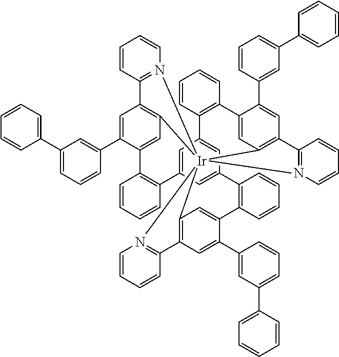

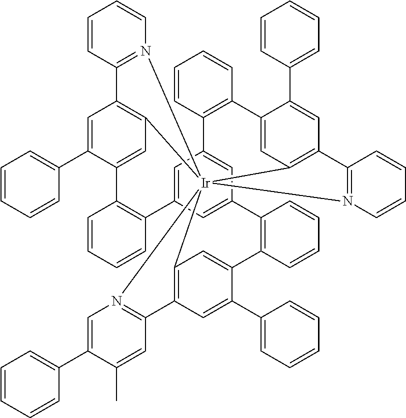

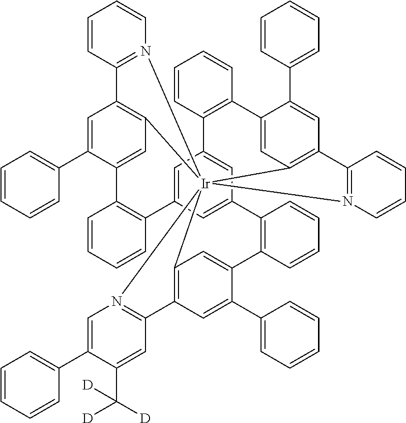

In a particular aspect of the present invention, the compounds of the invention can be used as hole blocker material, preferably in a hole blocker layer, where the compounds of the invention used as hole blocker material comprise at least one electron transport group. In a preferred embodiment, the compounds of the invention used as hole blocker material comprise fewer hole transport groups than electron transport groups, more preferably no hole transport groups.

The above-described compound comprising structures of the formula (Ia) and/or (Ib) or the above-detailed preferred embodiments can preferably be used as active component in an electronic device. An electronic device is understood to mean any device comprising anode, cathode and at least one layer between anode and cathode, said layer comprising at least one organic or organometallic compound. The electronic device of the invention thus comprises anode, cathode and at least one layer in between containing at least one compound comprising structures of the formula (Ia) and/or (Ib). Preferred electronic devices here are selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), organic electrical sensors, light-emitting electrochemical cells (LECs), organic laser diodes (O-lasers) and organic plasmon emitting devices (D. M. Koller et al., Nature Photonics 2008, 1-4), preferably organic electroluminescent devices (OLEDs, PLEDs), especially phosphorescent OLEDs, containing at least one compound comprising structures of the formula (Ia) and/or (Ib) in at least one layer. Particular preference is given to organic electroluminescent devices. Active components are generally the organic or inorganic materials introduced between the anode and cathode, for example charge injection, charge transport or charge blocker materials, but especially emission materials and matrix materials.

A preferred embodiment of the invention is organic electroluminescent devices. The organic electroluminescent device comprises cathode, anode and at least one emitting layer. Apart from these layers, it may comprise still further layers, for example in each case one or more hole injection layers, hole transport layers, hole blocker layers, electron transport layers, electron injection layers, exciton blocker layers, electron blocker layers, charge generation layers and/or organic or inorganic p/n junctions. At the same time, it is possible that one or more hole transport layers are p-doped, for example with metal oxides such as MoO₃ or WO₃ or with (per)fluorinated electron-deficient aromatic systems, and/or that one or more electron transport layers are n-doped. It is likewise possible for interlayers to be introduced between two emitting layers, these having, for example, an exciton-blocking function and/or controlling the charge balance in the electroluminescent device. However, it should be pointed out that not necessarily every one of these layers need be present.

In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are three-layer systems where the three layers exhibit blue, green and orange or red emission (for the basic construction see, for example, WO 2005/011013), or systems having more than three emitting layers. The system may also be a hybrid system wherein one or more layers fluoresce and one or more other layers phosphoresce.

In a preferred embodiment of the invention, the organic electroluminescent device contains the compound of the invention comprising structures of formula (Ia) and/or (Ib) or the above-detailed preferred embodiments as matrix material, preferably as electron-conducting matrix material, in one or more emitting layers, preferably in combination with a further matrix material, preferably a hole-conducting matrix material. In a further preferred embodiment of the invention, the further matrix material is a hole-transporting compound. In a further preferred embodiment of the invention, the further matrix material is an electron-conducting compound. In yet a further preferred embodiment, the further matrix material is a compound having a large band gap which is not involved to a significant degree, if at all, in the hole and electron transport in the layer. An emitting layer comprises at least one emitting compound.

Suitable matrix materials which can be used in combination with the compounds of formula (Ia) and/or (Ib) or according to the preferred embodiments are aromatic ketones, aromatic phosphine oxides or aromatic sulfoxides or sulfones, for example according to WO 2004/013080, WO 2004/093207, WO 2006/005627 or WO 2010/006680, triarylamines, especially monoamines, for example according to WO 2014/015935, carbazole derivatives, e.g. CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example according to WO 2007/063754 or WO 2008/056746, indenocarbazole derivatives, for example according to WO 2010/136109 and WO 2011/000455, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2010/015306, WO 2007/063754 or WO 2008/056746, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to US 2009/0136779, WO 2010/050778, WO 2011/042107, WO 2011/088877 or WO 2012/143080, triphenylene derivatives, for example according to WO 2012/048781, lactams, for example according to WO 2011/116865, WO 2011/137951 or WO 2013/064206, or 4-spirocarbazole derivatives, for example according to WO 2014/094963 or the as yet unpublished application EP 14002104.9. It is likewise possible for a further phosphorescent emitter which emits at a shorter wavelength than the actual emitter to be present as co-host in the mixture.

Preferred co-host materials are triarylamine derivatives, especially monoamines, indenocarbazole derivatives, 4-spirocarbazole derivatives, lactams and carbazole derivatives.

Preferred triarylamine derivatives which are used as co-host materials together with the compounds of the invention are selected from the compounds of the following formula (TA-1):

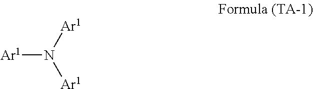

where Ar¹ is the same or different at each instance and is an aromatic or heteroaromatic ring system which has 6 to 40 carbon atoms and may be substituted in each case by one or more R² radicals, an aryloxy group which has 5 to 60 aromatic ring atoms and may be substituted by one or more R² radicals, or an aralkyl group which has 5 to 60 aromatic ring atoms and may be substituted in each case by one or more R² radicals, where two or more adjacent R² substituents may optionally form a mono- or polycyclic aliphatic, heteroaliphatic, aromatic or heteroaromatic ring system, preferably a mono- or polycyclic aliphatic ring system, which may be substituted by one or more R³ radicals, where the symbol R² has the definition given above, especially for formula (Ia) and/or (Ib). Preferably, Ar¹ is the same or different at each instance and is an aryl or heteroaryl group which has 5 to 24 and preferably 5 to 12 aromatic ring atoms, and which may be substituted in each case by one or more R² radicals, but is preferably unsubstituted.

Examples of suitable Ar¹ groups are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, each of which may be substituted by one or more R² radicals, but are preferably unsubstituted.

Preferably, the Ar¹ groups are the same or different at each instance and are selected from the abovementioned R¹-1 to R¹-177 groups, more preferably R¹-1 to R¹-64.

In a preferred embodiment of the compounds of the formula (TA-1), at least one Ar¹ group is selected from a biphenyl group, which may be an ortho-, meta- or para-biphenyl group. In a further preferred embodiment of the compounds of the formula (TA-1), at least one Ar¹ group is selected from a fluorene group or spirobifluorene group, where these groups may each be bonded to the nitrogen atom in the 1, 2, 3 or 4 position. In yet a further preferred embodiment of the compounds of the formula (TA-1), at least one Ar¹ group is selected from a phenylene or biphenyl group, where the group is an ortho-, meta- or para-bonded group, substituted by a dibenzofuran group, a dibenzothiophene group or a carbazole group, especially a dibenzofuran group, where the dibenzofuran or dibenzothiophene group is bonded to the phenylene or biphenyl group via the 1, 2, 3 or 4 position and where the carbazole group is bonded to the phenylene or biphenyl group via the 1, 2, 3 or 4 position or via the nitrogen atom.

In a particularly preferred embodiment of the compounds of the formula (TA-1), one Ar¹ group is selected from a fluorene or spirobifluorene group, especially a 4-fluorene or 4-spirobifluorene group, and one Ar¹ group is selected from a biphenyl group, especially a para-biphenyl group, or a fluorene group, especially a 2-fluorene group, and the third Ar¹ group is selected from a para-phenylene group or a para-biphenyl group, substituted by a dibenzofuran group, especially a 4-dibenzofuran group, or a carbazole group, especially an N-carbazole group or a 3-carbazole group.

Preferred indenocarbazole derivatives which are used as co-host materials together with the compounds of the invention are selected from the compounds of the following formula (TA-2):

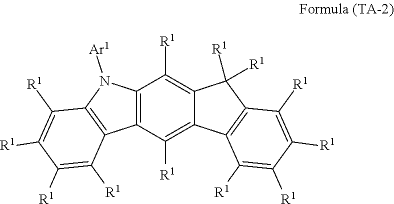

where Ar¹ and R¹ have the definitions listed above, especially for formulae (Ia), (Ib) and/or (TA-1). Preferred embodiments of the Ar¹ group are the above-listed structures R¹-1 to R¹-177, more preferably R¹-1 to R¹-64.

A preferred embodiment of the compounds of the formula (TA-2) is the compounds of the following formula (TA-2a):

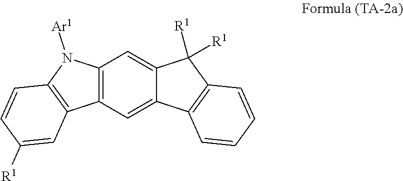

where Ar¹ and R¹ have the definitions listed above, especially for formulae (Ia), (Ib) and/or (TA-1). The two R¹ groups bonded to the indeno carbon atom here are preferably the same or different and are an alkyl group having 1 to 4 carbon atoms, especially methyl groups, or an aromatic ring system having 6 to 12 carbon atoms, especially phenyl groups. More preferably, the two R¹ groups bonded to the indeno carbon atom are methyl groups. Further preferably, the substituent R¹ bonded to the indenocarbazole base skeleton in formula (TA-2a) is H or a carbazole group which may be bonded to the indenocarbazole base skeleton via the 1, 2, 3 or 4 position or via the nitrogen atom, especially via the 3 position.

Preferred 4-spirocarbazole derivatives which are used as co-host materials together with the compounds of the invention are selected from the compounds of the following formula (TA-3):

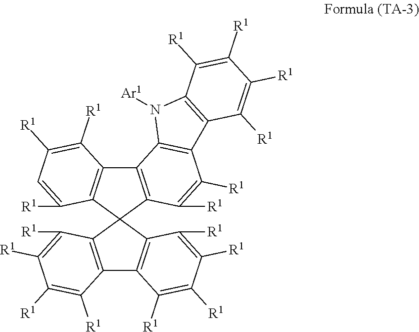

where Ar¹ and R¹ have the definitions listed above, especially for formulae (Ia), (Ib) and/or (Q-1). Preferred embodiments of the Ar¹ group are the above-listed structures R¹-1 to R¹-177, more preferably R¹-1 to R¹-64.

A preferred embodiment of the compounds of the formula (TA-3) is the compounds of the following formula (TA-3a):

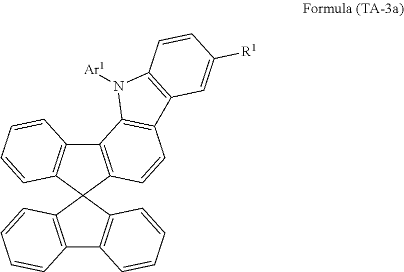

where Ar¹ and R¹ have the definitions listed above, especially for formulae (Ia), (Ib) and/or (Q-1). Preferred embodiments of the Ar¹ group are the above-listed structures R¹-1 to R¹-177, more preferably R¹-1 to R¹-64.

Preferred lactams which are used as co-host materials together with the compounds of the invention are selected from the compounds of the following formula (LAC-1):

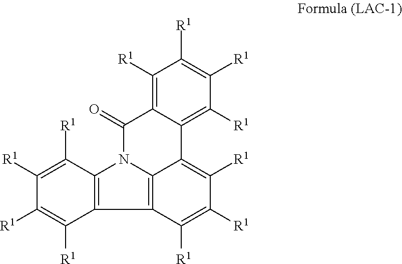

where R¹ has the definition listed above, especially for formula (Ia) and/or (Ib).

A preferred embodiment of the compounds of the formula (LAC-1) is the compounds of the following formula (LAC-1a):

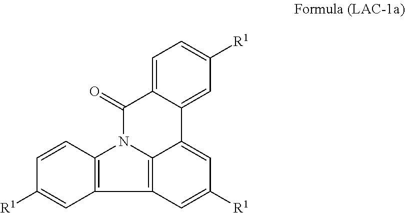

where R¹ has the definition given above, especially for formula (Ia) and/or (Ib). R¹ here is preferably the same or different at each instance and is H or an aromatic or heteroaromatic ring system which has 5 to 40 aromatic ring atoms and may be substituted by one or more R² radicals, where R² may have the definition given above, especially for formula (Ia) and/or (Ib). Most preferably, the substituents R¹ are selected from the group consisting of H or an aromatic or heteroaromatic ring system which has 6 to 18 aromatic ring atoms, preferably 6 to 13 aromatic ring atoms, and may be substituted in each case by one or more nonaromatic R² radicals, but is preferably unsubstituted. Examples of suitable substituents R¹ are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl and 1-, 2-, 3- or 4-carbazolyl, each of which may be substituted by one or more R² radicals, but are preferably unsubstituted. Suitable R¹ structures here are the same structures as depicted above for R-1 to R-177, more preferably R¹-1 to R¹-64.

Very particularly preferred co-host materials are the biscarbazoles.

It may also be preferable to use a plurality of different matrix materials as a mixture, especially at least one electron-conducting matrix material and at least one hole-conducting matrix material. Preference is likewise given to the use of a mixture of a charge-transporting matrix material and an electrically inert matrix material having no significant involvement, if any, in the charge transport, as described, for example, in WO 2010/108579.

It is further preferable to use a mixture of two or more triplet emitters together with a matrix. In this case, the triplet emitter having the shorter-wave emission spectrum serves as co-matrix for the triplet emitter having the longer-wave emission spectrum.

More preferably, a compound of the invention comprising structures of formula (Ia) and/or (Ib), in a preferred embodiment, can be used as matrix material in an emission layer of an organic electronic device, especially in an organic electroluminescent device, for example in an OLED or OLEC. In this case, the matrix material containing a compound comprising structures of formula (Ia) and/or (Ib) or the preferred embodiments recited above and hereinafter is present in the electronic device in combination with one or more dopants, preferably phosphorescent dopants.

The proportion of the matrix material in the emitting layer in this case is between 50.0% and 99.9% by volume, preferably between 80.0% and 99.5% by volume, and more preferably between 92.0% and 99.5% by volume for fluorescent emitting layers and between 85.0% and 97.0% by volume for phosphorescent emitting layers.

Correspondingly, the proportion of the dopant is between 0.1% and 50.0% by volume, preferably between 0.5% and 20.0% by volume, and more preferably between 0.5% and 8.0% by volume for fluorescent emitting layers and between 3.0% and 15.0% by volume for phosphorescent emitting layers.

An emitting layer of an organic electroluminescent device may also comprise systems comprising a plurality of matrix materials (mixed matrix systems) and/or a plurality of dopants. In this case too, the dopants are generally those materials having the smaller proportion in the system and the matrix materials are those materials having the greater proportion in the system. In individual cases, however, the proportion of a single matrix material in the system may be less than the proportion of a single dopant.

In a further preferred embodiment of the invention, the compound comprising structures of formula (Ia) and/or (Ib) or the preferred embodiments recited above and hereinafter is used as a component of mixed matrix systems. The mixed matrix systems preferably comprise two or three different matrix materials, more preferably two different matrix materials. Preferably, in this case, one of the two materials is a material having hole-transporting properties and the other material is a material having electron-transporting properties. The desired electron-transporting and hole-transporting properties of the mixed matrix components may, however, also be combined mainly or entirely in a single mixed matrix component, in which case the further mixed matrix component(s) fulfill(s) other functions. The two different matrix materials may be present here in a ratio of 1:50 to 1:1, preferably 1:20 to 1:1, more preferably 1:10 to 1:1 and most preferably 1:4 to 1:1. Preference is given to using mixed matrix systems in phosphorescent organic electroluminescent devices. One source of more detailed information about mixed matrix systems is the application WO 2010/108579.

The present invention further provides an electronic device, preferably an organic electroluminescent device, comprising one or more compounds of the invention and/or at least one oligomer, polymer or dendrimer of the invention in one or more hole-conducting layers, as hole-conducting compound.

In a preferred embodiment of the invention, the organic electroluminescent device comprises the compound of the invention comprising structures of formula (Ia) and/or (Ib) or the above-detailed preferred embodiments and/or at least one oligomer, polymer or dendrimer of the invention as electron-conducting compound in an electron-conducting layer.

The present invention further provides an electronic device, preferably an organic electroluminescence device, comprising one or more compounds of the invention and/or at least one oligomer, polymer or dendrimer of the invention in one or more electron transport layers, preferably in combination with a material having a high dielectric constant, for example alkali metal or alkaline earth metal fluorides, but also the corresponding oxides or carbonates (e.g. LiF, Li₂O, BaF₂, MgO, NaF, CsF, 052003, etc.), lanthanoid compounds (e.g. Yb₂O₃) or organic alkali metal complexes, e.g. Liq (lithium quinolinate), particular preference being given to a combination of organic alkali metal complexes, preferably Liq, with a compound of the invention and/or an oligomer, polymer or dendrimer of the invention. The two different materials in the electron transport layer may be present here in a ratio of 1:50 to 50:1, preferably 1:10 to 10:1, more preferably 1:4 to 4:1 and most preferably 1:2 to 2:1.

The present invention additionally provides an electronic device, preferably an organic electroluminescent device, comprising one or more compounds of the invention and/or at least one oligomer, polymer or dendrimer of the invention in emitting layers, as matrix material, preferably in combination with a phosphorescent emitter.

Preferred cathodes are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag, in which case combinations of the metals such as Mg/Ag, Ca/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). Likewise useful for this purpose are organic alkali metal complexes, e.g. Liq (lithium quinolinate). The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (O-SC) or the emission of light (OLED/PLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers, for example PEDOT, PANI or derivatives of these polymers. It is further preferable when a p-doped hole transport material is applied to the anode as hole injection layer, in which case suitable p-dopants are metal oxides, for example MoO₃ or WO₃, or (per)fluorinated electron-deficient aromatic systems. Further suitable p-dopants are HAT-CN (hexacyanohexaazatriphenylene) or the compound NPD9 from Novaled. Such a layer simplifies hole injection into materials having a low HOMO, i.e. a large HOMO in terms of magnitude.

In the further layers, it is generally possible to use any materials as used according to the prior art for the layers, and the person skilled in the art is able, without exercising inventive skill, to combine any of these materials with the materials of the invention in an electronic device.

The device is correspondingly (according to the application) structured, contact-connected and finally hermetically sealed, since the lifetime of such devices is severely shortened in the presence of water and/or air.

Additionally preferred is an electronic device, especially an organic electroluminescent device, which is characterized in that one or more layers are coated by a sublimation process. In this case, the materials are applied by vapor deposition in vacuum sublimation systems at an initial pressure of typically less than 10⁻⁵ mbar, preferably less than 10⁻⁶ mbar. It is also possible that the initial pressure is even lower or even higher, for example less than 10⁻⁷ mbar.

Preference is likewise given to an electronic device, especially an organic electroluminescent device, which is characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

Preference is additionally given to an electronic device, especially an organic electroluminescent device, which is characterized in that one or more layers are produced from solution, for example by spin-coating, or by any printing method, for example screen printing, flexographic printing, offset printing or nozzle printing, but more preferably LITI (light-induced thermal imaging, thermal transfer printing) or inkjet printing. For this purpose, soluble compounds are needed, which are obtained, for example, through suitable substitution.

The electronic device, especially the organic electroluminescent device, can also be produced as a hybrid system by applying one or more layers from solution and applying one or more other layers by vapor deposition. For example, it is possible to apply an emitting layer comprising a compound of the invention comprising structures of formula (Ia) and/or (Ib) and a matrix material from solution, and to apply a hole blocker layer and/or an electron transport layer thereto by vapor deposition under reduced pressure.

These methods are known in general terms to those skilled in the art and can be applied by those skilled in the art without difficulty to electronic devices, especially organic electroluminescent devices comprising compounds of the invention comprising structures of formula (Ia) and/or (Ib) or the above-detailed preferred embodiments.

The electronic devices of the invention, especially organic electroluminescent devices, are notable for one or more of the following surprising advantages over the prior art:
1. Electronic devices, especially organic electroluminescent devices, comprising compounds, oligomers, polymers or dendrimers having structures of formula (Ia) and/or (Ib) or the preferred embodiments recited above and hereinafter, especially as host material or as electron-conducting materials and/or hole-conducting materials, have a very good lifetime. In this context, these compounds especially bring about low roll-off, i.e. a small drop in power efficiency of the device at high luminances.
2. Electronic devices, especially organic electroluminescent devices, comprising compounds, oligomers, polymers or dendrimers having structures of formula (Ia) and/or (Ib) or the preferred embodiments recited above and hereinafter, as electron-conducting materials, hole-conducting materials and/or host materials, have excellent efficiency. In this context, compounds, oligomers, polymers or dendrimers of the invention having structures of formula (Ia) and/or (Ib) or the preferred embodiments recited above and hereinafter bring about a low operating voltage when used in electronic devices.
3. Electronic devices, especially organic electroluminescent devices, comprising compounds, oligomers, polymers or dendrimers having structures of formula (Ia) and/or (Ib) or the preferred embodiments recited above and hereinafter have excellent color purity.
4. The compounds, oligomers, polymers or dendrimers of the invention having structures of formula (Ia) and/or (Ib) or the preferred embodiments recited above and hereinafter exhibit very high stability and lifetime.
5. With compounds, oligomers, polymers or dendrimers having structures of formula (Ia) and/or (Ib) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
6. The use of compounds, oligomers, polymers or dendrimers having structures of formula (Ia) and/or (Ib) or the preferred embodiments recited above and hereinafter in layers of electronic devices, especially organic electroluminescent devices, leads to high mobility of the electron conductor structures.
7. Compounds, oligomers, polymers or dendrimers having structures of formula (Ia) and/or (Ib) or the preferred embodiments recited above and hereinafter feature excellent thermal stability, and compounds having a molar mass of less than about 1200 g/mol have good sublimability.
8. Compounds, oligomers, polymers or dendrimers having structures of formula (Ia) and/or (Ib) or the preferred embodiments recited above and hereinafter have excellent glass film formation.
9. Compounds, oligomers, polymers or dendrimers having structures of formula (Ia) and/or (Ib) or the preferred embodiments detailed above and hereinafter form very good films from solutions.
10. The compounds, oligomers, polymers or dendrimers comprising structures of formula (Ia) and/or (Ib) or the preferred embodiments recited above and hereinafter have a surprisingly high triplet level T₁.

These abovementioned advantages are not accompanied by a deterioration in the further electronic properties.

The compounds and mixtures of the invention are suitable for use in an electronic device. An electronic device is understood here to mean a device containing at least one layer containing at least one organic compound. The component may, however, also comprise inorganic materials or else layers formed entirely from inorganic materials.

The present invention therefore further provides for the use of the inventive compounds or mixtures in an electronic device, especially in an organic electroluminescent device, preferably as hole transport material, electron transport material or host material, more preferably as host material for a red-phosphorescing compound.

The present invention still further provides for the use of a compound of the invention and/or an oligomer, polymer or dendrimer of the invention in an electronic device as host material for phosphorescent emitters, electron transport material and/or hole transport material, preferably as host material for a red- or green-phosphorescing compound or as hole transport material or electron transport material in an organic electroluminescent device with a fluorescent emitter.

The present invention still further provides for the use of a compound of the invention and/or of an oligomer, polymer or dendrimer of the invention in an electronic device as part of an electron transport layer, especially in combination with a material having a high dielectric constant.

The present invention still further provides an electronic device comprising at least one of the above-detailed compounds or mixtures of the invention. In this case, the preferences detailed above for the compound also apply to the electronic devices. More preferably, the electronic device is selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), organic electrical sensors, light-emitting electrochemical cells (LECs), organic laser diodes (O-lasers) and organic plasmon emitting devices (D. M. Koller et al., Nature Photonics 2008, 1-4), preferably organic electroluminescent devices (OLEDs, PLEDs), especially phosphorescent OLEDs.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art is therefore able, without exercising inventive skill, to use any materials known for organic electroluminescent devices in combination with the inventive compounds of formula (Ia) and/or (Ib) or according to the preferred embodiments.

The compounds of the invention generally have very good properties on use in organic electroluminescent devices. Especially in the case of use of the compounds of the invention in organic electroluminescent devices, the lifetime is significantly better compared to similar compounds according to the prior art. At the same time, the further properties of the organic electroluminescent device, especially the efficiency and voltage, are likewise better or at least comparable.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive.

This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in detail by the examples which follow, without any intention of restricting it thereby.

The person skilled in the art will be able to use the details given, without exercising inventive skill, to produce further electronic devices of the invention and hence to execute the invention over the entire scope claimed.

### SYNTHESIS EXAMPLES

### a) 2-(4-Chlorophenyl)-3-phenylquinoline-4-carboxylic acid

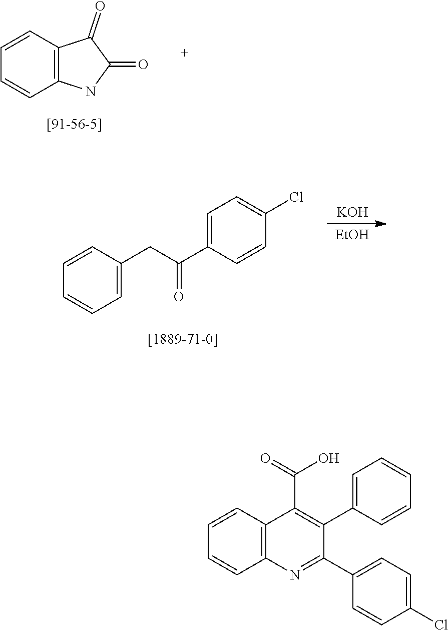

In a 1 L flask, 14.0 g (93.3 mmol; 1.00 eq; 98% pure) of isatin [CAS 91-56-5], 24.2 g (103 mmol; 1.10 eq) of 1-(4-chlorophenyl)-2-phenylethanone [CAS 1889-71-0] and 15.7 g (280 mmol; 3.00 eq) of potassium hydroxide powder [CAS 1310-58-3] are suspended in 630 mL of ethanol [CAS 64-17-5]. The reaction mixture is stirred at 100° C. for 48 hours. After cooling to room temperature, the solvent is removed under reduced pressure. The residue is dissolved in 200 mL of ethyl acetate [CAS 141-78-6] and 200 mL of water, and the phases are separated. After extracting the organic phase has been extracted with water (2×100 mL), the combined aqueous phases are washed with ethyl acetate (3×100 mL). The aqueous phase is adjusted to pH=1 with 25 mL of fuming hydrochloric acid [CAS 7647-01-0], and the precipitated solids are filtered off. After subsequent washing with ethanol, 22.5 g (62.7 mmol; 67% of theory) of product is obtained in the form of a white solid.

The following compounds can be obtained analogously:

| No. | Reactant 1 | Reactant 2 | Product | Yield |
|---|---|---|---|---|
| 1a | 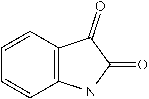 [SMILES: O=C1Nc2ccccc2C1=O] [91-56-5] | 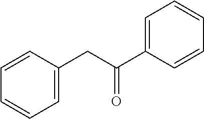 [SMILES: O=C(Cc1ccccc1)c1ccccc1] [451-40-1] | 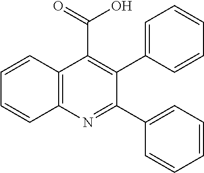 [SMILES: O=C(O)c1c(-c2ccccc2)c(-c2ccccc2)nc2ccccc12] | 73% |
| 2a | 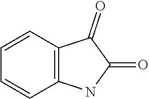 [SMILES: O=C1Nc2ccccc2C1=O] [91-56-5] | 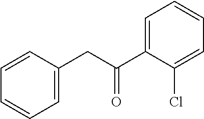 [SMILES: O=C(Cc1ccccc1)c1ccccc1Cl] [72867-72-2] | 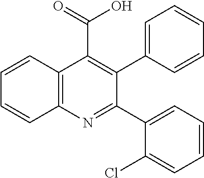 [SMILES: O=C(O)c1c(-c2ccccc2)c(-c2ccccc2Cl)nc2ccccc12] | 60% |
| 3a | 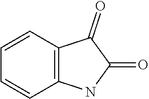 [SMILES: O=C1Nc2ccccc2C1=O] [91-56-5] | 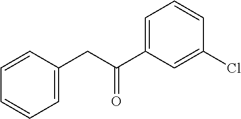 [SMILES: O=C(Cc1ccccc1)c1cccc(Cl)c1] [62482-45-5] | 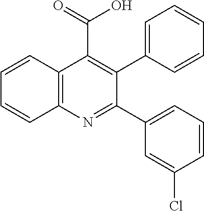 [SMILES: O=C(O)c1c(-c2ccccc2)c(-c2cccc(Cl)c2)nc2ccccc12] | 69% |
| 4a | 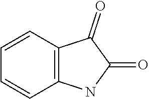 [SMILES: O=C1Nc2ccccc2C1=O] [91-56-5] | 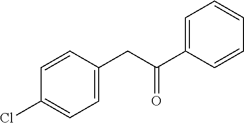 [SMILES: O=C(Cc1ccc(Cl)cc1)c1ccccc1] [6332-83-8] | 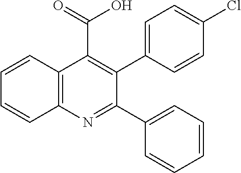 [SMILES: O=C(O)c1c(-c2ccc(Cl)cc2)c(-c2ccccc2)nc2ccccc12] | 66% |
| 5a | 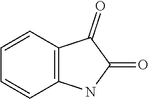 [SMILES: O=C1Nc2ccccc2C1=O] [91-56-5] | 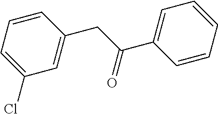 [SMILES: O=C(Cc1cccc(Cl)c1)c1ccccc1] [27798-43-2] | 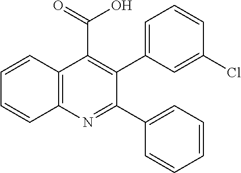 [SMILES: O=C(O)c1c(-c2cccc(Cl)c2)c(-c2ccccc2)nc2ccccc12] | 58% |
| 6a | 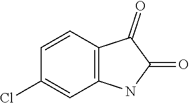 [SMILES: O=C1Nc2cc(Cl)ccc2C1=O] [6341-92-0] | 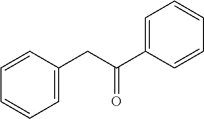 [SMILES: O=C(Cc1ccccc1)c1ccccc1] [451-40-1] | 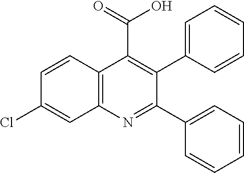 [SMILES: O=C(O)c1c(-c2ccccc2)c(-c2ccccc2)nc2cc(Cl)ccc12] | 71% |
| 7a | 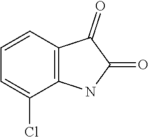 [SMILES: O=C1Nc2c(Cl)cccc2C1=O] [7477-63-6] | 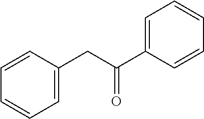 [SMILES: O=C(Cc1ccccc1)c1ccccc1] [451-40-1] | 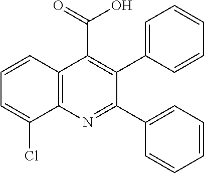 [SMILES: O=C(O)c1c(-c2ccccc2)c(-c2ccccc2)nc2c(Cl)cccc12] | 65% |
| 8a | 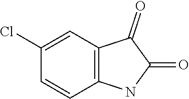 [SMILES: O=C1Nc2ccc(Cl)cc2C1=O] [17630-76-1] | 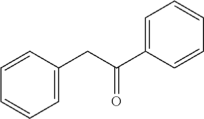 [SMILES: O=C(Cc1ccccc1)c1ccccc1] [451-40-1] | 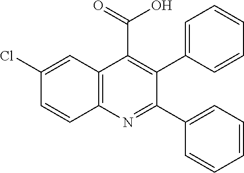 [SMILES: O=C(O)c1c(-c2ccccc2)c(-c2ccccc2)nc2ccc(Cl)cc12] | 68% |

### b) 6-(4-Chlorophenyl)indeno[1,2-c]quinolin-11-one

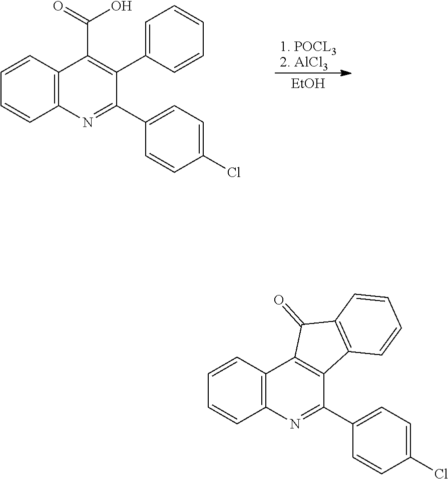 [SMILES: "EtOH" ; O=C(O)c1c(-c2ccccc2)c(-c2ccc(Cl)cc2)nc2ccccc12 ; "1. POCL32. AlCl3" ; O=C1c2ccccc2-c2c(-c3ccc(Cl)cc3)nc3ccccc3c21]

In a 500 mL flask, 21.0 g (58.4 mmol; 1.00 eq.) of 2-(4-chlorophenyl)-3-phenylquinoline-4-carboxylic acid and 32.1 mL (350 mmol; 6.00 eq.) of phosphoryl chloride [CAS 10025-87-3] are dissolved in 183 mL of chlorobenzene [CAS 108-90-7] and heated to 150° C. for 24 h. After checking the conversion, the reaction mixture is cooled down to room temperature, 8.17 g (61.3 mmol; 1.05 eq.) of anhydrous aluminum chloride [CAS 7446-70-0] is added and the mixture is heated to reflux for 3 h. After cooling, the reaction mixture is precipitated in ice-water. The water is decanted off, and EtOH is added to the crude product. The solids that occur are filtered off and dried. 15.53 g (44.9 mmol, 77% of theory) of product is converted without further purification.

The following compounds can be obtained analogously:

| No. | Reactant 1 | Product | Yield |
|---|---|---|---|
| 1b | 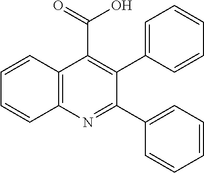 [SMILES: O=C(O)c1c(-c2ccccc2)c(-c2ccccc2)nc2ccccc12] | 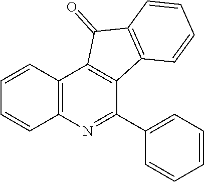 [SMILES: O=C1c2ccccc2-c2c(-c3ccccc3)nc3ccccc3c21] | 82% |
| 2b | 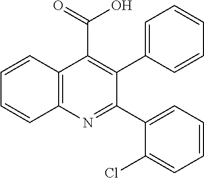 [SMILES: O=C(O)c1c(-c2ccccc2)c(-c2ccccc2Cl)nc2ccccc12] | 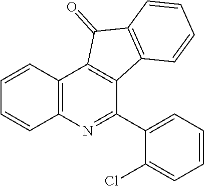 [SMILES: O=C1c2ccccc2-c2c(-c3ccccc3Cl)nc3ccccc3c21] | 75% |
| 3b | 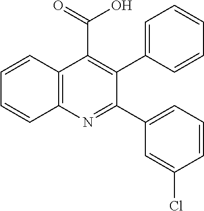 [SMILES: O=C(O)c1c(-c2ccccc2)c(-c2cccc(Cl)c2)nc2ccccc12] | 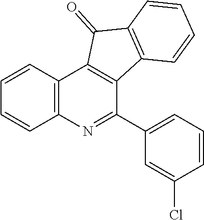 [SMILES: O=C1c2ccccc2-c2c(-c3cccc(Cl)c3)nc3ccccc3c21] | 86% |
| 4b | 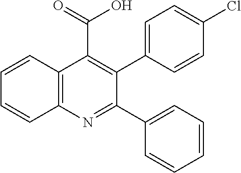 [SMILES: O=C(O)c1c(-c2ccc(Cl)cc2)c(-c2ccccc2)nc2ccccc12] | 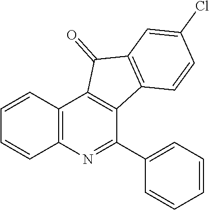 [SMILES: O=C1c2cc(Cl)ccc2-c2c(-c3ccccc3)nc3ccccc3c21] | 63% |
| 5b | 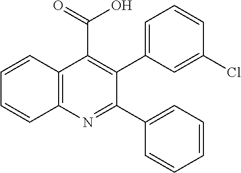 [SMILES: O=C(O)c1c(-c2cccc(Cl)c2)c(-c2ccccc2)nc2ccccc12] | 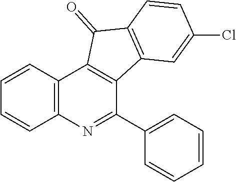 [SMILES: O=C1c2ccc(Cl)cc2-c2c(-c3ccccc3)nc3ccccc3c21] | 71% |
| 6b | 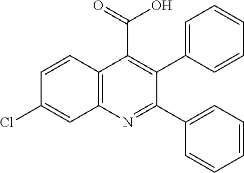 [SMILES: O=C(O)c1c(-c2ccccc2)c(-c2ccccc2)nc2cc(Cl)ccc12] | 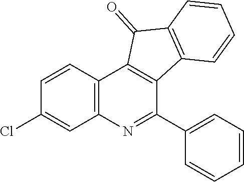 [SMILES: O=C1c2ccccc2-c2c(-c3ccccc3)nc3cc(Cl)ccc3c21] | 80% |
| 7b | 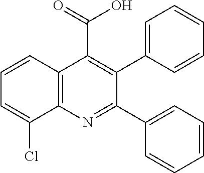 [SMILES: O=C(O)c1c(-c2ccccc2)c(-c2ccccc2)nc2c(Cl)cccc12] | 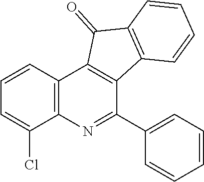 [SMILES: O=C1c2ccccc2-c2c(-c3ccccc3)nc3c(Cl)cccc3c21] | 69% |
| 8b | 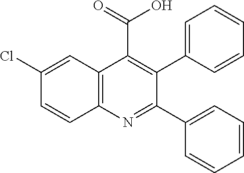 [SMILES: O=C(O)c1c(-c2ccccc2)c(-c2ccccc2)nc2ccc(Cl)cc12] | 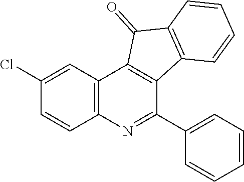 [SMILES: O=C1c2ccccc2-c2c(-c3ccccc3)nc3ccc(Cl)cc3c21] | 77% |

### c) 11-Biphenyl-2-yl-6-(4-chlorophenyl)-11H-indeno[1,2-c]quinolin-11-ol

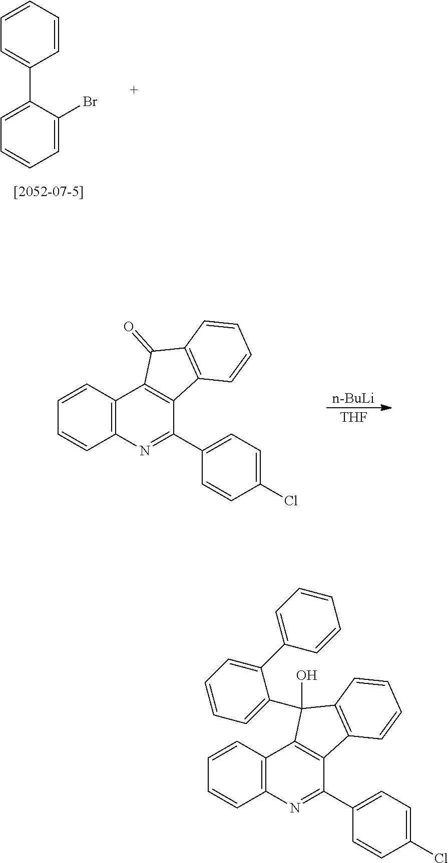

In a 500 mL flask, under protective gas, 13.0 g (53.3 mmol; 1.24 eq) of 2-bromobiphenyl [CAS 2052-07-5] is dissolved in 80 mL of dry THF [CAS 109-99-9] and cooled to −76° C. Then 20.7 mL (2.5 mol/L; 51.7 mmol; 1.20 eq) of n-butyllithium [CAS 109-72-8] is added dropwise and the mixture is stirred for a further 1 hour. A suspension of 14.8 g (43.1 mmol, 1.00 eq) of 6-(4-chlorophenyl)indeno[1,2-c]quinolin-11-one in 370 mL of dry THF [CAS 109-99-9] is added dropwise to this mixture at −40° C. The resulting mixture is warmed gradually to room temperature and stirred for a further 72 hours. The reaction is quenched by addition of 100 mL of water and the resultant phases are separated. After the aqueous phase has been extracted with ethyl acetate (3×150 mL) [CAS 141-78-6], the combined organic phases are washed with water (3×150 mL). The organic phase is concentrated under reduced pressure and precipitated in heptane [CAS 142-82-5]. The crude product is purified by means of column chromatography, and 7.65 g (15.4 mmol; 36% of theory) of the product is obtained in solid form.

The following compounds can be obtained analogously:

| No. | Reactant 1 | Reactant 2 |
|---|---|---|
| 1c | 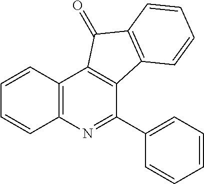 [SMILES: O=C1c2ccccc2-c2c(-c3ccccc3)nc3ccccc3c21] | 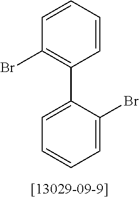 [SMILES: Brc1ccccc1-c1ccccc1Br] |
| 2c | 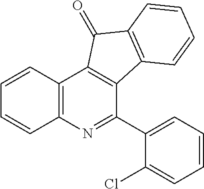 [SMILES: O=C1c2ccccc2-c2c(-c3ccccc3Cl)nc3ccccc3c21] | 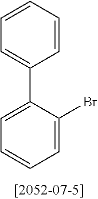 [SMILES: Brc1ccccc1-c1ccccc1] |
| 3c | 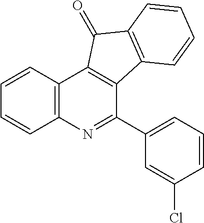 [SMILES: O=C1c2ccccc2-c2c(-c3cccc(Cl)c3)nc3ccccc3c21] | 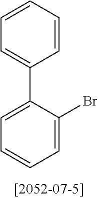 [SMILES: Brc1ccccc1-c1ccccc1] |
| 4c | 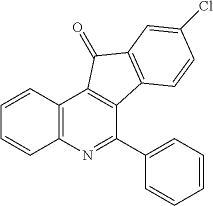 [SMILES: O=C1c2cc(Cl)ccc2-c2c(-c3ccccc3)nc3ccccc3c21] | 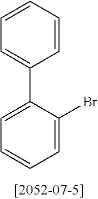 [SMILES: Brc1ccccc1-c1ccccc1] |
| 5c | 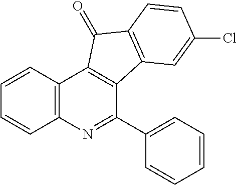 [SMILES: O=C1c2ccc(Cl)cc2-c2c(-c3ccccc3)nc3ccccc3c21] | 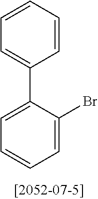 [SMILES: Brc1ccccc1-c1ccccc1] |
| 6c | 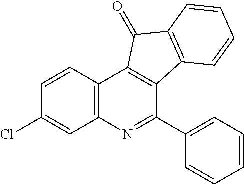 [SMILES: O=C1c2ccccc2-c2c(-c3ccccc3)nc3cc(Cl)ccc3c21] | 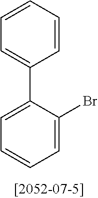 [SMILES: Brc1ccccc1-c1ccccc1] |
| 7c | 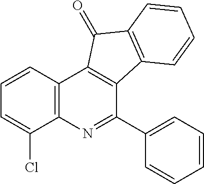 [SMILES: O=C1c2ccccc2-c2c(-c3ccccc3)nc3c(Cl)cccc3c21] | 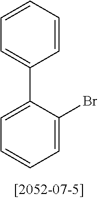 [SMILES: Brc1ccccc1-c1ccccc1] |
| 8c | 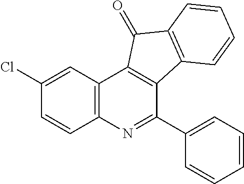 [SMILES: O=C1c2ccccc2-c2c(-c3ccccc3)nc3ccc(Cl)cc3c21] | 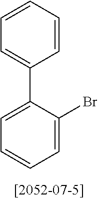 [SMILES: Brc1ccccc1-c1ccccc1] |
| 9c | 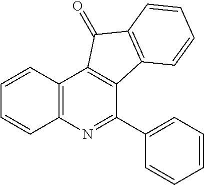 [SMILES: O=C1c2ccccc2-c2c(-c3ccccc3)nc3ccccc3c21] | 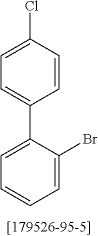 [SMILES: Clc1ccc(-c2ccccc2Br)cc1] |
| 10c | 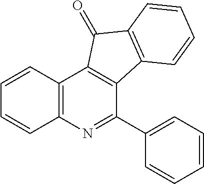 [SMILES: O=C1c2ccccc2-c2c(-c3ccccc3)nc3ccccc3c21] | 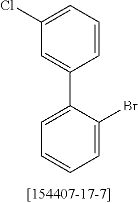 [SMILES: Clc1cccc(-c2ccccc2Br)c1] |
| 11c | 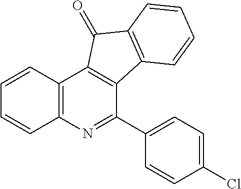 [SMILES: O=C1c2ccccc2-c2c(-c3ccc(Cl)cc3)nc3ccccc3c21] | 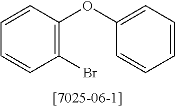 [SMILES: Brc1ccccc1Oc1ccccc1] |
| No. | Product | Yield |
| 1c | 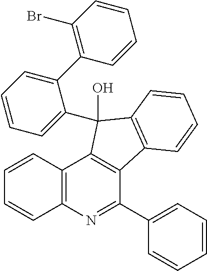 [SMILES: OC1(c2ccccc2-c2ccccc2Br)c2ccccc2-c2c(-c3ccccc3)nc3ccccc3c21] | 55% |
| 2c | 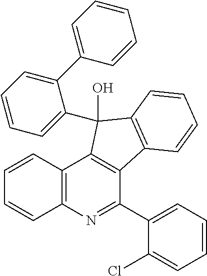 [SMILES: OC1(c2ccccc2-c2ccccc2)c2ccccc2-c2c(-c3ccccc3Cl)nc3ccccc3c21] | 40% |
| 3c | 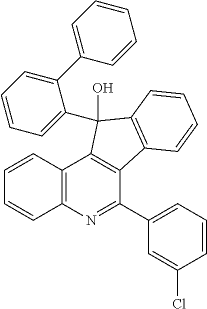 [SMILES: OC1(c2ccccc2-c2ccccc2)c2ccccc2-c2c(-c3cccc(Cl)c3)nc3ccccc3c21] | 47% |
| 4c | 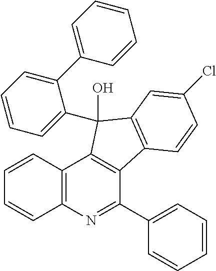 [SMILES: OC1(c2ccccc2-c2ccccc2)c2cc(Cl)ccc2-c2c(-c3ccccc3)nc3ccccc3c21] | 33% |
| 5c | 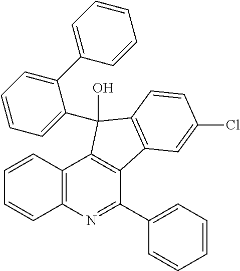 [SMILES: OC1(c2ccccc2-c2ccccc2)c2ccc(Cl)cc2-c2c(-c3ccccc3)nc3ccccc3c21] | 51% |
| 6c | 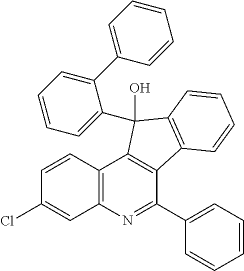 [SMILES: OC1(c2ccccc2-c2ccccc2)c2ccccc2-c2c(-c3ccccc3)nc3cc(Cl)ccc3c21] | 48% |
| 7c | 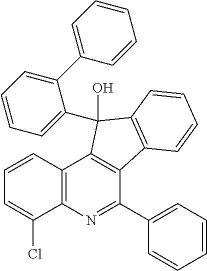 [SMILES: OC1(c2ccccc2-c2ccccc2)c2ccccc2-c2c(-c3ccccc3)nc3c(Cl)cccc3c21] | 30% |
| 8c | 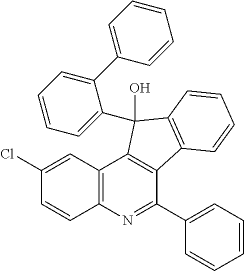 [SMILES: OC1(c2ccccc2-c2ccccc2)c2ccccc2-c2c(-c3ccccc3)nc3ccc(Cl)cc3c21] | 27% |
| 9c | 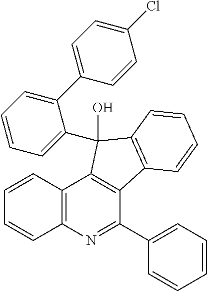 [SMILES: OC1(c2ccccc2-c2ccc(Cl)cc2)c2ccccc2-c2c(-c3ccccc3)nc3ccccc3c21] | 35% |
| 10c | 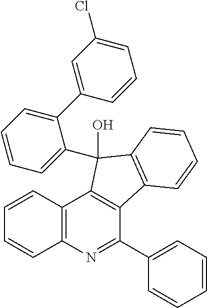 [SMILES: OC1(c2ccccc2-c2cccc(Cl)c2)c2ccccc2-c2c(-c3ccccc3)nc3ccccc3c21] | 40% |
| 11c | 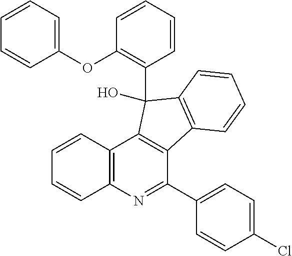 [SMILES: OC1(c2ccccc2Oc2ccccc2)c2ccccc2-c2c(-c3ccc(Cl)cc3)nc3ccccc3c21] | 51% |

### d) 6′-(4-Chlorophenyl)spiro[fluorene-9,11′-indeno[1,2-c]quinoline]

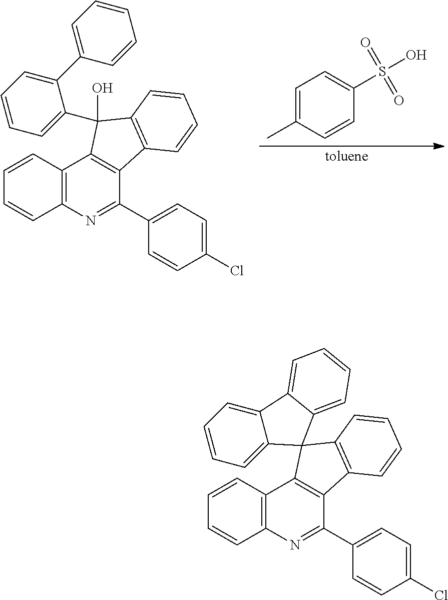

In a 500 mL flask, 7.50 g (15.1 mmol; 1.00 eq) of 11-biphenyl-2-yl-6-(4-chlorophenyl)-11H-indeno[1,2-c]quinolin-11-ol and 28.8 g (150 mmol; 10.0 eq) of toluenesulfonic acid monohydrate [CAS 6192-52-5] are suspended in 250 mL of toluene [CAS 108-88-3], and the mixture is stirred at 115° C. for 24 hours. On completion of conversion, the mixture is cooled down to room temperature and the reaction solution is admixed with water. After phase separation, the aqueous phase is extracted with dichloromethane [CAS 75-09-2] (3×150 mL). The combined organic phases are washed with water (3×150 mL). The organic solvent is removed on a rotary evaporator and the solids obtained are dried. 7.00 g of the product (14.6 mmol; 97% of theory) is converted without further purification.

The following compounds can be obtained analogously:

| No. | Reactant 1 | Product | Yield |
|---|---|---|---|
| 1d | 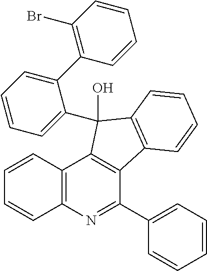 [SMILES: OC1(c2ccccc2-c2ccccc2Br)c2ccccc2-c2c(-c3ccccc3)nc3ccccc3c21] | 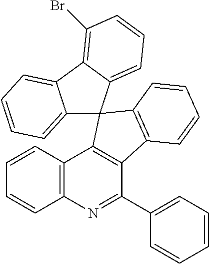 [SMILES: Brc1cccc2c1-c1ccccc1C21c2ccccc2-c2c(-c3ccccc3)nc3ccccc3c21] | 94% |
| 2d | 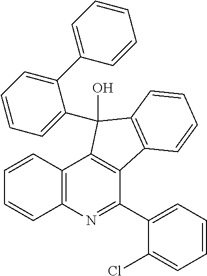 [SMILES: OC1(c2ccccc2-c2ccccc2)c2ccccc2-c2c(-c3ccccc3Cl)nc3ccccc3c21] | 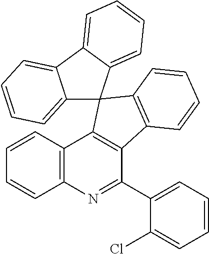 [SMILES: Clc1ccccc1-c1nc2ccccc2c2c1-c1ccccc1C21c2ccccc2-c2ccccc21] | 93% |
| 3d | 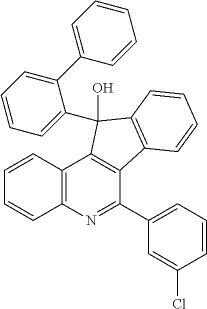 [SMILES: OC1(c2ccccc2-c2ccccc2)c2ccccc2-c2c(-c3cccc(Cl)c3)nc3ccccc3c21] | 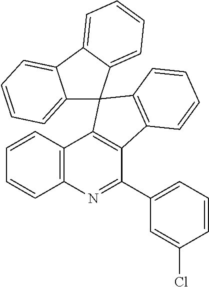 [SMILES: Clc1cccc(-c2nc3ccccc3c3c2-c2ccccc2C32c3ccccc3-c3ccccc32)c1] | 89% |
| 4d | 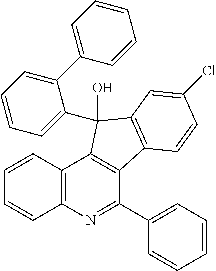 [SMILES: OC1(c2ccccc2-c2ccccc2)c2cc(Cl)ccc2-c2c(-c3ccccc3)nc3ccccc3c21] | 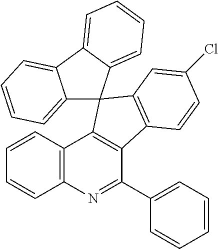 [SMILES: Clc1ccc2c(c1)C1(c3ccccc3-c3ccccc31)c1c-2c(-c2ccccc2)nc2ccccc12] | 91% |
| 5d | 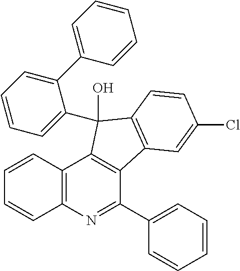 [SMILES: OC1(c2ccccc2-c2ccccc2)c2ccc(Cl)cc2-c2c(-c3ccccc3)nc3ccccc3c21] | 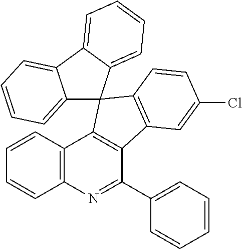 [SMILES: Clc1ccc2c(c1)-c1c(-c3ccccc3)nc3ccccc3c1C21c2ccccc2-c2ccccc21] | 85% |
| 6d | 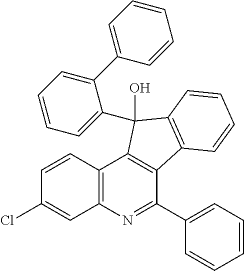 [SMILES: OC1(c2ccccc2-c2ccccc2)c2ccccc2-c2c(-c3ccccc3)nc3cc(Cl)ccc3c21] | 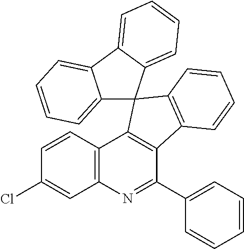 [SMILES: Clc1ccc2c3c(c(-c4ccccc4)nc2c1)-c1ccccc1C31c2ccccc2-c2ccccc21] | 95% |
| 7d | 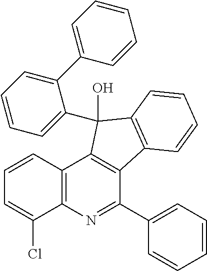 [SMILES: OC1(c2ccccc2-c2ccccc2)c2ccccc2-c2c(-c3ccccc3)nc3c(Cl)cccc3c21] | 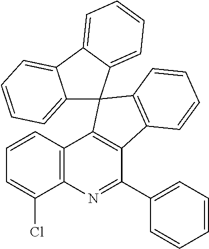 [SMILES: Clc1cccc2c3c(c(-c4ccccc4)nc12)-c1ccccc1C31c2ccccc2-c2ccccc21] | 93% |
| 8d | 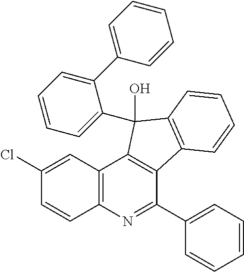 [SMILES: OC1(c2ccccc2-c2ccccc2)c2ccccc2-c2c(-c3ccccc3)nc3ccc(Cl)cc3c21] | 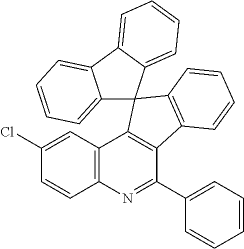 [SMILES: Clc1ccc2nc(-c3ccccc3)c3c(c2c1)C1(c2ccccc2-c2ccccc21)c1ccccc1-3] | 98% |
| 9d | 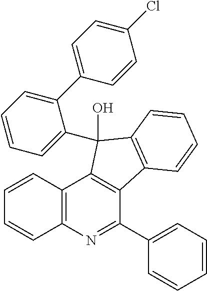 [SMILES: OC1(c2ccccc2-c2ccc(Cl)cc2)c2ccccc2-c2c(-c3ccccc3)nc3ccccc3c21] | 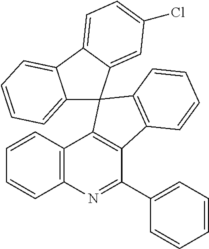 [SMILES: Clc1ccc2c(c1)C1(c3ccccc3-2)c2ccccc2-c2c(-c3ccccc3)nc3ccccc3c21] | 88% |
| 10d | 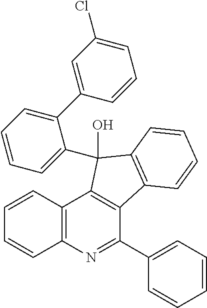 [SMILES: OC1(c2ccccc2-c2cccc(Cl)c2)c2ccccc2-c2c(-c3ccccc3)nc3ccccc3c21] | 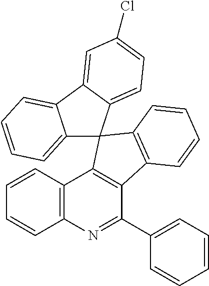 [SMILES: Clc1ccc2c(c1)-c1ccccc1C21c2ccccc2-c2c(-c3ccccc3)nc3ccccc3c21] | 87% |
| 11d | 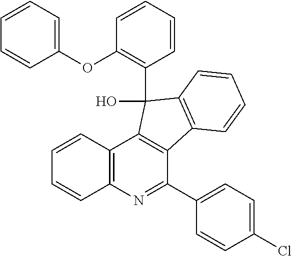 [SMILES: OC1(c2ccccc2Oc2ccccc2)c2ccccc2-c2c(-c3ccc(Cl)cc3)nc3ccccc3c21] | 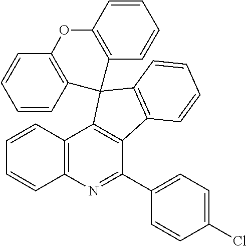 [SMILES: Clc1ccc(-c2nc3ccccc3c3c2-c2ccccc2C32c3ccccc3Oc3ccccc32)cc1] | 80% |

### e) 6′-[4-(4,4,5,5-Tetramethyl-1,3,2-dioxaborolan-2-yl)phenyl]spiro[fluorene-9,11′-indeno[1,2-c]quinoline]

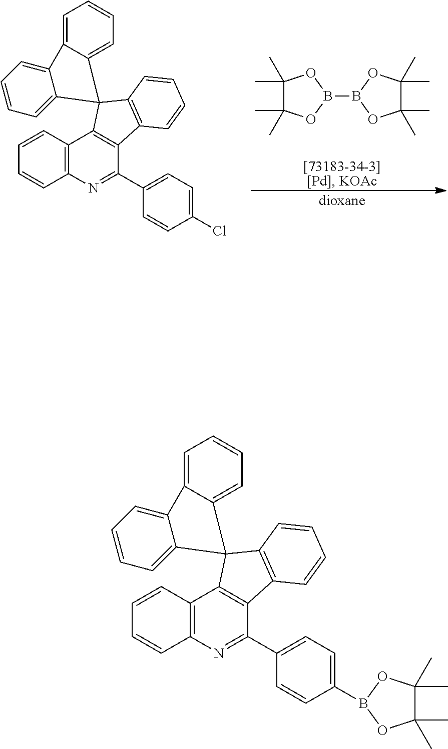

In a 500 mL flask, under protective gas, 6.34 g (13.3 mmol; 1.00 eq.) of 6′-(4-chlorophenyl)spiro[fluorene-9,11′-indeno[1,2-c]quinoline] and 4.30 g (16.9 mmol; 1.28 eq) of bis(pinacolato)diborane [CAS 73183-34-3] are dissolved in 200 mL of dry dioxane [CAS 123-91-1] and the mixture is degassed for 45 min. Subsequently, 3.20 g (32.6 mmol; 2.46 eq.) of potassium acetate [CAS 127-08-2] and 510 mg (0.69 mmol; 0.05 eq.) of bis(tricyclohexylphosphine)palladium dichloride [CAS 29934-17-6] are added, and the mixture is heated to 110° C. overnight. On completion of conversion, and after cooling to room temperature, 300 mL of ethyl acetate [CAS 141-78-6] and 300 mL of water are added to the reaction. After phase separation and extraction of the aqueous phase with ethyl acetate, the combined organic phases are concentrated and washed with water. After the solvent has been removed, the desired product (7.60 g; 12.3 mmol; 93% of theory) is obtained.

The following compounds can be obtained analogously:

| No. | Reactant 1 | Product | Yield |
|---|---|---|---|
| 1e | 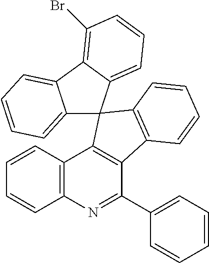 [SMILES: Brc1cccc2c1-c1ccccc1C21c2ccccc2-c2c(-c3ccccc3)nc3ccccc3c21] | 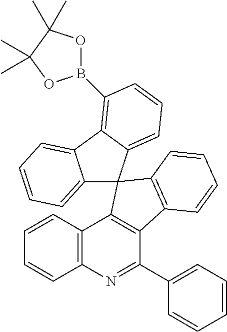 [SMILES: CC1(C)OB(c2cccc3c2-c2ccccc2C32c3ccccc3-c3c(-c4ccccc4)nc4ccccc4c32)OC1(C)C] | 95% |
| 2e | 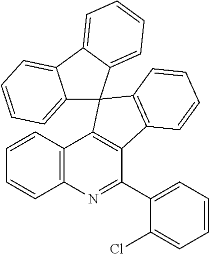 [SMILES: Clc1ccccc1-c1nc2ccccc2c2c1-c1ccccc1C21c2ccccc2-c2ccccc21] | 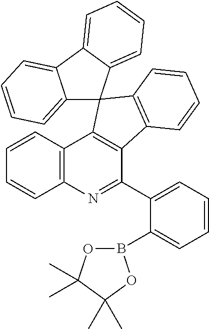 [SMILES: CC1(C)OB(c2ccccc2-c2nc3ccccc3c3c2-c2ccccc2C32c3ccccc3-c3ccccc32)OC1(C)C] | 85% |
| 3e | 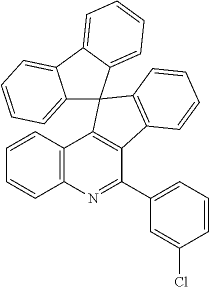 [SMILES: Clc1cccc(-c2nc3ccccc3c3c2-c2ccccc2C32c3ccccc3-c3ccccc32)c1] | 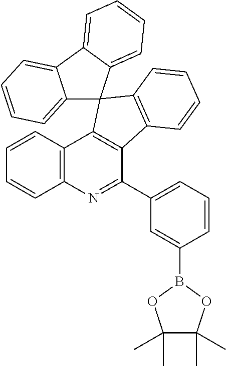 [SMILES: CC1(C)OB(c2cccc(-c3nc4ccccc4c4c3-c3ccccc3C43c4ccccc4-c4ccccc43)c2)OC1(C)C] | 95% |
| 4e | 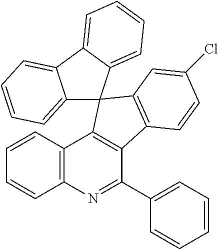 [SMILES: Clc1ccc2c(c1)C1(c3ccccc3-c3ccccc31)c1c-2c(-c2ccccc2)nc2ccccc12] | 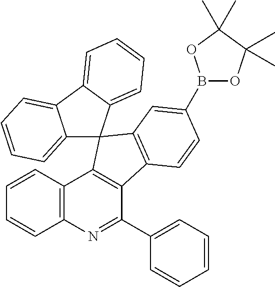 [SMILES: CC1(C)OB(c2ccc3c(c2)C2(c4ccccc4-c4ccccc42)c2c-3c(-c3ccccc3)nc3ccccc23)OC1(C)C] | 89% |
| 5e | 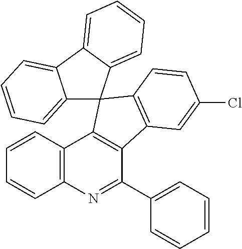 [SMILES: Clc1ccc2c(c1)-c1c(-c3ccccc3)nc3ccccc3c1C21c2ccccc2-c2ccccc21] | 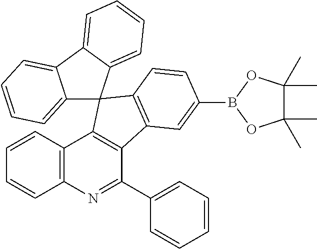 [SMILES: CC1(C)OB(c2ccc3c(c2)-c2c(-c4ccccc4)nc4ccccc4c2C32c3ccccc3-c3ccccc32)OC1(C)C] | 91% |
| 6e | 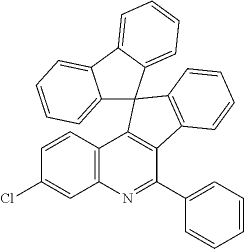 [SMILES: Clc1ccc2c3c(c(-c4ccccc4)nc2c1)-c1ccccc1C31c2ccccc2-c2ccccc21] | 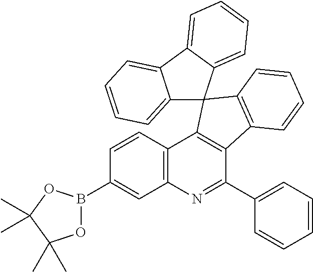 [SMILES: CC1(C)OB(c2ccc3c4c(c(-c5ccccc5)nc3c2)-c2ccccc2C42c3ccccc3-c3ccccc32)OC1(C)C] | 88% |
| 7e | 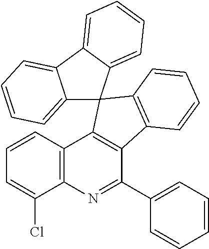 [SMILES: Clc1cccc2c3c(c(-c4ccccc4)nc12)-c1ccccc1C31c2ccccc2-c2ccccc21] | 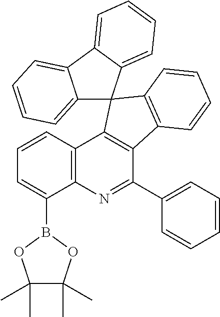 [SMILES: CC1(C)OB(c2cccc3c4c(c(-c5ccccc5)nc23)-c2ccccc2C42c3ccccc3-c3ccccc32)OC1(C)C] | 93% |
| 8e | 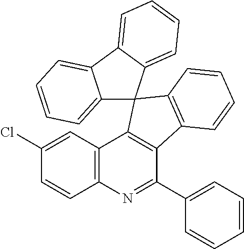 [SMILES: Clc1ccc2nc(-c3ccccc3)c3c(c2c1)C1(c2ccccc2-c2ccccc21)c1ccccc1-3] | 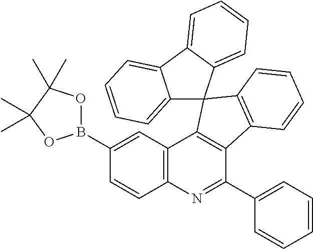 [SMILES: CC1(C)OB(c2ccc3nc(-c4ccccc4)c4c(c3c2)C2(c3ccccc3-c3ccccc32)c2ccccc2-4)OC1(C)C] | 81% |
| 9e | 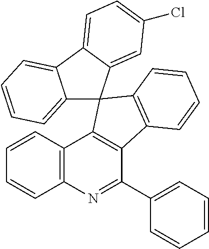 [SMILES: Clc1ccc2c(c1)C1(c3ccccc3-2)c2ccccc2-c2c(-c3ccccc3)nc3ccccc3c21] | 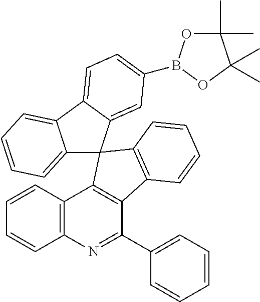 [SMILES: CC1(C)OB(c2ccc3c(c2)C2(c4ccccc4-3)c3ccccc3-c3c(-c4ccccc4)nc4ccccc4c32)OC1(C)C] | 84% |
| 10e | 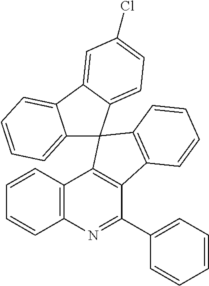 [SMILES: Clc1ccc2c(c1)-c1ccccc1C21c2ccccc2-c2c(-c3ccccc3)nc3ccccc3c21] | 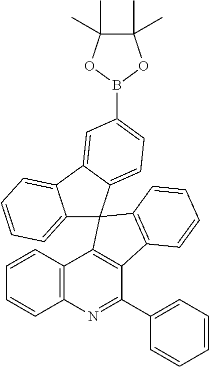 [SMILES: CC1(C)OB(c2ccc3c(c2)-c2ccccc2C32c3ccccc3-c3c(-c4ccccc4)nc4ccccc4c32)OC1(C)C] | 95% |
| 11e | 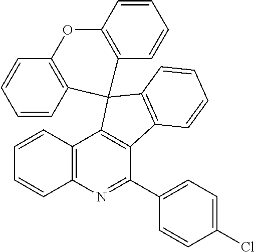 [SMILES: Clc1ccc(-c2nc3ccccc3c3c2-c2ccccc2C32c3ccccc3Oc3ccccc32)cc1] | 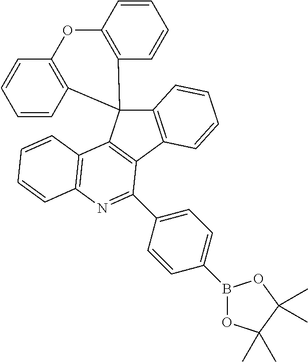 [SMILES: CC1(C)OB(c2ccc(-c3nc4ccccc4c4c3-c3ccccc3C43c4ccccc4Oc4ccccc43)cc2)OC1(C)C] | 93% |

### f) 6′-[4-(4,6-Diphenyl-1,3,5-triazin-2-yl)phenyl]spiro[fluorene-9,11′-indeno[1,2-c]quinoline]

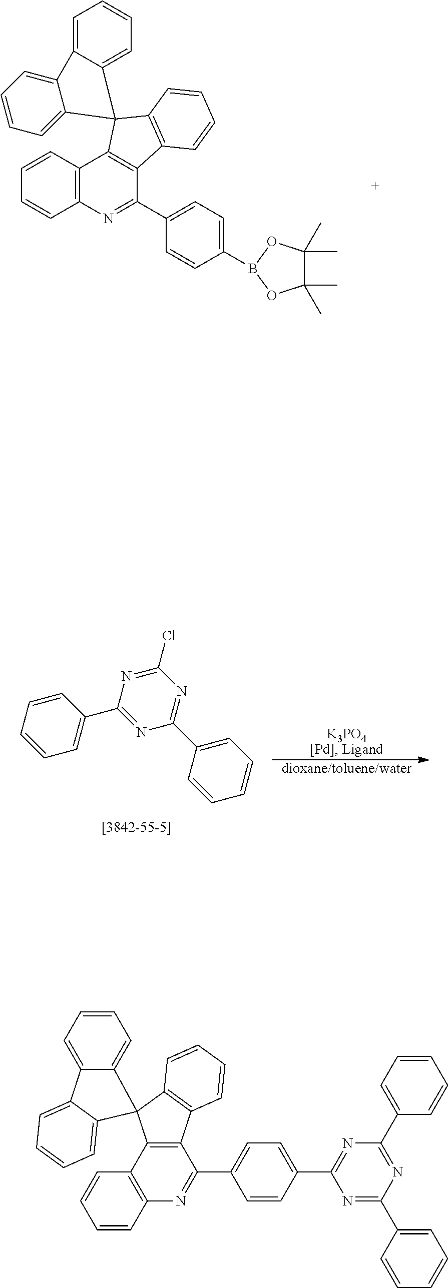

7.00 g (11.3 mmol; 1.05 eq.) of 6′-[4-(4,4,5,5-tetramethyl-1,3,2-dioxaborolan-2-yl)phenyl]spiro[fluorene-9,11′-indeno[1,2-c]quinoline], 2.89 g (10.8 mmol; 1.00 eq.) of 2-chloro-4,6-diphenyl-[1,3,5]triazine [CAS 3842-55-5] and 8.04 g (32.4 mmol; 3.00 eq.) of tripotassium phosphate [CAS 7778-53-2] are suspended in 75 mL of dioxane [CAS 123-91-1], 75 mL of toluene [CAS 108-88-3] and 75 mL of water. To this suspension are added 121 mg (0.54 mmol; 0.05 eq.) of palladium(II) acetate [CAS 3375-31-3] and 329 mg (1.08 mmol; 0.10 eq.) of triorthotolylphosphine [CAS 6163-58-2], and the reaction mixture is heated under reflux for 16 h. After cooling, the solvent is removed and the residue is dissolved in dichloromethane [CAS 75-09-2] and water, and the phases are separated. After the aqueous phase has been extracted with dichloromethane (2×150 mL), the combined organic phases are washed with water (2×150 mL) and then concentrated to dryness. Purification by means of Soxhlet extraction, washing with ethyl acetate [CAS 141-78-6] and vacuum sublimation gives the desired product (1.78 g; 2.64 mmol; 24% of theory).

The following compounds can be obtained analogously:

| No. | Reactant 1 | Reactant 2 |
|---|---|---|
| 1f | 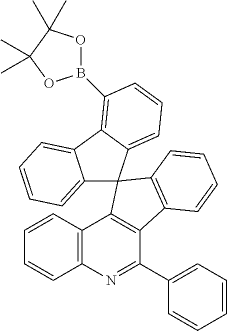 [SMILES: CC1(C)OB(c2cccc3c2-c2ccccc2C32c3ccccc3-c3c(-c4ccccc4)nc4ccccc4c32)OC1(C)C] | 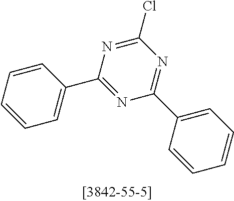 [SMILES: Clc1nc(-c2ccccc2)nc(-c2ccccc2)n1] |
| 2f | 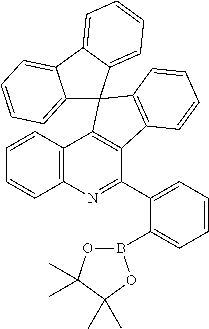 [SMILES: CC1(C)OB(c2ccccc2-c2nc3ccccc3c3c2-c2ccccc2C32c3ccccc3-c3ccccc32)OC1(C)C] | 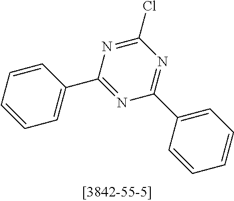 [SMILES: Clc1nc(-c2ccccc2)nc(-c2ccccc2)n1] |
| 3f | 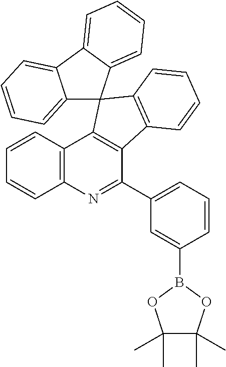 [SMILES: CC1(C)OB(c2cccc(-c3nc4ccccc4c4c3-c3ccccc3C43c4ccccc4-c4ccccc43)c2)OC1(C)C] | 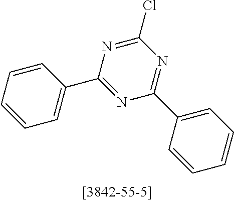 [SMILES: Clc1nc(-c2ccccc2)nc(-c2ccccc2)n1] |
| 4f | 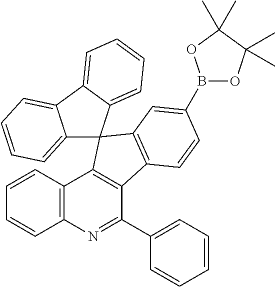 [SMILES: CC1(C)OB(c2ccc3c(c2)C2(c4ccccc4-c4ccccc42)c2c-3c(-c3ccccc3)nc3ccccc23)OC1(C)C] | 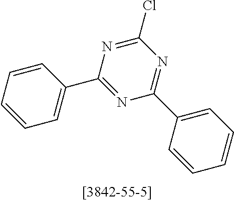 [SMILES: Clc1nc(-c2ccccc2)nc(-c2ccccc2)n1] |
| 5f | 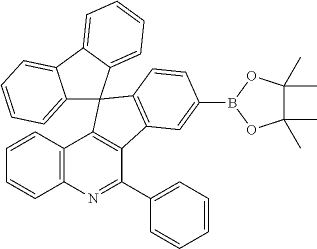 [SMILES: CC1(C)OB(c2ccc3c(c2)-c2c(-c4ccccc4)nc4ccccc4c2C32c3ccccc3-c3ccccc32)OC1(C)C] | 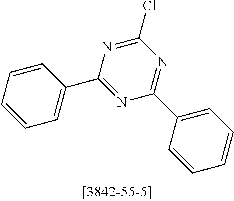 [SMILES: Clc1nc(-c2ccccc2)nc(-c2ccccc2)n1] |
| 6f | 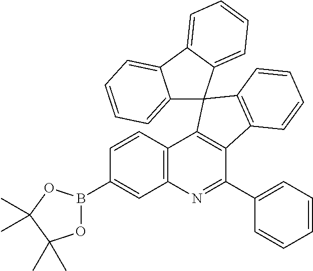 [SMILES: CC1(C)OB(c2ccc3c4c(c(-c5ccccc5)nc3c2)-c2ccccc2C42c3ccccc3-c3ccccc32)OC1(C)C] | 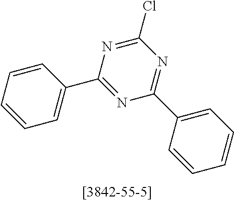 [SMILES: Clc1nc(-c2ccccc2)nc(-c2ccccc2)n1] |
| 7f | 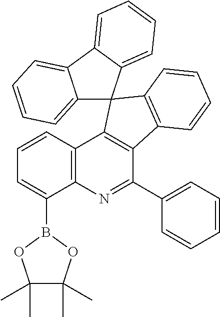 [SMILES: CC1(C)OB(c2cccc3c4c(c(-c5ccccc5)nc23)-c2ccccc2C42c3ccccc3-c3ccccc32)OC1(C)C] | 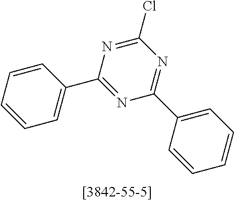 [SMILES: Clc1nc(-c2ccccc2)nc(-c2ccccc2)n1] |
| 8f | 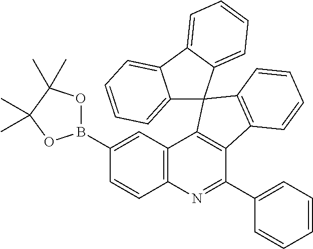 [SMILES: CC1(C)OB(c2ccc3nc(-c4ccccc4)c4c(c3c2)C2(c3ccccc3-c3ccccc32)c2ccccc2-4)OC1(C)C] | 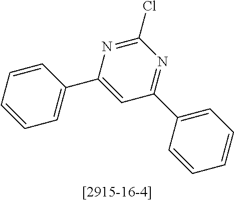 [SMILES: Clc1nc(-c2ccccc2)cc(-c2ccccc2)n1] |
| 9f | 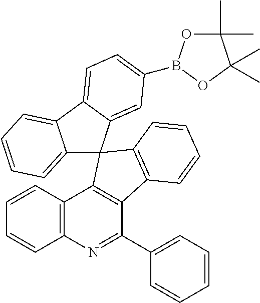 [SMILES: CC1(C)OB(c2ccc3c(c2)C2(c4ccccc4-3)c3ccccc3-c3c(-c4ccccc4)nc4ccccc4c32)OC1(C)C] | 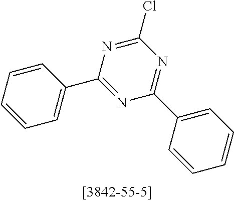 [SMILES: Clc1nc(-c2ccccc2)nc(-c2ccccc2)n1] |
| 10f | 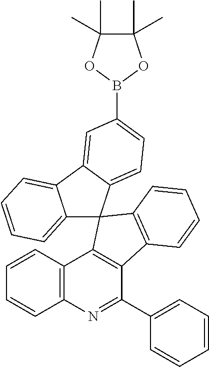 [SMILES: CC1(C)OB(c2ccc3c(c2)-c2ccccc2C32c3ccccc3-c3c(-c4ccccc4)nc4ccccc4c32)OC1(C)C] | 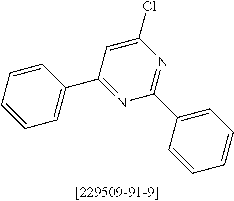 [SMILES: Clc1cc(-c2ccccc2)nc(-c2ccccc2)n1] |
| 11f | 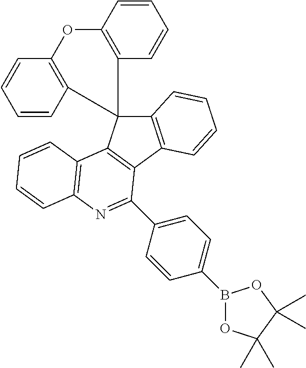 [SMILES: CC1(C)OB(c2ccc(-c3nc4ccccc4c4c3-c3ccccc3C43c4ccccc4Oc4ccccc43)cc2)OC1(C)C] | 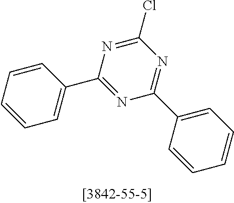 [SMILES: Clc1nc(-c2ccccc2)nc(-c2ccccc2)n1] |
| 12f | 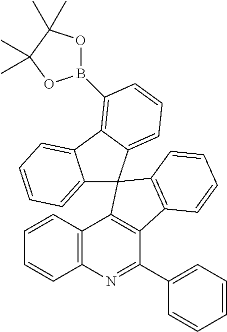 [SMILES: CC1(C)OB(c2cccc3c2-c2ccccc2C32c3ccccc3-c3c(-c4ccccc4)nc4ccccc4c32)OC1(C)C] | 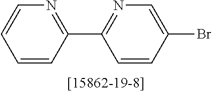 [SMILES: Brc1ccc(-c2ccccn2)nc1] |
| 13f | 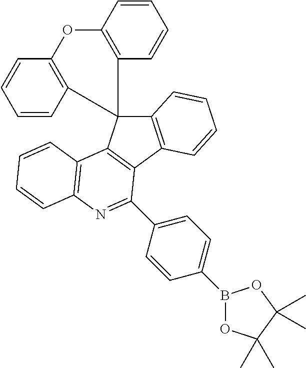 [SMILES: CC1(C)OB(c2ccc(-c3nc4ccccc4c4c3-c3ccccc3C43c4ccccc4Oc4ccccc43)cc2)OC1(C)C] | 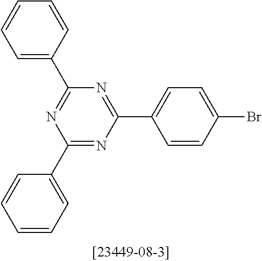 [SMILES: Brc1ccc(-c2nc(-c3ccccc3)nc(-c3ccccc3)n2)cc1] |
| 14f | 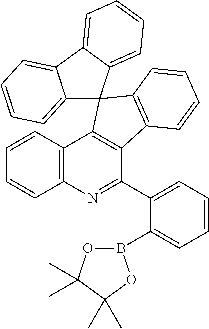 [SMILES: CC1(C)OB(c2ccccc2-c2nc3ccccc3c3c2-c2ccccc2C32c3ccccc3-c3ccccc32)OC1(C)C] | 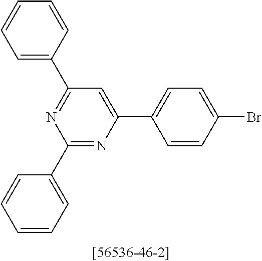 [SMILES: Brc1ccc(-c2cc(-c3ccccc3)nc(-c3ccccc3)n2)cc1] |
| 15f | 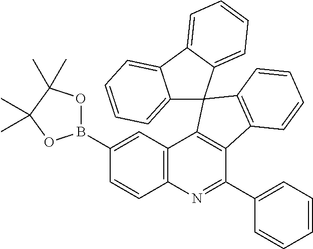 [SMILES: CC1(C)OB(c2ccc3nc(-c4ccccc4)c4c(c3c2)C2(c3ccccc3-c3ccccc32)c2ccccc2-4)OC1(C)C] | 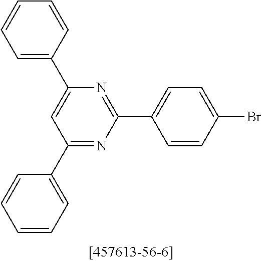 [SMILES: Brc1ccc(-c2nc(-c3ccccc3)cc(-c3ccccc3)n2)cc1] |
| 16f | 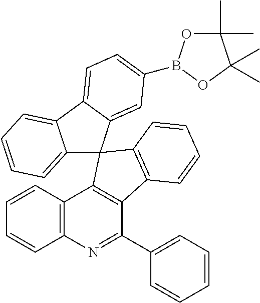 [SMILES: CC1(C)OB(c2ccc3c(c2)C2(c4ccccc4-3)c3ccccc3-c3c(-c4ccccc4)nc4ccccc4c32)OC1(C)C] | 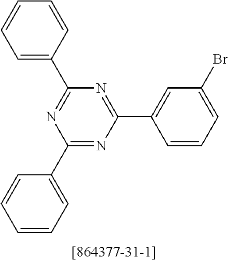 [SMILES: Brc1cccc(-c2nc(-c3ccccc3)nc(-c3ccccc3)n2)c1] |
| 17f | 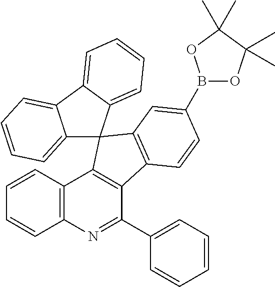 [SMILES: CC1(C)OB(c2ccc3c(c2)C2(c4ccccc4-c4ccccc42)c2c-3c(-c3ccccc3)nc3ccccc23)OC1(C)C] | 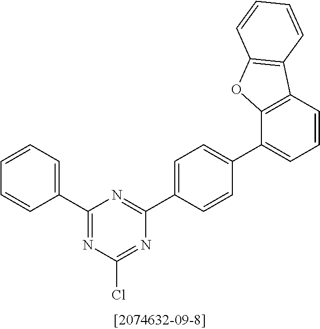 [SMILES: Clc1nc(-c2ccccc2)nc(-c2ccc(-c3cccc4c3oc3ccccc34)cc2)n1] |
| 18f | 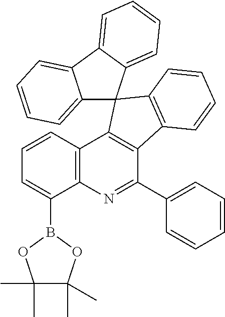 [SMILES: CC1(C)OB(c2cccc3c4c(c(-c5ccccc5)nc23)-c2ccccc2C42c3ccccc3-c3ccccc32)OC1(C)C] | 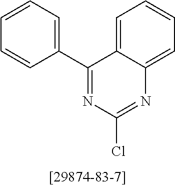 [SMILES: Clc1nc(-c2ccccc2)c2ccccc2n1] |
| 19f | 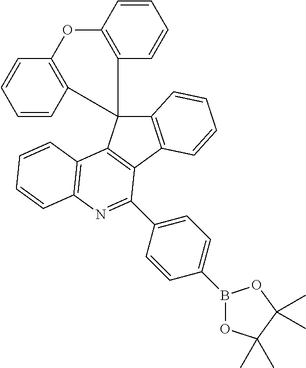 [SMILES: CC1(C)OB(c2ccc(-c3nc4ccccc4c4c3-c3ccccc3C43c4ccccc4Oc4ccccc43)cc2)OC1(C)C] | 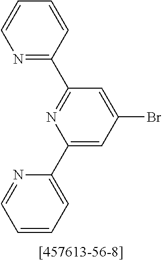 [SMILES: Brc1cc(-c2ccccn2)nc(-c2ccccn2)c1] |
| 20f | 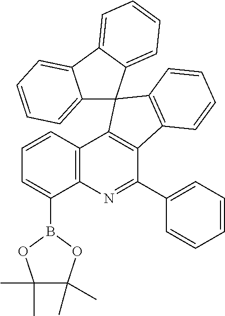 [SMILES: CC1(C)OB(c2cccc3c4c(c(-c5ccccc5)nc23)-c2ccccc2C42c3ccccc3-c3ccccc32)OC1(C)C] | 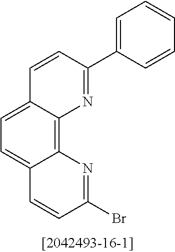 [SMILES: Brc1ccc2ccc3ccc(-c4ccccc4)nc3c2n1] |
| 21f | 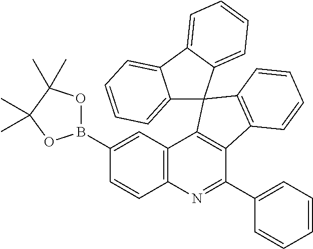 [SMILES: CC1(C)OB(c2ccc3nc(-c4ccccc4)c4c(c3c2)C2(c3ccccc3-c3ccccc32)c2ccccc2-4)OC1(C)C] | 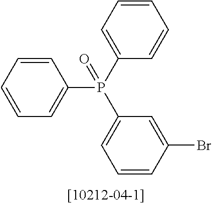 [SMILES: O=P(c1ccccc1)(c1ccccc1)c1cccc(Br)c1] |
| 22f | 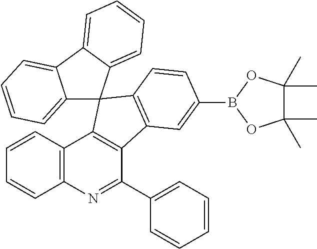 [SMILES: CC1(C)OB(c2ccc3c(c2)-c2c(-c4ccccc4)nc4ccccc4c2C32c3ccccc3-c3ccccc32)OC1(C)C] | 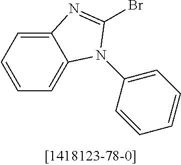 [SMILES: Brc1nc2ccccc2n1-c1ccccc1] |
| No. | Product | Yield |
| 1f | 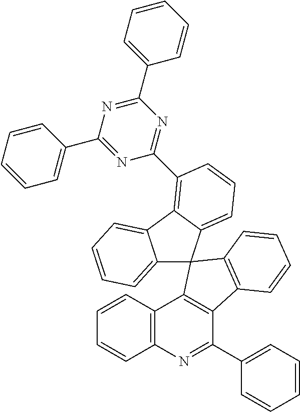 [SMILES: c1ccc(-c2nc(-c3ccccc3)nc(-c3cccc4c3-c3ccccc3C43c4ccccc4-c4c(-c5ccccc5)nc5ccccc5c43)n2)cc1] | 38% |
| 2f | 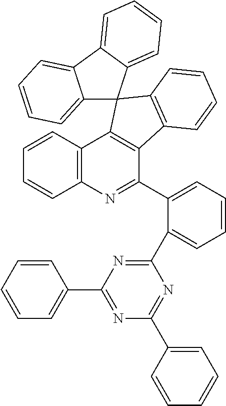 [SMILES: c1ccc(-c2nc(-c3ccccc3)nc(-c3ccccc3-c3nc4ccccc4c4c3-c3ccccc3C43c4ccccc4-c4ccccc43)n2)cc1] | 44% |
| 3f | 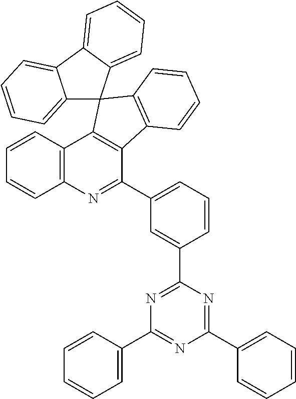 [SMILES: c1ccc(-c2nc(-c3ccccc3)nc(-c3cccc(-c4nc5ccccc5c5c4-c4ccccc4C54c5ccccc5-c5ccccc54)c3)n2)cc1] | 35% |
| 4f | 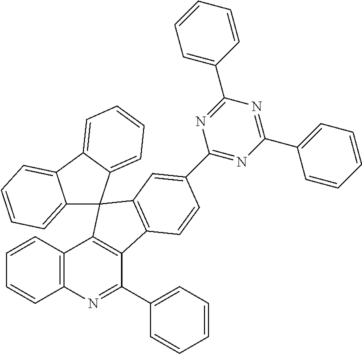 [SMILES: c1ccc(-c2nc(-c3ccccc3)nc(-c3ccc4c(c3)C3(c5ccccc5-c5ccccc53)c3c-4c(-c4ccccc4)nc4ccccc34)n2)cc1] | 49% |
| 5f | 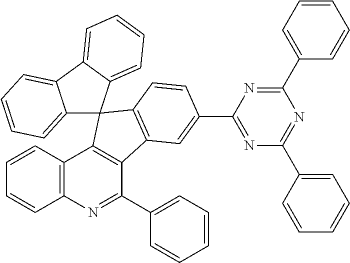 [SMILES: c1ccc(-c2nc(-c3ccccc3)nc(-c3ccc4c(c3)-c3c(-c5ccccc5)nc5ccccc5c3C43c4ccccc4-c4ccccc43)n2)cc1] | 47% |
| 6f | 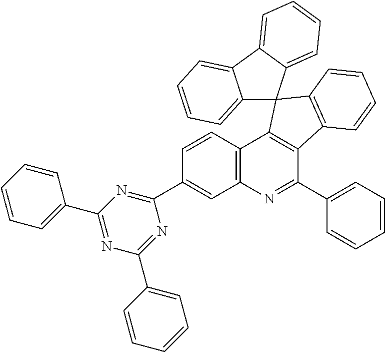 [SMILES: c1ccc(-c2nc(-c3ccccc3)nc(-c3ccc4c5c(c(-c6ccccc6)nc4c3)-c3ccccc3C53c4ccccc4-c4ccccc43)n2)cc1] | 41% |
| 7f | 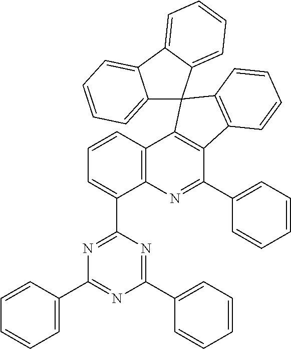 [SMILES: c1ccc(-c2nc(-c3ccccc3)nc(-c3cccc4c5c(c(-c6ccccc6)nc34)-c3ccccc3C53c4ccccc4-c4ccccc43)n2)cc1] | 58% |
| 8f | 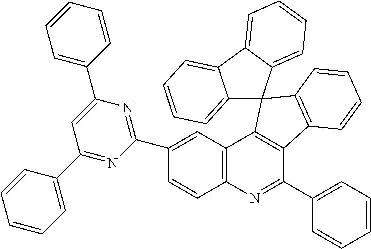 [SMILES: c1ccc(-c2cc(-c3ccccc3)nc(-c3ccc4nc(-c5ccccc5)c5c(c4c3)C3(c4ccccc4-c4ccccc43)c3ccccc3-5)n2)cc1] | 20% |
| 9f | 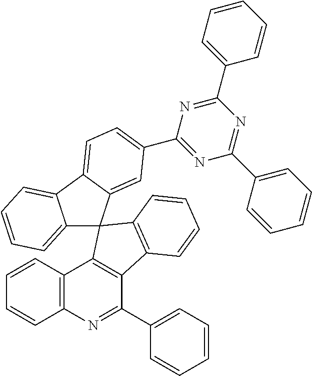 [SMILES: c1ccc(-c2nc(-c3ccccc3)nc(-c3ccc4c(c3)C3(c5ccccc5-4)c4ccccc4-c4c(-c5ccccc5)nc5ccccc5c43)n2)cc1] | 36% |
| 10f | 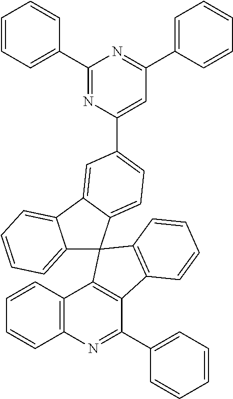 [SMILES: c1ccc(-c2cc(-c3ccc4c(c3)-c3ccccc3C43c4ccccc4-c4c(-c5ccccc5)nc5ccccc5c43)nc(-c3ccccc3)n2)cc1] | 29% |
| 11f | 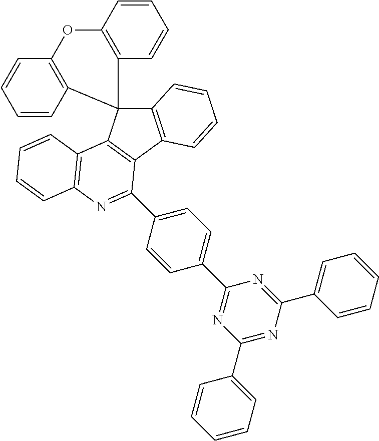 [SMILES: c1ccc(-c2nc(-c3ccccc3)nc(-c3ccc(-c4nc5ccccc5c5c4-c4ccccc4C54c5ccccc5Oc5ccccc54)cc3)n2)cc1] | 27% |
| 12f | 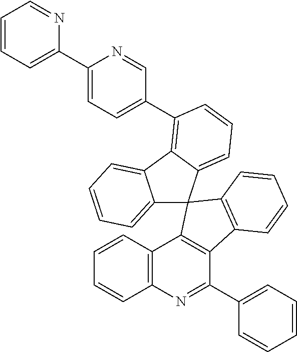 [SMILES: c1ccc(-c2nc3ccccc3c3c2-c2ccccc2C32c3ccccc3-c3c(-c4ccc(-c5ccccn5)nc4)cccc32)cc1] | 34% |
| 13f | 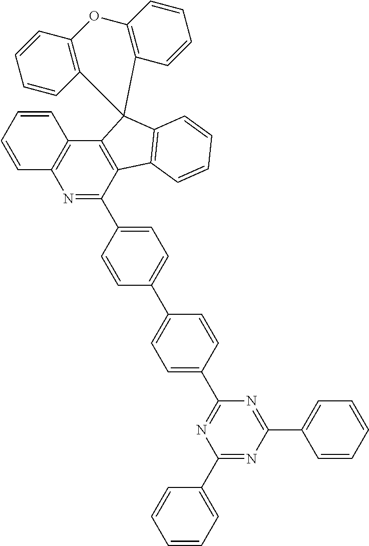 [SMILES: c1ccc(-c2nc(-c3ccccc3)nc(-c3ccc(-c4ccc(-c5nc6ccccc6c6c5-c5ccccc5C65c6ccccc6Oc6ccccc65)cc4)cc3)n2)cc1] | 39% |
| 14f | 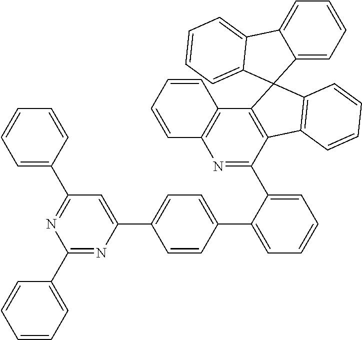 [SMILES: c1ccc(-c2cc(-c3ccc(-c4ccccc4-c4nc5ccccc5c5c4-c4ccccc4C54c5ccccc5-c5ccccc54)cc3)nc(-c3ccccc3)n2)cc1] | 29% |
| 15f | 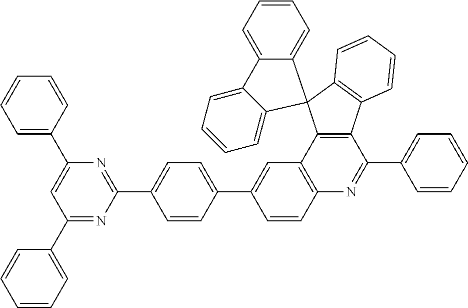 [SMILES: c1ccc(-c2cc(-c3ccccc3)nc(-c3ccc(-c4ccc5nc(-c6ccccc6)c6c(c5c4)C4(c5ccccc5-c5ccccc54)c4ccccc4-6)cc3)n2)cc1] | 29% |
| 16f | 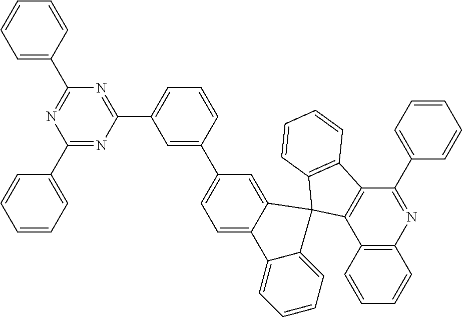 [SMILES: c1ccc(-c2nc(-c3ccccc3)nc(-c3cccc(-c4ccc5c(c4)C4(c6ccccc6-5)c5ccccc5-c5c(-c6ccccc6)nc6ccccc6c54)c3)n2)cc1] | 35% |
| 17f | 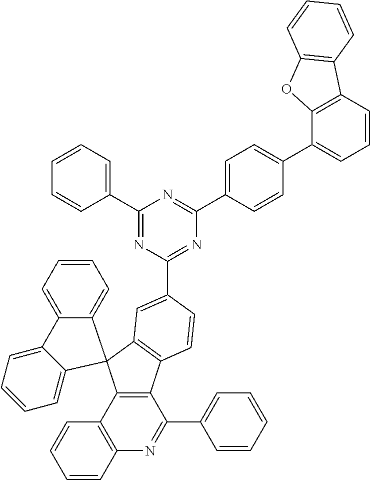 [SMILES: c1ccc(-c2nc(-c3ccc(-c4cccc5c4oc4ccccc45)cc3)nc(-c3ccc4c(c3)C3(c5ccccc5-c5ccccc53)c3c-4c(-c4ccccc4)nc4ccccc34)n2)cc1] | 38% |
| 18f | 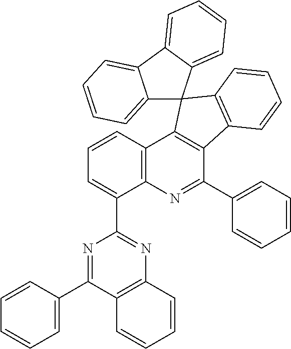 [SMILES: c1ccc(-c2nc3c(-c4nc(-c5ccccc5)c5ccccc5n4)cccc3c3c2-c2ccccc2C32c3ccccc3-c3ccccc32)cc1] | 41% |
| 19f | 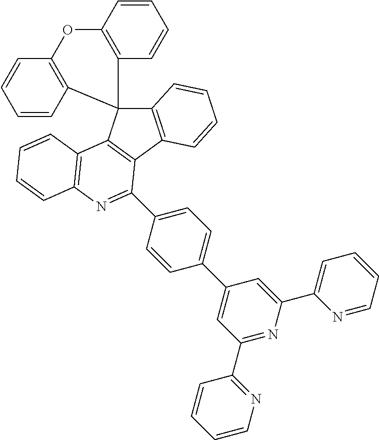 [SMILES: c1ccc(-c2cc(-c3ccc(-c4nc5ccccc5c5c4-c4ccccc4C54c5ccccc5Oc5ccccc54)cc3)cc(-c3ccccn3)n2)nc1] | 48% |
| 20f | 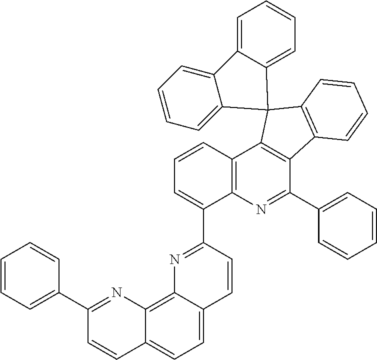 [SMILES: c1ccc(-c2ccc3ccc4ccc(-c5cccc6c7c(c(-c8ccccc8)nc56)-c5ccccc5C75c6ccccc6-c6ccccc65)nc4c3n2)cc1] | 42% |
| 21f | 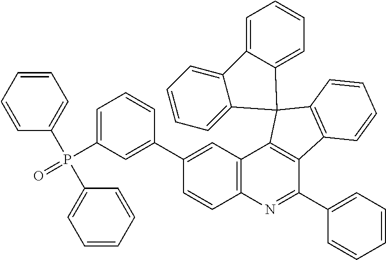 [SMILES: O=P(c1ccccc1)(c1ccccc1)c1cccc(-c2ccc3nc(-c4ccccc4)c4c(c3c2)C2(c3ccccc3-c3ccccc32)c2ccccc2-4)c1] | 22% |
| 22f | 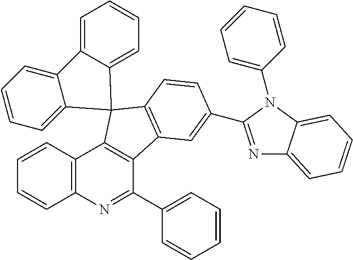 [SMILES: c1ccc(-c2nc3ccccc3c3c2-c2cc(-c4nc5ccccc5n4-c4ccccc4)ccc2C32c3ccccc3-c3ccccc32)cc1] | 25% |

Isoquinoline derivatives of formula (Ia) can be obtained in accordance with the above-cited examples, with some corresponding isoquinolines being commercially available, or these being obtainable via known reactions, for example the Bischler-Napieralski reaction, the Pictet-Gams reaction or the Pictet-Spengler reaction.

OLEDs

The compounds of the invention can be used in organic electroluminescent devices, especially in OLEDs. These are produced and characterized by methods that are well known to the person skilled in the art. OLEDs containing the compounds of the invention have the abovementioned advantages over known OLEDs. Especially the performance data such as voltage, lifetime, stability and efficiency of the OLEDs can be enhanced by using the compounds of the invention.

General production process for the OLEDs and characterization of the OLEDs: Glass plates which have been coated with structured ITO (indium tin oxide) in a thickness of 50 nm form the substrates to which the OLEDs are applied.

The OLEDs basically have the following layer structure: substrate/hole injection layer (HIL)/hole transport layer (HTL)/electron blocker layer (EBL)/emission layer (EML)/electron transport layer (ETL)/electron injection layer (EIL) and finally a cathode. The cathode is formed by an aluminum layer of thickness 100 nm. Table 1 shows typical materials that can be used for production of OLEDs.

TABLE 1
Materials used

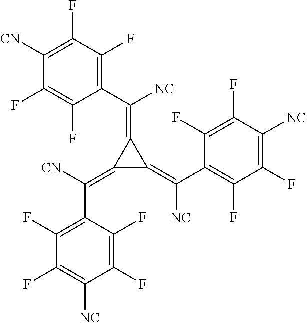

p-Dopant

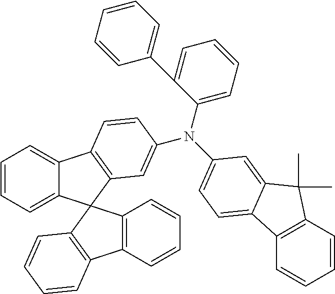 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

HTM

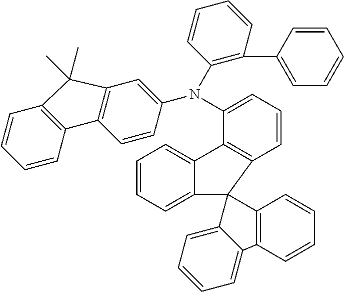 [SMILES: CC1(C)c2ccccc2-c2ccc(N(c3ccccc3-c3ccccc3)c3cccc4c3-c3ccccc3C43c4ccccc4-c4ccccc43)cc21]

EBM

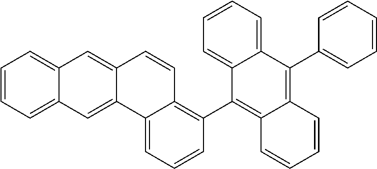 [SMILES: c1ccc(-c2c3ccccc3c(-c3cccc4c3ccc3cc5ccccc5cc34)c3ccccc23)cc1]

H

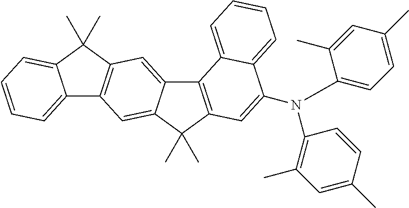 [SMILES: Cc1ccc(N(c2ccc(C)cc2C)c2cc3c(c4ccccc24)-c2cc4c(cc2C3(C)C)-c2ccccc2C4(C)C)c(C)c1]

SEB

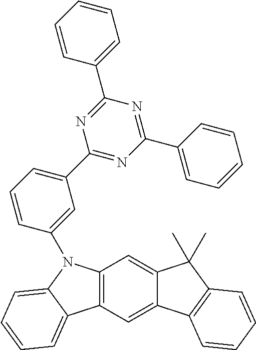 [SMILES: CC1(C)c2ccccc2-c2cc3c4ccccc4n(-c4cccc(-c5nc(-c6ccccc6)nc(-c6ccccc6)n5)c4)c3cc21]

TMM-1

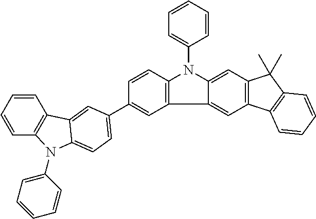 [SMILES: CC1(C)c2ccccc2-c2cc3c4cc(-c5ccc6c(c5)c5ccccc5n6-c5ccccc5)ccc4n(-c4ccccc4)c3cc21]

TMM-2

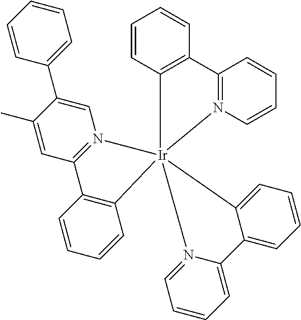

TEG

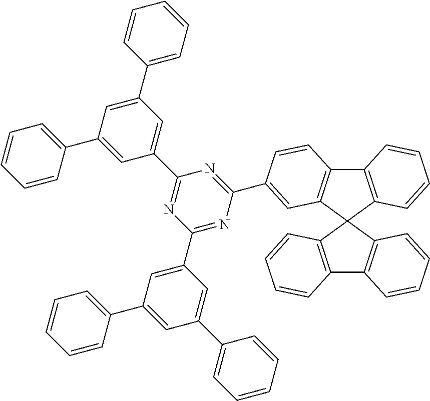 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ETM

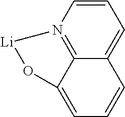

LiQ

All materials are applied by thermal vapor deposition in a vacuum chamber. In this case, the emission layer consists of at least one matrix material (host material) and an emitting dopant (emitter) which is added to the matrix material(s) in a particular proportion by volume by co-evaporation. Details given in such a form as H:SEB (95%:5%) mean here that the material H is present in the layer in a proportion by volume of 95% and SEB in a proportion of 5%.

In an analogous manner, the electron transport layer and the hole injection layer also consist of a mixture of two materials.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @ 10 mA/cm² refers to the external quantum efficiency which is attained at 10 mA/cm². The parameter U @ 10 mA/cm² refers to the operating voltage at 10 mA/cm². The lifetime LT is defined as the time after which the luminance drops from the starting luminance to a certain proportion in the course of operation with constant current density. An LT80 figure means here that the lifetime reported corresponds to the time after which the luminance has dropped to 80% of its starting value. The figure @60 or 40 mA/cm² means here that the lifetime in question is measured at 60 or 40 mA/cm².

It is found that OLEDs containing the compounds of the invention have very good performance data; in particular, they show distinctly improved lifetimes over comparable OLEDs from the prior art. Moreover, voltages and efficiencies are at a very high level.

Device Examples: Production of the OLEDs

OLEDs of the invention and OLEDs according to the prior art are produced by a general method according to WO 04/058911, which is adapted to the circumstances described here (variation in layer thickness, materials).

In the examples which follow (see tables 2 to 4), the data of various OLEDs are presented. Substrates used are glass substrates coated with structured ITO (indium tin oxide) of thickness 50 nm. The OLEDs basically have the following layer structure:
substrate,
ITO (50 nm),
buffer (20 nm),
hole injection layer (HTL 95%, HIL 5%) (20 nm),
hole transport layer (HTL) (195 nm),
emission layer (EML) (20 nm),
electron transport layer (ETL 50%, EIL 50%) (30 nm),
electron injection layer (EIL) (1 nm),
cathode.

The cathode is formed by an aluminum layer of thickness 100 nm. The buffer applied by spin-coating is a 20 nm-thick layer of Clevios P VP Al 4083 (sourced from Heraeus Clevios GmbH, Leverkusen). All the rest of the materials are applied by thermal vapor deposition in a vacuum chamber. The structure of the OLEDs is shown in table 2. The materials used are shown in table 4.

The emission layer always consists of at least one matrix material (host=H) and an emitting dopant (=D) which is added to the matrix material in a particular proportion by volume by co-evaporation. Details given in such a form as H:D (97%:3%) mean here that the material H is present in the layer in a proportion by volume of 97% and D in a proportion of 3%.

Analogously, the electron transport layer may also consist of a mixture of two materials.

The OLEDs are characterized in a standard manner. For this purpose, the electroluminescence spectra are recorded, and the current efficiency (measured in cd/A) and the external quantum efficiency (EQE, measured in percent) are calculated as a function of luminance, assuming Lambertian emission characteristics, from current-voltage-luminance characteristics (IUL characteristics), and finally the lifetime of the components is determined. The electroluminescence spectra are recorded at a luminance of 1000 cd/m², and the CIE 1931 x and y color coordinates are calculated therefrom. The parameter EQE @ 1000 cd/m² refers to the external quantum efficiency at an operating luminance of 1000 cd/m². The lifetime is the time LD95 @ 1000 cd/m² that elapses before the starting brightness of 1000 cd/m² has fallen by 5%. The data obtained for the various OLEDs are collated in table 3.

Use of Materials of the Invention as Electron Transport Material in OLEDs

The materials of the invention are suitable for use as electron transport material (ETL) in OLEDs and result in excellent performance data; see examples E1 to E4.

TABLE 2
Structure of the OLEDs

| | EML | ETL |
|---|---|---|
| Ex. | Thickness/nm | Thickness/nm |
| E1 | H(97%):D(3%) 20 nm | EG1(50%):EIL(50%) |
| E2 | H(97%):D(3%) 20 nm | EG2(50%):EIL(50%) |
| E3 | H(97%):D(3%) 20 nm | EG3(50%):EIL(50%) |
| E4 | H(97%):D(3%) 20 nm | EG4(50%):EIL(50%) |

TABLE 3
Data of the OLEDs

| | EQE | LD95 | | |
|---|---|---|---|---|
| | @ | @ 1000 | | |
| | 1000 cd/m² | cd/m² | CIE | |
| Ex. | % | [h] | x | y |
| E1 | 5.3 | 120 | 0.13 | 0.14 |
| E2 | 5.9 | 90 | 0.13 | 0.14 |
| E3 | 5.5 | 110 | 0.13 | 0.14 |
| E4 | 5.2 | 100 | 0.13 | 0.14 |

TABLE 4
Structures of the materials used

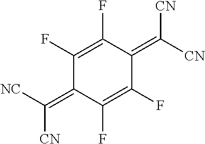 [SMILES: N#CC(C#N)=c1c(F)c(F)c(=C(C#N)C#N)c(F)c1F]

HIL

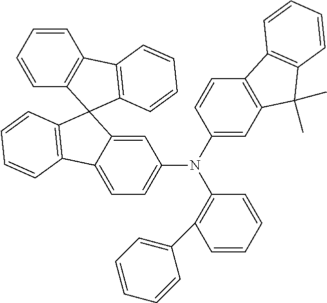 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

HTL

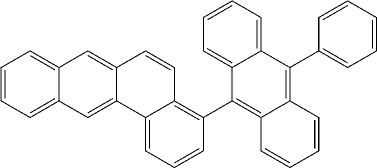 [SMILES: c1ccc(-c2c3ccccc3c(-c3cccc4c3ccc3cc5ccccc5cc34)c3ccccc23)cc1]

H

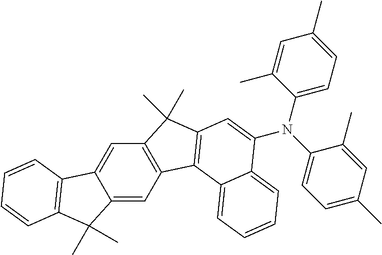 [SMILES: Cc1ccc(N(c2ccc(C)cc2C)c2cc3c(c4ccccc24)-c2cc4c(cc2C3(C)C)-c2ccccc2C4(C)C)c(C)c1]

D

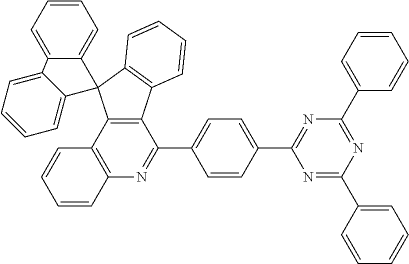 [SMILES: c1ccc(-c2nc(-c3ccccc3)nc(-c3ccc(-c4nc5ccccc5c5c4-c4ccccc4C54c5ccccc5-c5ccccc54)cc3)n2)cc1]

EG1

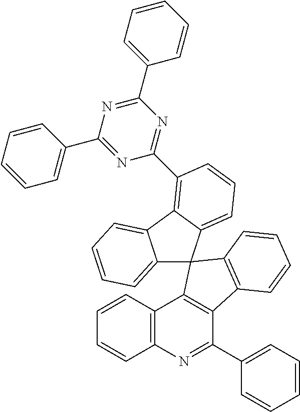 [SMILES: c1ccc(-c2nc(-c3ccccc3)nc(-c3cccc4c3-c3ccccc3C43c4ccccc4-c4c(-c5ccccc5)nc5ccccc5c43)n2)cc1]

EG2

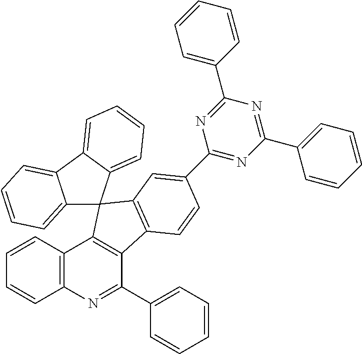 [SMILES: c1ccc(-c2nc(-c3ccccc3)nc(-c3ccc4c(c3)C3(c5ccccc5-c5ccccc53)c3c-4c(-c4ccccc4)nc4ccccc34)n2)cc1]

EG3

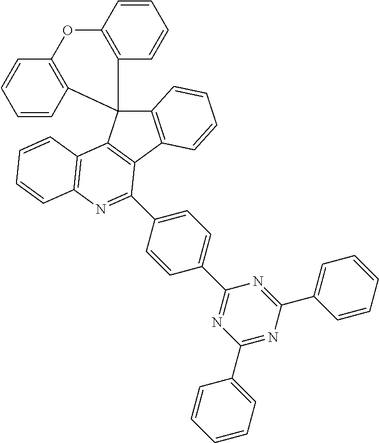 [SMILES: c1ccc(-c2nc(-c3ccccc3)nc(-c3ccc(-c4nc5ccccc5c5c4-c4ccccc4C54c5ccccc5Oc5ccccc54)cc3)n2)cc1]

EG4

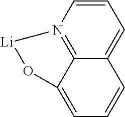

EIL

1.-19. (canceled)

20. A compound having at least one structure of formula (Ia) and/or (Ib): 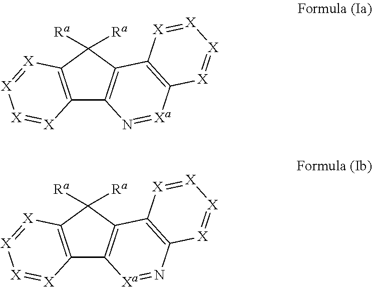
where the symbols used are as follows:
X is the same or different at each instance and is N or CR;
X^{a} is the same or different at each instance and is N or CR^{a};
R^{a} is the same or different at each instance and is H, D, OH, F, Cl, Br, I, CN, NO₂, N(Ar^{a})₂, N(R)₂, C(═O)Ar^{a}, C(═O)R², P(═O)(Ar^{a})₂, P(Ar^{a})₂, B(Ar^{a})₂, B(OR)₂, Si(Ar^{a})₃, Si(R)₃, Ge(R)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R radicals, where one or more nonadjacent CH₂ groups may be replaced by —RC═CR—, —C≡C—, Si(R)₂, Ge(R)₂, Sn(R)₂, C═O, C═S, —O—, —Se—, —S—, C═Se, —C(═O)O—, —C(═O)NR—, C═NR, NR, P(═O)(R), SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R radicals, or an aryloxy or heteroaryloxy group which has 5 to 60 aromatic ring atoms and may be substituted by one or more R radicals, or an aralkyl or heteroaralkyl group which has 5 to 60 aromatic ring atoms and may be substituted by one or more R radicals, or a combination of these systems; at the same time, two or more, adjacent R^{a} radicals may form a ring system with one another or with an R radical;
Ar^{a} is the same or different at each instance and is an aromatic or heteroaromatic ring system which has 5 to 30 aromatic ring atoms and may be substituted by one or more, R radicals; at the same time, it is possible for two Ar^{a} radicals bonded to the same silicon atom, nitrogen atom, phosphorus atom or boron atom also to be joined together via a bridge by a single bond or a bridge selected from B(R), C(R)₂, Si(R)₂, Ge(R)₂, C═O, C═NR, C═C(R)₂, O, S, Se, S═O, SO₂, N(R), P(R) and P(═O)R;
R is the same or different at each instance and is H, D, OH, F, Cl, Br, I, CN, NO₂, N(Ar)₂, N(R¹)₂, C(═O)Ar, C(═O)R′, P(═O)(Ar)₂, P(Ar)₂, B(Ar)₂, B(OR¹)₂, Si(Ar)₃, Si(R¹)₃, Ge(R¹)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R¹C═CR¹—, —C≡C—, Si(R¹)₂, Ge(R¹)₂, Sn(R¹)₂, C═O, C═S, C═Se, —C(═O)O—, —C(═O)NR¹—, C═NR¹, NR′, P(═O)(R′), —O—, —S—, —Se—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R¹ radicals, or an aryloxy or heteroaryloxy group which has 5 to 60 aromatic ring atoms and may be substituted by one or more R¹ radicals, or an aralkyl or heteroaralkyl group which has 5 to 60 aromatic ring atoms and may be substituted by one or more R¹ radicals, or a combination of these systems; at the same time, two or more R radicals together may form a ring system;
Ar is the same or different at each instance and is an aromatic or heteroaromatic ring system which has 5 to 30 aromatic ring atoms and may be substituted by one or more, R¹ radicals; at the same time, it is possible for two Ar radicals bonded to the same silicon atom, nitrogen atom, phosphorus atom or boron atom also to be joined together via a bridge by a single bond or a bridge selected from B(R′), C(R¹)₂, Si(R¹)₂, Ge(R¹)₂, C═O, C═NR¹, C═C(R¹)₂, O, S, Se, S═O, SO₂, N(R¹), P(R¹) and P(═O)R¹;
R¹ is the same or different at each instance and is H, D, OH, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R²C═CR²—, Si(R²)₂, Ge(R²)₂, Sn(R²)₂, C═O, C═S, C═Se, C═NR², —C(═O)O—, —C(═O)NR²—, NR², P(═O)(R²), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more R¹ radicals together may form a ring system;
Ar¹ is the same or different at each instance and is an aromatic or heteroaromatic ring system which has 5 to 30 aromatic ring atoms and may be substituted by one or more, R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, Ge(R²)₂, C═O, C═NR², C═C(R²)₂, O, S, Se, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, Si(R³)₂, Ge(R³)₂, Sn(R³)₂, C═O, C═S, C═NR³, —C(═O)O—, —C(═O)NR³—, NR³, P(═O)(R³), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more substituents R² together may form a with one another;
R³ is the same or different at each instance and is selected from the group consisting of H, D, F, CN, an aliphatic hydrocarbyl radical having 1 to 20 carbon atoms, and an aromatic or heteroaromatic ring system having 5 to 30 aromatic ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents to form a ring system with one another.

21. A compound as claimed in claim 20, comprising at least one structure of formula (II-1), (II-2), (II-3), (II-4), (II-5), (II-6), (II-7), (II-8), (II-9), (II-10), (II-11), (II-12), (II-13), (II-14), (II-15) or (II-16): 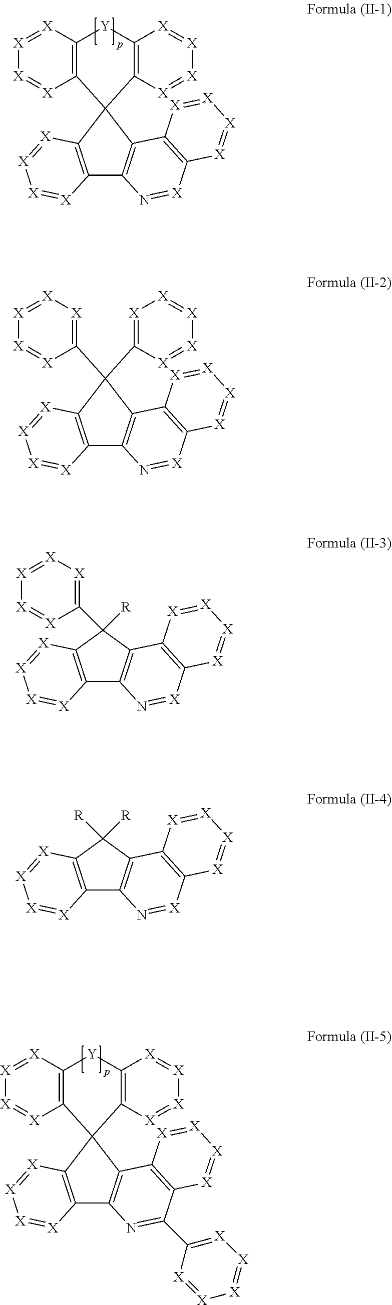 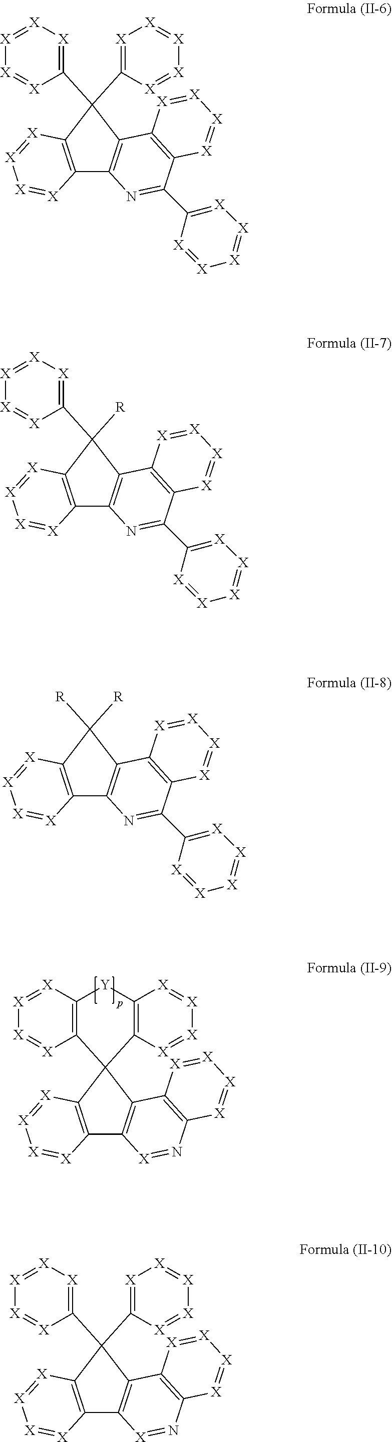 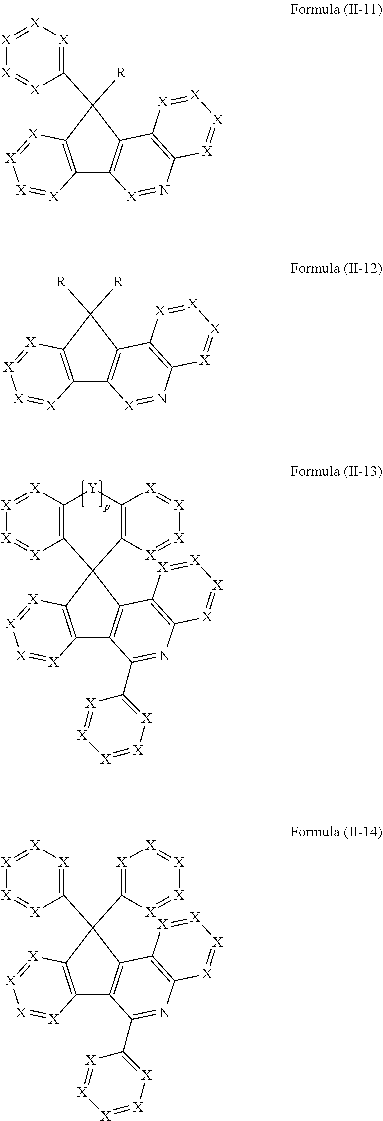 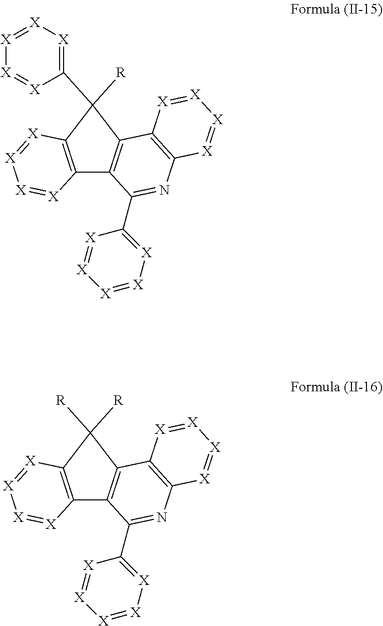
where the symbols R and X have the definition given in claim 20, p is 0 or 1 and Y represents B(R), C(R)₂, Si(R)₂, C═O, C═NR, C═C(R)₂, O, S, Se, S═O, SO₂, N(R), P(R) and P(═O)R, where, when p=0, there is a bond between the aromatic or heteroaromatic rings shown.

22. A compound as claimed in claim 20, comprising at least one structure of the formula (III-1), (III-2), (III-3), (III-4), (III-5), (III-6), (III-7), (III-8), (III-9), (III-10), (III-11), (III-12), (III-13), (III-14), (III-15) or (III-16): 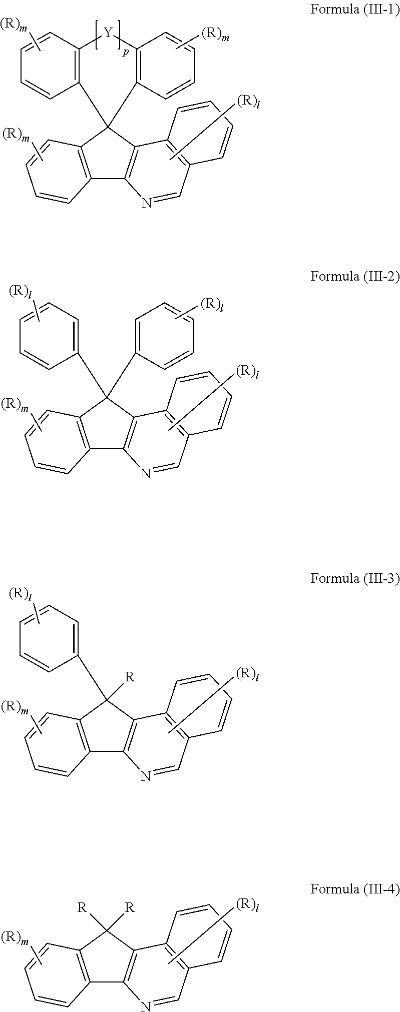 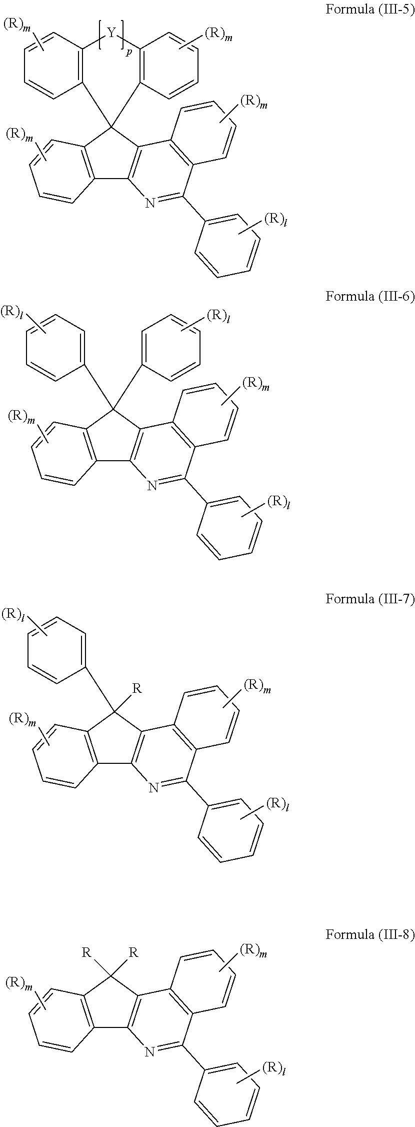 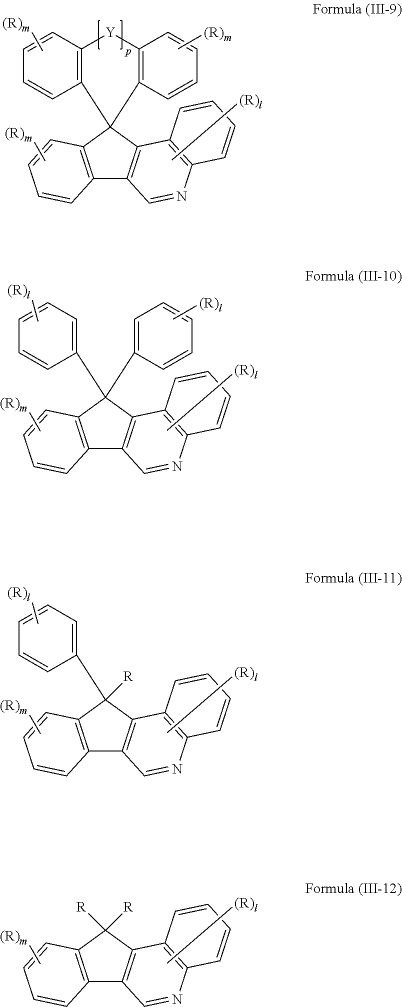 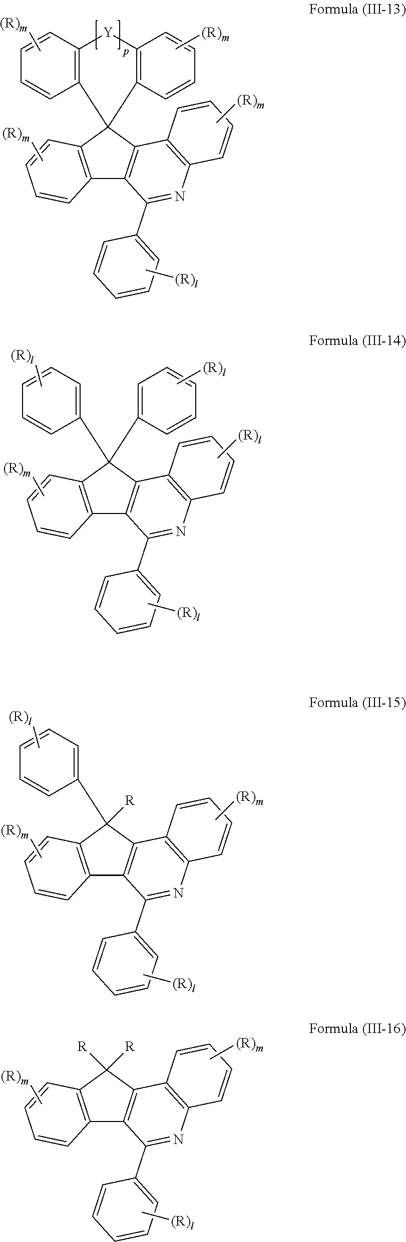
where the symbol R has the definition given in claim 20, the symbols p is 0 or 1, and Y represents B(R), C(R)₂, Si(R)₂, C═O, C═NR, C═C(R)₂, O, S, Se, S═O, SO₂, N(R), P(R) and P(═O)R, where, when p=0, there is a bond between the aromatic or heteroaromatic rings shown, 1 is 1, 2, 3, 4 or 5, and m is 0, 1, 2, 3 or 4.

23. A compound as claimed in claim 20, wherein the compound comprises a hole transport group.

24. A compound as claimed in claim 20, that the compound comprises an electron transport group-comprising radical.

25. A compound as claimed in claim 20, wherein at least one R^{a} or R group in the formulae (Ia), (Ib), (II-1), (II-2), (II-3), (II-4), (II-5), (II-6), (II-7), (II-8), (II-9), (II-10), (II-11), (II-12), (II-13), (II-14), (II-15), (II-16), (III-1), (III-2), (III-3), (III-4), (III-5), (III-6), (III-7), (III-8), (III-9), (III-10), (III-11), (III-12), (III-13), (III-14), (III-15) or (III-16) is a group that can be represented by the formula L¹-Z in which L¹ represents a bond or an aromatic or heteroaromatic ring system which has 5 to 40, aromatic ring atoms and may be substituted by one or more R¹ radicals, Z represents R¹, Ar or a group of the formula Z^{a} or Z^{b} in which the symbols Ar and R¹ have the definition given in claim 20 and Z^{a} or Z^{b} are 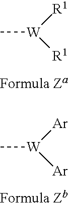
in which W is the same or different at each instance and is an aromatic or heteroaromatic ring system which has 5 to 30 aromatic ring atoms and may be substituted by one or more R¹ radicals, a nitrogen atom, a boron atom, a phosphorus atom or a phosphane oxide group, the dotted bond marks the position of attachment and the symbols Ar and R¹ have the definition given in claim 20.

26. A compound as claimed in claim 25, comprising at least one structure of the formula (IV-1), (IV-2), (IV-3), (IV-4), (IV-5), (IV-6), (IV-7), (IV-8), (IV-9), (IV-10), (IV-11), (IV-12), (IV-13), (IV-14), (IV-15), (IV-16), (IV-17), (IV-18), (IV-19), (IV-20), (IV-21), (IV-22), (IV-23) or (IV-24): 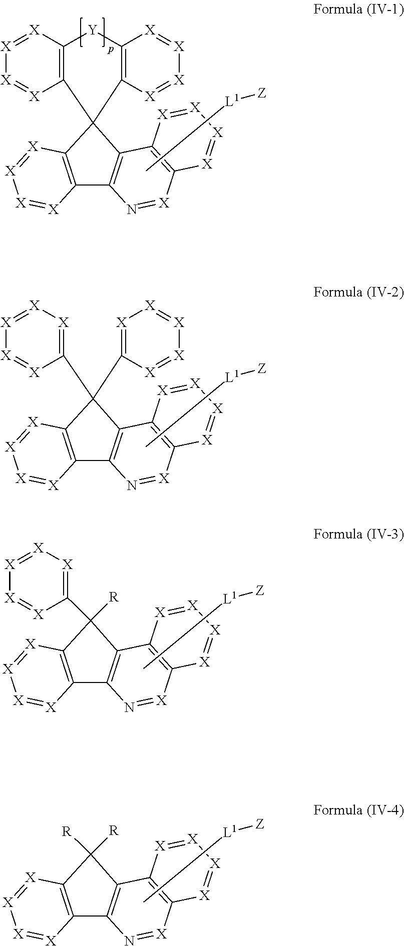 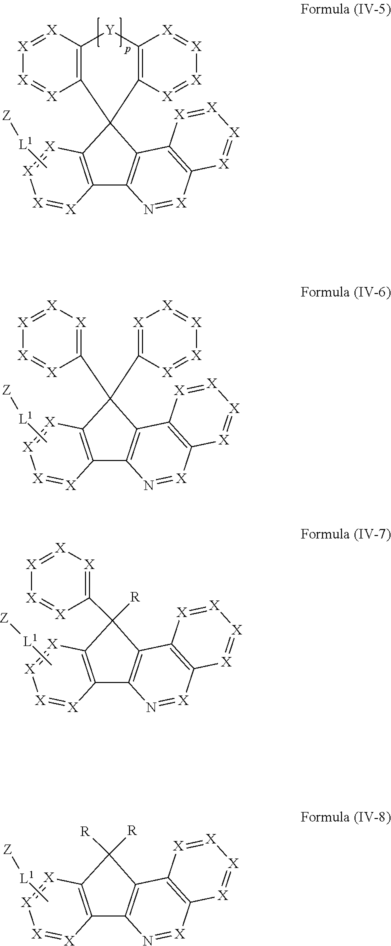 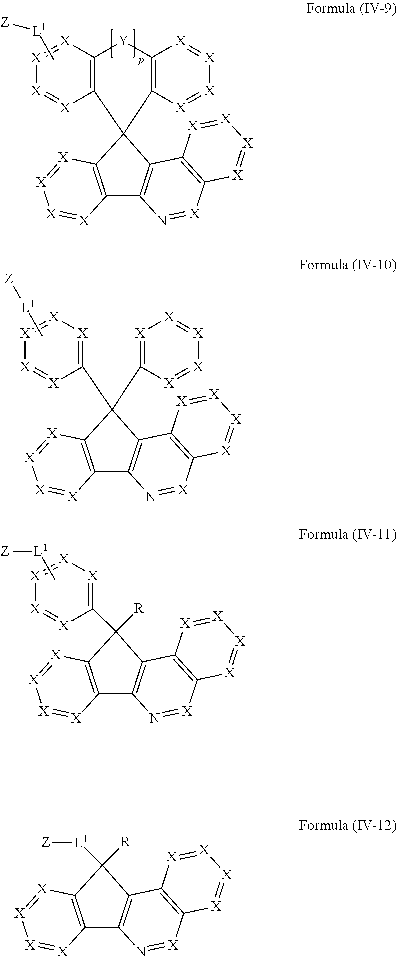 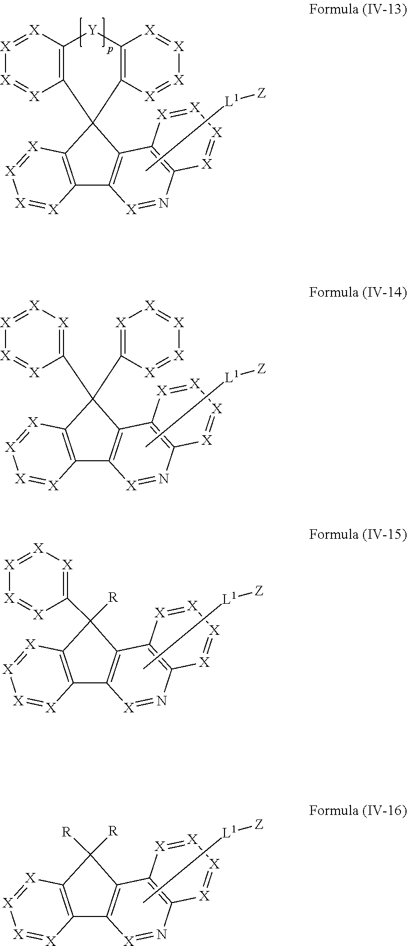 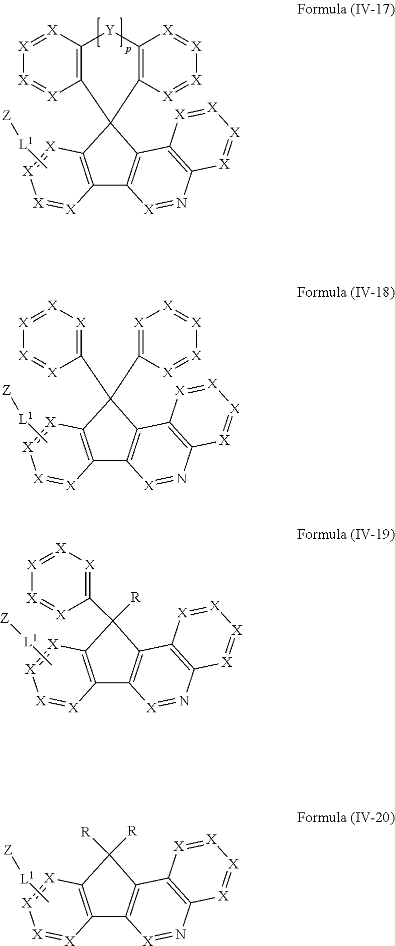 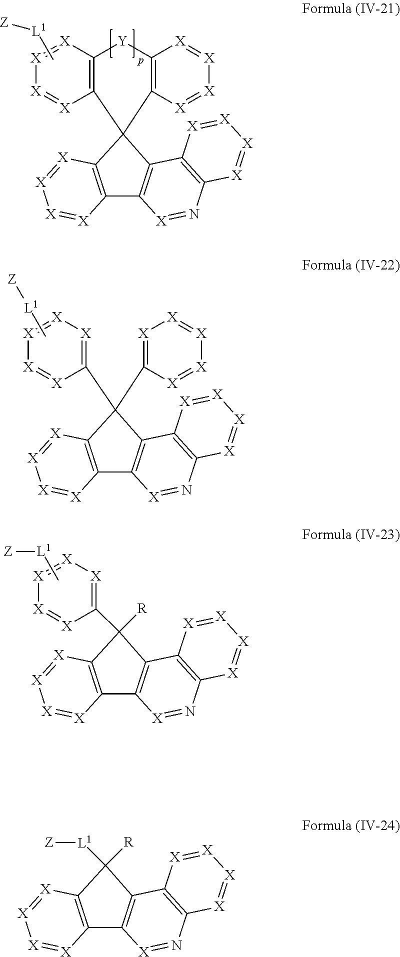
where the symbol X represents N, CR or C if the -L¹-Z group is bonded to X,
R is the same or different at each instance and is H, D, OH, F, Cl, Br, I, CN, NO₂, N(Ar)₂, N(R¹)₂, C(═O)Ar, C(═O)R¹, P(═O)(Ar)₂, P(Ar)₂, B(Ar)₂, B(OR¹)₂, Si(Ar)₃, Si(R¹)₃, Ge(R¹)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R¹C═CR¹—, —C≡C—, Si(R¹)₂, Ge(R¹)₂, Sn(R¹)₂, C═O, C═S, C═Se, —C(═O)O—, —C(═O)NR¹—, C═NR¹, NR¹, P(═O)(R¹), —O—, —S—, —Se—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R¹ radicals, or an aryloxy or heteroaryloxy group which has 5 to 60 aromatic ring atoms and may be substituted by one or more R¹ radicals, or an aralkyl or heteroaralkyl group which has 5 to 60 aromatic ring atoms and may be substituted by one or more R¹ radicals, or a combination of these systems; at the same time, two or more R radicals together may form a ring system;
Ar is the same or different at each instance and is an aromatic or heteroaromatic ring system which has 5 to 30 aromatic ring atoms and may be substituted by one or more, R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, Se, S═O, SO₂, N(R¹), P(R¹) and P(═O)R¹;
R¹ is the same or different at each instance and is H, D, OH, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R²C═CR²—, Si(R²)₂, Ge(R²)₂, Sn(R²)₂, C═O, C═S, C═Se, C═NR², —C(═O)O—, —C(═O)NR²—, NR², P(═O)(R²), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more R¹ radicals together may form a ring system;
Ar¹ is the same or different at each instance and is an aromatic or heteroaromatic ring system which has 5 to 30 aromatic ring atoms and may be substituted by one or more, R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, Ge(R²)₂, C═O, C═NR², C═C(R²)₂, O, S, Se, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, Si(R³)₂, Ge(R³)₂, Sn(R³)₂, C═O, C═S, C═NR³, —C(═O)O—, —C(═O)NR³—, NR³, P(═O)(R³), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more substituents R² together may form a with one another;
R³ is the same or different at each instance and is selected from the group consisting of H, D, F, CN, an aliphatic hydrocarbyl radical having 1 to 20 carbon atoms, and an aromatic or heteroaromatic ring system having 5 to 30 aromatic ring atoms in which one or more hydrogen atoms may be replaced by D, F, Cl, Br, I or CN and which may be substituted by one or more alkyl groups each having 1 to 4 carbon atoms; at the same time, it is possible for two or more R³ substituents to form a ring system with one another.
and the -L¹-Z group has the definition given in claim 25.

27. A compound as claimed in claim 25, comprising at least one structure of the formula (V-1), (V-2), (V-3), (V-4), (V-5), (V-6), (V-7), (V-8), (V-9), (V-10), (V-11), (V-12), (V-13), (V-14), (V-15), (V-16), (V-17), (V-18), (V-19), (V-20), (V-21), (V-22), (V-23), (V-24), (V-25), (V-26), (V-27), (V-28), (V-29), (V-30), (V-31) or (V-32): 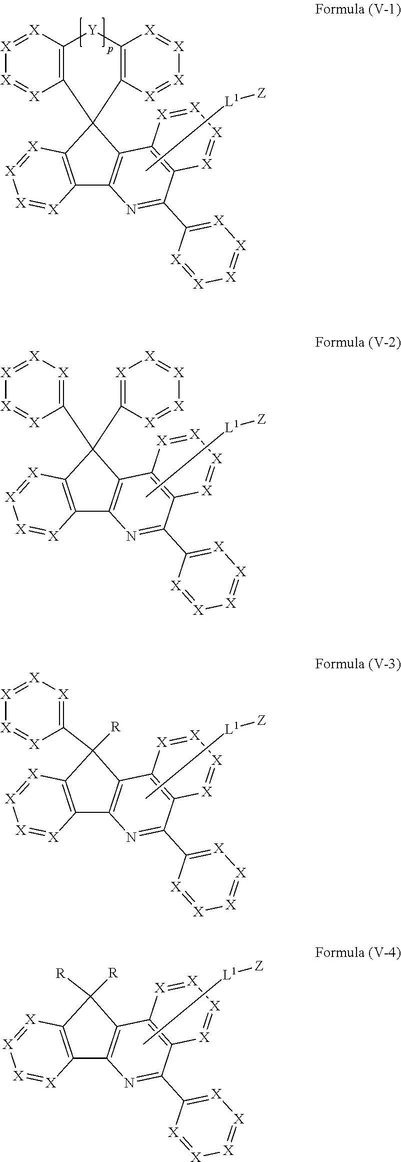 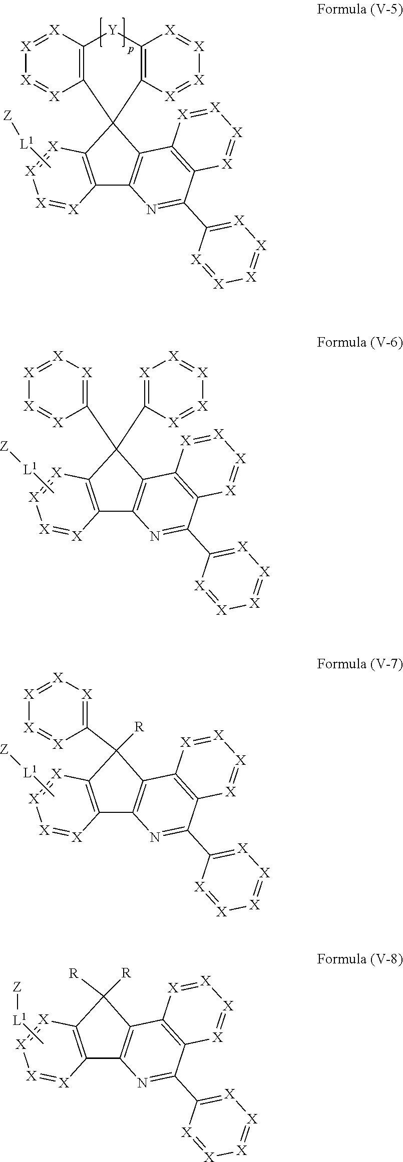 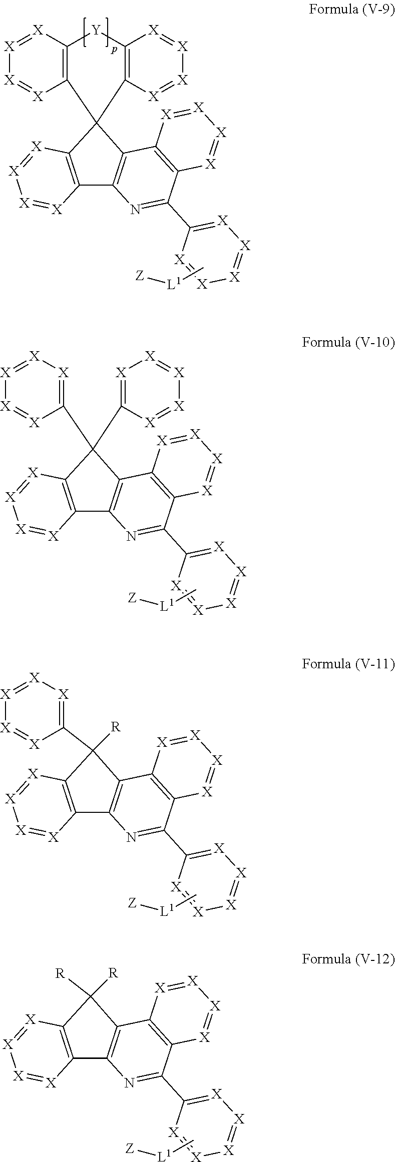 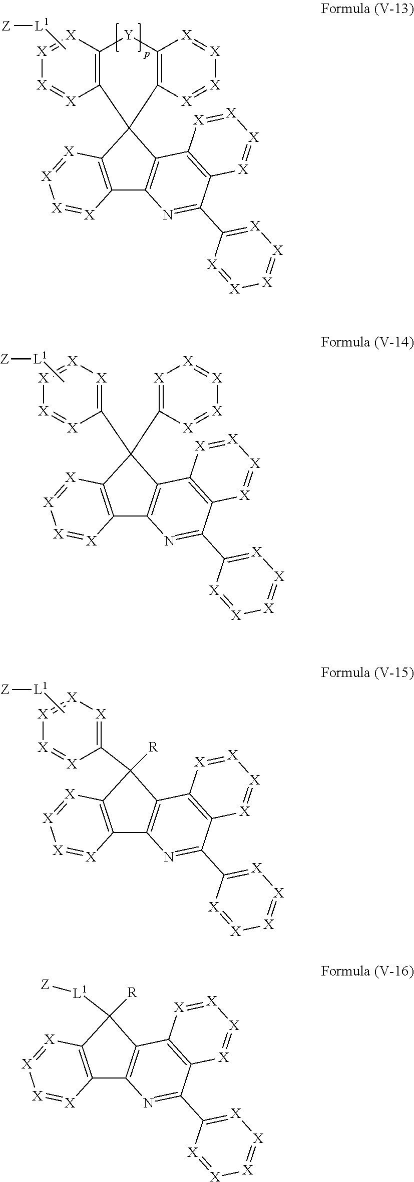 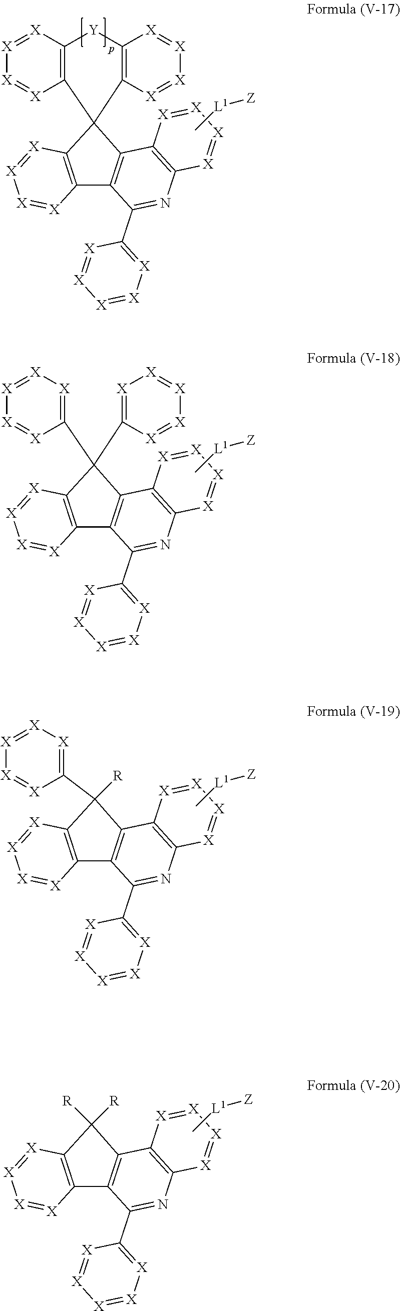 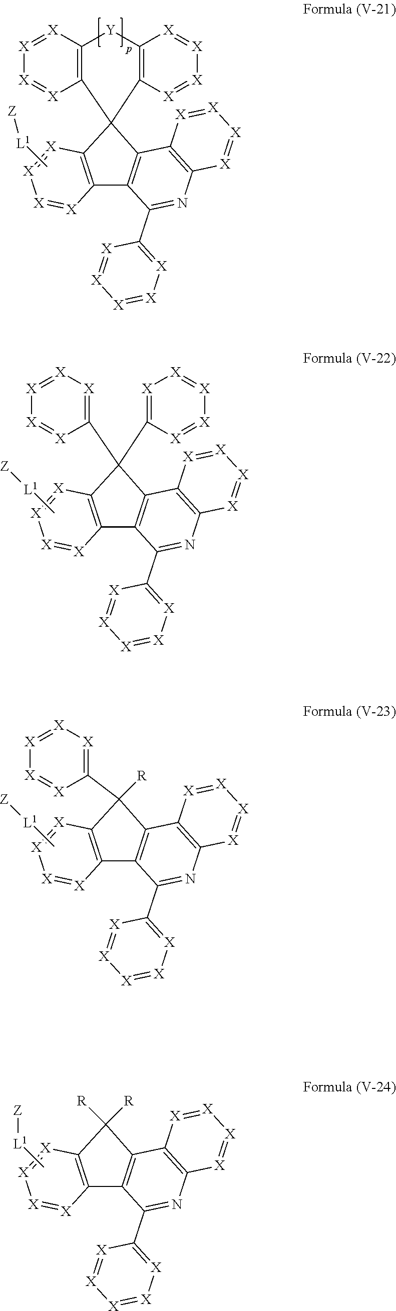 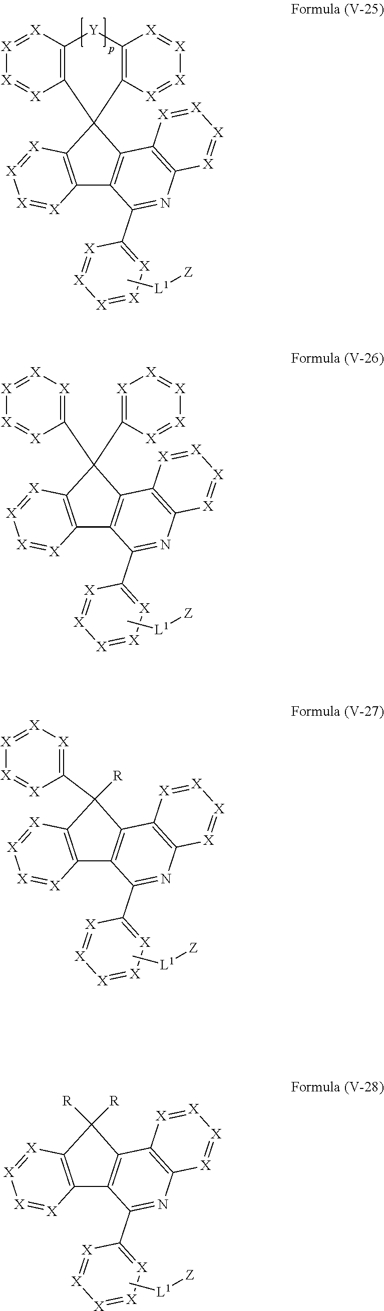 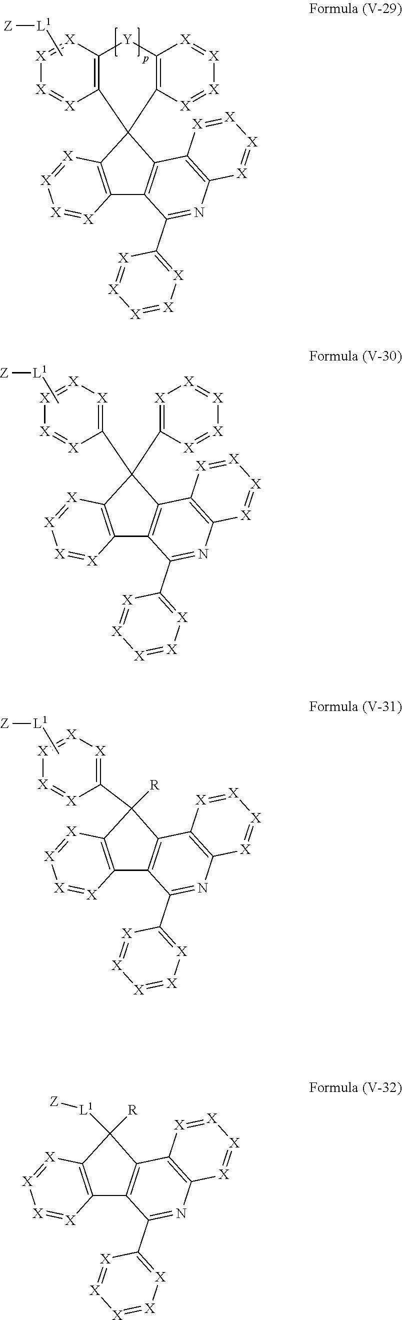
where the symbol X represents N, CR or C if the -L¹-Z group is bonded to X,
R is the same or different at each instance and is H, D, OH, F, Cl, Br, I, CN, NO₂, N(Ar)₂, N(R¹)₂, C(═O)Ar, C(═O)R¹, P(═O)(Ar)₂, P(Ar)₂, B(Ar)₂, B(OR¹)₂, Si(Ar)₃, Si(R¹)₃, Ge(R¹)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R¹C═CR¹—, —C≡C—, Si(R¹)₂, Ge(R¹)₂, Sn(R¹)₂, C═O, C═S, C═Se, —C(═O)O—, —C(═O)NR¹—, C═NR¹, NR¹, P(═O)(R¹), —O—, —S—, —Se—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R¹ radicals, or an aryloxy or heteroaryloxy group which has 5 to 60 aromatic ring atoms and may be substituted by one or more R¹ radicals, or an aralkyl or heteroaralkyl group which has 5 to 60 aromatic ring atoms and may be substituted by one or more R¹ radicals, or a combination of these systems; at the same time, two or more R radicals together may form a ring system;
Ar is the same or different at each instance and is an aromatic or heteroaromatic ring system which has 5 to 30 aromatic ring atoms and may be substituted by one or more R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, Se, S═O, SO₂, N(R¹), P(R¹) and P(═O)R¹;
R¹ is the same or different at each instance and is H, D, OH, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R²C═CR²—, Si(R²)₂, Ge(R²)₂, Sn(R²)₂, C═O, C═S, C═Se, C═NR², —C(═O)O—, —C(═O)NR²—, NR², P(═O)(R²), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more R¹ radicals together may form a ring system;
Ar¹ is the same or different at each instance and is an aromatic or heteroaromatic ring system which has 5 to 30 aromatic ring atoms and may be substituted by one or more, R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, Ge(R²)₂, C═O, C═NR², C═C(R²)₂, O, S, Se, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, Si(R³)₂, Ge(R³)₂, Sn(R³)₂, C═O, C═S, C═NR³, —C(═O)O—, —C(═O)NR³—, NR³, P(═O)(R³), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more substituents R² together may form a with one another;
R³ is the same or different at each instance and is selected from the group consisting of H, D, F, CN, an aliphatic hydrocarbyl radical having 1 to 20 carbon atoms, and an aromatic or heteroaromatic ring system having 5 to 30 aromatic ring atoms in which one or more hydrogen atoms may be replaced by D, F, Cl, Br, I or CN and which may be substituted by one or more alkyl groups each having 1 to 4 carbon atoms; at the same time, it is possible for two or more R³ substituents to form a ring system with one another,
and
p is 0 or 1 and Y represents B(R), C(R)₂, Si(R)₂, C═O, C═NR, C═C(R)₂, O, S, Se, S═O, SO₂, N(R), P(R) and P(═O)R, where, when p=0, there is a bond between the aromatic or heteroaromatic rings shown.

28. A compound as claimed in claim 27, comprising at least one structure of the formula (VI-1), (VI-2), (VI-3), (VI-4), (VI-5), (VI-6), (VI-7) or (VI-8): 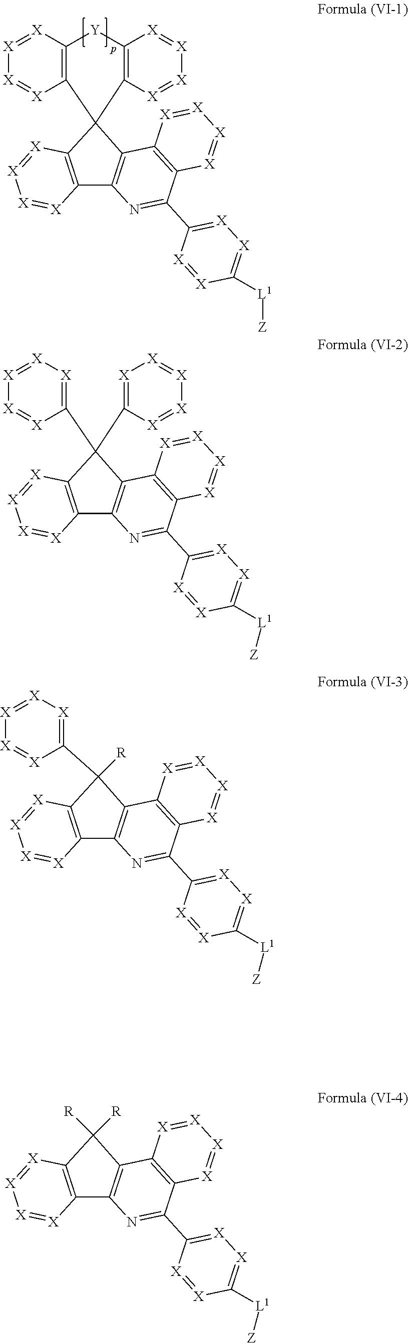 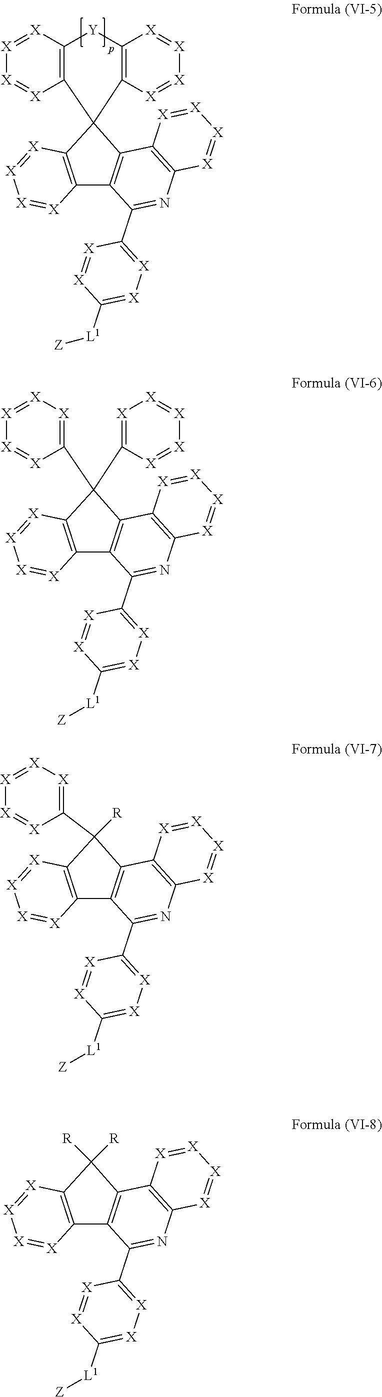
where the symbol X represents N, CR or C if the -L¹-Z group is bonded to X.

29. A compound as claimed in claim 25, comprising at least one structure of the formula (VII-1), (VII-2), (VII-3), (VII-4), (VII-5), (VII-6), (VII-7) or (VII-8): 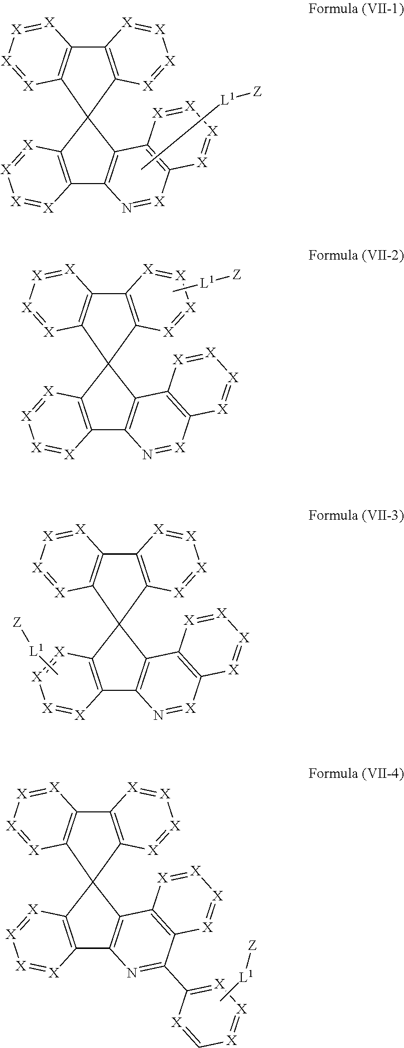 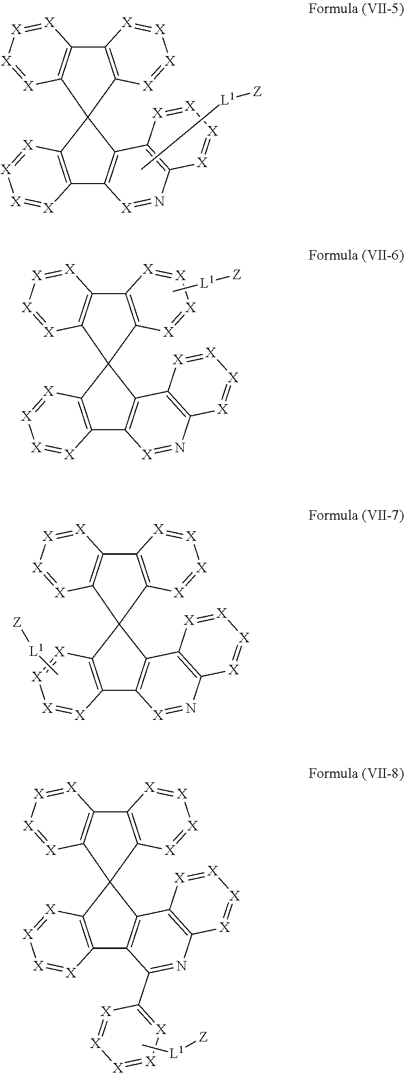
where the symbol X represents N, CR or C if the -L¹-Z group is bonded to X,
R is the same or different at each instance and is H, D, OH, F, Cl, Br, I, CN, NO₂, N(Ar)₂, N(R¹)₂, C(═O)Ar, C(═O)R¹, P(═O)(Ar)₂, P(Ar)₂, B(Ar)₂, B(OR¹)₂, Si(Ar)₃, Si(R¹)₃, Ge(R¹)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R¹C═CR¹—, —C≡C—, Si(R¹)₂, Ge(R¹)₂, Sn(R¹)₂, C═O, C═S, C═Se, —C(═O)O—, —C(═O)NR¹—, C═NR¹, NR¹, P(═O)(R¹), —O—, —S—, —Se—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R¹ radicals, or an aryloxy or heteroaryloxy group which has 5 to 60 aromatic ring atoms and may be substituted by one or more R¹ radicals, or an aralkyl or heteroaralkyl group which has 5 to 60 aromatic ring atoms and may be substituted by one or more R¹ radicals, or a combination of these systems; at the same time, two or more R radicals together may form a ring system;
Ar is the same or different at each instance and is an aromatic or heteroaromatic ring system which has 5 to 30 aromatic ring atoms and may be substituted by one or more R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, Se, S═O, SO₂, N(R¹), P(R¹) and P(═O)R¹;
R¹ is the same or different at each instance and is H, D, OH, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R²C═CR²—, Si(R²)₂, Ge(R²)₂, Sn(R²)₂, C═O, C═S, C═Se, C═NR², —C(═O)O—, —C(═O)NR²—, NR², P(═O)(R²), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more R¹ radicals together may form a ring system;
Ar¹ is the same or different at each instance and is an aromatic or heteroaromatic ring system which has 5 to 30 aromatic ring atoms and may be substituted by one or more R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, Ge(R²)₂, C═O, C═NR², C═C(R²)₂, O, S, Se, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, Si(R³)₂, Ge(R³)₂, Sn(R³)₂, C═O, C═S, C═NR³, —C(═O)O—, —C(═O)NR³—, NR³, P(═O)(R³), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more, adjacent substituents R² together may form a with one another;
R³ is the same or different at each instance and is selected from the group consisting of H, D, F, CN, an aliphatic hydrocarbyl radical having 1 to 20 carbon atoms, and an aromatic or heteroaromatic ring system having 5 to 30 aromatic ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents to form a ring system with one another.

30. A compound as claimed in claim 25, comprising at least one structure of the formula (VIII-9) or (VIII-10): 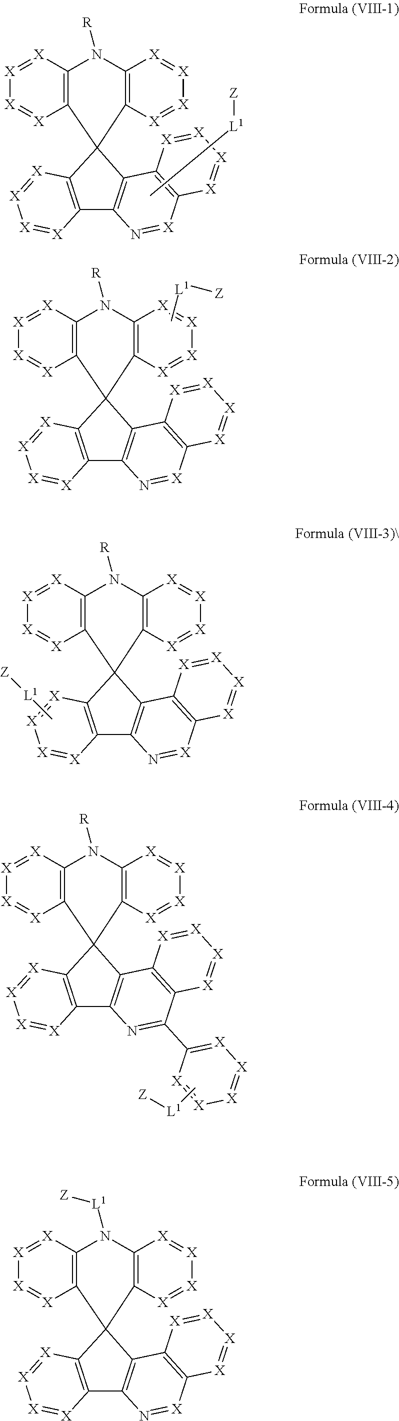 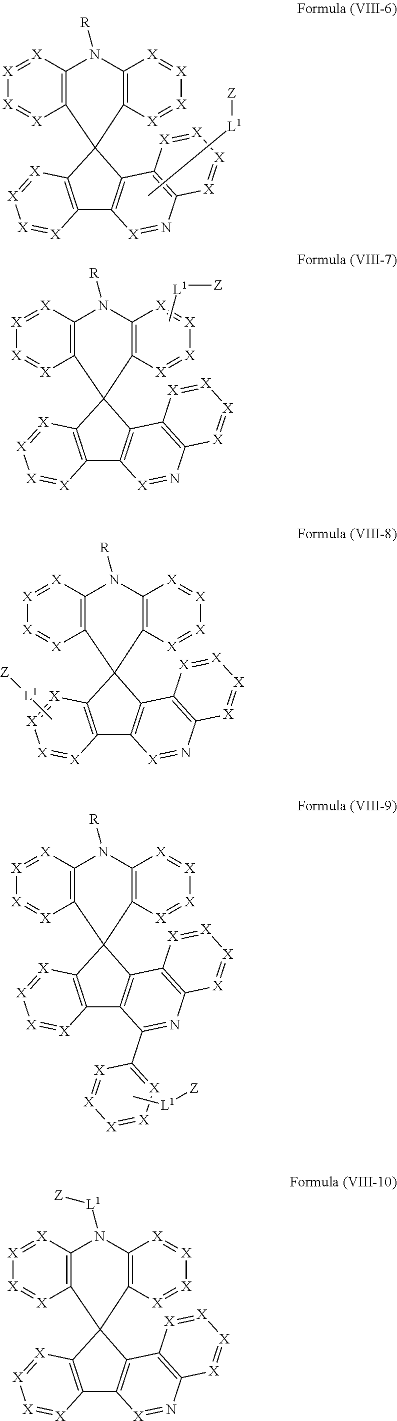
where the symbol X represents N, CR or C if the -L¹-Z group is bonded to X, and
R is the same or different at each instance and is H, D, OH, F, Cl, Br, I, CN, NO₂, N(Ar)₂, N(R¹)₂, C(═O)Ar, C(═O)R¹, P(═O)(Ar)₂, P(Ar)₂, B(Ar)₂, B(OR¹)₂, Si(Ar)₃, Si(R¹)₃, Ge(R¹)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R¹C═CR¹—, —C≡C—, Si(R¹)₂, Ge(R¹)₂, Sn(R¹)₂, C═O, C═S, C═Se, —C(═O)O—, —C(═O)NR¹—, C═NR¹, NR¹, P(═O)(R¹), —O—, —S—, —Se—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R¹ radicals, or an aryloxy or heteroaryloxy group which has 5 to 60 aromatic ring atoms and may be substituted by one or more R¹ radicals, or an aralkyl or heteroaralkyl group which has 5 to 60 aromatic ring atoms and may be substituted by one or more R¹ radicals, or a combination of these systems; at the same time, two or more R radicals together may form a ring system;
Ar is the same or different at each instance and is an aromatic or heteroaromatic ring system which has 5 to 30 aromatic ring atoms and may be substituted by one or more R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, Se, S═O, SO₂, N(R¹), P(R¹) and P(═O)R¹;
R¹ is the same or different at each instance and is H, D, OH, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R²C═CR²—, Si(R²)₂, Ge(R²)₂, Sn(R²)₂, C═O, C═S, C═Se, C═NR², —C(═O)O—, —C(═O)NR²—, NR², P(═O)(R²), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more R¹ radicals together may form a ring system;
Ar¹ is the same or different at each instance and is an aromatic or heteroaromatic ring system which has 5 to 30 aromatic ring atoms and may be substituted by one or more R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, Ge(R²)₂, C═O, C═NR², C═C(R²)₂, O, S, Se, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, Si(R³)₂, Ge(R³)₂, Sn(R³)₂, C═O, C═S, C═NR³, —C(═O)O—, —C(═O)NR³—, NR³, P(═O)(R³), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more, adjacent substituents R² together may form a with one another;
R³ is the same or different at each instance and is selected from the group consisting of H, D, F, CN, an aliphatic hydrocarbyl radical having 1 to 20 carbon atoms, and an aromatic or heteroaromatic ring system having 5 to 30 aromatic ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents to form a ring system with one another.

31. A compound as claimed in claim 25, comprising at least one structure of the formula (IX-1), (IX-2), (IX-3), (IX-4), (IX-5), (IX-6), (IX-7) or (IX-8): 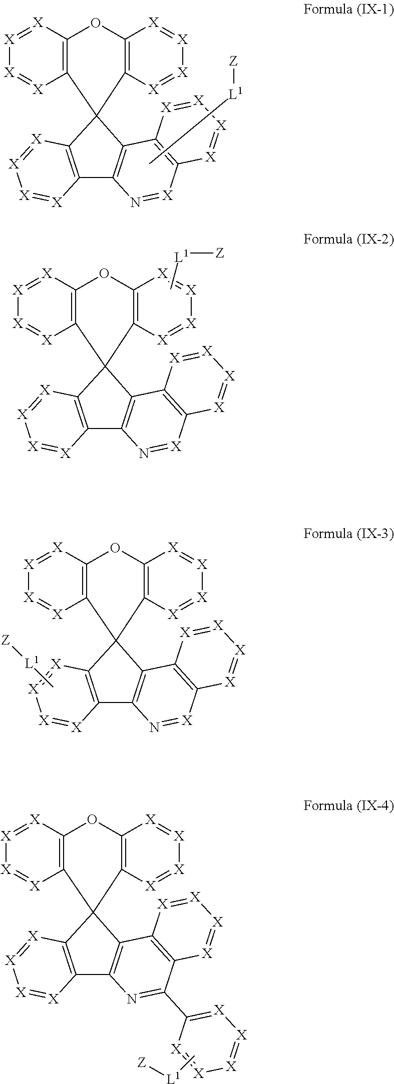 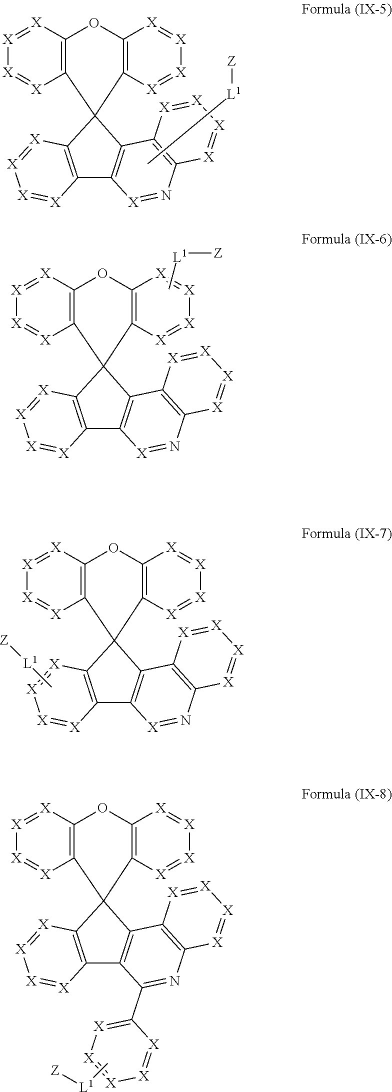
where the symbol X represents N, CR or C if the -L¹-Z group is bonded to X, and
R is the same or different at each instance and is H, D, OH, F, Cl, Br, I, CN, NO₂, N(Ar)₂, N(R¹)₂, C(═O)Ar, C(═O)R¹, P(═O)(Ar)₂, P(Ar)₂, B(Ar)₂, B(OR¹)₂, Si(Ar)₃, Si(R¹)₃, Ge(R¹)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R¹C═CR¹—, —C≡C—, Si(R¹)₂, Ge(R¹)₂, Sn(R¹)₂, C═O, C═S, C═Se, —C(═O)O—, —C(═O)NR¹—, C═NR¹, NR¹, P(═O)(R¹), —O—, —S—, —Se—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R¹ radicals, or an aryloxy or heteroaryloxy group which has 5 to 60 aromatic ring atoms and may be substituted by one or more R¹ radicals, or an aralkyl or heteroaralkyl group which has 5 to 60 aromatic ring atoms and may be substituted by one or more R¹ radicals, or a combination of these systems; at the same time, two or more R radicals together may form a ring system;
Ar is the same or different at each instance and is an aromatic or heteroaromatic ring system which has 5 to 30 aromatic ring atoms and may be substituted by one or more R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, Se, S═O, SO₂, N(R¹), P(R¹) and P(═O)R¹;
R¹ is the same or different at each instance and is H, D, OH, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R²C═CR²—, Si(R²)₂, Ge(R²)₂, Sn(R²)₂, C═O, C═S, C═Se, C═NR², —C(═O)O—, —C(═O)NR²—, NR², P(═O)(R²), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more R¹ radicals together may form a ring system;
Ar¹ is the same or different at each instance and is an aromatic or heteroaromatic ring system which has 5 to 30 aromatic ring atoms and may be substituted by one or more R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, Ge(R²)₂, C═O, C═NR², C═C(R²)₂, O, S, Se, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, Si(R³)₂, Ge(R³)₂, Sn(R³)₂, C═O, C═S, C═NR³, —C(═O)O—, —C(═O)NR³—, NR³, P(═O)(R³), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more, adjacent substituents R² together may form a with one another;
R³ is the same or different at each instance and is selected from the group consisting of H, D, F, CN, an aliphatic hydrocarbyl radical having 1 to 20 carbon atoms, and an aromatic or heteroaromatic ring system having 5 to 30 aromatic ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents to form a ring system with one another.

32. A compound as claimed in claim 25, comprising at least one structure of the formula (XII-1), (XII-2), (XII-3), (XII-4), (XII-5), (XII-6), (XII-7), (XII-8), (XII-9), (XII-10), (XII-11), (XII-12), (XII-13), (XII-14), (XII-15), (XII-16), (XII-17), (XII-18), (XII-19), (XII-20), (XII-21), (XII-22), (XII-23) or (XII-24): 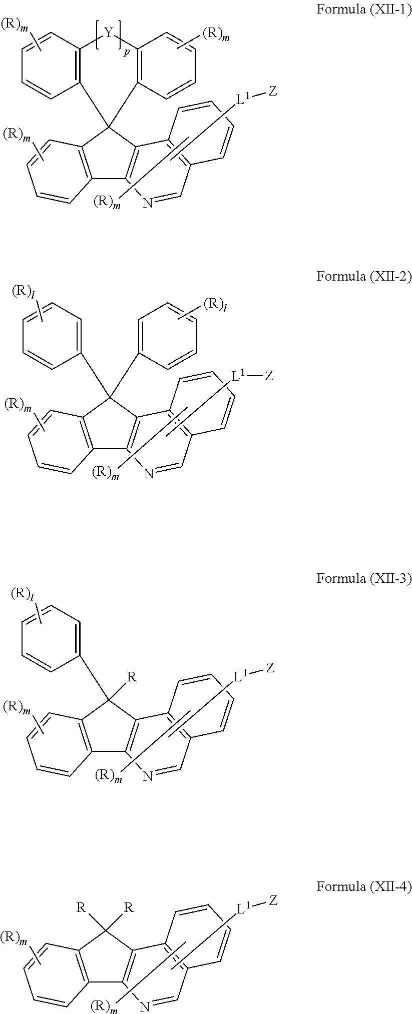 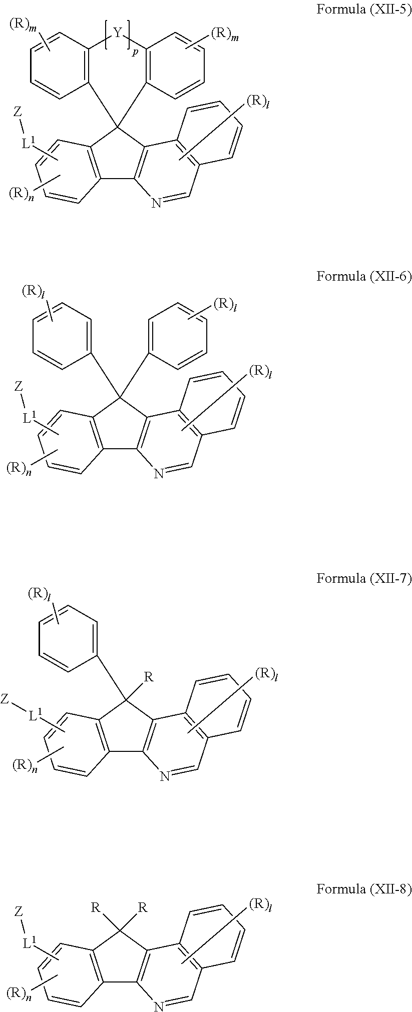 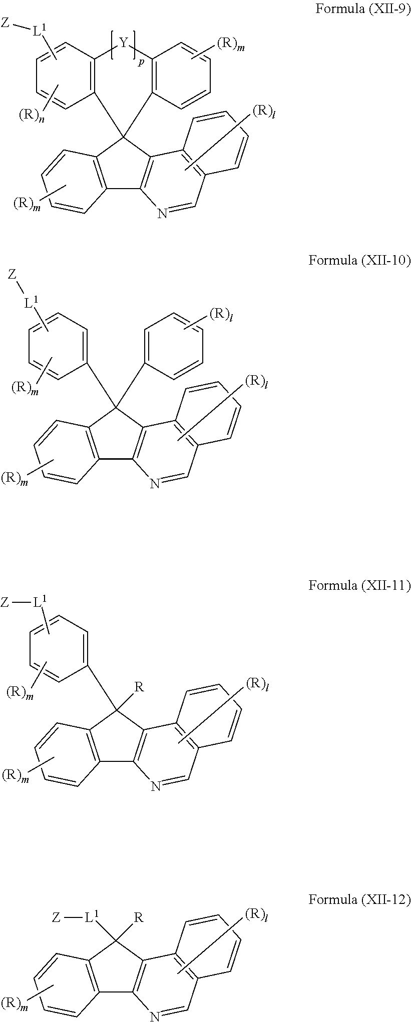 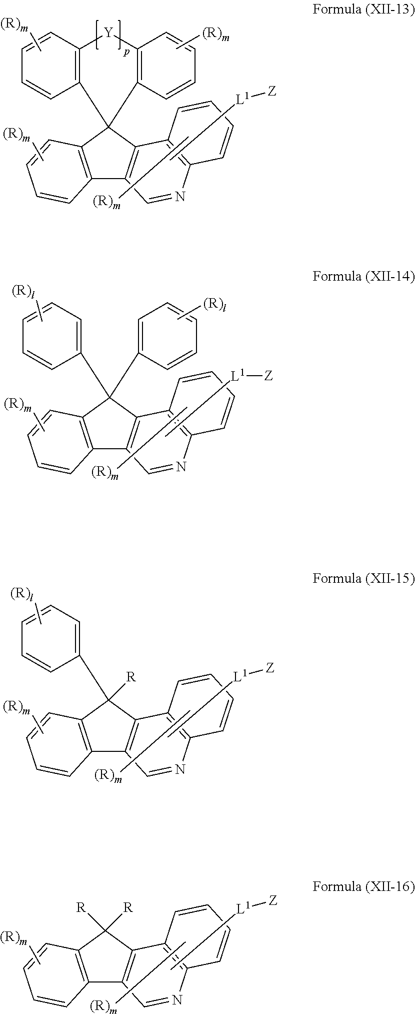 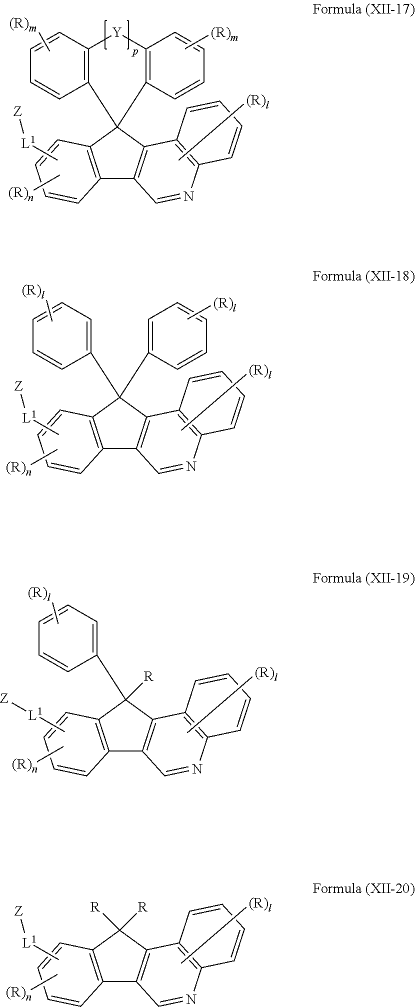 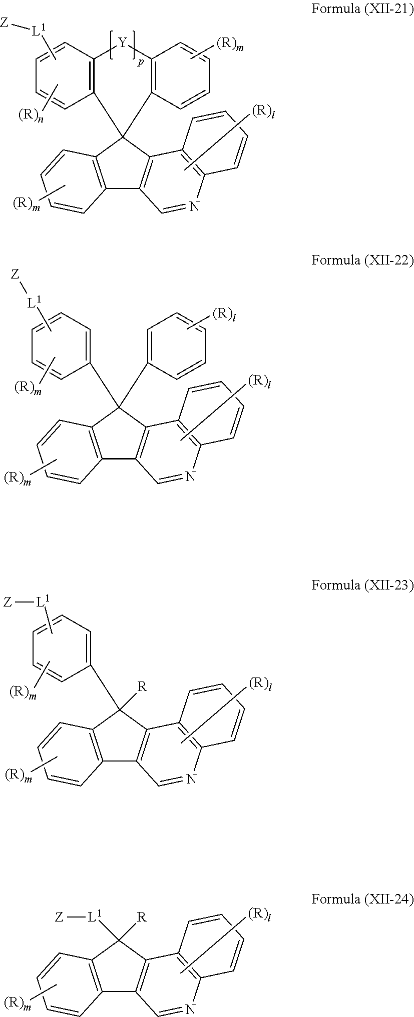
where
R is the same or different at each instance and is H, D, OH, F, Cl, Br, I, CN, NO₂, N(Ar)₂, N(R¹)₂, C(═O)Ar, C(═O)R¹, P(═O)(Ar)₂, P(Ar)₂, B(Ar)₂, B(OR¹)₂, Si(Ar)₃, Si(R¹)₃, Ge(R¹)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R¹C═CR¹—, —C≡C—, Si(R¹)₂, Ge(R¹)₂, Sn(R¹)₂, C═O, C═S, C═Se, —C(═O)O—, —C(═O)NR¹—, C═NR¹, NR¹, P(═O)(R¹), —O—, —S—, —Se—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R¹ radicals, or an aryloxy or heteroaryloxy group which has 5 to 60 aromatic ring atoms and may be substituted by one or more R¹ radicals, or an aralkyl or heteroaralkyl group which has 5 to 60 aromatic ring atoms and may be substituted by one or more R¹ radicals, or a combination of these systems; at the same time, two or more R radicals together may form a ring system;
Ar is the same or different at each instance and is an aromatic or heteroaromatic ring system which has 5 to 30 aromatic ring atoms and may be substituted by one or more R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, Se, S═O, SO₂, N(R¹), P(R¹) and P(═O)R¹;
R¹ is the same or different at each instance and is H, D, OH, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl or alkynyl group having 2 to 40 carbon atoms, each of which may be substituted by one or more R² radicals, where one or more nonadjacent CH₂ groups may be replaced by —R²C═CR²—, Si(R²)₂, Ge(R²)₂, Sn(R²)₂, C═O, C═S, C═Se, C═NR², —C(═O)O—, —C(═O)NR²—, NR², P(═O)(R²), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more R¹ radicals together may form a ring system;
Ar¹ is the same or different at each instance and is an aromatic or heteroaromatic ring system which has 5 to 30 aromatic ring atoms and may be substituted by one or more R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, Ge(R²)₂, C═O, C═NR², C═C(R²)₂, O, S, Se, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, Si(R³)₂, Ge(R³)₂, Sn(R³)₂, C═O, C═S, C═NR³, —C(═O)O—, —C(═O)NR³—, NR³, P(═O)(R³), —O—, —S—, —Se—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more, adjacent substituents R² together may form a with one another;
R³ is the same or different at each instance and is selected from the group consisting of H, D, F, CN, an aliphatic hydrocarbyl radical having 1 to 20 carbon atoms, and an aromatic or heteroaromatic ring system having 5 to 30 aromatic ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents to form a ring system with one another,
p is 0 or 1 and Y represents B(R), C(R)₂, Si(R)₂, C═O, C═NR, C═C(R)₂, O, S, Se, S═O, SO₂, N(R), P(R) and P(═O)R, where, when p=0, there is a bond between the aromatic or heteroaromatic rings shown.

33. An oligomer, polymer or dendrimer containing one or more compounds as claimed in claim 20, wherein, rather than a hydrogen atom or a substituent, the compounds have one or more bonds to the polymer, oligomer or dendrimer.

34. A composition comprising at least one compound as claimed in claim 20 and at least one further compound selected from the group consisting of fluorescent emitters, phosphorescent emitters, emitters that exhibit TADF (thermally activated delayed fluorescence), host materials, electron transport materials, electron injection materials, hole conductor materials, hole injection materials, electron blocker materials and hole blocker materials.

35. A formulation comprising at least one compound as claimed in claim 20 and at least one solvent.

36. A method comprising incorporating the compound as claimed in claim 20 in an electronic device as host material.

37. A process for preparing a compound as claimed in claim 20 wherein, in a coupling reaction, a compound comprising at least one nitrogen-containing heterocyclic group is joined to a compound comprising at least one aromatic or heteroaromatic group.

38. An electronic device comprising at least one compound as claimed in claim 20, wherein the electronic device is selected from the group consisting of organic electroluminescent devices, organic integrated circuits, organic field-effect transistors, organic thin-film transistors, organic light-emitting transistors, organic solar cells, organic optical detectors, organic photoreceptors, organic field-quench devices, light-emitting electrochemical cells and organic laser diodes.
